SEQUENCE LISTING (251 sequences)

SEQ ID NO: 1 - DNA, 720 bp, synthetic construct
  misc_feature 1..720: note = Description of Artificial Sequence: Synthetic clone 1 polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatcgatttctcagcggggtcggaagacactgtacgcagactccgtgaagggccggttcaccatctccagagacaattccaagaacacgctgtatctgcaaatgaacagcctgagagccgaggacacggccgtatattactgtgcgaaaggtctgcgtcgttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatatggcatcccatttgcaaagtggggtcccatcaaggttcagtggcagtggatctgggacagatttcactctcaccatcagcagtctgcaacctgaagattttgcaacttactactgtcaacagccgcgttctacgcctttgacgttcggtcaagggaccaaggtggaaatcaaacgg

SEQ ID NO: 2 - protein, 240 aa, synthetic construct
  REGION 1..240: note = Description of Artificial Sequence: Synthetic clone 1 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSISQRGRKTLYADSVKGRFTISRDNSKNTLYLQMNSLRAEDTAVYYCAKGLRRFDYWGQGTLVTVSSGGGGSGGGGSGGGGSTDIQMTQSPSSLSASVGDRVTITCRASQSISSYLNWYQQKPGKAPKLLIYMASHLQSGVPSRFSGSGSGTDFTLTISSLQPEDFATYYCQQPRSTPLTFGQGTKVEIKR

SEQ ID NO: 3 - protein, 116 aa, synthetic construct
  REGION 1..116: note = Description of Artificial Sequence: Synthetic clone 1 variableheavy chain polypeptide
EVQLLESGGGLVQPGGSLRLSCAASGFTFSSYAMSWVRQAPGKGLEWVSSISQRGRKTLYADSVKGRFTISRDNSKNTLYLQMNSLRAEDTAVYYCAKGLRRFDYWGQGTLVTVSS

SEQ ID NO: 4 - protein, 5 aa, synthetic construct
  REGION 1..5: note = Description of Artificial Sequence: Synthetic clone 1 variableheavy chain CDR1 peptide
SYAMS

SEQ ID NO: 5 - protein, 20 aa, synthetic construct
  REGION 1..20: note = Description of Artificial Sequence: Synthetic clone 1 variableheavy chain CDR2 peptide
SISQRGRKTLYADSVKGRFT

SEQ ID NO: 6 - protein, 7 aa, synthetic construct
  REGION 1..7: note = Description of Artificial Sequence: Synthetic clone 1 variableheavy chain CDR3 peptide
GLRRFDY

SEQ ID NO: 7 - protein, 108 aa, synthetic construct
  REGION 1..108: note = Description of Artificial Sequence: Synthetic clone 1 variablelight chain polypeptide
DIQMTQSPSSLSASVGDRVTITCRASQSISSYLNWYQQKPGKAPKLLIYMASHLQSGVPSRFSGSGSGTDFTLTISSLQPEDFATYYCQQPRSTPLTFGQGTKVEIKR

SEQ ID NO: 8 - protein, 11 aa, synthetic construct
  REGION 1..11: note = Description of Artificial Sequence: Synthetic clone 1 variablelight chain CDR1 peptide
RASQSISSYLN

SEQ ID NO: 9 - protein, 7 aa, synthetic construct
  REGION 1..7: note = Description of Artificial Sequence: Synthetic clone 1 variablelight chain CDR2 peptide
MASHLQS

SEQ ID NO: 10 - protein, 7 aa, synthetic construct
  REGION 1..7: note = Description of Artificial Sequence: Synthetic clone 1 variablelight chain CDR3 peptide
PRSTPLT

SEQ ID NO: 11 - DNA, 720 bp, synthetic construct
  misc_feature 1..720: note = Description of Artificial Sequence: Synthetic clone 2polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattactactactggtaccgcgacatggtacgcagactccgtgaagggccggttcaccatctccagagacaattccaagaacacgctgtatctgcaaatgaacagcctgagagccgaggacacggccgtatattactgtgcgaaaactcttcagcgttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgaggcatccgtgttgcaaagtggggtcccatcaaggttcagtggcagtggatctgggacagatttcactctcaccatcagcagtctgcaacccgaagattttgcaacttactactgtcaacagccgaggtctctgcctatgacgttcggccaagggaccaaggtggaaatcaaacgg

SEQ ID NO: 12 - protein, 240 aa, synthetic construct
  REGION 1..240: note = Description of Artificial Sequence: Synthetic clone 2 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSTITTTGTATWYADSVKGRFTISRDNSKNTLYLQMNSLRAEDTAVYYCAKTLQRFDYWGQGTLVTVSSGGGGSGGGGSGGGGSTDIQMTQSPSSLSASVGDRVTITCRASQSISSYLNWYQQKPGKAPKLLIYEASVLQSGVPSRFSGSGSGTDFTLTISSLQPEDFATYYCQQPRSLPMTFGQGTKVEIKR

SEQ ID NO: 13 - protein, 116 aa, synthetic construct
  REGION 1..116: note = Description of Artificial Sequence: Synthetic clone 2 variableheavy chain polypeptide
EVQLLESGGGLVQPGGSLRLSCAASGFTFSSYAMSWVRQAPGKGLEWVSTITTTGTATWYADSVKGRFTISRDNSKNTLYLQMNSLRAEDTAVYYCAKTLQRFDYWGQGTLVTVSS

SEQ ID NO: 14 - protein, 5 aa, synthetic construct
  REGION 1..5: note = Description of Artificial Sequence: Synthetic clone 2 variableheavy chain CDR1 peptide
SYAMS

SEQ ID NO: 15 - protein, 20 aa, synthetic construct
  REGION 1..20: note = Description of Artificial Sequence: Synthetic clone 2 variableheavy chain CDR2 peptide
TITTTGTATWYADSVKGRFT

SEQ ID NO: 16 - protein, 7 aa, synthetic construct
  REGION 1..7: note = Description of Artificial Sequence: Synthetic clone 2 variableheavy chain CDR3 peptide
TLQRFDY

SEQ ID NO: 17 - protein, 108 aa, synthetic construct
  REGION 1..108: note = Description of Artificial Sequence: Synthetic clone 2 variablelight chain polypeptide
DIQMTQSPSSLSASVGDRVTITCRASQSISSYLNWYQQKPGKAPKLLIYEASVLQSGVPSRFSGSGSGTDFTLTISSLQPEDFATYYCQQPRSLPMTFGQGTKVEIKR

SEQ ID NO: 18 - protein, 11 aa, synthetic construct
  REGION 1..11: note = Description of Artificial Sequence: Synthetic clone 2 variablelight chain CDR1 peptide
RASQSISSYLN

SEQ ID NO: 19 - protein, 7 aa, synthetic construct
  REGION 1..7: note = Description of Artificial Sequence: Synthetic clone 2 variablelight chain CDR2 peptide
EASVLQS

SEQ ID NO: 20 - protein, 7 aa, synthetic construct
  REGION 1..7: note = Description of Artificial Sequence: Synthetic clone 2 variablelight chain CDR3 peptide
PRSLPMT

SEQ ID NO: 21 - DNA, 720 bp, synthetic construct
  misc_feature 1..720: note = Description of Artificial Sequence: Synthetic clone 3polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagggattgggcttagtggtggggggacacagtacgcagactccgtgaagggcaggttcaccatctccagagacaattccaagaacacgctgtatctgcaaatgaacagcctgagagccgaggacacggccgtatattactgtgcgaaaggtactactcggtttgactactggggccagggaaccctggtcaccgtctcgagcggtggaggcggttcaggcggaggtggcagcggcggtggcgggtcgacgaacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccgtttgcaaagtggggtcccatcaaggttcagtggcagtggatctgggacagatttcactctcaccatcagcagtctgcaacctgaagattttgcaacttactactgtcaacagtttttggttcttcctcgtacgttcggccaagggaccaaggtggaaatcaaacgg

SEQ ID NO: 22 - protein, 240 aa, synthetic construct
  REGION 1..240: note = Description of Artificial Sequence: Synthetic clone 3 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSGIGLSGGGTQYADSVKGRFTISRDNSKNTLYLQMNSLRAEDTAVYYCAKGTTRFDYWGQGTLVTVSSGGGGSGGGGSGGGGSTNIQMTQSPSSLSASVGDRVTITCRASQSISSYLNWYQQKPGKAPKLLIYKASRLQSGVPSRFSGSGSGTDFTLTISSLQPEDFATYYCQQFLVLPRTFGQGTKVEIKR

SEQ ID NO: 23 - protein, 116 aa, synthetic construct
  REGION 1..116: note = Description of Artificial Sequence: Synthetic clone 3 variableheavy chain polypeptide
EVQLLESGGGLVQPGGSLRLSCAASGFTFSSYAMSWVRQAPGKGLEWVSGIGLSGGGTQYADSVKGRFTISRDNSKNTLYLQMNSLRAEDTAVYYCAKGTTRFDYWGQGTLVTVSS

SEQ ID NO: 24 - protein, 5 aa, synthetic construct
  REGION 1..5: note = Description of Artificial Sequence: Synthetic clone 3 variableheavy chain CDR1 peptide
SYAMS

SEQ ID NO: 25 - protein, 20 aa, synthetic construct
  REGION 1..20: note = Description of Artificial Sequence: Synthetic clone 3 variableheavy chain CDR2 peptide
GIGLSGGGTQYADSVKGRFT

SEQ ID NO: 26 - protein, 7 aa, synthetic construct
  REGION 1..7: note = Description of Artificial Sequence: Synthetic clone 3 variableheavy chain CDR3 peptide
GTTRFDY

SEQ ID NO: 27 - protein, 108 aa, synthetic construct
  REGION 1..108: note = Description of Artificial Sequence: Synthetic clone 3 variablelight chain polypeptide
NIQMTQSPSSLSASVGDRVTITCRASQSISSYLNWYQQKPGKAPKLLIYKASRLQSGVPSRFSGSGSGTDFTLTISSLQPEDFATYYCQQFLVLPRTFGQGTKVEIKR

SEQ ID NO: 28 - protein, 11 aa, synthetic construct
  REGION 1..11: note = Description of Artificial Sequence: Synthetic clone 3 variablelight chain CDR1 peptide
RASQSISSYLN

SEQ ID NO: 29 - protein, 7 aa, synthetic construct
  REGION 1..7: note = Description of Artificial Sequence: Synthetic clone 3 variablelight chain CDR2 peptide
KASRLQS

SEQ ID NO: 30 - protein, 7 aa, synthetic construct
  REGION 1..7: note = Description of Artificial Sequence: Synthetic clone 3 variablelight chain CDR3 peptide
FLVLPRT

SEQ ID NO: 31 - DNA, 720 bp, synthetic construct
  misc_feature 1..720: note = Description of Artificial Sequence: Synthetic clone 4polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaagtattaatggttatggtgataatacagattacgcagactccgtgaagggcaggttcaccatctccagagacaattccaagaacacgctgtatctgcaaatgaacagcctgagagccgaggacacggccgtatattactgtgcgaaaggttcttctgattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctctttgcaaagtggggtcccatcaaggttcagtggcagtggatctgggacagatttcactctcaccatcagcagtctgcaacctgaagattttgcaacttactactgtcaacaggctggttatgatcctactacgttcggccaagggaccaaggtggaaatcaaacgg

SEQ ID NO: 32 - protein, 240 aa, synthetic construct
  REGION 1..240: note = Description of Artificial Sequence: Synthetic clone 4 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSINGYGDNTDYADSVKGRFTISRDNSKNTLYLQMNSLRAEDTAVYYCAKGSSDFDYWGQGTLVTVSSGGGGSGGGGSGGGGSTDIQMTQSPSSLSASVGDRVTITCRASQSISSYLNWYQQKPGKAPKLLIYGASSLQSGVPSRFSGSGSGTDFTLTISSLQPEDFATYYCQQAGYDPTTFGQGTKVEIKR

SEQ ID NO: 33 - protein, 116 aa, synthetic construct
  REGION 1..116: note = Description of Artificial Sequence: Synthetic clone 4 variableheavy chain polypeptide
EVQLLESGGGLVQPGGSLRLSCAASGFTFSSYAMSWVRQAPGKGLEWVSSINGYGDNTDYADSVKGRFTISRDNSKNTLYLQMNSLRAEDTAVYYCAKGSSDFDYWGQGTLVTVSS

SEQ ID NO: 34 - protein, 5 aa, synthetic construct
  REGION 1..5: note = Description of Artificial Sequence: Synthetic clone 4 variableheavy chain CDR1 peptide
SYAMS

SEQ ID NO: 35 - protein, 20 aa, synthetic construct
  REGION 1..20: note = Description of Artificial Sequence: Synthetic clone 4 variableheavy chain CDR2 peptide
SINGYGDNTDYADSVKGRFT

SEQ ID NO: 36 - protein, 7 aa, synthetic construct
  REGION 1..7: note = Description of Artificial Sequence: Synthetic clone 4 variableheavy chain CDR3 peptide
GSSDFDY

SEQ ID NO: 37 - protein, 109 aa, synthetic construct
  REGION 1..109: note = Description of Artificial Sequence: Synthetic clone 4 variablelight chain polypeptide
TDIQMTQSPSSLSASVGDRVTITCRASQSISSYLNWYQQKPGKAPKLLIYGASSLQSGVPSRFSGSGSGTDFTLTISSLQPEDFATYYCQQAGYDPTTFGQGTKVEIKR

SEQ ID NO: 38 - protein, 11 aa, synthetic construct
  REGION 1..11: note = Description of Artificial Sequence: Synthetic clone 4 variablelight chain CDR1 peptide
RASQSISSYLN

SEQ ID NO: 39 - protein, 7 aa, synthetic construct
  REGION 1..7: note = Description of Artificial Sequence: Synthetic clone 4 variablelight chain CDR2 peptide
GASSLQS

SEQ ID NO: 40 - protein, 7 aa, synthetic construct
  REGION 1..7: note = Description of Artificial Sequence: Synthetic clone 4 variablelight chain CDR3 peptide
AGYDPTT

SEQ ID NO: 41 - DNA, 720 bp, synthetic construct
  misc_feature 1..720: note = Description of Artificial Sequence: Synthetic clone 5polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattgctaatgctggtgctactacaaattacgcagactccgtgaagggcaggttcaccatctccagagacaattccaagaacacgctgtatctgcaaatgaacagcctgagagccgaggacacggccgtatattactgtgcgaaaggtagtagtgattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccaatttgcaaagtggggtcccatcaaggttcagtggcagtggatctgggacagatttcactctcaccatcagcagtctgcaacctgaagattttgcaacttactactgtcaacagagtggttatgatcctagtacgttcggccaagggaccaaggtggaaatcaaacgg

SEQ ID NO: 42 - protein, 240 aa, synthetic construct
  REGION 1..240: note = Description of Artificial Sequence: Synthetic clone 5 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSAIANAGATTNYADSVKGRFTISRDNSKNTLYLQMNSLRAEDTAVYYCAKGSSDFDYWGQGTLVTVSSGGGGSGGGGSGGGGSTDIQMTQSPSSLSASVGDRVTITCRASQSISSYLNWYQQKPGKAPKLLIYGASNLQSGVPSRFSGSGSGTDFTLTISSLQPEDFATYYCQQSGYDPSTFGQGTKVEIKR

SEQ ID NO: 43 - protein, 116 aa, synthetic construct
  REGION 1..116: note = Description of Artificial Sequence: Synthetic clone 5 variableheavy chain polypeptide
EVQLLESGGGLVQPGGSLRLSCAASGFTFSSYAMSWVRQAPGKGLEWVSAIANAGATTNYADSVKGRFTISRDNSKNTLYLQMNSLRAEDTAVYYCAKGSSDFDYWGQGTLVTVSS

SEQ ID NO: 44 - protein, 5 aa, synthetic construct
  REGION 1..5: note = Description of Artificial Sequence: Synthetic clone 5 variableheavy chain CDR1 peptide
SYAMS

SEQ ID NO: 45 - protein, 20 aa, synthetic construct
  REGION 1..20: note = Description of Artificial Sequence: Synthetic clone 5 variableheavy chain CDR2 peptide
AIANAGATTNYADSVKGRFT

SEQ ID NO: 46 - protein, 7 aa, synthetic construct
  REGION 1..7: note = Description of Artificial Sequence: Synthetic clone 5 variableheavy chain CDR3 peptide
GSSDFDY

SEQ ID NO: 47 - protein, 109 aa, synthetic construct
  REGION 1..109: note = Description of Artificial Sequence: Synthetic clone 5 variablelight chain polypeptide
TDIQMTQSPSSLSASVGDRVTITCRASQSISSYLNWYQQKPGKAPKLLIYGASNLQSGVPSRFSGSGSGTDFTLTISSLQPEDFATYYCQQSGYDPSTFGQGTKVEIKR

SEQ ID NO: 48 - protein, 11 aa, synthetic construct
  REGION 1..11: note = Description of Artificial Sequence: Synthetic clone 5 variablelight chain CDR1 peptide
RASQSISSYLN

SEQ ID NO: 49 - protein, 7 aa, synthetic construct
  REGION 1..7: note = Description of Artificial Sequence: Synthetic clone 5 variablelight chain CDR2 peptide
GASNLQS

SEQ ID NO: 50 - protein, 7 aa, synthetic construct
  REGION 1..7: note = Description of Artificial Sequence: Synthetic clone 5 variablelight chain CDR3 peptide
SGYDPST

SEQ ID NO: 51 - DNA, 720 bp, synthetic construct
  misc_feature 1..720: note = Description of Artificial Sequence: Synthetic clone 9polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattcaggggcgtggtcataggacagcgtacgcagactccgtgaagggccggttcaccatctccagagacaattccaagaacacgctgtatctgcaaatgaacagcctgagagccgaggacacggccgtatattactgtgcgaaatcgtcgcttgtttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatccagattgcaaagtggggtcccatcaaggttcagtggcagtggatctgggacagatttcactctcaccatcagcagtctgcaacctgaagattttgcaacttactactgtcaacagagtatgctgaagcctccgacgttcggccaagggaccaaggtggaaatcaaacgg

SEQ ID NO: 52 - protein, 240 aa, synthetic construct
  REGION 1..240: note = Description of Artificial Sequence: Synthetic clone 9 polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSTIQGRGHRTAYADSVKGRFTISRDNSKNTLYLQMNSLRAEDTAVYYCAKSSLVFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQSMLKPPTFGQGTKVEIKR

SEQ ID NO: 53 - protein, 116 aa, synthetic construct
  REGION 1..116: note = Description of Artificial Sequence: Synthetic clone 9 variableheavy chain polypeptide
EVQLLESGGGLVQPGGSLRLSCAASGFTFSSYAMSWVRQAPGKGLEWVSTIQGRGHRTAYADSVKGRFTISRDNSKNTLYLQMNSLRAEDTAVYYCAKSSLVFDYWGQGTLVTVSS

SEQ ID NO: 54 - protein, 5 aa, synthetic construct
  REGION 1..5: note = Description of Artificial Sequence: Synthetic clone 9 variableheavy chain CDR1 peptide
SYAMS

SEQ ID NO: 55 - protein, 20 aa, synthetic construct
  REGION 1..20: note = Description of Artificial Sequence: Synthetic clone 9 variableheavy chain CDR2 peptide
TIQGRGHRTAYADSVKGRFT

SEQ ID NO: 56 - protein, 7 aa, synthetic construct
  REGION 1..7: note = Description of Artificial Sequence: Synthetic clone 9 variableheavy chain CDR3 peptide
SSLVFDY

SEQ ID NO: 57 - protein, 109 aa, synthetic construct
  REGION 1..109: note = Description of Artificial Sequence: Synthetic clone 9 variablelight chain polypeptide
TDIQMTQSPSSLSASVGDRVTITCRASQSISSYLNWYQQKPGKAPKLLIYKASRLQSGVPSRFSGSGSGTDFTLTISSLQPEDFATYYCQQSMLKPPTFGQGTKVEIKR

SEQ ID NO: 58 - protein, 11 aa, synthetic construct
  REGION 1..11: note = Description of Artificial Sequence: Synthetic clone 9 variablelight chain CDR1 peptide
RASQSISSYLN

SEQ ID NO: 59 - protein, 7 aa, synthetic construct
  REGION 1..7: note = Description of Artificial Sequence: Synthetic clone 9 variablelight chain CDR2 peptide
KASRLQS

SEQ ID NO: 60 - protein, 7 aa, synthetic construct
  REGION 1..7: note = Description of Artificial Sequence: Synthetic clone 9 variablelight chain CDR3 peptide
SMLKPPT

SEQ ID NO: 61 - DNA, 720 bp, synthetic construct
  misc_feature 1..720: note = Description of Artificial Sequence: Synthetic clone 11polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagtgctactggtgctactacaggttacgcagactccgtgaagggccggttcaccatctccagagacaattccaagaacacgctgtatctgcaaatgaacagcctgagagccgaggacacggccgtatattactgtgcgaaatctggtactgattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagattttgcaacttactactgtcaacagagtggtaatgatcctactacgttcggccaagggaccaaggtggaaatcaaacgg

SEQ ID NO: 62 - protein, 240 aa, synthetic construct
  REGION 1..240: note = Description of Artificial Sequence: Synthetic clone 11polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSISATGATTGYADSVKGRFTISRDNSKNTLYLQMNSLRAEDTAVYYCAKSGTDFDYWGQGTLVTVSSGGGGSGGGGSGGGGSTDIQMTQSPSSLSASVGDRVTITCRASQSISSYLNWYQQKPGKAPKLLIYGASYLQSGVPSRFSGSGSGTDFTLTISSLQPEDFATYYCQQSGNDPTTFGQGTKVEIKR

SEQ ID NO: 63 - protein, 116 aa, synthetic construct
  REGION 1..116: note = Description of Artificial Sequence: Synthetic clone 11 variableheavy chain polypeptide
EVQLLESGGGLVQPGGSLRLSCAASGFTFSSYAMSWVRQAPGKGLEWVSSISATGATTGYADSVKGRFTISRDNSKNTLYLQMNSLRAEDTAVYYCAKSGTDFDYWGQGTLVTVSS

SEQ ID NO: 64 - protein, 5 aa, synthetic construct
  REGION 1..5: note = Description of Artificial Sequence: Synthetic clone 11 variableheavy chain CDR1 peptide
SYAMS

SEQ ID NO: 65 - protein, 20 aa, synthetic construct
  REGION 1..20: note = Description of Artificial Sequence: Synthetic clone 11 variableheavy chain CDR2 peptide
SISATGATTGYADSVKGRFT

SEQ ID NO: 66 - protein, 7 aa, synthetic construct
  REGION 1..7: note = Description of Artificial Sequence: Synthetic clone 11 variableheavy chain CDR3 peptide
SGTDFDY

SEQ ID NO: 67 - protein, 109 aa, synthetic construct
  REGION 1..109: note = Description of Artificial Sequence: Synthetic clone 11 variablelight chain polypeptide
TDIQMTQSPSSLSASVGDRVTITCRASQSISSYLNWYQQKPGKAPKLLIYGASYLQSGVPSRFSGSGSGTDFTLTISSLQPEDFATYYCQQSGNDPTTFGQGTKVEIKR

SEQ ID NO: 68 - protein, 11 aa, synthetic construct
  REGION 1..11: note = Description of Artificial Sequence: Synthetic clone 11 variablelight chain CDR1 peptide
RASQSISSYLN

SEQ ID NO: 69 - protein, 7 aa, synthetic construct
  REGION 1..7: note = Description of Artificial Sequence: Synthetic clone 11 variablelight chain CDR2 peptide
GASYLQS

SEQ ID NO: 70 - protein, 7 aa, synthetic construct
  REGION 1..7: note = Description of Artificial Sequence: Synthetic clone 11 variablelight chain CDR3 peptide
SGNDPTT

SEQ ID NO: 71 - DNA, 720 bp, synthetic construct
  misc_feature 1..720: note = Description of Artificial Sequence: Synthetic clone 13polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctatttatccgcagggtctgcggacatattacgcagactccgtgaagggccggttcaccatctccagagacaattccaagaacacgctgtatctgcaaatgaacagcctgagagccgaggacacggccgtatattactgtgcgaaaaatcttgctaagtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatccactttgcaaagtggggtcccatcaaggttcagtggcagtggatctgggacagatttcactctcaccatcagcagtctgcaacctgaagattttgcaacttactactgtcaacagcctcgggcgcttcctggtacgttcggccaagggaccaaggtggaaatcaaacgg

SEQ ID NO: 72 - protein, 240 aa, synthetic construct
  REGION 1..240: note = Description of Artificial Sequence: Synthetic clone 13polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSIYPQGLRTYYADSVKGRFTISRDNSKNTLYLQMNSLRAEDTAVYYCAKNLAKFDYWGQGTLVTVSSGGGGSGGGGSGGGGSTDIQMTQSPSSLSASVGDRVTITCRASQSISSYLNWYQQKPGKAPKLLIYRASTLQSGVPSRFSGSGSGTDFTLTISSLQPEDFATYYCQQPRALPGTFGQGTKVEIKR

SEQ ID NO: 73 - protein, 116 aa, synthetic construct
  REGION 1..116: note = Description of Artificial Sequence: Synthetic clone 13 variableheavy chain polypeptide
EVQLLESGGGLVQPGGSLRLSCAASGFTFSSYAMSWVRQAPGKGLEWVSSIYPQGLRTYYADSVKGRFTISRDNSKNTLYLQMNSLRAEDTAVYYCAKNLAKFDYWGQGTLVTVSS

SEQ ID NO: 74 - protein, 5 aa, synthetic construct
  REGION 1..5: note = Description of Artificial Sequence: Synthetic clone 13 variableheavy chain CDR1 peptide
SYAMS

SEQ ID NO: 75 - protein, 20 aa, synthetic construct
  REGION 1..20: note = Description of Artificial Sequence: Synthetic clone 13 variableheavy chain CDR2 peptide
SIYPQGLRTYYADSVKGRFT

SEQ ID NO: 76 - protein, 7 aa, synthetic construct
  REGION 1..7: note = Description of Artificial Sequence: Synthetic clone 13 variableheavy chain CDR3 peptide
NLAKFDY

SEQ ID NO: 77 - protein, 109 aa, synthetic construct
  REGION 1..109: note = Description of Artificial Sequence: Synthetic clone 13 variablelight chain polypeptide
TDIQMTQSPSSLSASVGDRVTITCRASQSISSYLNWYQQKPGKAPKLLIYRASTLQSGVPSRFSGSGSGTDFTLTISSLQPEDFATYYCQQPRALPGTFGQGTKVEIKR

SEQ ID NO: 78 - protein, 11 aa, synthetic construct
  REGION 1..11: note = Description of Artificial Sequence: Synthetic clone 13 variablelight chain CDR1 peptide
RASQSISSYLN

SEQ ID NO: 79 - protein, 7 aa, synthetic construct
  REGION 1..7: note = Description of Artificial Sequence: Synthetic clone 13 variablelight chain CDR2 peptide
RASTLQS

SEQ ID NO: 80 - protein, 7 aa, synthetic construct
  REGION 1..7: note = Description of Artificial Sequence: Synthetic clone 13 variablelight chain CDR3 peptide
PRALPGT

SEQ ID NO: 81 - DNA, 720 bp, synthetic construct
  misc_feature 1..720: note = Description of Artificial Sequence: Synthetic clone 16polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcacggattactggttctggtggtcagacaacttacgcagactccgtgaagggccggttcaccatctccagagacaattccaagaacacgctgtatctgcaaatgaacagcctgagagccgaggacacggccgtatattactgtgcgaaaggtatgcgtcagtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatttatcgtgcatcctctttgcaaagtggggtcccatcaaggttcagtggcagtggatctgggacagatttcactctcaccatcagcagtctgcaacctgaagattttgcaacttactactgtcaacaggcgcgtgtgccgcctactacgttcggccaagggaccaaggtggaaatcaaacgg

SEQ ID NO: 82 - protein, 240 aa, synthetic construct
  REGION 1..240: note = Description of Artificial Sequence: Synthetic clone 16polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSRITGSGGQTTYADSVKGRFTISRDNSKNTLYLQMNSLRAEDTAVYYCAKGMRQFDYWGQGTLVTVSSGGGGSGGGGSGGGGSTDIQMTQSPSSLSASVGDRVTITCRASQSISSYLNWYQQKPGKAPKLLIYRASSLQSGVPSRFSGSGSGTDFTLTISSLQPEDFATYYCQQARVPPTTFGQGTKVEIKR

SEQ ID NO: 83 - protein, 116 aa, synthetic construct
  REGION 1..116: note = Description of Artificial Sequence: Synthetic clone 16 variableheavy chain polypeptide
EVQLLESGGGLVQPGGSLRLSCAASGFTFSSYAMSWVRQAPGKGLEWVSRITGSGGQTTYADSVKGRFTISRDNSKNTLYLQMNSLRAEDTAVYYCAKGMRQFDYWGQGTLVTVSS

SEQ ID NO: 84 - protein, 5 aa, synthetic construct
  REGION 1..5: note = Description of Artificial Sequence: Synthetic clone 16 variableheavy chain CDR1 peptide
SYAMS

SEQ ID NO: 85 - protein, 20 aa, synthetic construct
  REGION 1..20: note = Description of Artificial Sequence: Synthetic clone 16 variableheavy chain CDR2 peptide
RITGSGGQTTYADSVKGRFT

SEQ ID NO: 86 - protein, 7 aa, synthetic construct
  REGION 1..7: note = Description of Artificial Sequence: Synthetic clone 16 variableheavy chain CDR3 peptide
GMRQFDY

SEQ ID NO: 87 - protein, 109 aa, synthetic construct
  REGION 1..109: note = Description of Artificial Sequence: Synthetic clone 16 variablelight chain polypeptide
TDIQMTQSPSSLSASVGDRVTITCRASQSISSYLNWYQQKPGKAPKLLIYRASSLQSGVPSRFSGSGSGTDFTLTISSLQPEDFATYYCQQARVPPTTFGQGTKVEIKR

SEQ ID NO: 88 - protein, 11 aa, synthetic construct
  REGION 1..11: note = Description of Artificial Sequence: Synthetic clone 16 variablelight chain CDR1 peptide
RASQSISSYLN

SEQ ID NO: 89 - protein, 7 aa, synthetic construct
  REGION 1..7: note = Description of Artificial Sequence: Synthetic clone 16 variablelight chain CDR2 peptide
RASSLQS

SEQ ID NO: 90 - protein, 7 aa, synthetic construct
  REGION 1..7: note = Description of Artificial Sequence: Synthetic clone 16 variablelight chain CDR3 peptide
ARVPPTT

SEQ ID NO: 91 - DNA, 720 bp, synthetic construct
  misc_feature 1..720: note = Description of Artificial Sequence: Synthetic clone 17polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatcgattctgggttatggtaggcagacaatgtacgcagactccgtgaagggccggttcaccatctccagagacaattccaagaacacgctgtatctgcaaatgaacagcctgagagccgaggacacggccgtatattactgtgcgaaatggtatcgtgcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctaatacgttcggccaagggaccaaggtggaaatcaaacgg

SEQ ID NO: 92 - protein, 240 aa, synthetic construct
  REGION 1..240: note = Description of Artificial Sequence: Synthetic clone 17polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSILGYGRQTMYADSVKGRFTISRDNSKNTLYLQMNSLRAEDTAVYYCAKWYRAFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 93 - protein, 116 aa, synthetic construct
  REGION 1..116: note = Description of Artificial Sequence: Synthetic clone 17 variableheavy chain polypeptide
EVQLLESGGGLVQPGGSLRLSCAASGFTFSSYAMSWVRQAPGKGLEWVSSILGYGRQTMYADSVKGRFTISRDNSKNTLYLQMNSLRAEDTAVYYCAKWYRAFDYWGQGTLVTVSS

SEQ ID NO: 94 - protein, 5 aa, synthetic construct
  REGION 1..5: note = Description of Artificial Sequence: Synthetic clone 17 variableheavy chain CDR1 peptide
SYAMS

SEQ ID NO: 95 - protein, 20 aa, synthetic construct
  REGION 1..20: note = Description of Artificial Sequence: Synthetic clone 17 variableheavy chain CDR2 peptide
SILGYGRQTMYADSVKGRFT

SEQ ID NO: 96 - protein, 7 aa, synthetic construct
  REGION 1..7: note = Description of Artificial Sequence: Synthetic clone 17 variableheavy chain CDR3 peptide
WYRAFDY

SEQ ID NO: 97 - protein, 109 aa, synthetic construct
  REGION 1..109: note = Description of Artificial Sequence: Synthetic clone 17 variablelight chain polypeptide
TDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 98 - protein, 11 aa, synthetic construct
  REGION 1..11: note = Description of Artificial Sequence: Synthetic clone 17 variablelight chain CDR1 peptide
RASQSISSYLN

SEQ ID NO: 99 - protein, 7 aa, synthetic construct
  REGION 1..7: note = Description of Artificial Sequence: Synthetic clone 17 variablelight chain CDR2 peptide
AASSLQS

SEQ ID NO: 100 - protein, 7 aa, synthetic construct
  REGION 1..7: note = Description of Artificial Sequence: Synthetic clone 17 variablelight chain CDR3 peptide
SYSTPNT

SEQ ID NO: 101 - DNA, 720 bp, synthetic construct
  misc_feature 1..720: note = Description of Artificial Sequence: Synthetic clone 18polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattcataagacgggttcgccgacatattacgcagactccgtgaagggccggttcaccatctccagagacaattccaagaacacgctgtatctgcaaatgaacagcctgagagccgaggacacggccgtatattactgtgcgaagtctttgaagtattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcccatttgcaaagtggggtcccatcaaggttcagtggcagtggatctgggacagatttcactctcaccatcagcagtctgcaacctgaagattttgcaacttactactgtcaacagccgcggagtctgcctgctacgttcggccaagggaccaaggtggaaatcaaacgg

SEQ ID NO: 102 - protein, 240 aa, synthetic construct
  REGION 1..240: note = Description of Artificial Sequence: Synthetic clone 18polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSTIHKTGSPTYYADSVKGRFTISRDNSKNTLYLQMNSLRAEDTAVYYCAKSLKYFDYWGQGTLVTVSSGGGGSGGGGSGGGGSTDIQMTQSPSSLSASVGDRVTITCRASQSISSYLNWYQQKPGKAPKLLIYRASHLQSGVPSRFSGSGSGTDFTLTISSLQPEDFATYYCQQPRSLPATFGQGTKVEIKR

SEQ ID NO: 103 - protein, 116 aa, synthetic construct
  REGION 1..116: note = Description of Artificial Sequence: Synthetic clone 18 variableheavy chain polypeptide
EVQLLESGGGLVQPGGSLRLSCAASGFTFSSYAMSWVRQAPGKGLEWVSTIHKTGSPTYYADSVKGRFTISRDNSKNTLYLQMNSLRAEDTAVYYCAKSLKYFDYWGQGTLVTVSS

SEQ ID NO: 104 - protein, 5 aa, synthetic construct
  REGION 1..5: note = Description of Artificial Sequence: Synthetic clone 18 variableheavy chain CDR1 peptide
SYAMS

SEQ ID NO: 105 - protein, 20 aa, synthetic construct
  REGION 1..20: note = Description of Artificial Sequence: Synthetic clone 18 variableheavy chain CDR2 peptide
TIHKTGSPTYYADSVKGRFT

SEQ ID NO: 106 - protein, 7 aa, synthetic construct
  REGION 1..7: note = Description of Artificial Sequence: Synthetic clone 18 variableheavy chain CDR3 peptide
SLKYFDY

SEQ ID NO: 107 - protein, 109 aa, synthetic construct
  REGION 1..109: note = Description of Artificial Sequence: Synthetic clone 18 variablelight chain polypeptide
TDIQMTQSPSSLSASVGDRVTITCRASQSISSYLNWYQQKPGKAPKLLIYRASHLQSGVPSRFSGSGSGTDFTLTISSLQPEDFATYYCQQPRSLPATFGQGTKVEIKR

SEQ ID NO: 108 - protein, 11 aa, synthetic construct
  REGION 1..11: note = Description of Artificial Sequence: Synthetic clone 18 variablelight chain CDR1 peptide
RASQSISSYLN

SEQ ID NO: 109 - protein, 7 aa, synthetic construct
  REGION 1..7: note = Description of Artificial Sequence: Synthetic clone 18 variablelight chain CDR2 peptide
RASHLQS

SEQ ID NO: 110 - protein, 7 aa, synthetic construct
  REGION 1..7: note = Description of Artificial Sequence: Synthetic clone 18 variablelight chain CDR3 peptide
PRSLPAT

SEQ ID NO: 111 - DNA, 720 bp, synthetic construct
  misc_feature 1..720: note = Description of Artificial Sequence: Synthetic clone 21polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactatttatcgtgcgggtcagaatacaaggtacgcagactccgtgaagggccggttcaccatctccagagacaattccaagaacacgctgtatctgcaaatgaacagcctgagagccgaggacacggccgtatattactgtgcgaaatcgagtcggacttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcctcgttgcaaagtggggtcccatcaaggttcagtggcagtggatctgggacagatttcactctcaccatcagcagtctgcaacctgaagattttgcaacttactactgtcaacaggcgacgctgcagcctcggacgttcggccaagggaccaaggtggaaatcaaacgg

SEQ ID NO: 112 - protein, 240 aa, synthetic construct
  REGION 1..240: note = Description of Artificial Sequence: Synthetic clone 21polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSTIYRAGQNTRYADSVKGRFTISRDNSKNTLYLQMNSLRAEDTAVYYCAKSSRTFDYWGQGTLVTVSSGGGGSGGGGSGGGGSTDIQMTQSPSSLSASVGDRVTITCRASQSISSYLNWYQQKPGKAPKLLIYKASSLQSGVPSRFSGSGSGTDFTLTISSLQPEDFATYYCQQATLQPRTFGQGTKVEIKR

SEQ ID NO: 113 - protein, 116 aa, synthetic construct
  REGION 1..116: note = Description of Artificial Sequence: Synthetic clone 21 variableheavy chain polypeptide
EVQLLESGGGLVQPGGSLRLSCAASGFTFSSYAMSWVRQAPGKGLEWVSTIYRAGQNTRYADSVKGRFTISRDNSKNTLYLQMNSLRAEDTAVYYCAKSSRTFDYWGQGTLVTVSS

SEQ ID NO: 114 - protein, 5 aa, synthetic construct
  REGION 1..5: note = Description of Artificial Sequence: Synthetic clone 21 variableheavy chain CDR1 peptide
SYAMS

SEQ ID NO: 115 - protein, 20 aa, synthetic construct
  REGION 1..20: note = Description of Artificial Sequence: Synthetic clone 21 variableheavy chain CDR2 peptide
TIYRAGQNTRYADSVKGRFT

SEQ ID NO: 116 - protein, 7 aa, synthetic construct
  REGION 1..7: note = Description of Artificial Sequence: Synthetic clone 21 variableheavy chain CDR3 peptide
SSRTFDY

SEQ ID NO: 117 - protein, 109 aa, synthetic construct
  REGION 1..109: note = Description of Artificial Sequence: Synthetic clone 21 variablelight chain polypeptide
TDIQMTQSPSSLSASVGDRVTITCRASQSISSYLNWYQQKPGKAPKLLIYKASSLQSGVPSRFSGSGSGTDFTLTISSLQPEDFATYYCQQATLQPRTFGQGTKVEIKR

SEQ ID NO: 118 - protein, 11 aa, synthetic construct
  REGION 1..11: note = Description of Artificial Sequence: Synthetic clone 21 variablelight chain CDR1 peptide
RASQSISSYLN

SEQ ID NO: 119 - protein, 7 aa, synthetic construct
  REGION 1..7: note = Description of Artificial Sequence: Synthetic clone 21 variablelight chain CDR2 peptide
KASSLQS

SEQ ID NO: 120 - protein, 7 aa, synthetic construct
  REGION 1..7: note = Description of Artificial Sequence: Synthetic clone 21 variablelight chain CDR3 peptide
ATLQPRT

SEQ ID NO: 121 - DNA, 720 bp, synthetic construct
  misc_feature 1..720: note = Description of Artificial Sequence: Synthetic clone 23polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagtgcttctggtacttctacaaattacgcagactccgtgaagggccggttcaccatctccagagacaattccaagaacacgctgtatctgcaaatgaacagcctgagagccgaggacacggccgtatattactgtgcgaaaagtaatagtgattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagattttgcaacttactactgtcaacagtctggttatgatcctgctacgttcggccaagggaccaaggtggaaatcaaacgg

SEQ ID NO: 122 - protein, 240 aa, synthetic construct
  REGION 1..240: note = Description of Artificial Sequence: Synthetic clone 23polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSSISASGTSTNYADSVKGRFTISRDNSKNTLYLQMNSLRAEDTAVYYCAKSNSDFDYWGQGTLVTVSSGGGGSGGGGSGGGGSTDIQMTQSPSSLSASVGDRVTITCRASQSISSYLNWYQQKPGKAPKLLIYGASYLQSGVPSRFSGSGSGTDFTLTISSLQPEDFATYYCQQSGYDPATFGQGTKVEIKR

SEQ ID NO: 123 - protein, 116 aa, synthetic construct
  REGION 1..116: note = Description of Artificial Sequence: Synthetic clone 23 variableheavy chain polypeptide
EVQLLESGGGLVQPGGSLRLSCAASGFTFSSYAMSWVRQAPGKGLEWVSSISASGTSTNYADSVKGRFTISRDNSKNTLYLQMNSLRAEDTAVYYCAKSNSDFDYWGQGTLVTVSS

SEQ ID NO: 124 - protein, 5 aa, synthetic construct
  REGION 1..5: note = Description of Artificial Sequence: Synthetic clone 23 variableheavy chain CDR1 peptide
SYAMS

SEQ ID NO: 125 - protein, 20 aa, synthetic construct
  REGION 1..20: note = Description of Artificial Sequence: Synthetic clone 23 variableheavy chain CDR2 peptide
SISASGTSTNYADSVKGRFT

SEQ ID NO: 126 - protein, 7 aa, synthetic construct
  REGION 1..7: note = Description of Artificial Sequence: Synthetic clone 23 variableheavy chain CDR3 peptide
SNSDFDY

SEQ ID NO: 127 - protein, 109 aa, synthetic construct
  REGION 1..109: note = Description of Artificial Sequence: Synthetic clone 23 variablelight chain polypeptide
TDIQMTQSPSSLSASVGDRVTITCRASQSISSYLNWYQQKPGKAPKLLIYGASYLQSGVPSRFSGSGSGTDFTLTISSLQPEDFATYYCQQSGYDPATFGQGTKVEIKR

SEQ ID NO: 128 - protein, 11 aa, synthetic construct
  REGION 1..11: note = Description of Artificial Sequence: Synthetic clone 23 variablelight chain CDR1 peptide
RASQSISSYLN

SEQ ID NO: 129 - protein, 7 aa, synthetic construct
  REGION 1..7: note = Description of Artificial Sequence: Synthetic clone 23 variablelight chain CDR2 peptide
GASYLQS

SEQ ID NO: 130 - protein, 7 aa, synthetic construct
  REGION 1..7: note = Description of Artificial Sequence: Synthetic clone 23 variablelight chain CDR3 peptide
SGYDPAT

SEQ ID NO: 131 - DNA, 720 bp, synthetic construct
  misc_feature 1..720: note = Description of Artificial Sequence: Synthetic clone 24polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattgataatagtggtgcttatacatcttacgcagactccgtgaagggcaggttcaccatctccagagacaattccaagaacacgctgtatctgcaaatgaacagcctgagagccgaggacacggccgtatattactgtgcgaaaggtagttctgattttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctctttgcaaagtggggtcccatcaaggttcagtggcagtggatctgggacagatttcactctcaccatcagcagtctgcaacctgaagattttgcaacttactactgtcaacaggctggtaatgatccttctacgttcggccaagggaccaaggtggaaatcaaacgg

SEQ ID NO: 132 - protein, 240 aa, synthetic construct
  REGION 1..240: note = Description of Artificial Sequence: Synthetic clone 24polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 100..105: note = misc_feature - Variable heavy chain CDR3
  REGION 132..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSAIDNSGAYTSYADSVKGRFTISRDNSKNTLYLQMNSLRAEDTAVYYCAKGSSDFDYWGQGTLVTVSSGGGGSGGGGSGGGGSTDIQMTQSPSSLSASVGDRVTITCRASQSISSYLNWYQQKPGKAPKLLIYGASSLQSGVPSRFSGSGSGTDFTLTISSLQPEDFATYYCQQAGNDPSTFGQGTKVEIKR

SEQ ID NO: 133 - protein, 116 aa, synthetic construct
  REGION 1..116: note = Description of Artificial Sequence: Synthetic clone 24 variableheavy chain polypeptide
EVQLLESGGGLVQPGGSLRLSCAASGFTFSSYAMSWVRQAPGKGLEWVSAIDNSGAYTSYADSVKGRFTISRDNSKNTLYLQMNSLRAEDTAVYYCAKGSSDFDYWGQGTLVTVSS

SEQ ID NO: 134 - protein, 5 aa, synthetic construct
  REGION 1..5: note = Description of Artificial Sequence: Synthetic clone 24 variableheavy chain CDR1 peptide
SYAMS

SEQ ID NO: 135 - protein, 20 aa, synthetic construct
  REGION 1..20: note = Description of Artificial Sequence: Synthetic clone 24 variableheavy chain CDR2 peptide
AIDNSGAYTSYADSVKGRFT

SEQ ID NO: 136 - protein, 6 aa, synthetic construct
  REGION 1..6: note = Description of Artificial Sequence: Synthetic clone 24 variableheavy chain CDR3 peptide
SSDFDY

SEQ ID NO: 137 - protein, 109 aa, synthetic construct
  REGION 1..109: note = Description of Artificial Sequence: Synthetic clone 24 variablelight chain polypeptide
TDIQMTQSPSSLSASVGDRVTITCRASQSISSYLNWYQQKPGKAPKLLIYGASSLQSGVPSRFSGSGSGTDFTLTISSLQPEDFATYYCQQAGNDPSTFGQGTKVEIKR

SEQ ID NO: 138 - protein, 11 aa, synthetic construct
  REGION 1..11: note = Description of Artificial Sequence: Synthetic clone 24 variablelight chain CDR1 peptide
RASQSISSYLN

SEQ ID NO: 139 - protein, 7 aa, synthetic construct
  REGION 1..7: note = Description of Artificial Sequence: Synthetic clone 24 variablelight chain CDR2 peptide
GASSLQS

SEQ ID NO: 140 - protein, 7 aa, synthetic construct
  REGION 1..7: note = Description of Artificial Sequence: Synthetic clone 24 variablelight chain CDR3 peptide
AGNDPST

SEQ ID NO: 141 - DNA, 720 bp, synthetic construct
  misc_feature 1..720: note = Description of Artificial Sequence: Synthetic clone 26polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcgattactgcgccgggtcctcggacacagtacgcagactccgtgaagggccggttcaccatctccagagacaattccaagaacacgctgtatctgcaaatgaacagcctgagagccgaggacacggccgtatattactgtgcgaaagggacgcgtacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcccatttgcaaagtggggtcccatcaaggttcagtggcagtggatctgggacagatttcactctcaccatcagcagtctgcaacctgaagattttgcaacttactactgtcaacagccgacgactacgcctcgtacgttcggccaagggaccaaggtggaaatcaaacgg

SEQ ID NO: 142 - protein, 240 aa, synthetic construct
  REGION 1..240: note = Description of Artificial Sequence: Synthetic clone 26polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..240: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSAITAPGPRTQYADSVKGRFTISRDNSKNTLYLQMNSLRAEDTAVYYCAKGTRTFDYWGQGTLVTVSSGGGGSGGGGSGGGGSTDIQMTQSPSSLSASVGDRVTITCRASQSISSYLNWYQQKPGKAPKLLIYGASHLQSGVPSRFSGSGSGTDFTLTISSLQPEDFATYYCQQPTTTPRTFGQGTKVEIKR

SEQ ID NO: 143 - protein, 116 aa, synthetic construct
  REGION 1..116: note = Description of Artificial Sequence: Synthetic clone 26 variableheavy chain polypeptide
EVQLLESGGGLVQPGGSLRLSCAASGFTFSSYAMSWVRQAPGKGLEWVSAITAPGPRTQYADSVKGRFTISRDNSKNTLYLQMNSLRAEDTAVYYCAKGTRTFDYWGQGTLVTVSS

SEQ ID NO: 144 - protein, 5 aa, synthetic construct
  REGION 1..5: note = Description of Artificial Sequence: Synthetic clone 26 variableheavy chain CDR1 peptide
SYAMS

SEQ ID NO: 145 - protein, 20 aa, synthetic construct
  REGION 1..20: note = Description of Artificial Sequence: Synthetic clone 26 variableheavy chain CDR2 peptide
AITAPGPRTQYADSVKGRFT

SEQ ID NO: 146 - protein, 7 aa, synthetic construct
  REGION 1..7: note = Description of Artificial Sequence: Synthetic clone 26 variableheavy chain CDR3 peptide
GTRTFDY

SEQ ID NO: 147 - protein, 108 aa, synthetic construct
  REGION 1..108: note = Description of Artificial Sequence: Synthetic clone 26 variablelight chain polypeptide
DIQMTQSPSSLSASVGDRVTITCRASQSISSYLNWYQQKPGKAPKLLIYGASHLQSGVPSRFSGSGSGTDFTLTISSLQPEDFATYYCQQPTTTPRTFGQGTKVEIKR

SEQ ID NO: 148 - protein, 11 aa, synthetic construct
  REGION 1..11: note = Description of Artificial Sequence: Synthetic clone 26 variablelight chain CDR1 peptide
RASQSISSYLN

SEQ ID NO: 149 - protein, 7 aa, synthetic construct
  REGION 1..7: note = Description of Artificial Sequence: Synthetic clone 26 variablelight chain CDR2 peptide
GASHLQS

SEQ ID NO: 150 - protein, 7 aa, synthetic construct
  REGION 1..7: note = Description of Artificial Sequence: Synthetic clone 26 variablelight chain CDR3 peptide
PTTTPRT

SEQ ID NO: 151 - DNA, 723 bp, synthetic construct
  misc_feature 1..723: note = Description of Artificial Sequence: Synthetic clone 27polynucleotide
  source 1..723: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaaagattaagccgcagggtgatgcgacacggtacgcagactccgtgaagggccggttcaccatctccagagacaattccaagaacacgctgtatctgcaaatgaacagcctgagagccgaggacacggccgtatattactgtgcgaaaggggctcggaggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcccgtttgcaaagtggggtcccatcaaggttcagtggcagtggatctgggacagatttcactctcaccatcagcagtctgcaacctgaagattttgcaacttactactgtcaacagactaagcggcctcctcagacgttcggccaagggaccaaggtggaaatcaaacgggcg

SEQ ID NO: 152 - protein, 241 aa, synthetic construct
  REGION 1..241: note = Description of Artificial Sequence: Synthetic clone 27polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..241: note = misc_feature - Variable light chain
  REGION 156..166: note = misc_feature - Variable light chain CDR1
  REGION 182..188: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSKIKPQGDATRYADSVKGRFTISRDNSKNTLYLQMNSLRAEDTAVYYCAKGARRFDYWGQGTLVTVSSGGGGSGGGGSGGGGSTDIQMTQSPSSLSASVGDRVTITCRASQSISSYLNWYQQKPGKAPKLLIYRASRLQSGVPSRFSGSGSGTDFTLTISSLQPEDFATYYCQQTKRPPQTFGQGTKVEIKRA

SEQ ID NO: 153 - protein, 116 aa, synthetic construct
  REGION 1..116: note = Description of Artificial Sequence: Synthetic clone 27 variableheavy chain polypeptide
EVQLLESGGGLVQPGGSLRLSCAASGFTFSSYAMSWVRQAPGKGLEWVSKIKPQGDATRYADSVKGRFTISRDNSKNTLYLQMNSLRAEDTAVYYCAKGARRFDYWGQGTLVTVSS

SEQ ID NO: 154 - protein, 5 aa, synthetic construct
  REGION 1..5: note = Description of Artificial Sequence: Synthetic clone 27 variableheavy chain CDR1 peptide
SYAMS

SEQ ID NO: 155 - protein, 20 aa, synthetic construct
  REGION 1..20: note = Description of Artificial Sequence: Synthetic clone 27 variableheavy chain CDR2 peptide
KIKPQGDATRYADSVKGRFT

SEQ ID NO: 156 - protein, 7 aa, synthetic construct
  REGION 1..7: note = Description of Artificial Sequence: Synthetic clone 27 variableheavy chain CDR3 peptide
GARRFDY

SEQ ID NO: 157 - protein, 109 aa, synthetic construct
  REGION 1..109: note = Description of Artificial Sequence: Synthetic clone 27 variablelight chain polypeptide
DIQMTQSPSSLSASVGDRVTITCRASQSISSYLNWYQQKPGKAPKLLIYRASRLQSGVPSRFSGSGSGTDFTLTISSLQPEDFATYYCQQTKRPPQTFGQGTKVEIKRA

SEQ ID NO: 158 - protein, 11 aa, synthetic construct
  REGION 1..11: note = Description of Artificial Sequence: Synthetic clone 27 variablelight chain CDR1 peptide
RASQSISSYLN

SEQ ID NO: 159 - protein, 7 aa, synthetic construct
  REGION 1..7: note = Description of Artificial Sequence: Synthetic clone 27 variablelight chain CDR2 peptide
RASRLQS

SEQ ID NO: 160 - protein, 7 aa, synthetic construct
  REGION 1..7: note = Description of Artificial Sequence: Synthetic clone 27 variablelight chain CDR3 peptide
TKRPPQT

SEQ ID NO: 161 - DNA, 720 bp, synthetic construct
  misc_feature 1..720: note = Description of Artificial Sequence: Synthetic clone 41polynucleotide
  source 1..720: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattaagcagacgggtacgcagacaaagtacgcagactccgtgaagggccggttcaccatctccagagacaattccaagaacacgctgtatctgcaaatgaacagcctgagagccgaggacacggccgtatattactgtgcgaaaagtggtcggcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccgtttgcaaagtggggtcccatcaaggttcagtggcagtggatctgggacagatttcactctcaccatcagcagtctgcaacctgaagattttgcaacttactactgtcaacaggctagtcttacgcctccgacgttcggccaagggaccaaggtggaaatcaaacgg

SEQ ID NO: 162 - protein, 240 aa, synthetic construct
  REGION 1..240: note = Description of Artificial Sequence: Synthetic clone 41polypeptide
  REGION 1..116: note = misc_feature - Variable heavy chain
  REGION 31..35: note = misc_feature - Variable heavy chain CDR1
  REGION 50..69: note = misc_feature - Variable heavy chain CDR2
  REGION 99..105: note = misc_feature - Variable heavy chain CDR3
  REGION 133..240: note = misc_feature - Variable light chain
  REGION 156..166: note = misc_feature - Variable light chain CDR1
  REGION 182..187: note = misc_feature - Variable light chain CDR2
  REGION 223..229: note = misc_feature - Variable light chain CDR3
EVQLLESGGGLVQPGGSLRLSCAASGFTFSSYAMSWVRQAPGKGLEWVSTIKQTGTQTKYADSVKGRFTISRDNSKNTLYLQMNSLRAEDTAVYYCAKSGRRFDYWGQGTLVTVSSGGGGSGGGGSGGGGSTDIQMTQSPSSLSASVGDRVTITCRASQSISSYLNWYQQKPGKAPKLLIYKASRLQSGVPSRFSGSGSGTDFTLTISSLQPEDFATYYCQQASLTPPTFGQGTKVEIKR

SEQ ID NO: 163 - protein, 116 aa, synthetic construct
  REGION 1..116: note = Description of Artificial Sequence: Synthetic clone 41 variableheavy chain polypeptide
EVQLLESGGGLVQPGGSLRLSCAASGFTFSSYAMSWVRQAPGKGLEWVSTIKQTGTQTKYADSVKGRFTISRDNSKNTLYLQMNSLRAEDTAVYYCAKSGRRFDYWGQGTLVTVSS

SEQ ID NO: 164 - protein, 5 aa, synthetic construct
  REGION 1..5: note = Description of Artificial Sequence: Synthetic clone 41 variableheavy chain CDR1 peptide
SYAMS

SEQ ID NO: 165 - protein, 20 aa, synthetic construct
  REGION 1..20: note = Description of Artificial Sequence: Synthetic clone 41 variableheavy chain CDR2 peptide
TIKQTGTQTKYADSVKGRFT

SEQ ID NO: 166 - protein, 7 aa, synthetic construct
  REGION 1..7: note = Description of Artificial Sequence: Synthetic clone 41 variableheavy chain CDR3 peptide
SGRRFDY

SEQ ID NO: 167 - protein, 108 aa, synthetic construct
  REGION 1..108: note = Description of Artificial Sequence: Synthetic clone 41 variablelight chain polypeptide
DIQMTQSPSSLSASVGDRVTITCRASQSISSYLNWYQQKPGKAPKLLIYKASRLQSGVPSRFSGSGSGTDFTLTISSLQPEDFATYYCQQASLTPPTFGQGTKVEIKR

SEQ ID NO: 168 - protein, 11 aa, synthetic construct
  REGION 1..11: note = Description of Artificial Sequence: Synthetic clone 41 variablelight chain CDR1 peptide
RASQSISSYLN

SEQ ID NO: 169 - protein, 6 aa, synthetic construct
  REGION 1..6: note = Description of Artificial Sequence: Synthetic clone 41 variablelight chain CDR2 peptide
KASRLQ

SEQ ID NO: 170 - protein, 7 aa, synthetic construct
  REGION 1..7: note = Description of Artificial Sequence: Synthetic clone 41 variablelight chain CDR3 peptide
ASLTPPT

SEQ ID NO: 171 - DNA, 990 bp, Homo sapiens
  misc_feature 1..990: note = human IgG1
  source 1..990: mol_type = other DNA
gctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 172 - protein, 330 aa, Homo sapiens [sequence omitted]

SEQ ID NO: 173 - DNA, 318 bp, Homo sapiens
  misc_feature 1..318: note = human CK
  source 1..318: mol_type = other DNA
accgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 174 - protein, 106 aa, Homo sapiens
  REGION 1..106: note = MISC_FEATURE - human CK
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - DNA, 1338 bp, synthetic construct
  misc_feature 1..1338: note = Description of Artificial Sequence: Synthetic clone 1 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatcgatttctcagcggggtcggaagacactgtacgcagactccgtgaagggccggttcaccatctccagagacaattccaagaacacgctgtatctgcaaatgaacagcctgagagccgaggacacggccgtatattactgtgcgaaaggtctgcgtcgttttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 176 - protein, 446 aa, synthetic construct
  REGION 1..446: note = Description of Artificial Sequence: Synthetic clone 1 VH-CH123polypeptide
EVQLLESGGGLVQPGGSLRLSCAASGFTFSSYAMSWVRQAPGKGLEWVSSISQRGRKTLYADSVKGRFTISRDNSKNTLYLQMNSLRAEDTAVYYCAKGLRRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - DNA, 642 bp, synthetic construct
  misc_feature 1..642: note = Description of Artificial Sequence: Synthetic clone 1 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatatggcatcccatttgcaaagtggggtcccatcaaggttcagtggcagtggatctgggacagatttcactctcaccatcagcagtctgcaacctgaagattttgcaacttactactgtcaacagccgcgttctacgcctttgacgttcggtcaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 178 - protein, 214 aa, synthetic construct
  REGION 1..214: note = Description of Artificial Sequence: Synthetic clone 1 VL-CKpolypeptide
DIQMTQSPSSLSASVGDRVTITCRASQSISSYLNWYQQKPGKAPKLLIYMASHLQSGVPSRFSGSGSGTDFTLTISSLQPEDFATYYCQQPRSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - DNA, 1338 bp, synthetic construct
  misc_feature 1..1338: note = Description of Artificial Sequence: Synthetic clone 2 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattactactactggtaccgcgacatggtacgcagactccgtgaagggccggttcaccatctccagagacaattccaagaacacgctgtatctgcaaatgaacagcctgagagccgaggacacggccgtatattactgtgcgaaaactcttcagcgttttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 180 - protein, 446 aa, synthetic construct
  REGION 1..446: note = Description of Artificial Sequence: Synthetic clone 2 VH-CH123polypeptide
EVQLLESGGGLVQPGGSLRLSCAASGFTFSSYAMSWVRQAPGKGLEWVSTITTTGTATWYADSVKGRFTISRDNSKNTLYLQMNSLRAEDTAVYYCAKTLQRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 642 bp, synthetic construct
  misc_feature 1..642: note = Description of Artificial Sequence: Synthetic clone 2 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgaggcatccgtgttgcaaagtggggtcccatcaaggttcagtggcagtggatctgggacagatttcactctcaccatcagcagtctgcaacccgaagattttgcaacttactactgtcaacagccgaggtctctgcctatgacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 182 - protein, 214 aa, synthetic construct
  REGION 1..214: note = Description of Artificial Sequence: Synthetic clone 2 VL-CKpolypeptide
DIQMTQSPSSLSASVGDRVTITCRASQSISSYLNWYQQKPGKAPKLLIYEASVLQSGVPSRFSGSGSGTDFTLTISSLQPEDFATYYCQQPRSLPMTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 1338 bp, synthetic construct
  misc_feature 1..1338: note = Description of Artificial Sequence: Synthetic clone 3 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagggattgggcttagtggtggggggacacagtacgcagactccgtgaagggcaggttcaccatctccagagacaattccaagaacacgctgtatctgcaaatgaacagcctgagagccgaggacacggccgtatattactgtgcgaaaggtactactcggtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 184 - protein, 446 aa, synthetic construct
  REGION 1..446: note = Description of Artificial Sequence: Synthetic clone 3 VH-CH123polypeptide
EVQLLESGGGLVQPGGSLRLSCAASGFTFSSYAMSWVRQAPGKGLEWVSGIGLSGGGTQYADSVKGRFTISRDNSKNTLYLQMNSLRAEDTAVYYCAKGTTRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - DNA, 642 bp, synthetic construct
  misc_feature 1..642: note = Description of Artificial Sequence: Synthetic clone 3 VL-CKpolynucleotide
  source 1..642: mol_type = other DNA
aacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccgtttgcaaagtggggtcccatcaaggttcagtggcagtggatctgggacagatttcactctcaccatcagcagtctgcaacctgaagattttgcaacttactactgtcaacagtttttggttcttcctcgtacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 186 - protein, 214 aa, synthetic construct
  REGION 1..214: note = Description of Artificial Sequence: Synthetic clone 3 VL-CKpolypeptide
NIQMTQSPSSLSASVGDRVTITCRASQSISSYLNWYQQKPGKAPKLLIYKASRLQSGVPSRFSGSGSGTDFTLTISSLQPEDFATYYCQQFLVLPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - DNA, 1338 bp, synthetic construct
  misc_feature 1..1338: note = Description of Artificial Sequence: Synthetic clone 4 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaagtattaatggttatggtgataatacagattacgcagactccgtgaagggcaggttcaccatctccagagacaattccaagaacacgctgtatctgcaaatgaacagcctgagagccgaggacacggccgtatattactgtgcgaaaggttcttctgattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 188 - protein, 446 aa, synthetic construct
  REGION 1..446: note = Description of Artificial Sequence: Synthetic clone 4 VH-CH123polypeptide
EVQLLESGGGLVQPGGSLRLSCAASGFTFSSYAMSWVRQAPGKGLEWVSSINGYGDNTDYADSVKGRFTISRDNSKNTLYLQMNSLRAEDTAVYYCAKGSSDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - DNA, 642 bp, synthetic construct
  misc_feature 1..642: note = Description of Artificial Sequence: Synthetic clone 4 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctctttgcaaagtggggtcccatcaaggttcagtggcagtggatctgggacagatttcactctcaccatcagcagtctgcaacctgaagattttgcaacttactactgtcaacaggctggttatgatcctactacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 190 - protein, 214 aa, synthetic construct
  REGION 1..214: note = Description of Artificial Sequence: Synthetic clone 4 VL-CKpolypeptide
DIQMTQSPSSLSASVGDRVTITCRASQSISSYLNWYQQKPGKAPKLLIYGASSLQSGVPSRFSGSGSGTDFTLTISSLQPEDFATYYCQQAGYDPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 191 - DNA, 1338 bp, synthetic construct
  misc_feature 1..1338: note = Description of Artificial Sequence: Synthetic clone 5 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattgctaatgctggtgctactacaaattacgcagactccgtgaagggcaggttcaccatctccagagacaattccaagaacacgctgtatctgcaaatgaacagcctgagagccgaggacacggccgtatattactgtgcgaaaggtagtagtgattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 192 - protein, 446 aa, synthetic construct
  REGION 1..446: note = Description of Artificial Sequence: Synthetic clone 5 VH-CH123polypeptide
EVQLLESGGGLVQPGGSLRLSCAASGFTFSSYAMSWVRQAPGKGLEWVSAIANAGATTNYADSVKGRFTISRDNSKNTLYLQMNSLRAEDTAVYYCAKGSSDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - DNA, 642 bp, synthetic construct
  misc_feature 1..642: note = Description of Artificial Sequence: Synthetic clone 5 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccaatttgcaaagtggggtcccatcaaggttcagtggcagtggatctgggacagatttcactctcaccatcagcagtctgcaacctgaagattttgcaacttactactgtcaacagagtggttatgatcctagtacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 194 - protein, 214 aa, synthetic construct
  REGION 1..214: note = Description of Artificial Sequence: Synthetic clone 5 VL-CKpolypeptide
DIQMTQSPSSLSASVGDRVTITCRASQSISSYLNWYQQKPGKAPKLLIYGASNLQSGVPSRFSGSGSGTDFTLTISSLQPEDFATYYCQQSGYDPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 195 - DNA, 1338 bp, synthetic construct
  misc_feature 1..1338: note = Description of Artificial Sequence: Synthetic clone 9 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattcaggggcgtggtcataggacagcgtacgcagactccgtgaagggccggttcaccatctccagagacaattccaagaacacgctgtatctgcaaatgaacagcctgagagccgaggacacggccgtatattactgtgcgaaatcgtcgcttgtttttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 196 - protein, 446 aa, synthetic construct
  REGION 1..446: note = Description of Artificial Sequence: Synthetic clone 9 VH-CH123polypeptide
EVQLLESGGGLVQPGGSLRLSCAASGFTFSSYAMSWVRQAPGKGLEWVSTIQGRGHRTAYADSVKGRFTISRDNSKNTLYLQMNSLRAEDTAVYYCAKSSLVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - DNA, 642 bp, synthetic construct
  misc_feature 1..642: note = Description of Artificial Sequence: Synthetic clone 9 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatccagattgcaaagtggggtcccatcaaggttcagtggcagtggatctgggacagatttcactctcaccatcagcagtctgcaacctgaagattttgcaacttactactgtcaacagagtatgctgaagcctccgacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 198 - protein, 214 aa, synthetic construct
  REGION 1..214: note = Description of Artificial Sequence: Synthetic clone 9 VL-CKpolypeptide
DIQMTQSPSSLSASVGDRVTITCRASQSISSYLNWYQQKPGKAPKLLIYKASRLQSGVPSRFSGSGSGTDFTLTISSLQPEDFATYYCQQSMLKPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 199 - DNA, 1338 bp, synthetic construct
  misc_feature 1..1338: note = Description of Artificial Sequence: Synthetic clone 11 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagtgctactggtgctactacaggttacgcagactccgtgaagggccggttcaccatctccagagacaattccaagaacacgctgtatctgcaaatgaacagcctgagagccgaggacacggccgtatattactgtgcgaaatctggtactgattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 200 - protein, 446 aa, synthetic construct
  REGION 1..446: note = Description of Artificial Sequence: Synthetic clone 11 VH-CH123polypeptide
EVQLLESGGGLVQPGGSLRLSCAASGFTFSSYAMSWVRQAPGKGLEWVSSISATGATTGYADSVKGRFTISRDNSKNTLYLQMNSLRAEDTAVYYCAKSGTDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - DNA, 642 bp, synthetic construct
  misc_feature 1..642: note = Description of Artificial Sequence: Synthetic clone 11 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagattttgcaacttactactgtcaacagagtggtaatgatcctactacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 202 - protein, 214 aa, synthetic construct
  REGION 1..214: note = Description of Artificial Sequence: Synthetic clone 11 VL-CKpolypeptide
DIQMTQSPSSLSASVGDRVTITCRASQSISSYLNWYQQKPGKAPKLLIYGASYLQSGVPSRFSGSGSGTDFTLTISSLQPEDFATYYCQQSGNDPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 1338 bp, synthetic construct
  misc_feature 1..1338: note = Description of Artificial Sequence: Synthetic clone 13 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctatttatccgcagggtctgcggacatattacgcagactccgtgaagggccggttcaccatctccagagacaattccaagaacacgctgtatctgcaaatgaacagcctgagagccgaggacacggccgtatattactgtgcgaaaaatcttgctaagtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 204 - protein, 446 aa, synthetic construct
  REGION 1..446: note = Description of Artificial Sequence: Synthetic clone 13 VH-CH123polypeptide
EVQLLESGGGLVQPGGSLRLSCAASGFTFSSYAMSWVRQAPGKGLEWVSSIYPQGLRTYYADSVKGRFTISRDNSKNTLYLQMNSLRAEDTAVYYCAKNLAKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - DNA, 642 bp, synthetic construct
  misc_feature 1..642: note = Description of Artificial Sequence: Synthetic clone 13 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatccactttgcaaagtggggtcccatcaaggttcagtggcagtggatctgggacagatttcactctcaccatcagcagtctgcaacctgaagattttgcaacttactactgtcaacagcctcgggcgcttcctggtacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 206 - protein, 214 aa, synthetic construct
  REGION 1..214: note = Description of Artificial Sequence: Synthetic clone 13 VL-CKpolypeptide
DIQMTQSPSSLSASVGDRVTITCRASQSISSYLNWYQQKPGKAPKLLIYRASTLQSGVPSRFSGSGSGTDFTLTISSLQPEDFATYYCQQPRALPGTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 207 - DNA, 1338 bp, synthetic construct
  misc_feature 1..1338: note = Description of Artificial Sequence: Synthetic clone 16 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcacggattactggttctggtggtcagacaacttacgcagactccgtgaagggccggttcaccatctccagagacaattccaagaacacgctgtatctgcaaatgaacagcctgagagccgaggacacggccgtatattactgtgcgaaaggtatgcgtcagtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 208 - protein, 446 aa, synthetic construct
  REGION 1..446: note = Description of Artificial Sequence: Synthetic clone 16 VH-CH123polypeptide
EVQLLESGGGLVQPGGSLRLSCAASGFTFSSYAMSWVRQAPGKGLEWVSRITGSGGQTTYADSVKGRFTISRDNSKNTLYLQMNSLRAEDTAVYYCAKGMRQFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - DNA, 642 bp, synthetic construct
  misc_feature 1..642: note = Description of Artificial Sequence: Synthetic clone 16 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatttatcgtgcatcctctttgcaaagtggggtcccatcaaggttcagtggcagtggatctgggacagatttcactctcaccatcagcagtctgcaacctgaagattttgcaacttactactgtcaacaggcgcgtgtgccgcctactacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 210 - protein, 214 aa, synthetic construct
  REGION 1..214: note = Description of Artificial Sequence: Synthetic clone 16 VL-CKpolypeptide
DIQMTQSPSSLSASVGDRVTITCRASQSISSYLNWYQQKPGKAPKLLIYRASSLQSGVPSRFSGSGSGTDFTLTISSLQPEDFATYYCQQARVPPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 211 - DNA, 1338 bp, synthetic construct
  misc_feature 1..1338: note = Description of Artificial Sequence: Synthetic clone 17 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatcgattctgggttatggtaggcagacaatgtacgcagactccgtgaagggccggttcaccatctccagagacaattccaagaacacgctgtatctgcaaatgaacagcctgagagccgaggacacggccgtatattactgtgcgaaatggtatcgtgcttttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 212 - protein, 446 aa, synthetic construct
  REGION 1..446: note = Description of Artificial Sequence: Synthetic clone 17 VH-CH123polypeptide
EVQLLESGGGLVQPGGSLRLSCAASGFTFSSYAMSWVRQAPGKGLEWVSSILGYGRQTMYADSVKGRFTISRDNSKNTLYLQMNSLRAEDTAVYYCAKWYRAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 642 bp, synthetic construct
  misc_feature 1..642: note = Description of Artificial Sequence: Synthetic clone 17 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctaatacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 214 - protein, 214 aa, synthetic construct
  REGION 1..214: note = Description of Artificial Sequence: Synthetic clone 17 VL-CKpolypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - DNA, 1338 bp, synthetic construct
  misc_feature 1..1338: note = Description of Artificial Sequence: Synthetic clone 18 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattcataagacgggttcgccgacatattacgcagactccgtgaagggccggttcaccatctccagagacaattccaagaacacgctgtatctgcaaatgaacagcctgagagccgaggacacggccgtatattactgtgcgaagtctttgaagtattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 216 - protein, 446 aa, synthetic construct
  REGION 1..446: note = Description of Artificial Sequence: Synthetic clone 18 VH-CH123polypeptide
EVQLLESGGGLVQPGGSLRLSCAASGFTFSSYAMSWVRQAPGKGLEWVSTIHKTGSPTYYADSVKGRFTISRDNSKNTLYLQMNSLRAEDTAVYYCAKSLKYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - DNA, 642 bp, synthetic construct
  misc_feature 1..642: note = Description of Artificial Sequence: Synthetic clone 18 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcccatttgcaaagtggggtcccatcaaggttcagtggcagtggatctgggacagatttcactctcaccatcagcagtctgcaacctgaagattttgcaacttactactgtcaacagccgcggagtctgcctgctacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 218 - protein, 214 aa, synthetic construct
  REGION 1..214: note = Description of Artificial Sequence: Synthetic clone 18 VL-CKpolypeptide
DIQMTQSPSSLSASVGDRVTITCRASQSISSYLNWYQQKPGKAPKLLIYRASHLQSGVPSRFSGSGSGTDFTLTISSLQPEDFATYYCQQPRSLPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 219 - DNA, 1338 bp, synthetic construct
  misc_feature 1..1338: note = Description of Artificial Sequence: Synthetic clone 21 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactatttatcgtgcgggtcagaatacaaggtacgcagactccgtgaagggccggttcaccatctccagagacaattccaagaacacgctgtatctgcaaatgaacagcctgagagccgaggacacggccgtatattactgtgcgaaatcgagtcggacttttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 220 - protein, 446 aa, synthetic construct
  REGION 1..446: note = Description of Artificial Sequence: Synthetic clone 21 VH-CH123polypeptide
EVQLLESGGGLVQPGGSLRLSCAASGFTFSSYAMSWVRQAPGKGLEWVSTIYRAGQNTRYADSVKGRFTISRDNSKNTLYLQMNSLRAEDTAVYYCAKSSRTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - DNA, 642 bp, synthetic construct
  misc_feature 1..642: note = Description of Artificial Sequence: Synthetic clone 21 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcctcgttgcaaagtggggtcccatcaaggttcagtggcagtggatctgggacagatttcactctcaccatcagcagtctgcaacctgaagattttgcaacttactactgtcaacaggcgacgctgcagcctcggacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 222 - protein, 214 aa, synthetic construct
  REGION 1..214: note = Description of Artificial Sequence: Synthetic clone 21 VL-CKpolypeptide
DIQMTQSPSSLSASVGDRVTITCRASQSISSYLNWYQQKPGKAPKLLIYKASSLQSGVPSRFSGSGSGTDFTLTISSLQPEDFATYYCQQATLQPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 223 - DNA, 1338 bp, synthetic construct
  misc_feature 1..1338: note = Description of Artificial Sequence: Synthetic clone 23 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattagtgcttctggtacttctacaaattacgcagactccgtgaagggccggttcaccatctccagagacaattccaagaacacgctgtatctgcaaatgaacagcctgagagccgaggacacggccgtatattactgtgcgaaaagtaatagtgattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 224 - protein, 446 aa, synthetic construct
  REGION 1..446: note = Description of Artificial Sequence: Synthetic clone 23 VH-CH123polypeptide
EVQLLESGGGLVQPGGSLRLSCAASGFTFSSYAMSWVRQAPGKGLEWVSSISASGTSTNYADSVKGRFTISRDNSKNTLYLQMNSLRAEDTAVYYCAKSNSDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - DNA, 642 bp, synthetic construct
  misc_feature 1..642: note = Description of Artificial Sequence: Synthetic clone 23 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctatttgcaaagtggggtcccatcaaggttcagtggcagtggatctgggacagatttcactctcaccatcagcagtctgcaacctgaagattttgcaacttactactgtcaacagtctggttatgatcctgctacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 226 - protein, 214 aa, synthetic construct
  REGION 1..214: note = Description of Artificial Sequence: Synthetic clone 23 VL-CKpolypeptide
DIQMTQSPSSLSASVGDRVTITCRASQSISSYLNWYQQKPGKAPKLLIYGASYLQSGVPSRFSGSGSGTDFTLTISSLQPEDFATYYCQQSGYDPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 227 - DNA, 1338 bp, synthetic construct
  misc_feature 1..1338: note = Description of Artificial Sequence: Synthetic clone 24 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattgataatagtggtgcttatacatcttacgcagactccgtgaagggcaggttcaccatctccagagacaattccaagaacacgctgtatctgcaaatgaacagcctgagagccgaggacacggccgtatattactgtgcgaaaggtagttctgattttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 228 - protein, 446 aa, synthetic construct
  REGION 1..446: note = Description of Artificial Sequence: Synthetic clone 24 VH-CH123polypeptide
EVQLLESGGGLVQPGGSLRLSCAASGFTFSSYAMSWVRQAPGKGLEWVSAIDNSGAYTSYADSVKGRFTISRDNSKNTLYLQMNSLRAEDTAVYYCAKGSSDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - DNA, 642 bp, synthetic construct
  misc_feature 1..642: note = Description of Artificial Sequence: Synthetic clone 24 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcctctttgcaaagtggggtcccatcaaggttcagtggcagtggatctgggacagatttcactctcaccatcagcagtctgcaacctgaagattttgcaacttactactgtcaacaggctggtaatgatccttctacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 230 - protein, 214 aa, synthetic construct
  REGION 1..214: note = Description of Artificial Sequence: Synthetic clone 24 VL-CKpolypeptide
DIQMTQSPSSLSASVGDRVTITCRASQSISSYLNWYQQKPGKAPKLLIYGASSLQSGVPSRFSGSGSGTDFTLTISSLQPEDFATYYCQQAGNDPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 231 - DNA, 1338 bp, synthetic construct
  misc_feature 1..1338: note = Description of Artificial Sequence: Synthetic clone 26 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcgattactgcgccgggtcctcggacacagtacgcagactccgtgaagggccggttcaccatctccagagacaattccaagaacacgctgtatctgcaaatgaacagcctgagagccgaggacacggccgtatattactgtgcgaaagggacgcgtacgtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 232 - protein, 446 aa, synthetic construct
  REGION 1..446: note = Description of Artificial Sequence: Synthetic clone 26 VH-CH123polypeptide
EVQLLESGGGLVQPGGSLRLSCAASGFTFSSYAMSWVRQAPGKGLEWVSAITAPGPRTQYADSVKGRFTISRDNSKNTLYLQMNSLRAEDTAVYYCAKGTRTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - DNA, 642 bp, synthetic construct
  misc_feature 1..642: note = Description of Artificial Sequence: Synthetic clone 26 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatcccatttgcaaagtggggtcccatcaaggttcagtggcagtggatctgggacagatttcactctcaccatcagcagtctgcaacctgaagattttgcaacttactactgtcaacagccgacgactacgcctcgtacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 234 - protein, 214 aa, synthetic construct
  REGION 1..214: note = Description of Artificial Sequence: Synthetic clone 26 VL-CKpolypeptide
DIQMTQSPSSLSASVGDRVTITCRASQSISSYLNWYQQKPGKAPKLLIYGASHLQSGVPSRFSGSGSGTDFTLTISSLQPEDFATYYCQQPTTTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - DNA, 1338 bp, synthetic construct
  misc_feature 1..1338: note = Description of Artificial Sequence: Synthetic clone 27 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaaagattaagccgcagggtgatgcgacacggtacgcagactccgtgaagggccggttcaccatctccagagacaattccaagaacacgctgtatctgcaaatgaacagcctgagagccgaggacacggccgtatattactgtgcgaaaggggctcggaggtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 236 - protein, 446 aa, synthetic construct
  REGION 1..446: note = Description of Artificial Sequence: Synthetic clone 27 VH-CH123polypeptide
EVQLLESGGGLVQPGGSLRLSCAASGFTFSSYAMSWVRQAPGKGLEWVSKIKPQGDATRYADSVKGRFTISRDNSKNTLYLQMNSLRAEDTAVYYCAKGARRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - DNA, 642 bp, synthetic construct
  misc_feature 1..642: note = Description of Artificial Sequence: Synthetic clone 27 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcccgtttgcaaagtggggtcccatcaaggttcagtggcagtggatctgggacagatttcactctcaccatcagcagtctgcaacctgaagattttgcaacttactactgtcaacagactaagcggcctcctcagacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 238 - protein, 214 aa, synthetic construct
  REGION 1..214: note = Description of Artificial Sequence: Synthetic clone 27 VL-CKpolypeptide
DIQMTQSPSSLSASVGDRVTITCRASQSISSYLNWYQQKPGKAPKLLIYRASRLQSGVPSRFSGSGSGTDFTLTISSLQPEDFATYYCQQTKRPPQTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - DNA, 1338 bp, synthetic construct
  misc_feature 1..1338: note = Description of Artificial Sequence: Synthetic clone 41 VH-CH123polynucleotide
  source 1..1338: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaacgattaagcagacgggtacgcagacaaagtacgcagactccgtgaagggccggttcaccatctccagagacaattccaagaacacgctgtatctgcaaatgaacagcctgagagccgaggacacggccgtatattactgtgcgaaaagtggtcggcggtttgactactggggccagggaaccctggtcaccgtctcgagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatctcgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 240 - protein, 446 aa, synthetic construct
  REGION 1..446: note = Description of Artificial Sequence: Synthetic clone 41 VH-CH123polypeptide
EVQLLESGGGLVQPGGSLRLSCAASGFTFSSYAMSWVRQAPGKGLEWVSTIKQTGTQTKYADSVKGRFTISRDNSKNTLYLQMNSLRAEDTAVYYCAKSGRRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - DNA, 642 bp, synthetic construct
  misc_feature 1..642: note = Description of Artificial Sequence: Synthetic clone 41 VL-CKpolynucleotide
  source 1..642: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccgtttgcaaagtggggtcccatcaaggttcagtggcagtggatctgggacagatttcactctcaccatcagcagtctgcaacctgaagattttgcaacttactactgtcaacaggctagtcttacgcctccgacgttcggccaagggaccaaggtggaaatcaaacggaccgtggcggcgccatctgtcttcatcttcccgccatctgatgagcagttgaaatctggtaccgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 242 - protein, 214 aa, synthetic construct
  REGION 1..214: note = Description of Artificial Sequence: Synthetic clone 41 VL-CKpolypeptide
DIQMTQSPSSLSASVGDRVTITCRASQSISSYLNWYQQKPGKAPKLLIYKASRLQSGVPSRFSGSGSGTDFTLTISSLQPEDFATYYCQQASLTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - DNA, 1407 bp, Homo sapiens
  source 1..1407: mol_type = other DNA
aagaggcctcaaagaaagagctgcggtgcaggaattcgtgtgccggatttggttagctgagcccaccgagagggtaagtgacagctgctcctgcgcttgccatggcaccagcggggaggctggggtcaaggctgagcctccatccctgtcccccacatggggggacaggggtccaggtccaggggcagatcctactccctccatgggccggatcttccccacagggcagggctgatccagctgtgggtctcttggttccctctttcagcgcctgcaggatgaaagctctctgtctcctcctcctccctgtcctggggctgttggtgtctagcaagaccctgtgctccatggaagaagccatcaatgagaggatccaggaggtcgccggctccctaagtgaggaccccccacttgggcaagctccccaagggtctcagagacctcactgatccctggcacagacctgactccaacccagccccagcgctcaccaaatctcatcctcaaatccaaccagatcataaattcaaccccaactccactcccaacccctccgactgtccccaccttatccacggctccaaacccaatccccgctctcactccaaaccttcccttactccaaaacacccaactcaagacagggtcctggaggccagtgagctcctatgcccacagggacctagctccaaaccaacagggctaggggaggatgggggagggaccgtttggtctcacagctccccctgtctcctttcctcctgccccccagtatttagggcaataagcagcattggcctggagtgccagagcgtcacctccaggggggacctggctacttgcccccgaggtgagtgcaggagactgttgtccaggcgcccatttctgttccaagtcccctgggaatgccccctccccgccacgttccccgtgtccagcctctactcccctaggatcttggtcctgactcccagccttctccgcccaccatctggacactggtgtccaccctcactccctgcctccagtgcccattcagtggttggagcctccagccgtccccgtccccacccccgcccccccaacccccctccgcgctccccacccccctcccgctcccaccctcagcctcccagctcagagtccacgctcctgtgttccgggctgcaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctgaggtcgcgcgcagcgcgtgcacagcgcgggcggaggcggctccaggtccggaggggttgcgggggagctggaaataaacctggagatgatgatgatgatgatgatga

SEQ ID NO: 244 - DNA, 327 bp, Homo sapiens
  source 1..327: mol_type = other DNA
atgaaagctctctgtctcctcctcctccctgtcctggggctgttggtgtctagcaagaccctgtgctccatggaagaagccatcaatgagaggatccaggaggtcgccggctccctaatatttagggcaataagcagcattggcctggagtgccagagcgtcacctccaggggggacctggctacttgcccccgaggcttcgccgtcaccggctgcacttgtggctccgcctgtggctcgtgggatgtgcgcgccgagaccacatgtcactgccagtgcgcgggcatggactggaccggagcgcgctgctgtcgtgtgcagccctga

SEQ ID NO: 245 - protein, 108 aa, Homo sapiens
MKALCLLLLPVLGLLVSSKTLCSMEEAINERIQEVAGSLIFRAISSIGLECQSVTSRGDLATCPRGFAVTGCTCGSACGSWDVRAETTCHCQCAGMDWTGARCCRVQP

SEQ ID NO: 246 - DNA, 65 bp, synthetic construct
  misc_feature 1..65: note = Resistin shRNA
  source 1..65: mol_type = other DNA
aattctccctaatatttagggcaataatcacgtaacttattgccctaaatattagggaaataaag

SEQ ID NO: 247 - DNA, 65 bp, synthetic construct
  misc_feature 1..65: note = Resistin shRNA
  source 1..65: mol_type = other DNA
gatcctttatttccctaatatttagggcaataagttacgtgattattgccctaaatattagggag

SEQ ID NO: 248 - DNA, 336 bp, Mus musculus
  source 1..336: mol_type = other DNA
atggggccgtcctcttgcctccttctcatcctaatcccccttctccagctgatcaacccggggagtactcagtgttccttagactccgttatggataagaagatcaaggatgttctcaacagtctagagtacagtccctctcctataagcaagaagctctcgtgtgctagtgtcaaaagccaaggcagaccgtcctcctgccctgctgggatggctgtcactggctgtgcttgtggctatggctgtggttcgtgggatgttcagctggaaaccacctgccactgccagtgcagtgtggtggactggaccactgcccgctgctgccacctgacctga

SEQ ID NO: 249 - protein, 111 aa, Mus musculus
MGPSSCLLLILIPLLQLINPGSTQCSLDSVMDKKIKDVLNSLEYSPSPISKKLSCASVKSQGRPSSCPAGMAVTGCACGYGCGSWDVQLETTCHCQCSVVDWTTARCCHLT

SEQ ID NO: 250 - DNA, 65 bp, synthetic construct
  misc_feature 1..65: note = RELM-Beta shRNA
  source 1..65: mol_type = other DNA
aattcaacagtctagagtacagtcccttcacgtaacagggactgtactctagactgttaataaag

SEQ ID NO: 251 - DNA, 65 bp, synthetic construct
  misc_feature 1..65: note = RELM-Beta shRNA
  source 1..65: mol_type = other DNA
gatcctttattaacagtctagagtacagtccctgttacgtgaagggactgtactctagactgttg

(19) United States
(12) Patent Application Publication
(10) Pub. No.: US 2026/0297180 A1
(43) Pub. Date: Oct. 1, 2026

(54) COMPOSITIONS AND METHODS FOR TREATING DISEASES AND CONDITIONS ASSOCIATED WITH ACTIVATION OF THE NLRP3 INFLAMMASOME

(71) Applicant: THE JOHNS HOPKINS UNIVERSITY, Baltimore, MD (US)

(72) Inventor: Roger A. JOHNS, Reisterstown, MD (US)

(21) Appl. No.: 18/996,429

(22) PCT Filed: Aug. 9, 2023
(86) PCT No.: PCT/US2023/071908

§ 371 Date: Jan. 17, 2025

Related U.S. Application Data
(60) Provisional application No. 63/370,851, filed on Aug. 9, 2022.

(51) Int. Cl. C07K 16/26; A61P 9/12; C12N 15/113; C12Q 1/6883; G01N 33/74

(52) U.S. Cl. CPC C07K 16/26; A61P 9/12; C12N 15/1136; C12Q 1/6883; G01N 33/74; C07K 2317/622; C12N 2310/14; C12N 2310/531; C12Q 2600/156; G01N 2333/575; G01N 2800/321; G01N 2800/52

(57) ABSTRACT

The present invention relates to the field of inflammation. More specifically, the present invention provides compositions and methods for treating diseases and conditions associated with the priming and activation of the NLR Family Pyrin Domain Containing 3 (NLRP3) inflammasome. In particular embodiments, the method comprises the step of administering to the patient an isolated, recombinant antibody or antigen-binding fragment thereof that binds human Resistin.

### CROSS-REFERENCE TO RELATED APPLICATIONS

This application claims the benefit of U.S. Provisional Application No. 63/370,851, filed Aug. 9, 2022, which is incorporated herein by reference in its entirety.

### GOVERNMENT SUPPORT CLAUSE

This invention was made with government support under grant no. HL138497, awarded by the National Institutes of Health. The government has certain rights in the invention.

### FIELD OF THE INVENTION

The present invention relates to the field of inflammation. More specifically, the present invention provides compositions and methods for treating diseases and conditions associated with the priming and activation of the NLR Family Pyrin Domain Containing 3 (NLRP3) inflammasome.

### REFERENCE TO AN ELECTRONIC SEQUENCE LISTING

The text of the computer readable sequence listing filed herewith, titled “P17933-01”, created Aug. 9, 2023, having a file size of 449,471 bytes, is hereby incorporated by reference in its entirety.

### BACKGROUND OF THE INVENTION

The NLRP3 inflammasome is a cellular machinery endowed with the capacity for rapid proteolytic processing of the pro-inflammatory cytokines IL-1β and IL-18 in response to exogenous Pathogen-Associated Molecular Pattern molecules (PAMPs) and endogenous Damage-Associated Molecular Pattern molecules (DAMPs).49 The NLRP3 inflammasome consists of a sensor (NLRP3), an adaptor (ASC; also known as PYCARD) and an effector (caspase-1).49 Inflammasome activation is considered to be a two-step process—to be ‘primed’ and then activated. The PAMP or DAMP signaling can induce the priming through NF-κB, leading to upregulated expression of inflammasome components NLRP3, pro-caspase-1, pro-IL-18 and pro-IL-1β. Following priming, NLRP3 assembly results in the full inflammasome activation, leading to caspase 1-dependent cleavage of IL-1β and IL-18.⁴⁹ NLRP3 inflammasome activation has been linked to various inflammasome-related diseases/disorders, immune diseases, inflammatory diseases, auto-immune diseases and auto-inflammatory diseases. A great need exists for compositions and methods useful for treating NLRP3 inflammasome mediated diseases, disorders and conditions.

### SUMMARY OF THE INVENTION

The present invention is based, at least in part, on the inventor's demonstration that human Resistin (hResistin)/RELMα (the equivalent rodent form of hResistin) is a critical regulator of the priming and activation stages of the NLRP3 inflammasome (FIG. 1). The present inventors show that hResistin/RELMα is critical to both NLRP3, IL1-β and IL-18 priming (via HMGB1)¹¹ and NLRP3 activation (via critical BTK¹² phosphorylation of four specific NLRP3 tyrosine residues) leading to cleavage of pro-versions of IL-1β and IL-18 to active cytokines via caspase-1. Thus, hResistin/RELMα plays a major, novel and essential role in engaging the NLRP3 inflammasome in the innate and adaptive immune response to injury.

As described further herein, the present inventors found that hResistin activates HMGB1 and BTK signaling in macrophages and other cells that use the inflammasome (FIG. 1). hResistin treatment induced the expression of HMGB1 and total BTK in the human macrophages, which were prevented by the pretreatment of the anti-hResistin neutralizing antibody. hResistin-HMGB1 also induces NLRP3 priming in macrophages. hResistin induced the gene and protein expression of NLRP3, pro-casase-1 and pro-IL-1β in human macrophages, which were prevented by the pretreatment of the anti-hResistin neutralizing antibody and the HMGB1 antagonist Box-A.

The present inventors also found that hResistin-BTK induces NLRP3 activation through four critical tyrosine phosphorylations, changing its structure and leading to caspase-1 cleavage of the pro-versions of IL-1B and IL-18 to active forms and their secretion in macrophages. hResistin induced the activities of caspase-1 in human macrophages, which were prevented by the BTK inhibitor ibrutinib. Moreover, hResistin induced the IL-1β and IL-18 production and secretion of human macrophages, which were also prevented by the pretreatment of ibrutinib, demonstrating a dependence on hResistin activated BTK. In addition, the macrophage-derived hResistin activates HMGB1, BTK and NLRP3 signaling in B cells. hResistin production was not found in human B cells (CT values>39 as tested by q-RT-PCR). Conditioned medium from the hypoxia-treated macrophages induced the expression of NLRP3 and IL-1B in B cells, which were prevented by the pretreatment of the anti-hResistin neutralizing antibody, indicating that NLRP3 and IL-1β were induced by the macrophage-derived hResistin. This suggests a role in the transition from innate to adaptive immunity.

The present inventors further found that in vivo RELMα induces HMGB1, BTK and NLRP3 inflammasome in the hypoxia PH mouse model. Hypoxia upregulated expression of BTK and NLRP3 in the PH mouse lung tissues during the inflammatory stage (post-hypoxic day-4), which were prevented by the anti-RELMα/hResistin therapeutic antibody. In addition, the macrophage-derived hResistin-BTK signaling induces PV-SMC proliferation. Conditioned medium from the hResistin (200 ng/mL)-treated macrophages induced the proliferation of primary human PV-SMCs. Intriguingly, blocking IL-1β or IL-18 in these PV-SMCs causes them resistant to the above-mentioned conditioned medium. Moreover, phosphorylation of AKT and ERK1/2, which are the key indicators of proliferation, was induced by the above conditioned medium and prevented by neutralizing IL-1β or IL-18. Direct stimulation of the same dose of hResistin failed to induce PV-SMC proliferation. These findings collectively indicate the pro-proliferative activities of the IL-1β and IL-18 secreted from the hResistin-stimulated macrophages.

The macrophage is a very important source of the NLRP3 inflammasome, but far from the only cell type. Neutophils, as well as other immune cells such as B cells and T-cells may utilize the inflammasome.

Accordingly, in one aspect, the present invention provides compositions and methods for treating diseases and conditions associated with the priming and activation of the NLR Family Pyrin Domain Containing 3 (NLRP3) inflammasome. In particular embodiments, the method comprises the step of administering to the patient an isolated, recombinant antibody or antigen-binding fragment thereof that binds human Resistin. The anti-Resistin antibody or antigen-binding fragment thereof can comprise a single chain variable fragment (scFv), a dimeric scFv, a Fab, a Fab′, a F(ab′) 2 fragment or a full length antibody.

In particular embodiments, the hResistin therapeutic antibody comprises clone 13, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 73 or a conservative substitution at up to 5 amino acid positions of SEQ ID NO:73, and the light chain variable region comprises SEQ ID NO:77 or a conservative substitution at up to 5 amino acid positions of SEQ ID NO:77. In other embodiments, the hResistin antibody or antigen-binding fragment thereof comprises (a) a heavy chain variable region comprising CDRs 1, 2 and 3 comprising SEQ ID NOS: 74-76, respectively, or a conservative substitution at up to 2 amino acids of one or more of SEQ ID NOS: 74-76, and (b) a light chain variable region comprising CDRs 1, 2, and 3 comprising SEQ ID NOS: 78-80, respectively, or a conservative substitution at up to 2 amino acids of one or more of SEQ ID NOS: 78-80. In further embodiments, the hResistin antibody or antigen-binding fragment thereof comprises a single chain variable fragment (scFv) or antigen-binding fragment thereof that binds Resistin, wherein the scFv comprises SEQ ID NO:72 or a conservative substitution at up to 5 amino acids of SEQ ID NO: 72.

In particular embodiments, the hResistin therapeutic antibody comprises clone 42, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 163 or a conservative substitution at up to 5 amino acid positions of SEQ ID NO: 163, and the light chain variable region comprises SEQ ID NO: 167 or a conservative substitution at up to 5 amino acid positions of SEQ ID NO:167. In other embodiments, the hResistin antibody or antigen-binding fragment thereof comprises (a) a heavy chain variable region comprising CDRs 1, 2 and 3 comprising SEQ ID NOS: 164-166, respectively, or a conservative substitution at up to 2 amino acids of one or more of SEQ ID NOS: 164-166, and (b) a light chain variable region comprising CDRs 1, 2, and 3 comprising SEQ ID NOS: 168-170, respectively, or a conservative substitution at up to 2 amino acids of one or more of SEQ ID NOS: 168-170. In further embodiments, the hResistin antibody or antigen-binding fragment thereof comprises a single chain variable fragment (scFv) or antigen-binding fragment thereof that binds Resistin, wherein the scFv comprises SEQ ID NO: 162 or a conservative substitution at up to 5 amino acids of SEQ ID NO: 162.

In particular embodiments, the hResistin therapeutic antibody comprises clone 2, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 13 or a conservative substitution at up to 5 amino acid positions of SEQ ID NO:13, and the light chain variable region comprises SEQ ID NO: 17 or a conservative substitution at up to 5 amino acid positions of SEQ ID NO:17. In other embodiments, the hResistin antibody or antigen-binding fragment thereof comprises (a) a heavy chain variable region comprising CDRs 1, 2 and 3 comprising SEQ ID NOS: 14-16, respectively, or a conservative substitution at up to 2 amino acids of one or more of SEQ ID NOS: 14-16, and (b) a light chain variable region comprising CDRs 1, 2, and 3 comprising SEQ ID NOS: 18-20, respectively, or a conservative substitution at up to 2 amino acids of one or more of SEQ ID NOS: 18-20. In further embodiments, the hResistin antibody or antigen-binding fragment thereof comprises a single chain variable fragment (scFv) or antigen-binding fragment thereof that binds Resistin, wherein the scFv comprises SEQ ID NO: 12 or a conservative substitution at up to 5 amino acids of SEQ ID NO: 12.

In particular embodiments, the hResistin therapeutic antibody comprises clone 11, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 63 or a conservative substitution at up to 5 amino acid positions of SEQ ID NO:63, and the light chain variable region comprises SEQ ID NO:67 or a conservative substitution at up to 5 amino acid positions of SEQ ID NO:67. In other embodiments, the hResistin antibody or antigen-binding fragment thereof comprises (a) a heavy chain variable region comprising CDRs 1, 2 and 3 comprising SEQ ID NOS: 64-66, respectively, or a conservative substitution at up to 2 amino acids of one or more of SEQ ID NOS.64-66, and (b) a light chain variable region comprising CDRs 1, 2, and 3 comprising SEQ ID NOS.68-70, respectively, or a conservative substitution at up to 2 amino acids of one or more of SEQ ID NOS: 68-70. In further embodiments, the hResistin antibody or antigen-binding fragment thereof comprises a single chain variable fragment (scFv) or antigen-binding fragment thereof that binds Resistin, wherein the scFv comprises SEQ ID NO:62 or a conservative substitution at up to 5 amino acids of SEQ ID NO: 62.

In certain embodiments, the anti-Resistin antibody or antigen-binding fragment thereof comprising a heavy chain variable region and a light chain variable region, wherein: (a) the heavy chain variable region comprises SEQ ID NO:3 or a conservative substitution at up to 5 amino acid positions of SEQ ID NO:3, and the light chain variable region comprises SEQ ID NO: 7 or a conservative substitution at up to 5 amino acid positions of SEQ ID NO:7; (b) the heavy chain variable region comprises SEQ ID NO:13 or a conservative substitution at up to 5 amino acid positions of SEQ ID NO:13, and the light chain variable region comprises SEQ ID NO: 17 or a conservative substitution at up to 5 amino acid positions of SEQ ID NO:17; (c) the heavy chain variable region comprises SEQ ID NO:23 or a conservative substitution at up to 5 amino acid positions of SEQ ID NO:23, and the light chain variable region comprises SEQ ID NO: 27 or a conservative substitution at up to 5 amino acid positions of SEQ ID NO:27; (d) the heavy chain variable region comprises SEQ ID NO:33 or a conservative substitution at up to 5 amino acid positions of SEQ ID NO:33 and the light chain variable region comprises SEQ ID NO: 37 or a conservative substitution at up to 5 amino acid positions of SEQ ID NO:37; (e) the heavy chain variable region comprises SEQ ID NO:43 or a conservative substitution at up to 5 amino acid positions of SEQ ID NO:43, and the light chain variable region comprises SEQ ID NO: 47 or a conservative substitution at up to 5 amino acid positions of SEQ ID NO:47; (f) the heavy chain variable region comprises SEQ ID NO:53 or a conservative substitution at up to 5 amino acid positions of SEQ ID NO:53, and the light chain variable region comprises SEQ ID NO: 57 or a conservative substitution at up to 5 amino acid positions of SEQ ID NO:57; (g) the heavy chain variable region comprises SEQ ID NO:63 or a conservative substitution at up to 5 amino acid positions of SEQ ID NO:63 and the light chain variable region comprises SEQ ID NO: 67 or a conservative substitution at up to 5 amino acid positions of SEQ ID NO:67; (h) the heavy chain variable region comprises SEQ ID NO:73 or a conservative substitution at up to 5 amino acid positions of SEQ ID NO:73, and the light chain variable region comprises SEQ ID NO: 77 or a conservative substitution at up to 5 amino acid positions of SEQ ID NO:77; (i) the heavy chain variable region comprises SEQ ID NO:83 or a conservative substitution at up to 5 amino acid positions of SEQ ID NO:83, and the light chain variable region comprises SEQ ID NO: 87 or a conservative substitution at up to 5 amino acid positions of SEQ ID NO:87; (j) the heavy chain variable region comprises SEQ ID NO:93 or a conservative substitution at up to 5 amino acid positions of SEQ ID NO:93 and the light chain variable region comprises SEQ ID NO: 97 or a conservative substitution at up to 5 amino acid positions of SEQ ID NO:97; (k) the heavy chain variable region comprises SEQ ID NO: 103 or a conservative substitution at up to 5 amino acid positions of SEQ ID NO:103, and the light chain variable region comprises SEQ ID NO: 107 or a conservative substitution at up to 5 amino acid positions of SEQ ID NO: 107; (1) the heavy chain variable region comprises SEQ ID NO:113 or a conservative substitution at up to 5 amino acid positions of SEQ ID NO:113 and the light chain variable region comprises SEQ ID NO: 117 or a conservative substitution at up to 5 amino acid positions of SEQ ID NO:117; (m) the heavy chain variable region comprises SEQ ID NO: 123 or a conservative substitution at up to 5 amino acid positions of SEQ ID NO: 123, and the light chain variable region comprises SEQ ID NO: 127 or a conservative substitution at up to 5 amino acid positions of SEQ ID NO: 127; (n) the heavy chain variable region comprises SEQ ID NO: 133 or a conservative substitution at up to 5 amino acid positions of SEQ ID NO:133, and the light chain variable region comprises SEQ ID NO: 137 or a conservative substitution at up to 5 amino acid positions of SEQ ID NO: 137; (0) the heavy chain variable region comprises SEQ ID NO:143 or a conservative substitution at up to 5 amino acid positions of SEQ ID NO:143, and the light chain variable region comprises SEQ ID NO: 147 or a conservative substitution at up to 5 amino acid positions of SEQ ID NO: 147; (p) the heavy chain variable region comprises SEQ ID NO: 153 or a conservative substitution at up to 5 amino acid positions of SEQ ID NO: 153, and the light chain variable region comprises SEQ ID NO: 157 or a conservative substitution at up to 5 amino acid positions of SEQ ID NO:157; or (q) the heavy chain variable region comprises SEQ ID NO:163 or a conservative substitution at up to 5 amino acid positions of SEQ ID NO:163 and the light chain variable region comprises SEQ ID NO:167 or a conservative substitution at up to 5 amino acid positions of SEQ ID NO: 167.

In other embodiments, the anti-Resistin antibody or antigen-binding fragment thereof comprises: (a) a heavy chain variable region comprising complementarity determining regions (CDRs) 1, 2 and 3 comprising SEQ ID NOS: 4-6, respectively, or a conservative substitution at up to 2 amino acids of one or more of SEQ ID NOS: 4-6, and a light chain variable region comprising CDRs 1, 2, and 3 comprising SEQ ID NOS: 8-10, respectively, or a conservative substitution at up to 2 amino acids of one or more of SEQ ID NOS: 8-10; (b) a heavy chain variable region comprising CDRs 1, 2 and 3 comprising SEQ ID NOS: 14-16, respectively, or a conservative substitution at up to 2 amino acids of one or more of SEQ ID NOS: 14-16, and a light chain variable region comprising CDRs 1, 2, and 3 comprising SEQ ID NOS: 18-20, respectively, or a conservative substitution at up to 2 amino acids of one or more of SEQ ID NOS: 18-20; (c) a heavy chain variable region comprising CDRs 1, 2 and 3 comprising SEQ ID NOS: 24-26, respectively, or a conservative substitution at up to 2 amino acids of one or more of SEQ ID NOS: 24-26, and a light chain variable region comprising CDRs 1, 2, and 3 comprising SEQ ID NOS: 28-30, respectively, or a conservative substitution at up to 2 amino acids of one or more of SEQ ID NOS: 28-30; (d) a heavy chain variable region comprising CDRs 1, 2 and 3 comprising SEQ ID NOS: 34-36, respectively, or a conservative substitution at up to 2 amino acids of one or more of SEQ ID NOS: 34-36, and a light chain variable region comprising CDRs 1, 2, and 3 comprising SEQ ID NOS: 38-40, respectively, or a conservative substitution at up to 2 amino acids of one or more of SEQ ID NOS: 38-40; (e) a heavy chain variable region comprising CDRs 1, 2 and 3 comprising SEQ ID NOS: 44-46, respectively, or a conservative substitution at up to 2 amino acids of one or more of SEQ ID NOS: 44-46, and a light chain variable region comprising CDRs 1, 2, and 3 comprising SEQ ID NOS: 48-50, respectively, or a conservative substitution at up to 2 amino acids of one or more of SEQ ID NOS: 48-50; (f) a heavy chain variable region comprising CDRs 1, 2 and 3 comprising SEQ ID NOS: 54-56, respectively, or a conservative substitution at up to 2 amino acids of one or more of SEQ ID NOS: 54-56, and a light chain variable region comprising CDRs 1, 2, and 3 comprising SEQ ID NOS: 58-60, respectively, or a conservative substitution at up to 2 amino acids of one or more of SEQ ID NOS: 58-60; (g) a heavy chain variable region comprising CDRs 1, 2 and 3 comprising SEQ ID NOS: 64-66, respectively, or a conservative substitution at up to 2 amino acids of one or more of SEQ ID NOS: 64-66, and a light chain variable region comprising CDRs 1, 2, and 3 comprising SEQ ID NOS: 68-70, respectively, or a conservative substitution at up to 2 amino acids of one or more of SEQ ID NOS: 68-70; (h) a heavy chain variable region comprising CDRs 1, 2 and 3 comprising SEQ ID NOS: 74-76, respectively, or a conservative substitution at up to 2 amino acids of one or more of SEQ ID NOS: 74-76, and a light chain variable region comprising CDRs 1, 2, and 3 comprising SEQ ID NOS: 78-80, respectively, or a conservative substitution at up to 2 amino acids of one or more of SEQ ID NOS: 78-80; (i) a heavy chain variable region comprising CDRs 1, 2 and 3 comprising SEQ ID NOS: 84-86, respectively, or a conservative substitution at up to 2 amino acids of one or more of SEQ ID NOS: 84-86, and a light chain variable region comprising CDRs 1, 2, and 3 comprising SEQ ID NOS: 88-90, respectively, or a conservative substitution at up to 2 amino acids of one or more of SEQ ID NOS: 88-90; (j) a heavy chain variable region comprising CDRs 1, 2 and 3 comprising SEQ ID NOS: 94-96, respectively, or a conservative substitution at up to 2 amino acids of one or more of SEQ ID NOS: 94-96, and a light chain variable region comprising CDRs 1, 2, and 3 comprising SEQ ID NOS: 98-100, respectively, or a conservative substitution at up to 2 amino acids of one or more of SEQ ID NOS: 98-100; (k) a heavy chain variable region comprising CDRs 1, 2 and 3 comprising SEQ ID NOS: 104-106, respectively, or a conservative substitution at up to 2 amino acids of one or more of SEQ ID NOS: 104-106, and a light chain variable region comprising CDRs 1, 2, and 3 comprising SEQ ID NOS: 108-110, respectively, or a conservative substitution at up to 2 amino acids of one or more of SEQ ID NOS: 108-110; (1) a heavy chain variable region comprising CDRs 1, 2 and 3 comprising SEQ ID NOS: 114-116, respectively, or a conservative substitution at up to 2 amino acids of one or more of SEQ ID NOS: 114-116, and a light chain variable region comprising CDRs 1, 2, and 3 comprising SEQ ID NOS: 118-120, respectively, or a conservative substitution at up to 2 amino acids of one or more of SEQ ID NOS: 118-120; (m) a heavy chain variable region comprising CDRs 1, 2 and 3 comprising SEQ ID NOS: 124-126, respectively, or a conservative substitution at up to 2 amino acids of one or more of SEQ ID NOS: 124-126, and a light chain variable region comprising CDRs 1, 2, and 3 comprising SEQ ID NOS: 128-130, respectively, or a conservative substitution at up to 2 amino acids of one or more of SEQ ID NOS: 128-130; (n) a heavy chain variable region comprising CDRs 1, 2 and 3 comprising SEQ ID NOS: 134-136, respectively, or a conservative substitution at up to 2 amino acids of one or more of SEQ ID NOS: 134-136, and a light chain variable region comprising CDRs 1, 2, and 3 comprising SEQ ID NOS: 138-140, respectively, or a conservative substitution at up to 2 amino acids of one or more of SEQ ID NOS: 138-140; (o) a heavy chain variable region comprising CDRs 1, 2 and 3 comprising SEQ ID NOS: 144-146, respectively, or a conservative substitution at up to 2 amino acids of one or more of SEQ ID NOS: 144-146, and a light chain variable region comprising CDRs 1, 2, and 3 comprising SEQ ID NOS: 148-150, respectively, or a conservative substitution at up to 2 amino acids of one or more of SEQ ID NOS: 148-150; (p) a heavy chain variable region comprising CDRs 1, 2 and 3 comprising SEQ ID NOS: 154-156, respectively, or a conservative substitution at up to 2 amino acids of one or more of SEQ ID NOS: 154-156, and a light chain variable region comprising CDRs 1, 2, and 3 comprising SEQ ID NOS: 158-160, respectively, or a conservative substitution at up to 2 amino acids of one or more of SEQ ID NOS: 158-160; or (q) a heavy chain variable region comprising CDRs 1, 2 and 3 comprising SEQ ID NOS: 164-166, respectively, or a conservative substitution at up to 2 amino acids of one or more of SEQ ID NOS: 164-166, and a light chain variable region comprising CDRs 1, 2, and 3 comprising SEQ ID NOS: 168-170, or a conservative substitution at up to 2 amino acids of one or more of SEQ ID NOS: 168-170.

In specific embodiments, the anti-Resistin antibody or antigen-binding fragment thereof further comprises a heavy chain constant region comprising SEQ ID NO:172 and a light chain constant region comprising SEQ ID NO:174.

In alternative embodiments, the anti-Resistin antibody or antigen-binding fragment thereof comprises an scfv or antigen-binding fragment thereof that binds Resistin, wherein the scFv comprises SEQ ID NO:2 or a conservative substitution at up to 5 amino acids of SEQ ID NO: 2; SEQ ID NO:12 or a conservative substitution at up to 5 amino acids of SEQ ID NO:12; SEQ ID NO:22 or a conservative substitution at up to 5 amino acids of SEQ ID NO:22; SEQ ID NO: 32 or a conservative substitution at up to 5 amino acids of SEQ ID NO:32; SEQ ID NO:42 or a conservative substitution at up to 5 amino acids of SEQ ID NO:42; SEQ ID NO:52 or a conservative substitution at up to 5 amino acids of SEQ ID NO:52; SEQ ID NO:62 or a conservative substitution at up to 5 amino acids of SEQ ID NO:62; SEQ ID NO:72 or a conservative substitution at up to 5 amino acids of SEQ ID NO:72; SEQ ID NO:82 or a conservative substitution at up to 5 amino acids of SEQ ID NO:82; SEQ ID NO:92 or a conservative substitution at up to 5 amino acids of SEQ ID NO:92; SEQ ID NO:102 or a conservative substitution at up to 5 amino acids of SEQ ID NO: 102; SEQ ID NO: 112 or a conservative substitution at up to 5 amino acids of SEQ ID NO:112; SEQ ID NO:122 or a conservative substitution at up to 5 amino acids of SEQ ID NO: 122; SEQ ID NO:132 or a conservative substitution at up to 5 amino acids of SEQ ID NO: 132; SEQ ID NO: 142 or a conservative substitution at up to 5 amino acids of SEQ ID NO:142; SEQ ID NO: 152 or a conservative substitution at up to 5 amino acids of SEQ ID NO:152; or SEQ ID NO: 162 or a conservative substitution at up to 5 amino acids of SEQ ID NO:162.

In particular embodiments, the NLRP3 inflammasome mediated disease, disorder or condition comprises autoimmune disease; age-related macular degeneration (AMD); autoinflammatory diseases; inflammatory responses; inflammatory skin diseases; sepsis; psoriasis; dermatitis; systemic scleroderma; sclerosis; inflammatory bowel disease; Crohn's disease; ulcerative colitis; respiratory distress syndrome; adult respiratory distress syndrome; meningitis; encephalitis; uveitis; colitis; glomerulonephritis; eczema; asthma; atherosclerosis; leukocyte adhesion deficiency; rheumatoid arthritis; systemic lupus erythematosus (SLE); lupus nephritis (LN); diabetes mellitus; multiple sclerosis, Reynaud's syndrome; autoimmune thyroiditis; allergic encephalomyelitis; Sjorgen's syndrome; juvenile onset diabetes; pernicious anemia; diseases involving leukocyte diapedesis; central nervous system (CNS) inflammatory disorder; multiple organ injury syndrome; hemolytic anemia; cryoglobinemia; Coombs positive anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopeni purpura (ITP) or autoimmune thrombocytopenia; cryopyrin-associated periodic syndromes (CAPS); Alzheimer disease; myocardial infarction; gout; non-alcoholic fatty liver disease; nonalcoholic steatohepatitis; experimental autoimmune encephalitis; oxalate-induced nephropathy; hyperinflammation following influenza infection; stroke; silicosis; myelodysplastic syndrome; contact hypersensitivity; traumatic brain injury; familial cold autoinflammatory syndrome (FCAS); Muckle-Wells syndrome (MWS); neonatal onset multisystem inflammatory disorder (NOMID); crystalline arthropathies; silicosis; asbestosis; gout; pseudogout; diabetes mellitus; familial Mediterranean fever (FMF); pyogenic arthritis with pyodema gangrenosum and acne (PAPA) syndrome; hyperimmunoglobulinemia D with periodic fever syndrome (HIDS); Mevalonate kinase deficiency (MKD); Schnitzler's syndrome (SS); Aspergillus fumigatus keratitis; stargardt disease type 1; Alzheimer's disease; atherosclerosis; atrial fibrillation; osteoarthritis; cancer; pulmonary hypertension; right or left heart failure; and lung inflammation due to bacterial, viral or parasitic infections.

In another aspect, the present invention provides RNA interference compositions. In particular embodiments, a ribonucleic acid (RNA) interfering (RNAi) composition comprises about 18-25 nucleotides that is complementary to SEQ ID NO:244, wherein the RNAi composition is capable of inhibiting the expression of human Resistin. In specific embodiments, RNAi composition is a small interfering RNA (siRNA), a short hairpin RNA (shRNA), double stranded RNA (dsRNA), and RNA construct or an anti-sense oligonucleotide. In more specific embodiments, the present invention provides A shRNA for knocking down Resistin expression comprising SEQ ID NO:246 or SEQ ID NO:247. In specific embodiments, the present invention provides a method for treating an NLRP3 inflammasome mediated disease, disorder or condition in a patient comprises the step of administering to the patient a pharmaceutical composition comprising the RNAi composition or shRNA described herein.

In a further aspect, the present invention provides methods for predicting disease severity in a patient having an NLRP3 inflammasome mediated disease, disorder or condition. In one embodiment, the method comprises (a) measuring the level of Resistin in a sample obtained from the patient; (b) comparing the level measured in step (a) to a reference; and (c) predicting disease severity in the patient.

In another aspect, the present invention provides methods for risk stratification of progressing to severe disease of a patient having an NLRP3 inflammasome mediated disease, disorder or condition. In one embodiment, the method comprises (a) measuring the level of Resistin in a sample obtained from the patient; (b) comparing the level measured in step (a) to a reference; and (c) stratifying the risk of the patient.

In a further aspect, the present invention provides methods for monitoring disease progression in a patient having an NLRP3 inflammasome mediated disease, disorder or condition. In one embodiment, the method comprises (a) measuring the level of Resistin in a first sample obtained from the patient; (b) measuring the level of Resistin in a second sample from the patient that has been obtained after the first sample; (c) comparing the level measured in step (a) to the level measured in step (b); and (d) monitoring disease progression in the patient based on the results of step (c).

In further embodiments, the methods for predicting disease, risk stratifying and/or monitoring disease progression can further comprise the step of screening DNA obtained from the patient for the presence of a Resistin polymorphism associated with the NLRP3 inflammasome mediated disease, disorder or condition. In a specific embodiment, a Resistin polymorphism comprises rs10402265 (C>G). In another specific embodiment, a Resistin polymorphism comprises rs12459044 (C>G).

In particular embodiments, the level of Resistin can be measured using an antibody or antigen-binding fragment thereof described herein.

### BRIEF DESCRIPTION OF THE FIGURES

FIG. 1A-1B. Hresistin upregulates the expression of BTK, HMGB-1, NLRP3, pro-IL-1β, pro-IL-18 and pro-Caspase-1 and cleavage of pro-IL-1β, pro-IL-18 and pro-Caspase-1 in human macrophages. Human THP-1 derived macrophages were treated with 1: media only, 2:20 nM Hresistin, 3: 20 nM Hresistin+HMGB1 Box A antagonist (1 ug/mL), 4: 20 nM Hresistin+Ibrutinib (100 ng/ml), 5. 20 nM Hresistin+200 nM MCC950, 6. 20 nM Hresistin+300 ng/ml Resistin antibody, 7: 20 nM Hresistin+300 ng/mL control antibody, 6. FIG. 1A. Western blot images on BTK (SAB4502936), HMGB-1, NLRP3 (ab214185), ASC speck, pro and cleaved caspase-1 (ab207802), pro and cleaved IL-18, pro and cleaved IL-1β (ab254360) were displayed in the upper left panel. FIG. 1B. Quantitative analysis of each protein was also presented. Data represent means+SD (n=6 per each group. *p<0.05, **P<0.01.

FIG. 2A-2D. RELMα upregulates pro-inflammatory phenotypes in hypoxic mice lungs. FIG. 2A. Lung tissues were collected from WT C57/BL6 mice, kept in a 4 days hypoxia condition with or without Ibrutinib or Resistin antibody or control antibody (n=3 for each group). WT C57/BL6 mice were used as a control group. q-RT-PCR analysis of NLRP3 and Pro-IL-1Bgene expression in mice lung tissues. Mouse normal:-Normoxic mice, Mouse 4DH:-WT C57/BL6 mice kept in 4 days of hypoxic condition, IB4DH:-WT C57/BL6 mice treated with Ibrutinib and kept in 4 days of hypoxic condition, ConAb4DH:-WT C57/BL6 mice treated with one dose of Control Ab and kept in 4 days of hypoxic condition, ResAb4DH:-WT C57/BL6 mice treated with one dose of Resistin Ab and kept in 4 days of hypoxic condition. Data represent means+SD (n=3 per each group). *p<0.05. Resistin-Like Molecule Alpha (RELMα) is an upstream activator of HMGB1 and BTK and NLRP3. Lung tissues were collected from WT and HIMF-K/O C57/BL6 mice, kept in a 4 days hypoxia and in normal condition (n=3 for each group). FIG. 2B. q-RT-PCR analysis of BTK, HMGB-1 and NLRP3 gene expression in mice lung tissues in both normal and hypoxic condition. FIG. 2C. Images of western blots on BTK (SAB4502936), HMGB-1 (ab79823) and NLRP3 (ab214185) protein expression. FIG. 2D. Quantitative analysis of data in B. Data represent means+SD (n=3 per each group). *p<0.05.

FIG. 3A-3D. Hresistin bind to BTK and upregulate the BTK autophosphorylation in human macrophages. FIG. 3A. qRTPCRanalysisofHresistingeneexpressioninTHP-1 differentiated macrophages in both normoxic and hypoxic condition (24 hrs). Data represent means+SD (n=5 per each group. *p<0.05. FIG. 3B. Hresistin activates BTK and HMGB1 in human B cells. Human B cell line (HCC1937 BL) was stimulated with Hresistin recombinant protein at 100 and 200 ng/ml for 24 hrs. Cell pellet and medium were collected. FIG. 3A. Western blotting analysis of BTK and HMGB1 protein expression. Hresistin induces production of BTK and HMGB1, and the secretion of HMGB1 in human cells. FIG. 3B. Quantitative analysis of data in FIG. 3A. Data represent means+SEM (n=5). *p<0.05. FIG. 3C. THP-1 macrophages were kept in hypoxia for 24 hrs. Co-IP assays using Pierce™ Co-IP kit (26149) were performed with anti-Flag antibody coupled resin. Flag-Hresistin proteins with B cell lysates were used as inputs. Eluted proteins were visualized by western blot with BTK (SAB4502936) and Flag antibodies (F1804). Input: before passing through the anti-flag antibody embedded resin; E1: first elusion; E2: second elusion; (+): recombinant BTK and H resistin proteins. FIG. 3D. Hresistin upregulates the autophosphorylation of BTK in human macrophages. Human THP-1 derived macrophages were treated with 1: media only, 2:20 nM Hresistin, 3: 20 nM Hresistin+100 nM MCC950 4: 20 nM Hresistin+Ibrutinib (100 ng/ml), 5: 20 nM Hresistin+300 ng/mL Resistin antibody. Western blot images on p-BTK (Cell Signaling #18805), BTK (SAB4502936) and β-actin (Cell Signaling #3700) were displayed in the right left panel. Quantitative analysis of each protein was also presented. Data represent means+SD (n=6 per each group. *p<0.05, **P<0.01.

FIG. 4A-4B. FIG. 4A. Hresistin stimulate NLRP3 phosphorylation, which was blocked by Ibrutinib, MCC950 and Hresistin ab. Human macrophages were plated at 2×106cells/well in 6 well dishes treated with 1. media only, 2. Hresistin 20 nM, 3. Hresistin 20 nM+MCC950 100 nM, 4. 3. Hresistin 20 nM+Ibrutinib100 ng/ml, 5. Hresistin 20 nM+Hresistin ab (300 ng/ml for 15 minutes. Cells were washed, lysed, and Western blot images on p-Y (Cell Signaling #18805), NLRP3 (SAB4502936) were displayed in the right left panel and representative figure of n=6. FIG. 4B. Hresistin-BTK signaling induces NLRP3 inflammasome activation in human macrophages. Human macrophages were plated at 2×106cells/well in 6 well dishes treated with 1. media only, 2. Ibrutinib (100 ng/ml) 3. Hresistin 20 nM, 4. Hresistin 20 nM+Ibrutinib 100 ng/ml, 5. Hresistin 20 nM+Hresistin ab (300 ng/ml), 6. Combination of Hresistin 20 nM, Ibrutinib 100 ng/ml and Resistin ab (300 ng/ml) for 12 hrs. PBS was used as a control. Cells were washed, lysed, and 100 μg of lysates were assayed for their ability to cleave a fluorescent caspase-1 substrate, YVAD-AFC according to the manufacturer's protocol (cat. no. ab39412, Abcam, Cambridge, MA). Values were normalized to PBS controls. All conditions were run in duplicate wells and two independent experiments were performed. Error bars represent the mean±SD (n=4). **, p<0.01*, p<0.05 compared to PBS.

FIG. 5A-5B. FIG. 5A. mRELM-α upregulates BTK and NLRP3 co-localization in hypoxic mice lungs and NLRP3 and BTK co-localization was significant in RELMahypoxic mice compared to RELMaK/O hypoxic mice. Immunofluorescence images of NLRP3 and BTK in lung tissues of mice kept in either normal of 4 days of hypoxic condition. Lung sections were stained with anti-NLRP3 (ab214185, red) and BTK (, 8547 cell signaling). The images were shown in higher magnification (400×). The arrowheads point to the cells positively stained for BTK and NLRP3 in the sections from mice lungs. Separate channels displayed in the right panels. Representative photograph of n=6 mice per group. FIG. 5B. Human resistin colocalized with BTK and NLRP3 in PH patients. Immunofluorescence images of lung tissue slices from PH patients. Sections were stained with anti-human resistin (green) and co-stained with anti-BTK (red) and anti-NLRP3 (blue) antibodies. The arrowheads point to the cells positively stained for human resistin, BTK and NLRP3 in the sections from PH patients. Separate channels displayed in the right panels. Original magnification: 100×, 200× and 400×.

FIG. 6A-6B. Macrophages are the main source of NLRP3 in mice hypoxic lungs and PH patients lungs. FIG. 6A. Immunofluorescence images of NLRP3 and immune cells markers in lung tissues of mice kept in 4 days of hypoxic condition and PH patients lung sections. Lung sections were stained with anti-NLRP3 (ab214185, red) and/or Mac2 (green, CL8942F Cedarlane), MPO (green, AF3667 R&D Systems), or CD79b (green, ab134147 abcam). The images were shown in lower magnification (100×). Representative photograph of n=6 mice per group and n=3 from human PH patients. FIG. 6B. Human resistin colocalized with NLRP3 in macrophages in PH patients. Immunofluorescence images of lung tissue slices from PH patients. Sections were stained with anti-human resistin (red) and co-stained with anti-Mac2 (green) and anti-NLRP3 (blue) antibodies. The arrowheads point to the cells positively stained for human resistin, BTK and NLRP3 in the sections from PH patients. Separate channels displayed in the right panels. Original magnification: 200×.

FIG. 7A-7E. RELMα activation induced the recruitment of B cells to the lungs from their spleens in hypoxic mice lungs. FIG. 7A. Hresistin/RELM-α recruits B cells to lung from the spleen. B cells were isolated from WT-normoxic and hypoxic mice and/RELM-α-K/O normoxic and hypoxic mice. Number of pan B cells were counted. Data represent means+SEM (n=3). *p<0.05. FIG. 7B-7C. Hresistin activates BTK and HMGB1 in human B cells. human B cell line from ATCC (HCC1739BL) were treated with 10 nM or 20 nM hresistin. After 24 hrs incubation, expression levels of Bruton's tyrosine kinase (BTK) and high mobility group box (HMGB) 1 were quantified using both qPCR (FIG. 7B) and western blot analysis (FIG. 7C). FIG. 7D. Quantitative analysis of data in FIG. 7C. Data represent means+SEM (n=2). *p<0.05. FIG. 7E. Human B cells were treated with 1. media only, 2. Macrophages conditioned media in 24 hrs hypoxia, 3. Macrophages conditioned media treated with Hresistin ab 300 ng/ml, 4. Macrophages conditioned media treated with 300 ng/ml control antibody for 24 hrs.

FIG. 8A-8D. Hresistin induced macrophage-derived mature IL-1β and IL-18 promote HPVSMC proliferation. FIG. 8A. Images of western blots on p-AKT (4060), t-AKT (9272), p-ERK1/2 (4370) and total ERK1/2 (4695) protein levels from Human SMCs (smooth muscle cells) which were starved for 24 hrs and were treated with: 1. media only, 2. Macrophages conditioned media treated with Hresistin 20 nM, 3. Macrophages conditioned media treated with Hresistin 20 nM+10 μM (4.04 ug/mL) MCC950, 4. Macrophages conditioned media treated with Hresistin 20 nM+300 ng/mL Resistin ab, 5. Macrophages conditioned media 120 ng/mL IL-1β antibody, 6. Macrophages conditioned media 120 ng/ml IL-18 antibody, 7. Starved media+5 ng/ml IL-1β protein, 8. Starved media+5 ng/mL IL-18 protein. After 30 minutes cells were collected for western blot analysis. FIG. 8B. Quantitative analysis of data in FIG. 8A. Data represent means+SD (n=6 per each group). FIG. 8C. SMC proliferation by IL-1 B and IL-18 derived from Hresistin treated macrophages. SMC proliferation induced by derived from Hresistin treated macrophages. Human SMCs were starved for 24 hrs and were treated with 1-3. media only (with 0%, 0.5%, 5% FBS), 4-5. Hresistin (20 nM and 3 ug), 6.PDGF 10 ng/ml, 7. Macrophages conditioned media-non-stimulated, 8. Macrophage conditioned media treated with Hresistin 20nMfor 30 mins. After 30 minutes remove medium and add 200 μl of smooth muscle cell basal media+0.5% FBS. 5-bromo-2′-deoxyuridine (BrdU) assay was performed next day. Data represent means+SD (n=3 per each group). *p<0.05. *p<0.05. FIG. 8D. Hresistin regulates MMP driven SMC migration and proliferation through IL-1β and IL-18 derived from Hresistin treated macrophages. Human SMCs were starved for 24 hrs and were treated with 1. media only, 2. Macrophages conditioned media treated with Hresistin 20 nM for 24 hrs, 3. Macrophages conditioned media 120 ng/ml IL-1β antibody, 4. Macrophages conditioned media 120 ng/ml IL-18antibody, 5. Starved media+5 ng/ml IL-1Bprotein, 6. Starved media+5 ng/mL IL-18 protein. After 24 hrs cells were collected for western blot analysis. Images of western blots on MMP1 and MMP3 protein levels. Quantitative analysis of data in FIG. 8A. Data represent means+SD (n=6 per each group). *p<0.05.

FIG. 9A-9F. Potency of lead antibodies for blocking hResistin-stimulated proliferation of human smooth muscle cells (SMCs). FIG. 9A. hResistin dose dependently induced human primary SMC proliferation. Human bronchial SMCs (Lonza) were stimulated with the present inventor's lab-made recombinant hResistin protein for 48 hours. Proliferation was quantified by BrdU ELISA (Roche). Data represent means+SEM (n=6). *p<0.05, **p<0.01. Cells without BrdU labelling served as the background control. Positive control consisted of 20 ng/ml platelet-derived growth factor (PDGF). FIG. 9B and FIG. 9C. Anti-hResistin antibody Ab-b blocked hResistin-induced proliferation of human SMCs. To test antibody blockade, the present inventor incubated 0.1-10 g/mL Ab-b with hResistin recombinant protein for 20 minutes at room temperature before adding the mixture to human bronchial SMCs (FIG. 9B). As measured by BrdU assay, Ab-b dose dependently blocked cell proliferation induced by hResistin. Data represent means+SEM (n=8). ****p<0.0001. In human pulmonary artery SMCs (hPASMCs; FIG. 9C) treated with 3 μg/mL hResistin and no Ab-b, proliferation was significantly elevated but was reversed by the addition of 2 μg/mL Ab-b. PDGF (20 ng/ml) served as a positive control. Data represent means+SEM (n=8). ***p<0.001, ****p<0.0001. FIG. 9D-9F. The other three lead anti-hResistin antibodies Ab-a (FIG. 9D), Ab-c (FIG. 9E), and Ab-d (FIG. 9F) were incubated at the indicated doses with 3 μg/mL recombinant hResistin for 30 minutes at room temperature before being applied to human primary SMCs. BrdU ELISA (Roche) was used to quantify cell proliferation. Data represent means+SEM (n=8). **p<0.01, ***p<0.001, ***p<0.0001.

FIG. 10A-10F. Size exclusion high-performance liquid chromatography (SE-HPLC) analysis of the purified lead antibodies. SE-HPLC was carried out for 1 mg/mL samples on a Zorbax GF-250 9.4 mm ID×25 cm column (Agilent) after Protein A purification and neutralization. The SE-HPLC chromatograms for antibodies Ab-a (FIG. 10A), Ab-b (FIG. 10B), Ab-c (FIG. 10C), Ab-d (FIG. 10D), control IgG1 (FIG. 10E), and the molecular weight standard (marker, FIG. 10F) are shown. AU, arbitrary units.

FIG. 11A-11F. Plasmon resonance binding kinetics of lead antibodies to hResistin. Octet surface plasmon resonance evaluation of the kinetics of anti-hResistin antibodies Ab-a. (FIG. 11A), Ab-b (FIG. 11B), Ab-c (FIG. 11C) and Ab-d (FIG. 11D) binding to recombinant hResistin. Binding experiments were performed on a Biacore 3000. Antibodies were immobilized onto anti-Human-IgG sensors and their binding of 7 dilutions of recombinant hResistin protein were monitored in real time (FIG. 11E). Association or disassociation with the surface causes a shift in wavelength of reflected light. Measuring the shift over time enabled the determination of binding kinetics. From the observed kₒₙ (ka) and k_{off} (kd), equilibrium affinity KD (kd/ka) was determined. 1:1 Curve Fits were applied and Global Fits were calculated and reported (FIG. 11F).

FIG. 12A-12E. Lead antibody binds to an active epitope of hResistin. FIG. 12A. Energy funnel plots were generated from Rosetta SnugDock protocol. Each point represents one candidate docked structure, the x-axis root-mean-squared-deviation of the antibody Ca coordinate relative to the lowest-energy model, and the y-axis is a Rosetta score representing the energy of the interface. Antibody (Ab-b) binding with the two epitope regions residues 50-65 and (left panels) residues 78-93 (right panels) in the hResistin protein. FIG. 12B. Structure of the antibody (Ab-b) with its highlighted complementarity determining region (CDR) loops in the light and heavy chains, as homology modeled using RosettaAntibody. FIG. 12C. Monomer structure of hResistin, predicted using homology modelling from PDB structure 1RFX of mouse-resistin.^{23, 49} FIG. 12D. Docked pose of the antibody Ab-b with hResistin; the epitope region is highlighted in pink. FIG. 12E. Detail of the interaction between the epitope and the CDR loops.

FIG. 13A-13C. Anti-hResistin antibodies bind to rat RELMα and block its induction of human smooth muscle cell (SMC) proliferation. FIG. 13A. Immunoprecipitation analysis of the binding of rat RELMα to human therapeutic antibody candidates. Two micrograms of generated anti-hResistin antibodies, Ab-a, Ab-b, Ab-c, and Ab-d were incubated with 100 ng of lab-made Flag-tagged recombinant rat RELMα protein and Dynabeads® Protein A (Thermo Fisher). The protein-antibody binding was detected by western blotting with anti-Flag antibodies (Sigma). Flag-tagged recombinant rat RELMα protein was loaded as the positive control. FIG. 13B. Rat RELMα protein dose dependently induced human SMC proliferation. Primary human bronchial SMCs were stimulated with lab-made recombinant rat RELMα protein for 48 hours. Then BrdU ELISA was performed to quantify cell proliferation. Data are presented as means+SEM (n=6). **p<0.01. FIG. 13C. The therapeutic antibody candidate Ab-b dose dependently inhibited SMC proliferation induced by rat RELMα. Anti-hResistin antibodies (Ab) were incubated with lab-made recombinant rat RELMα protein for 20 minutes before they were applied to human primary SMCs for 48 hours. BrdU ELISA kits were used to assess proliferation. Data are presented as means+SEM (n=6). *p<0.05, **p<0.01 vs. medium-treated control group.

FIG. 14. A schematic illustration of experimental strategy for developing human antibodies that target hResistin for pulmonary hypertension (PH) treatment. PAT/PET, pulmonary acceleration time/pulmonary ejection time; RV, right ventricle; LV+S, left ventricle plus septum; RVSP, right ventricular systolic pressure.

FIG. 15. Post-stability study analysis of Ab-b after incubation under conditions of low pH, agitation, high temperature or freeze-thawing. Reduced and non-reduced Ab-b were electrophoresed. SDS-PAGE analysis shows a protein species band between 98 kDa and 198 kDa under non-reduced conditions (lanes 2, 4, 6, 8, 10, 12), which is consistent with the estimated molecular weight of the full-length antibody (146 kDa) and comparable to the predominant band seen for the inter-assay control antibody (lane 14). Under reduced conditions (lanes 3, 5, 7, 9, 11, 13) two bands are observed just above 49 kDa and under 28 kDa corresponding to the heavy and light chains respectively and comparable to the results observed for the inter-assay control antibody under reduced conditions (lane 15). The results of the IgG1 control antibody (lanes 14 and 15) are consistent and as expected. It thus indicated that under all conditions tested product quality remained consistent.

FIG. 16A-16H. HPLC analysis of the protein stability of Ab-b. All samples were analyzed by SE-HPLC at 5 mg/ml on a Zorbax GF-250 9.4 mm ID×25 cm column (Agilent) as described in the Methods. Representative chromatography profile obtained for Ab-b under the conditions of control (stored at 5° C., FIG. 16A), high temperature (40° C., 14 days, FIG. 16B), low temperature (5° C., 14 days, FIG. 16C), low pH (3.5, 24 hours, FIG. 16D), agitation (14 days at RT, FIG. 16E), or freeze-thaw (14 days, FIG. 16F). FIG. 16G. Nonimmunized human IgG1 served as an isotype control. FIG. 16H. Monomer proportion and aggregation of Ab-b exposed to different incubation conditions were observed and summarized.

FIG. 17A-17F. cIEF analysis of the protein stability of Ab-b. cIEF data for the Ab-b samples were obtained as described in Methods. Detailed electropherograms were displayed for the Ab-b exposed to low pH (3.5, 24 hours, FIG. 17A), 40° C. (14 days, FIG. 17B), 5° C. (14 days, FIG. 17C), freeze-thaw (14 days, FIG. 17D), or agitation (14 days at RT, FIG. 17E). Results were summarized in FIG. 17F.

FIG. 18A-18D. Comparison of serum resistin levels and receiver operating characteristic (ROC) curves. FIG. 18A. Resistin levels were significantly higher in patients with IPAH (n=808, median [IQR]=6.2 ng/mL [4.63-8.3]) and SSc-PAH (n=313, 8.28 ng/mL [6.18-11.77]) than in controls (n=50, 3.84 ng/ml [2.64-4.78]). P<0.0001, Kruskal-Wallis test. FIG. 18B-18D. The specificity and sensitivity of serum resistin as a predictor for diagnosis of PAH (B: all PAH=1121, AUC=0.847; C: IPAH=808, AUC=0.821; D: SSc-PAH=313, AUC=0.914), all Ps<0.001.

FIG. 19A-19B. The Kaplan Meir mortality analysis of all PAH (n-998) (FIG. 19A) and IPAH (n=722) (FIG. 19B) patients by quartile of resistin level. Group 1, <25th percentile; group 2, 25th-50th percentile; group 3, 50th-75th percentile; group 4 (>75th percentile). Logrank test P=0.015 and 0.012, respectively.

FIG. 20A-20B. RETN single nucleotide polymorphisms (SNPs) are associated with resistin levels in IPAH patients (n=776). FIG. 20A. ENCODE regulation tracks on the RETN region (chromosome 19:7,669,049-7,670,455) with the two SNPs rs3219175 (located in proximal upstream) and rs3745367 (intronic region) highlighted. FIG. 20B. Association between genotypes of two RETN SNPs and resistin level. P=0.0001 and 0.0002, respectively.

FIG. 21A-21D. Evaluation of the predictive models derived from selected predictors and analysis of the importance of each feature in classifying mortality. The AU-ROC curves of the 5 models in the testing set derived from selected parameters excluding (FIG. 21A) or including (FIG. 21B) resistin level and RETN gene SNPs. Mean AUC values and 95% CIs of different prediction models are shown. FIG. 21C-21D. Corresponding histograms describing the relative importance of the top 10 features in the random forest model.

FIG. 22. Illustration of the role of resistin as a genetic and biological marker for predicting PAH severity and adverse outcomes. Abbreviations: PAH, pulmonary arterial hypertension; RV, right ventricle; LV, left ventricle; RETN, gene encodes resistin; SNP, single nucleotide polymorphism; ROC, receiver operating characteristic; AUC, area under the curve; RF, random forest; SVM, support vector machine; MLP, multilayer perceptron; mPAP, mean pulmonary artery pressure; Diastolic_grad, diastolic pulmonary gradient (DPG); REVEAL 2.0, REVEAL 2.0 risk score.

FIG. 23. Illustration showing Resistin regulates priming and activation of the NLRP3 inflammasome.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. Antibodies to Resistin and Resistin-Like Molecule Beta (RELMB)

### A. Definitions

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, gamma-carboxyglutamate, and O-phosphoserine, phosphothreonine.

An “amino acid analog” refers to a compound that has the same basic chemical structure as a naturally occurring amino acid, i.e., a carbon that is bound to a hydrogen, a carboxyl group, an amino group, and an R group (e.g., homoserine, norleucine, methionine sulfoxide, methionine methyl sulfonium), but that contains some alteration not found in a naturally occurring amino acid (e.g., a modified side chain). Amino acids and analogs are well known in the art. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The term “amino acid mimetic” refers to chemical compounds that have a structure that is different from the general chemical structure of an amino acid, but that function in a manner similar to a naturally occurring amino acid. Amino acid analogs may have modified R groups (for example, norleucine) or modified peptide backbones, but retain the same basic chemical structure as a naturally occurring amino acid. In certain embodiments, an amino acid analog is a D-amino acid, a beta-amino acid, or an N-methyl amino acid.

By “antibody” is meant any immunoglobulin polypeptide, or fragment thereof, having immunogen binding ability. As used herein, the terms “antibody fragments”, “fragment”, or “fragment thereof” refer to a portion of an intact antibody. Examples of antibody fragments include, but are not limited to, linear antibodies; single-chain antibody molecules; Fc or Fc′ peptides, Fab and Fab fragments, and multispecific antibodies formed from antibody fragments. In most embodiments, the terms also refer to fragments that bind an antigen of a target molecule (e.g., Resistin) and can be referred to as “antigen-binding fragments.”

The term “conjugate” refers to a complex of two molecules linked together, for example, linked together by a covalent bond. In one embodiment, an antibody is linked to an effector molecule; for example, an antibody that specifically binds to Resistin covalently linked to an effector molecule. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

The terms “conjugating,” “joining,” “bonding,” “labeling” or “linking” refer to making two molecules into one contiguous molecule; for example, linking two polypeptides into one contiguous polypeptide, or covalently attaching an effector molecule or detectable marker radionuclide or other molecule to a polypeptide, such as an scFv. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

“Conservative” amino acid substitutions are those substitutions that do not substantially decrease the binding affinity of an antibody for an antigen (for example, the binding affinity of an antibody for Resistin). For example, a human antibody that specifically binds Resistin can include at most about 1, at most about 2, at most about 5, at most about 10, or at most about 15 conservative substitutions and specifically bind the Resistin polypeptide. The term conservative variation also includes the use of a substituted amino acid in place of an unsubstituted parent amino acid, provided that antibody retains binding affinity for Resistin. Non-conservative substitutions are those that reduce an activity or binding to Resistin.

Conservative amino acid substitution tables providing functionally similar amino acids are well known to one of ordinary skill in the art. The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (I), Lysine ( )
5) Isoleucine (I), Leucine (L), Methionine (M), Valine (V); and
6) Phenylalanine (F), Tyrosine (Y), Tryptophan (W).

An “effector molecule” means a molecule intended to have or produce a desired effect; for example, a desired effect on a cell to which the effector molecule is targeted. Effector molecules include such molecules as polypeptides, radioisotopes and small molecules. Non-limiting examples of effector molecules include toxins, chemotherapeutic agents and anti-angiogenic agents. The skilled artisan will understand that some effector molecules may have or produce more than one desired effect. In one example, an effector molecule is the portion of a chimeric molecule, for example a chimeric molecule that includes a disclosed antibody or fragment thereof, that is intended to have a desired effect on a cell to which the chimeric molecule is targeted.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers.

By “fragment” is meant a portion (e.g., at least about 5, 10, 25, 50, 100, 125, 150, 200, 250, 300, 350, 400, or 500 amino acids or nucleic acids) of a protein or nucleic acid molecule that is substantially identical to a reference protein or nucleic acid and retains at least one biological activity of the reference. In some embodiments the portion retains at least 50%, 75%, or 80%, or more preferably 90%, 95%, or even 99% of the biological activity of the reference protein or nucleic acid described herein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

As used herein, “humanized” forms of non-human (e.g., murine) antibodies are chimeric antibodies that contain minimal sequence, or no sequence, derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues that are not found in the recipient antibody or in the donor antibody. These modifications are generally made to further refine antibody performance. In general, the humanized antibody will comprise substantially all of at least one, and typically two, variable domains, in which all or substantially all of the hypervariable loops correspond to those of a nonhuman immunoglobulin and all or substantially all of the FR residues are those of a human immunoglobulin sequence. The humanized antibody can also comprise at least a portion of an immunoglobulin constant region (Fc), typically that of a human immunoglobulin. Examples of methods used to generate humanized antibodies are described in U.S. Pat. No. 5,225,539.

The term “human antibody” as used herein means an antibody produced by a human or an antibody having an amino acid sequence corresponding to an antibody produced by a human made using any of the techniques known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

“Hybrid antibodies” are immunoglobulin molecules in which pairs of heavy and light chains from antibodies with different antigenic determinant regions are assembled together so that two different epitopes or two different antigens can be recognized and bound by the resulting tetramer.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified. Indeed, the term “purified” does not require the material to be present in a form exhibiting absolute purity, exclusive of the presence of other compounds. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

By “modulation” is meant a change (increase or decrease) in the expression level or biological activity of a gene or polypeptide as detected by standard methods known in the art. As used herein, modulation includes at least about 10% change, 25%, 40%, 50% or a greater change in expression levels or biological activity (e.g., about 75%, 85%, 95% or more).

The term “mimetic” means an agent having a structure that is different from the general chemical structure of a reference agent, but that has at least one biological function of the reference.

The term “neutralizing antibody” refers to an antibody that is able to specifically bind to a target protein in such a way as to inhibit a biological function associated with that target protein. In general, any protein that can perform this type of specific blocking activity is considered a neutralizing protein; neutralizing antibodies are therefore a specific class of neutralizing protein.

The term “nucleic acid” refers to an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases.

Specific examples of some nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Also preferred are oligonucleotides having morpholino backbone structures (Summerton, J. E. and Weller, D. D., U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199:254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂ NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group. Other preferred embodiments may include at least one modified base form. Some specific examples of such modified bases include 2-(amino) adenine, 2-(methylamino) adenine, 2-(imidazolylalkyl) adenine, 2-(aminoalklyamino) adenine, or other heterosubstituted alkyladenines.

The term “operably linked” means that a first polynucleotide is positioned adjacent to a second polynucleotide that directs transcription of the first polynucleotide when appropriate molecules (e.g., transcriptional activator proteins) are bound to the second polynucleotide.

By “recombinant” is meant the product of genetic engineering or chemical synthesis. By “positioned for expression” is meant that the polynucleotide of the present invention (e.g., a DNA molecule) is positioned adjacent to a DNA sequence that directs transcription and translation of the sequence (i.e., facilitates the production of, for example, a recombinant protein of the present invention, or an RNA molecule).

The terms “specifically binds to,” “specific for,” and related grammatical variants refer to that binding which occurs between such paired species as antibody/antigen, aptamer/target, enzyme/substrate, receptor/agonist and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, in certain embodiments, “specific binding” occurs between a paired species where there is interaction between the two which produces a bound complex having the characteristics of, for example, an antibody/antigen. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. In certain embodiments, the term refers to a molecule (e.g., an antibody) that binds to a target (e.g., Resistin) with at least five-fold greater affinity as compared to any non-targets, e.g., at least 10-, 20-, 50-, or 100-fold greater affinity.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e.sup.-3 and e.sup.-100 indicating a closely related sequence.

By “transformed cell” is meant a cell into which (or into an ancestor of which) has been introduced, by means of recombinant DNA techniques, a polynucleotide molecule encoding (as used herein) a protein of the present invention.

### B. Antibodies and Antigen-Binding Fragments Thereof

The present invention provides antibodies to Resistin. In some embodiments, the antibodies are also cross-reactive with Resistin-Like Molecule Beta (RELMB). An “antibody” is a polypeptide ligand including at least the complementarity determining regions (CDRs) of a light chain or heavy chain immunoglobulin variable region which specifically binds an epitope of an antigen or a fragment thereof. Antibodies include intact immunoglobulins and the variants of them well known in the art, such as Fab′, F (ab)′2 fragments, single chain Fv proteins (scFv), and disulfide stabilized Fv proteins (dsFv). A scFv protein is a fusion protein in which a light chain variable region of an antibody and a heavy chain variable region of an antibody are bound by a linker, while in dsFvs, the chains have been mutated to introduce a disulfide bond to stabilize the association of the chains. The term “antibody” also includes genetically engineered forms such as chimeric antibodies (for example, humanized murine antibodies) and heteroconjugate antibodies (such as, bispecific antibodies).

Typically, a naturally occurring immunoglobulin has heavy (H) chains and light (L) chains interconnected by disulfide bonds. There are two types of light chains, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region (the regions are also known as domains). References to “VH” or “VH” refer to the variable region of an immunoglobulin heavy chain, including that of an Fv, scFv, dsFv or Fab. References to “VL” or “VL” refer to the variable region of an immunoglobulin light chain, including that of an Fv, scFv, dsFv or Fab. In combination, the heavy and the light chain variable regions specifically bind the antigen. Light and heavy chain variable regions contain a framework region interrupted by three hypervariable regions, also called complementarity-determining regions or CDRs. The extent of the framework region and CDRs have been defined (see, for example, Kabat et al., (1991) Sequences of Proteins of Immunological Interest, 51 h Edition, U.S. Department of Health and Human Services, Public Health Service, National Institutes of Health, Bethesda, MD (NIH Publication No. 91-3242), which is hereby incorporated by reference). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), and Al-Lazikani et al., (1997) JMB 273,927-948 (“Chothia” numbering scheme). The CDRs of each chain are typically referred to as CDR1, CDR2, and CDR3, numbered sequentially starting from the N-terminus, and are also typically identified by the chain in which the particular CDR is located. Thus, a HCDR1 is the CDR 1 from the variable domain of the heavy chain of the antibody in which it is found, whereas a LCDR 1 is the CDR1 from the variable domain of the light chain of the antibody in which it is found. An antibody that specifically binds an antigen of interest has a specific VH region and VL region sequence, and thus specific CDR sequences. Antibodies with different specificities (due to different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

A single-chain antibody (scFv) is a genetically engineered molecule containing the VH and VL domains of one or more antibody (ies) linked by a suitable polypeptide linker as a genetically fused single chain molecule. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. A chimeric antibody is an antibody that contains one or more regions from one antibody and one or more regions from one or more other antibodies. An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

The antibodies disclosed herein specifically bind only to a defined target (or multiple targets, in the case of a bi-specific antibody). Thus, an antibody that specifically binds to Resistin is an antibody that binds substantially to Resistin, including cells or tissue expressing Resistin, substrate to which the Resistin is attached, or Resistin in a biological specimen. It is, of course, recognized that a certain degree of non-specific interaction may occur between an antibody or conjugate including an antibody (such as an antibody that specifically binds Resistin or conjugate including such antibody) and a non-target (such as a cell that does not express Resistin). Typically, specific binding results in a much stronger association between the antibody and protein or cells bearing the antigen than between the antibody and protein or cells lacking the antigen. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope.

In one embodiment, an antibody that binds Resistin is monoclonal. Alternatively, the Resistin antibody is a polyclonal antibody. The preparation and use of polyclonal antibodies are also known by the skilled artisan. The present invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(abα)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Faba fragments consist of a covalently bound antibody light chain and a portion of the antibody heavy chain, denoted “Fd.” The Fd fragments are the major determinants of antibody specificity (a single Fd fragment may be associated with up to ten different light chains without altering antibody specificity). Isolated Fd fragments retain the ability to specifically bind to immunogenic epitopes.

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated, such as from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different ways using recombinant DNA technology to generate alternative antibodies. In one embodiment, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Furthermore, site-directed or high-density mutagenesis of the variable region can be used to optimize specificity, affinity, etc. of a monoclonal antibody.

In some embodiments, of the present invention the monoclonal antibody against Resistin is a humanized antibody. Humanized antibodies are antibodies that contain minimal sequences from non-human (e.g., murine) antibodies within the variable regions. In practice, humanized antibodies are typically human antibodies with minimum to no non-human sequences. A human antibody is an antibody produced by a human or an antibody having an amino acid sequence corresponding to an antibody produced by a human.

Humanized antibodies can be produced using various techniques known in the art. An antibody can be humanized by substituting the CDR of a human antibody with that of a non-human antibody (e.g., mouse, rat, rabbit, hamster, etc.) having the desired specificity, affinity, and capability (Jones et al., 1986, Nature, 321:522-525; Riechmann et al., 1988, Nature, 332:323-327; Verhoeyen et al., 1988, Science, 239:1534-1536). The humanized antibody can be further modified by the substitution of additional residue either in the Fv framework region and/or within the replaced non-human residues to refine and optimize antibody specificity, affinity, and/or capability.

Human antibodies can be directly prepared using various techniques known in the art. Immortalized human B lymphocytes immunized in vitro or isolated from an immunized individual that produce an antibody directed against a target antigen can be generated (See, for example, Cole et al., Monoclonal Antibodies and Cancer Therapy, Alan R. Liss, p. 77 (1985); Boerner et al., 1991, J. Immunol., 147 (1): 86-95; and U.S. Pat. No. 5,750,373). Also, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Mol. Biol., 227:381; Marks et al., 1991, J. Mol. Biol., 222:581). Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments of the invention, it may be desirable to use an antibody fragment, rather than an intact antibody. Various techniques are known for the production of antibody fragments. Traditionally, these fragments are derived via proteolytic digestion of intact antibodies (for example Morimoto et al., 1993, Journal of Biochemical and Biophysical Methods 24:107-117 and Brennan et al., 1985, Science, 229:81). However, these fragments are now typically produced directly by recombinant host cells as described above. Thus Fab, Fv, and scFv antibody fragments can all be expressed in and secreted from E. coli or other host cells, thus allowing the production of large amounts of these fragments. Alternatively, such antibody fragments can be isolated from the antibody phage libraries discussed above. The antibody fragment can also be linear antibodies as described in U.S. Pat. No. 5,641,870, for example, and can be monospecific or bispecific. Other techniques for the production of antibody fragments will be apparent.

The present invention further embraces variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In particular embodiments, the hResistin therapeutic antibody comprises clone 13, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 73 or a conservative substitution at up to 5 amino acid positions of SEQ ID NO:73, and the light chain variable region comprises SEQ ID NO:77 or a conservative substitution at up to 5 amino acid positions of SEQ ID NO:77. In other embodiments, the hResistin antibody or antigen-binding fragment thereof comprises (a) a heavy chain variable region comprising CDRs 1, 2 and 3 comprising SEQ ID NOS: 74-76, respectively, or a conservative substitution at up to 2 amino acids of one or more of SEQ ID NOS: 74-76, and (b) a light chain variable region comprising CDRs 1, 2, and 3 comprising SEQ ID NOS: 78-80, respectively, or a conservative substitution at up to 2 amino acids of one or more of SEQ ID NOS: 78-80. In further embodiments, the hResistin antibody or antigen-binding fragment thereof comprises a single chain variable fragment (scFv) or antigen-binding fragment thereof that binds Resistin, wherein the scFv comprises SEQ ID NO:72 or a conservative substitution at up to 5 amino acids of SEQ ID NO: 72.

In particular embodiments, the hResistin therapeutic antibody comprises clone 42, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 163 or a conservative substitution at up to 5 amino acid positions of SEQ ID NO:163, and the light chain variable region comprises SEQ ID NO:167 or a conservative substitution at up to 5 amino acid positions of SEQ ID NO:167. In other embodiments, the hResistin antibody or antigen-binding fragment thereof comprises (a) a heavy chain variable region comprising CDRs 1, 2 and 3 comprising SEQ ID NOS: 164-166, respectively, or a conservative substitution at up to 2 amino acids of one or more of SEQ ID NOS: 164-166, and (b) a light chain variable region comprising CDRs 1, 2, and 3 comprising SEQ ID NOS: 168-170, respectively, or a conservative substitution at up to 2 amino acids of one or more of SEQ ID NOS: 168-170. In further embodiments, the hResistin antibody or antigen-binding fragment thereof comprises a single chain variable fragment (scFv) or antigen-binding fragment thereof that binds Resistin, wherein the scFv comprises SEQ ID NO: 162 or a conservative substitution at up to 5 amino acids of SEQ ID NO: 162.

In particular embodiments, the hResistin therapeutic antibody comprises clone 2, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 13 or a conservative substitution at up to 5 amino acid positions of SEQ ID NO:13, and the light chain variable region comprises SEQ ID NO:17 or a conservative substitution at up to 5 amino acid positions of SEQ ID NO:17. In other embodiments, the hResistin antibody or antigen-binding fragment thereof comprises (a) a heavy chain variable region comprising CDRs 1, 2 and 3 comprising SEQ ID NOS: 14-16, respectively, or a conservative substitution at up to 2 amino acids of one or more of SEQ ID NOS: 14-16, and (b) a light chain variable region comprising CDRs 1, 2, and 3 comprising SEQ ID NOS: 18-20, respectively, or a conservative substitution at up to 2 amino acids of one or more of SEQ ID NOS: 18-20. In further embodiments, the hResistin antibody or antigen-binding fragment thereof comprises a single chain variable fragment (scFv) or antigen-binding fragment thereof that binds Resistin, wherein the scFv comprises SEQ ID NO:12 or a conservative substitution at up to 5 amino acids of SEQ ID NO: 12.

In particular embodiments, the hResistin therapeutic antibody comprises clone 11, as described in U.S. Pat. No. 10,822,407. More specifically, the hResistin antibody comprises an anti-Resistin antibody or antigen-binding fragment thereof comprising a heavy chain variable region and a light chain variable region, wherein the heavy chain variable region comprises SEQ ID NO: 63 or a conservative substitution at up to 5 amino acid positions of SEQ ID NO:63, and the light chain variable region comprises SEQ ID NO:67 or a conservative substitution at up to 5 amino acid positions of SEQ ID NO:67. In other embodiments, the hResistin antibody or antigen-binding fragment thereof comprises (a) a heavy chain variable region comprising CDRs 1, 2 and 3 comprising SEQ ID NOS: 64-66, respectively, or a conservative substitution at up to 2 amino acids of one or more of SEQ ID NOS: 64-66, and (b) a light chain variable region comprising CDRs 1, 2, and 3 comprising SEQ ID NOS: 68-70, respectively, or a conservative substitution at up to 2 amino acids of one or more of SEQ ID NOS: 68-70. In further embodiments, the hResistin antibody or antigen-binding fragment thereof comprises a single chain variable fragment (scFv) or antigen-binding fragment thereof that binds Resistin, wherein the scFv comprises SEQ ID NO:62 or a conservative substitution at up to 5 amino acids of SEQ ID NO: 62.

In specific embodiments, the antibody comprises a variable heavy chain comprising SEQ ID NO: 3, SEQ ID NO: 13, SEQ ID NO:23, SEQ ID NO:33, SEQ ID NO:43, SEQ ID NO:53, SEQ ID NO:63, SEQ ID NO:73, SEQ ID NO:83, SEQ ID NO:93, SEQ ID NO: 103, SEQ ID NO: 113, SEQ ID NO: 123, SEQ ID NO: 133, SEQ ID NO: 143, SEQ ID NO: 153, SEQ ID NO: 163, or fragments thereof. In other embodiments, the antibody comprises a variable heavy chain that is at least 90% identical to SEQ ID NO:3, SEQ ID NO:13, SEQ ID NO:23, SEQ ID NO: 33, SEQ ID NO:43, SEQ ID NO:53, SEQ ID NO: 63, SEQ ID NO: 73, SEQ ID NO:83, SEQ ID NO: 93, SEQ ID NO: 103, SEQ ID NO:113, SEQ ID NO: 123, SEQ ID NO: 133, SEQ ID NO: 143, SEQ ID NO: 153, SEQ ID NO: 163, or fragments thereof.

In certain embodiments, the antibody comprises a light chain comprising SEQ ID NO:7, SEQ ID NO:17, SEQ ID NO:27, SEQ ID NO:37, SEQ ID NO:47, SEQ ID NO:57, SEQ ID NO: 67, SEQ ID NO: 77, SEQ ID NO: 87, SEQ ID NO:97, SEQ ID NO: 107, SEQ ID NO:117, SEQ ID NO:127, SEQ ID NO:137, SEQ ID NO: 147, SEQ ID NO: 157, SEQ ID NO: 167, or fragments thereof. Alternatively, the antibody comprises a light chain that is at least 90% identical to SEQ ID NO:7, SEQ ID NO:17, SEQ ID NO:27, SEQ ID NO:37, SEQ ID NO:47, SEQ ID NO:57, SEQ ID NO:67, SEQ ID NO:77, SEQ ID NO:87, SEQ ID NO:97, SEQ ID NO: 107, SEQ ID NO:117, SEQ ID NO: 127, SEQ ID NO: 137, SEQ ID NO: 147, SEQ ID NO: 157, SEQ ID NO:167, or fragments thereof.

The present invention also provides antibodies in which the variable domain of the heavy chain comprises one or more complementarity determining regions (CDRs) selected from the group consisting of SEQ ID NO:4, SEQ ID NO:5, SEQ ID NO:6, SEQ ID NO: 14, SEQ ID NO: 15, SEQ ID NO: 16, SEQ ID NO:24, SEQ ID NO:25, SEQ ID NO:26, SEQ ID NO:34, SEQ ID NO: 35, SEQ ID NO:36, SEQ ID NO:44, SEQ ID NO:45, SEQ ID NO:46, SEQ ID NO:54, SEQ ID NO:55, SEQ ID NO:56, SEQ ID NO:64, SEQ ID NO:65, SEQ ID NO:66, SEQ ID NO: 74, SEQ ID NO: 75, SEQ ID NO:76, SEQ ID NO:4, SEQ ID NO:85, SEQ ID NO:86, SEQ ID NO: 94, SEQ ID NO:95, SEQ ID NO:96, SEQ ID NO: 104, SEQ ID NO: 105, SEQ ID NO: 106, SEQ ID NO: 114, SEQ ID NO: 115, SEQ ID NO: 116, SEQ ID NO: 124, SEQ ID NO: 125, SEQ ID NO:126, SEQ ID NO: 134, SEQ ID NO: 135, SEQ ID NO: 136, SEQ ID NO: 144, SEQ ID NO:145, SEQ ID NO: 146, SEQ ID NO: 154, SEQ ID NO: 155, SEQ ID NO: 156, SEQ ID NO: 164, SEQ ID NO: 165, SEQ ID NO: 166, and fragments thereof. In other embodiments, the variable domain of the heavy chain comprises one or more complementarity determining regions (CDRs) that are at least 90% identical to a CDR selected from the group consisting of SEQ ID NO:4, SEQ ID NO:15, SEQ ID NO:6, SEQ ID NO:14, SEQ ID NO:5, SEQ ID NO:16, SEQ ID NO:24, SEQ ID NO:25, SEQ ID NO:26, SEQ ID NO:34, SEQ ID NO: 35, SEQ ID NO:36, SEQ ID NO:44, SEQ ID NO:45, SEQ ID NO:46, SEQ ID NO:54, SEQ ID NO: 55, SEQ ID NO:56, SEQ ID NO:64, SEQ ID NO:65, SEQ ID NO:66, SEQ ID NO: 74, SEQ ID NO:75, SEQ ID NO:76, SEQ ID NO:4, SEQ ID NO: 85, SEQ ID NO:86, SEQ ID NO: 94, SEQ ID NO:95, SEQ ID NO:96, SEQ ID NO: 104, SEQ ID NO: 105, SEQ ID NO: 106, SEQ ID NO:114, SEQ ID NO: 115, SEQ ID NO: 116, SEQ ID NO: 124, SEQ ID NO: 125, SEQ ID NO: 126, SEQ ID NO:134, SEQ ID NO: 135, SEQ ID NO: 136, SEQ ID NO: 144, SEQ ID NO: 145, SEQ ID NO:146, SEQ ID NO: 154, SEQ ID NO: 155, SEQ ID NO: 156, SEQ ID NO: 164, SEQ ID NO:165, SEQ ID NO: 166, and fragments thereof.

The present invention also provides antibodies in which the variable domain of the light chain comprises one or more CDRs selected from the group consisting of SEQ ID NO:8, SEQ ID NO: 9, SEQ ID NO: 10, SEQ ID NO: 18, SEQ ID NO: 19, SEQ ID NO:20, SEQ ID NO:28, SEQ ID NO: 29, SEQ ID NO:30, SEQ ID NO:38, SEQ ID NO:39, SEQ ID NO:40, SEQ ID NO:48, SEQ ID NO:49, SEQ ID NO:50, SEQ ID NO:58, SEQ ID NO:59, SEQ ID NO:60, SEQ ID NO: 68, SEQ ID NO: 69, SEQ ID NO: 70, SEQ ID NO: 78, SEQ ID NO: 79, SEQ ID NO:80, SEQ ID NO: 88, SEQ ID NO:89, SEQ ID NO:90, SEQ ID NO:98, SEQ ID NO:99, SEQ ID NO: 100, SEQ ID NO:108, SEQ ID NO: 109, SEQ ID NO:110, SEQ ID NO: 118, SEQ ID NO:119, SEQ ID NO: 120, SEQ ID NO:128, SEQ ID NO: 129, SEQ ID NO: 130, SEQ ID NO: 138, SEQ ID NO: 139, SEQ ID NO: 140, SEQ ID NO: 148, SEQ ID NO: 149, SEQ ID NO: 150, SEQ ID NO: 158, SEQ ID NO: 159, SEQ ID NO: 160, SEQ ID NO: 168, SEQ ID NO: 169, SEQ ID NO: 170, and fragments thereof. In alternative embodiments, the variable domain of the light chain comprises one or more CDRs that are at least 90% identical to a CDR selected from the group consisting of SEQ ID NO:8, SEQ ID NO:9, SEQ ID NO: 10, SEQ ID NO: 18, SEQ ID NO: 19, SEQ ID NO:20, SEQ ID NO:28, SEQ ID NO:29, SEQ ID NO:30, SEQ ID NO:38, SEQ ID NO: 39, SEQ ID NO:40, SEQ ID NO:48, SEQ ID NO:49, SEQ ID NO:50, SEQ ID NO:58, SEQ ID NO:59, SEQ ID NO:60, SEQ ID NO:68, SEQ ID NO:69, SEQ ID NO:70, SEQ ID NO: 78, SEQ ID NO: 79, SEQ ID NO:80, SEQ ID NO: 88, SEQ ID NO: 89, SEQ ID NO:90, SEQ ID NO: 98, SEQ ID NO:99, SEQ ID NO: 100, SEQ ID NO: 108, SEQ ID NO: 109, SEQ ID NO: 110, SEQ ID NO:118, SEQ ID NO: 119, SEQ ID NO: 120, SEQ ID NO: 128, SEQ ID NO: 129, SEQ ID NO:130, SEQ ID NO: 138, SEQ ID NO: 139, SEQ ID NO: 140, SEQ ID NO: 148, SEQ ID NO:149, SEQ ID NO: 150, SEQ ID NO: 158, SEQ ID NO: 159, SEQ ID NO: 160, SEQ ID NO: 168, SEQ ID NO: 169, SEQ ID NO: 170, and fragments thereof.

In specific embodiments, the present invention provides a scfv that binds human Resistin, wherein the scfv is encoded by SEQ ID NO:1, SEQ ID NO: 11, SEQ ID NO:21, SEQ ID NO:31, SEQ ID NO:41, SEQ ID NO:51, SEQ ID NO:61, SEQ ID NO:71, SEQ ID NO:81, SEQ ID NO: 91, SEQ ID NO: 101, SEQ ID NO: 111, SEQ ID NO:121, SEQ ID NO: 131, SEQ ID NO: 141, SEQ ID NO:151, SEQ ID NO:161 or fragments thereof. In other embodiments, the scfv is encoded by nucleotide sequence that is at least 90% identical to SEQ ID NO:1, SEQ ID NO:11, SEQ ID NO:21, SEQ ID NO:31, SEQ ID NO:41, SEQ ID NO:51, SEQ ID NO:61, SEQ ID NO: 71, SEQ ID NO:81, SEQ ID NO:91, SEQ ID NO: 101, SEQ ID NO: 111, SEQ ID NO: 121, SEQ ID NO:131, SEQ ID NO:141, SEQ ID NO: 151, SEQ ID NO: 161 or fragments thereof.

The present invention also provides a scfv that binds human Resistin, wherein the scfv comprises SEQ ID NO:2, SEQ ID NO:12, SEQ ID NO:22, SEQ ID NO:32, SEQ ID NO:42, SEQ ID NO:52, SEQ ID NO:62, SEQ ID NO:72, SEQ ID NO:82, SEQ ID NO:92, SEQ ID NO: 102, SEQ ID NO:112, SEQ ID NO: 122, SEQ ID NO: 132, SEQ ID NO: 142, SEQ ID NO: 152, SEQ ID NO:162 or fragments thereof. In alternative embodiments, the scfv comprises an amino acid sequence that is at least 90% identical to SEQ ID NO:2, SEQ ID NO:12, SEQ ID NO: 22, SEQ ID NO:32, SEQ ID NO:42, SEQ ID NO:52, SEQ ID NO:62, SEQ ID NO:72, SEQ ID NO: 82, SEQ ID NO:92, SEQ ID NO: 102, SEQ ID NO:112, SEQ ID NO: 122, SEQ ID NO: 132, SEQ ID NO:142, SEQ ID NO:152, SEQ ID NO: 162 or fragments thereof.

The antibodies of the present invention can further comprise a constant domain comprising SEQ ID NO: 172, SEQ ID NO:174 or a fragment thereof. In other embodiments, the antibodies can further comprise a constant domain that is at least 90% identical to SEQ ID NO: 172, SEQ ID NO:174 or a fragment thereof.

In specific embodiments, the present invention also provides a Resistin antibody comprising a heavy chain selected from the group consisting of SEQ ID NO:176, SEQ ID NO: 180, SEQ ID NO:184, SEQ ID NO: 188, SEQ ID NO: 192, SEQ ID NO: 196, SEQ ID NO: 200, SEQ ID NO:204, SEQ ID NO:208, SEQ ID NO:212, SEQ ID NO:216, SEQ ID NO: 220, SEQ ID NO:224, SEQ ID NO:228, SEQ ID NO:232, SEQ ID NO:236, and SEQ ID NO: 240. In additional embodiments, a Resistin antibody comprises a light chain selected from the group consisting of SEQ ID NO: 178, SEQ ID NO:182, SEQ ID NO: 186, SEQ ID NO: 190, SEQ ID NO:194, SEQ ID NO:198, SEQ ID NO:202, SEQ ID NO:206, SEQ ID NO:210, SEQ ID NO: 214, SEQ ID NO:218, SEQ ID NO:222, SEQ ID NO:226, SEQ ID NO:230, SEQ ID NO: 234, SEQ ID NO:238, and SEQ ID NO: 242. In further embodiments, the present invention provides a Resistin antibody comprising (a) a heavy chain selected from the group consisting of SEQ ID NO:176, SEQ ID NO: 180, SEQ ID NO: 184, SEQ ID NO: 188, SEQ ID NO: 192, SEQ ID NO: 196, SEQ ID NO:200, SEQ ID NO:204, SEQ ID NO:208, SEQ ID NO:212, SEQ ID NO: 216, SEQ ID NO:220, SEQ ID NO:224, SEQ ID NO:228, SEQ ID NO:232, SEQ ID NO: 236, and SEQ ID NO:240 and (b) a light chain selected from the group consisting of SEQ ID NO: 178, SEQ ID NO:182, SEQ ID NO: 186, SEQ ID NO: 190, SEQ ID NO: 194, SEQ ID NO: 198, SEQ ID NO:202, SEQ ID NO:206, SEQ ID NO:210, SEQ ID NO:214, SEQ ID NO: 218, SEQ ID NO:222, SEQ ID NO:226, SEQ ID NO:230, SEQ ID NO:234, SEQ ID NO: 238, and SEQ ID NO: 242.

In several embodiments, the present invention provides Resistin antibodies that are also cross-reactive with Resistin Like Molecule Beta (RELMB). In other embodiments, Resistin scfv are also cross-reactive with RELMB. In further embodiments, the antibodies and/or fragments thereof are recombinant.

II. RNA Interference Compositions for Targeting Resistin mRNA

In one aspect of the present invention, the expression of Resistin may be inhibited by the use of RNA interference techniques (RNAi). RNAi is a remarkably efficient process whereby double-stranded RNA (dsRNA) induces the sequence-specific degradation of homologous mRNA in animals and plant cells. RNAi can be triggered, for example, by nucleotide (nt) duplexes of small interfering RNA (siRNA), micro-RNAs (miRNA), functional small-hairpin RNA (shRNA), or other dsRNAs which are expressed in-vivo using DNA templates with RNA polymerase III promoters.

### A. Definitions

The terms “polynucleotide”, “oligonucleotide”, “nucleotide sequence” or “nucleic acid molecule” are used broadly herein to mean a sequence of two or more deoxyribonucleotides or ribonucleotides that are linked together by a phosphodiester bond. As such, the terms include RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the terms as used herein include naturally occurring nucleic acid molecules, which can be isolated from a cell, as well as synthetic polynucleotides, which can be prepared, for example, by methods of chemical synthesis or by enzymatic methods such as by the polymerase chain reaction (PCR). It should be recognized that the different terms are used only for convenience of discussion so as to distinguish, for example, different components of a composition.

As used herein, the term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi inhibits the gene by compromising the function of a target RNA, completely or partially. Both plants and animals mediate RNAi by the RNA-induced silencing complex (RISC); a sequence-specific, multicomponent nuclease that destroys messenger RNAs homologous to the silencing trigger. RISC is known to contain short RNAs derived from the double-stranded RNA trigger. The short RNA sequences are homologous to the target gene that is being suppressed.

As used herein, the term “shRNA” or “short hairpin RNA” refers to a sequence of ribonucleotides comprising a single-stranded RNA polymer that makes a tight hairpin turn on itself to provide a “double-stranded” or duplexed region. shRNA can be used to silence gene expression via RNA interference. shRNA hairpin is cleaved into short interfering RNAs (siRNA) by the cellular machinery and then bound to the RNA-induced silencing complex (RISC). The complex inhibits RNA as a consequence of the complexed siRNA hybridizing to and cleaving RNAs that match the siRNA that is bound thereto.

As used herein, the term “siRNA” refers to a short interfering RNA. The terms “small interfering RNA” and “siRNA” refer to short interfering RNA or silencing RNA, which are a class of short double-stranded RNA molecules that play a variety of biological roles. Most notably, siRNA is involved in the RNA interference (RNAi) pathway where the siRNA interferes with the expression of a specific gene. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. The strand complementary to a target RNA molecule is the “antisense strand”; the strand homologous to the target RNA molecule is the “sense strand”, and is also complementary to the siRNA antisense strand. siRNAs may also contain additional sequences; non-limiting examples of such sequences include linking sequences, or loops, as well as stem and other folded structures.

As noted, the term “antisense” refers to a polynucleotide or oligonucleotide molecule that is substantially complementary or 100% complementary to a particular polynucleotide or oligonucleotide molecule (RNA or DNA), i.e., a “sense” strand, or portion thereof. For example, an antisense molecule may be complementary in whole or in part to a molecule of messenger RNA, miRNA, pRNA, IRNA, IRNA of hnRNA, or a sequence of DNA that is either coding or non-coding.

Polynucleotides of the present invention may be of any suitable length. For example, one of skill in the art would understand what lengths are suitable for RNAi compositions/molecules to be used to regulate gene expression. Such molecules are typically from about 5 to 100, 5 to 50, 5 to 45, 5 to 40, 5 to 35, 5 to 30, 5 to 25, 5 to 20, or 10 to 20 nucleotides in length. For example the molecule may be about 5, 10, 15, 16, 17, 18, 19, 20, 21, 22, 23, 24, 25, 26, 27, 28, 29, 30, 31, 32, 33, 34, 35, 40, 45 or 50 nucleotides in length. Such polynucleotides may include from at least about 15 to more than about 120 nucleotides, including at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, at least about 100 nucleotides, at least about 110 nucleotides, at least about 120 nucleotides or greater than 120 nucleotides.

As used herein, the terms “complementary” or “complement” refer to a nucleic acid comprising a sequence of consecutive nucleobases or semi consecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In one embodiment “% identity” represents the number of amino acids or nucleotides which are identical at corresponding positions in two sequences of a protein or nucleic acids, respectively. For example, two amino acid sequences each having 100 residues will have 95% identity when 95 of the amino acids at corresponding positions are the same. Similarly, two nucleic acid sequences each having 100 bases will have 95% identity when 95 of the bases at corresponding positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448. Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3:266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

### B. Modifications

In certain embodiments, oligonucleotides of the present invention re synthesized using one or more modified nucleotides. As used herein, the terms “modified” and “modification” when used in the context of the constituents of a nucleotide monomer, i.e., sugar, nucleobase and intemnucleoside linkage (backbone), refer to non-natural changes to the chemical structure of these naturally occurring constituents or the substitutions of these constituents with non-naturally occurring ones, i.e., mimetics. For example, the “unmodified” or “naturally occurring” sugar ribose (of RNA) can be modified by replacing the hydrogen at the 2′-position of ribose with a methyl group. Similarly, the naturally occurring intemucleoside linkage of nucleic acids is a 3′ to 5′ phosphodiester linkage that can be modified, in one embodiment, by replacing one of the non-bridging oxygen atoms of the phosphate linker with a sulfur atom to create a phosphorothioate linkage. Modified oligonucleotides are structurally distinguishable, but functionally interchangeable with naturally occurring or synthetic unmodified oligonucleotides and usually have enhanced properties such as increased resistance to degradation by exonucleases and endonucleases, or increased binding affinity.

As noted above, in certain embodiments, modifications to the oligonucleotides of the present invention encompass substitutions or changes in internucleoside linkages, sugar moieties, or nucleobases. Where used herein in reference to an oligonucleotide, the term “non-natural” or “unnatural” refers to an oligonucleotide which comprises at least one modification in an internucleoside linkage, a sugar, and/or a nucleobase thereof, wherein such modified intemucleoside linkage, modified sugar, and/or modified nucleobase is not found naturally in DNA or RNA (unless specifically defined otherwise herein)

Non-naturally occurring intemucleoside linkages of the oligonucleotides of the present invention include those that contain a phosphorus atom and also those that do not contain a phosphorus atom. Numerous phosphoms-containing modified oligonucleotide backbones are known in the art and may be used in the oligonucleotides of the present invention. Examples of phosphoms-containing intemucleoside linkages of non-natural (modified) oligonucleotide backbones which may occur in the presently disclosed oligonucleotides include, but are not limited to, phosphorothioate, phosphorodithioate, phosphoramidite, phosphorodiamidate, morpholino, phosphotriester, aminoalkylphosphotriester, phosphonate, chiral phosphorothioates, methyl and other alkyl phosphonates including 3′-alkylene phosphonate, 5′-alkylene phosphonate and chiral phosphonate, phosphinate, phosphoramidates including 3′-amino phosphoramidate and aminoalkylphosphoramidate, thionophosphoramidate, thionoalkylphosphonate, thionoalkylphosphotriester, selenophosphates and boranophosphates having normal 3′-5′ linkages, 2′-5′ linked analogs of these, and those having inverted polarity wherein one or more intemucleotide linkages is a 3′ to 3′, 5′ to 5′ or 2′ to 2′ linkage, and oligonucleotides having inverted polarity comprise a single 3′ to 3′ linkage at the 3′-most intemucleotide linkage i.e., a single inverted nucleoside residue which may be abasic (the nucleobase is missing or has a hydroxyl group in place thereof) linkages. Examples of U.S. patents that teach the preparation of such phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321, 131; 5,399,676; 5,405,939; 5,453,496; 5,455,233; 5,466,677; 5,476,925; 5,519,126; 5,536,821; 5,541,306; 5,550, 111; 5,563,253; 5,571,799; 5,587,361; 5,194,599; 5,565,555; 5,527,899; 5,721,218; 5,672,697 and 5,625,050.

As noted above, in some embodiments, the intemucleoside linkages are without phosphorus atoms and may instead comprise short chain alkyl or cycloalkyl intemucleoside linkages, mixed heteroatom and alkyl or cycloalkyl intemucleoside linkages, or one or more short chain heteroatomic or heterocyclic intemucleoside linkages. In further embodiments, the non-naturally occurring intemucleoside linkages are uncharged and in others, the linkages are achiral. In some embodiments, the non-naturally occurring intemucleoside linkages are uncharged and achiral, such as peptide nucleic acids (PNAs).

It is understood that the sequence set forth in each sequence or SEQ ID NO contained herein is independent of any modification to sugar moieties, intemucleoside linkages, or nucleobases of the sequence, unless otherwise specified. As such, oligonucleotides of the present invention may be defined by a complementary correspondence to a sequence or SEQ ID NO disclosed herein, or segment thereof, and may comprise, independently, one or more modifications to a sugar moiety, an intemucleoside linkage, or a nucleobase. Other embodiments of oligonucleotide backbones include siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CPU component parts.

In certain oligonucleotides of the present invention, both the sugar moiety and the intemuclecoside linkage, i.e., the backbone, of the nucleotide units are replaced with non-natural groups. One such oligomeric compound is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. As noted elsewhere herein, the oligonucleotide can be further modified so as to be conjugated to an organic moiety such as a biogenic molecule that is selected to improve stability, distribution and/or cellular uptake of the oligonucleotide, e.g., cholesterol, forming the nucleic acid compound of the present invention. Such an organic moiety can be attached, e.g., to the 3′ or 5′ end of the oligonucleotide, and/or at the 2′ position of the sugar moiety of a nucleotide of the oligonucleotide, such as the 2′ ribose position.

The nucleic acid compound can further be in isolated form or can be part of a pharmaceutical composition, such as a pharmaceutical composition formulated for parental administration. The pharmaceutical compositions can contain one or more nucleic acid compounds, and in some embodiments can contain two or more inhibitory nucleic acid compounds, each one directed to a different target gene.

### C. Delivery

10The RNAi composition or oligonucleotide composition can be delivered in any of a variety of forms, including in liposomes and via expression vectors. The composition can be endogenously expressed from transcription units inserted into DNA or RNA vectors. The recombinant vectors can be DNA plasmids or viral vectors for example. Viral vectors suitable for producing the oligonucleotide composition capable of reducing Resistin expression or activity can be constructed based on, but not limited to, adeno-associated vims, retrovirus, lentivirus, adenovirus, or alphavirus. The recombinant vectors which contain a nucleic acid for expressing the oligonucleotide composition can be delivered as described above and can persist in target cells. Alternatively, viral vectors can be used that provide for transient expression of the oligonucleotides. Such vectors can be repeatedly administered as necessary. The delivery vehicles (vectors) for the oligonucleotide composition optionally comprise an expression construct which includes an enhancer sequence, a promoter sequence, and other sequences necessary for expression of the products of the Resistin oligonucleotide sequence. In a specific embodiment, the promoter is cell-specific. The term “cell-specific” means that the particular promoter selected for the recombinant vector can direct expression of the selected transgene only in a particular cell type.

Specific vectors which may be used include, but are not limited to, adeno-associated virus vectors, an attenuated or gutless adenoviral vectors, lentiviral vectors, retroviral vectors, herpes virus vectors, and sindbis virus vectors, papilloma virus vectors, as well as plasmids or synthetic (non-viral) vectors, and/or nanoparticles. The vectors may be either monocistronic, bicistronic, or multicistronic. A recombinant vector (e.g., lenti-, AAV) sequence can be packaged as a “particle” for subsequent infection (transduction) of a cell, ex vivo, in vitro or in vivo. Where a recombinant vector sequence is encapsulated or packaged into an AAV particle, the particle can also be referred to as a “rAAV.” Such particles include proteins that encapsulate or package the vector genome. Particular examples include viral envelope proteins, and in the case of AAV, capsid proteins.

Any suitable route of administration of the oligonucleotide-containing vector may be employed. For example, parenteral (subcutaneous, subretinal, suprachoroidal, intramuscular, intravenous, transdermal) and like forms of administration may be employed. Dosage formulations include injections, implants, or other known and effective gene therapy delivery methods.

Delivery of the oligonucleotide-expressing vectors can be systemic, such as by intravenous or intra-muscular administration, direct administration to a site, by administration to target cells ex-planted from a subject followed by reintroduction into the subject, or by any other means that would allow for introduction into the desired target cell. The therapeutic and/or pharmaceutical compositions, in non-limiting embodiments, contain viral particles per dose in a range of, for example, from about 104 to about 1011 particles, from about 105 to about 1010 particles, or from about 106 to about 109 particles. In the context of AAV vectors, vector genomes are provided in in a range of, for example, from about 104 to about 1014 vector genomes, from about 105 to about 1013 vector genomes, from about 106 to about 1013 vector genomes, from about 107 to about 1013 vector genomes, from about 108 to about 1013 vector genomes, or from about 109 to about 1013 vector genomes.

Because nucleases that cleave the phosphodiester linkages are expressed in almost every cell, unmodified nucleic acid molecules such as the inhibitory oligonucleotide compositions of the present invention may be modified to resist degradation, as described above for example. Other molecules may be conjugated to the oligonucleotides to improve their ability to resist degradation, target certain cells, or to cross barriers like cell membranes or the blood brain barrier. Examples of such molecules include lipids such as, but not limited to, stearic acid, palmitic acid, docosanoic acid, docosahexanoic acid, docosahexaenoic acid, cholesterol, tocopherol, and other C12-C22 saturated or unsaturated fatty acids; peptides such as but not limited to, cell-penetrating peptides (CPPs) such as penetratin, HIV-1 Tat peptides, pVEC-Cadherin 615-634, polyarginines (6-12), and transportan, linear and cyclic RGD-containing peptides, and SPACE peptide; receptor-specific ligands; aptamers (synthetic oligoribonucleotides); antibodies or antibody fragments; CpG-containing oligonucleotides; polyamines, such as spermine and spermidine; polymers such as dendrimers and polyethylene glycols (e.g., PEG 0.6 kDa-5,000 kDa); and saccharides such as N-acetylgalactosamine (GalNAc) and cyclodextrins. The molecule may be conjugated to the oligonucleotide composition by any suitable means, such as via linker or a cleavable bond such as but not limited to disulfide, thioether, pH sensitive (e.g., hydrazone or carboxymethylmaleic anhydride), or ethylene glycol.

In particular embodiments, the oligonucleotides or nucleic acid compositions of the present invention may be delivered in the form of nanoparticles and microparticles which encapsulate the nucleic acid compounds within liposomes of cationic lipids or within PEG, for example. These delivery systems can enhance intracellular delivery either by protecting the nucleic acid compound from nuclease degradation and/or by promoting absorptive endocytosis. Further, in particular embodiments, the addition of dioleylphosphatidylethanolamine to liposome delivery systems results in the destabilization of endosomal membranes and promotion of release of the oligonucleotide after endocytosis.

The nucleic acid compounds can be administered to cells by a variety of other methods known to those of skill in the art, including, but not limited to, ionophoresis, or by incorporation into other vehicles, such as hydrogels, cyclodextrins, biodegradable nanocapsules, and bioadhesive microspheres, or by proteinaceous vectors. In one example, the nucleic acid compounds can be delivered via the nanoparticle system shown in U.S. Patent Application Publication 2019/0255088. The liposomes may comprise amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323.

In certain embodiments, the nanoparticles which contain the nucleic acid compounds of the present invention may comprise a pharmaceutically acceptable carrier such as, but not limited to, poly(ethylene-co-vinyl acetate), PVA, partially hydrolyzed poly(ethylene-co-vinyl acetate), poly(ethylene-co-vinyl acetate-co-vinyl alcohol), a cross-linked poly/ethylene-co-vinyl acetate), a cross-linked partially hydrolyzed poly(ethylene-co-vinyl acetate), a cross-linked poly(ethylene-co-vinyl acetate-co-vinyl alcohol), poly-D,L-lactic acid, poly-L-lactic acid, polyglycolic acid, PGA, copolymers of lactic acid and glycolic acid, polycaprolactone, polyvalerolactone, poly(anhydrides), copolymers of polycaprolactone with polyethylene glycol, copolymers of polylactic acid with polyethylene glycol, polyethylene glycol; fibrin, Gelfoam™ (which is a water-insoluble, off-white, nonelastic, porous, pliable gel foam prepared from purified gelatin and water for injection), and combinations and blends thereof. Copolymers can comprise from about 1% to about 99% by weight of a first monomer unit such as ethylene oxide and from 99% to about 1% by weight of a second monomer unit such as propylene oxide. Blends of a first polymer such as gelatin and a second polymer such as poly-L-lactic acid or polyglycolic acid can comprise from about 1% to about 99% by weight of the first polymer and from about 99% to about 1% of the second polymer.

The nucleic acid compositions can be delivered directly by systemic administration such as using oral formulations or stereotactic injection into prostate or prostate tumor, typically in saline with chemical modifications to enable uptake, or other methods described elsewhere herein. In certain embodiments, such as when the oligonucleotide of the nucleic acid compound has a phosphorothioate backbone, the oligonucleotide binds to serum proteins, slowing excretion by the kidney. The aromatic nucleobases also interact with other hydrophobic molecules in serum and on cell surfaces. In certain embodiments, siRNA delivery systems involve complexing the RNA with cationic and neutral lipids, although encouraging results have also been obtained using peptide transduction domains and cationic polymers. Including PEGylated lipids in the formulation prolongs the circulating half-life of the particles.

As noted, one type of optimization of single-stranded DNA or RNA oligonucleotides is the use of chemical modifications to increase the nuclease resistance such as the introduction of phosphorothioate (“PS”) linkages in place of the phosphodiester bond. This modification improves protection from digestion by nucleases. PS linkages also improved binding to serum proteins in vivo, increasing half-life and permitting greater delivery of active compound to tissues. Chemical modifications to subunits of the nucleotides can also improve potency and selectivity by increasing binding affinity of oligonucleotides for their complementary sequences. Examples of such modifications to the nucleoside sugars include 2′-O-methyl(2′-O-Me), 2′-fluoro (2′-F), and 2′-O-methoxyethyl(2′-MOE) RNA, and others as discussed elsewhere herein. Even more affinity can be gained using oligonucleotides modified with locked nucleic acid (LNA), which contains a methylene bridge between the 2′ and 4′ position of the ribose. This bridge “locks” the ribose ring in a conformation that is ideal for binding, leading to high affinity for complementary sequences. Related bridged nucleic acid (BNA) compounds have been developed and share these favorable properties. Their high affinity has permitted the development of far shorter oligonucleotides than previously thought possible which nonetheless retain high potency. The chemistry for introducing 2′-O-Me, 2′-MOE, 2′-F, or LNA into oligonucleotides is compatible with DNA or RNA synthesis, allowing chimeras with DNA or RNA bases to be easily obtained. This compatibility allows the properties of chemically modified oligonucleotides to be fine-tuned for specific applications, which is a major advantage for development that makes LNAs and other BNAs convenient tools for many applications.

### D. Dendrimers

In particular embodiments, the therapeutic agents can be complexed, conjugated, encapsulated or otherwise associated with a dendrimer. Dendrimers are three-dimensional, hyperbranched, monodispersed, globular and polyvalent macromolecules including surface end groups. Due to their unique structural and physical features, dendrimers have shown unprecedented potential as nano-carriers for various biomedical applications including targeted drug/gene delivery, imaging and diagnosis. See U.S. Pat. Nos. 10,918,820 and 10,369,124, as well as U.S. Patent Application Publication No. 20220080056, No. 20220071923, No. 20210353823, No. 20210252153, No. 20200171200, No. 20200022938, No. 20190142964, No. 20170232120, No. 20170173172, No. 20170119899, No.20170119897, and No. 20170043027.

The term “dendrimer” includes, but is not limited to, a molecular architecture with an interior core and layers (or “generations”) of repeating units which are attached to and extend from this interior core, each layer having one or more branching points, and an exterior surface of terminal groups attached to the outermost generation. In some embodiments, dendrimers have regular dendrimeric or “starburst” molecular structures.

Generally, dendrimers have a diameter from about 1 nm up to about 50 nm, more preferably from about 1 nm to about 20 nm, from about 1 nm to about 10 nm, or from about 1 nm to about 5 nm. In some embodiments, the diameter is between about 1 nm to about 2 nm. In particular embodiments, the dendrimers have a diameter effective to cross the blood brain barrier (“BBB”) and to be retained in target cells for a prolonged period of time.

Exemplary dendrimers include, but are not limited to, polyamidoamine (PAMAM), polyester, polylysine, polypropylamine (POPAM), poly(propylene imine) (PPI), iptycene, aliphatic poly(ether), and/or aromatic polyether dendrimers. The dendrimers can have carboxylic, amine and/or hydroxyl terminations. Dendrimers can be any generation including, but not limited to, generation 1, generation 2, generation 3, generation 4, generation 5, generation 6, generation 7, generation 8, generation 9, or generation 10. In some embodiments, dendrimers are PAMAM dendrimers used as a platform and modified with surface groups for increased number of hydroxyl groups.

In particular embodiments, the dendrimers include a plurality of hydroxyl groups. Some exemplary high-density hydroxyl groups-containing dendrimers include commercially available polyester dendritic polymer such as hyperbranched 2,2-Bis(hydroxyl-methyl) propionic acid polyester polymer (for example, hyperbranched bis-MPA polyester-64-hydroxyl, generation 4), dendritic polyglycerols. In some embodiments, the high-density hydroxyl groups-containing dendrimers are oligo ethylene glycol (OEG)-like dendrimers.

Each dendrimer of a dendrimer complex may be of similar or different chemical nature than the other dendrimers (e.g., the first dendrimer may include a PAMAM dendrimer, while the second dendrimer may include a POPAM dendrimer). In some embodiments, the first or second dendrimer may further include an additional agent. The multiarm PEG polymer includes a polyethylene glycol having at least two branches bearing sulfhydryl or thiopyridine terminal groups; however, PEG polymers bearing other terminal groups such as succinimidyl or maleimide terminations can be used. The PEG polymers in the molecular weight range of 10 kDa to 80 kDa can be used.

The molecular weight of the dendrimers can be varied to prepare polymeric nanoparticles that form particles having properties, such as drug release rate, optimized for specific applications. The dendrimers can have a molecular weight of between about 150 Da and 1 MDa. In certain embodiments, the polymer has a molecular weight of between about 500 Da and about 100 kDa, more preferably between about 1 kDa and about 50 kDa, most preferably between about 1 kDa and about 20 kDa.

### III. Methods of Treatment

In another aspect, the present invention provides methods for using the antibodies described herein. In particular embodiments, the present invention provides a method for treating a disease, disorder or condition mediated by human Resistin in a patient comprising the step of administering to the patient an anti-Resistin antibody or antigen-binding fragment thereof described herein.

### A. Definitions

By “subject” or “patient” is meant a mammal, including, but not limited to, a human or non-human mammal, such as a bovine, equine, canine, ovine, or feline. In particular embodiments, a subject or patient is a human subject or patient.

By “an effective amount” is meant the amount of a required compound to ameliorate the symptoms of a disease relative to an untreated patient. The effective amount of active compound(s) used to practice the present invention for therapeutic treatment of an NLRP3 inflammasome mediated disease varies depending upon the manner of administration, the age, body weight, and general health of the subject. Ultimately, the attending physician or veterinarian will decide the appropriate amount and dosage regimen. Such amount is referred to as an “effective” amount.

As used herein, the terms “treat”, “treatment”, “treating”, or “amelioration” when used in reference to a disease, disorder or medical condition, refer to both therapeutic treatment and prophylactic or preventative measures, wherein the object is to reverse, alleviate, ameliorate, inhibit, lessen, slow down or stop the progression or severity of a symptom, a condition, a disease, or a disorder. The term “treating” includes reducing or alleviating at least one adverse effect or symptom of a condition, a disease, or a disorder. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition, disease, or disorder even if the treatment is ultimately unsuccessful. Those in need of treatment include those already with the condition, disease, or disorder as well as those prone to have the condition, disease, or disorder or those in whom the condition, disease, or disorder is to be prevented.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement an agent or a treatment as disclosed herein into a subject by a method or route which results in at least partial localization of the agent or treatment at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

The term “disease severity” characterizes the impact that a disease process has on the utilization of resources, comorbidities, and mortality. The disease severity reflects the degree of illness and risk of disease manifested by patients, based either on clinical data from the medical records or on hospital discharge/billing data.

The term “disease progression” refers to the course of a disease. The term reflects a disease or physical ailment whose course in most cases is the worsening, growth, or spread of the disease. This may happen until death, serious debility, or organ failure occurs. Some progressive diseases can be halted and reversed by treatment. Many can be slowed by medical therapy. Still, others cannot be altered by current treatments.

### B. NLRP3 Inflammasome Related Disease and Conditions

As used herein, the term “NLRP3 inflammasome mediated disorder” refers to a disease, disorder or condition that is related to diseases linked to NLRP3 activation, including abnormal activation and the consecutive IL-1β cytokine maturation.

NOD-like receptor protein 3 (NLRP3) belongs to the family of nucleotide-binding and oligomerization domain-like receptors (NLRs) and is also known as “pyrin domain-containing protein 3”. In response to inflammatory danger signals, NLRP3 interacts with an adapter protein, apoptosis-associated speck-like protein (ASC) and procaspase-1 to form the NLRP3 inflammasome. NLRP3 inflammasome activation then leads to the release of the inflammatory cytokines IL-1β and IL-18, and when dysregulated, can drive pathology in a number of disease settings.

NLRP3 inflammasome activation normally requires two steps. The first step involves a priming signal in which pathogen activated molecular patterns (PAMPs) or danger-activated molecular patterns (DAMPs) are recognized by Toll-like receptors, leading to activation of nuclear factor kappa B (NF-κB)-mediated signaling, which in turn up-regulates transcription of inflammasome-related components, including inactive NLRP3 and proIL-1β. The second step is the oligomerization of NLRP3 and subsequent assembly of NLRP3, ASC, and procaspase-1 into an inflammasome complex. This triggers the transformation of procaspase-1 to caspase-1, and the production and secretion of mature IL-1β and IL-18.

As described herein, hResistin/RELMα is a critical regulator of the priming and activation stages of the NLRP3 inflammasome. hResistin/RELMα is critical to both NLRP3 priming (via HMGB1) 11 and NLRP3 activation (via BTK) and production of IL-1β and IL-18 (via critical BTK¹² phosphorylation of four specific NLRP3 tyrosine residues) in both macrophages and B cells, leading to pulmonary vascular remodeling and PH. This work proves a major role for hResistin/RELMα in engaging the NLRP3 inflammasome in the innate immune response to injury and to a sustaining adaptive immune response in the long-term remodeling associated with PH. Because the NLRP3 inflammasome is regulated by hResistin-BTK signaling, the present invention is applicable to any NLRP3 inflammasome mediated disorder.

Accordingly, the NLRP3 inflammasome mediated disorder may comprise autoimmune disease; age-related macular degeneration (AMD); autoinflammatory diseases; inflammatory responses; inflammatory skin diseases; sepsis; psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis, systemic lupus erythematosus (SLE); lupus nephritis (LN); diabetes mellitus (e.g., Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis, Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenia purpura (ITP) or autoimmune thrombocytopenia; cryopyrin-associated periodic syndromes (CAPS); Alzheimer disease; atherosclerosis; myocardial infarction; allergic airway inflammation; gout; non-alcoholic fatty liver disease and nonalcoholic steatohepatitis; experimental autoimmune encephalitis; oxalate-induced nephropathy; hyperinflammation following influenza infection; stroke; silicosis; myelodysplastic syndrome; contact hypersensitivity; and traumatic brain injury.

In other embodiments, the NLRP3 inflammasome mediated disorder comprises autoinflammatory disorders including, but not limited to, cryopyrin-associated periodic syndrome (CAPS); familial cold autoinflammatory syndrome (FCAS); Muckle-Wells syndrome (MWS); neonatal onset multisystem inflammatory disorder (NOMID); complex or acquired inflammasomopathies including crystalline arthropathies, silicosis, asbestosis, gout, pseudogout and diabetes mellitus; NLRP3 extrinsic inflammasopathies including familial Mediterranean fever (FMF), pyogenic arthritis with pyodema gangrenosum and acne (PAPA) syndrome, hyperimmunoglobulinemia D with periodic fever syndrome (HIDS), Mevalonate kinase deficiency (MKD), and Schnitzler's syndrome (SS).

In particular embodiments, the NLRP3 inflammasome mediated disorder comprises inflammatory diseases including, but not limited to, Aspergillus fumigatus keratitis, stargardt disease type 1, Alzheimer's disease, atherosclerosis, atrial fibrillation, osteoarthritis and cancer.

In further embodiments, the NLRP3 inflammasome mediated disorder comprises pulmonary hypertension, right or left heart failure, lung inflammation including, but not limited to, bacterial, viral and parasitic infections.

### IV. Resistin Polymorphisms

In particular embodiments, Resistin polymorphisms can be used as a marker of an NLRP3 inflammasome mediated disease in a subject. In particular embodiments, Resistin polymorphisms can be used a marker or predictor of disease severity in a subject. In other embodiments, Resistin polymorphisms can be used a predictor of hospitalization. Resistin polymorphisms include, but are not limited to, rs10402265, C>G (disease severity) and rs12459044, C>G (hospitalization). Other polymorphisms can be used to predict, for example, ICU admission or treatment. For example, patients selected for ICU are expected to have complications of their clinical outcome within 24 and 72 hours.

The nucleotide sequence of the human Resistin gene is shown in SEQ ID NO:243. The coding/mRNA sequence is shown in SEQ ID NO:244. The amino acid sequence of human Resistin is shown in SEQ ID NO:245 (UniProt Q9HD89).

A subject can be one who has been previously diagnosed with or identified as suffering from or having a condition, disease, or disorder in need of treatment or one or more complications related to the condition, disease, or disorder, and optionally, have already undergone treatment for the condition, disease, disorder, or the one or more complications related to the condition, disease, or disorder. Alternatively, a subject can also be one who has not been previously diagnosed as having a condition, disease, or disorder or one or more complications related to the condition, disease, or disorder. For example, a subject can be one who exhibits one or more risk factors for a condition, disease, or disorder, or one or more complications related to the condition, disease, or disorder, or a subject who does not exhibit risk factors. A “subject in need” of treatment for a particular condition, disease, or disorder can be a subject suspected of having that condition, disease, or disorder, diagnosed as having that condition, disease, or disorder, already treated or being treated for that condition, disease, or disorder, not treated for that condition, disease, or disorder, or at risk of developing that condition, disease, or disorder.

In some embodiments, the subject is selected from the group consisting of a subject suspected of having a NLRP3 inflammasome mediated disease, a subject that has a NLRP3 inflammasome mediated disease, a subject diagnosed with a NLRP3 inflammasome mediated disease, a subject that has been treated for a NLRP3 inflammasome mediated disease, a subject that is being treated for a NLRP3 inflammasome mediated disease, and a subject that is at risk of developing a NLRP3 inflammasome mediated disease.

### A. Definitions

By “at risk of” is intended to mean at increased risk of, compared to a normal subject, or compared to a control group, e.g., a patient population. Thus, a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is increased by at least 10%, at least 20%, and even at least 50% over the control group with which the comparison is being made. In certain embodiments, a subject can be at risk of developing an NLRP3 inflammasome mediated disease.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue, a tissue print, a fingerprint, skin or hair; fragments and derivatives thereof. Non-limiting examples of samples or biological samples include cheek swab; mucus; whole blood, blood, serum; plasma; urine; saliva, semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. Subject samples or biological samples usually comprise derivatives of blood products, including blood, plasma and serum. In some embodiments, the sample is a biological sample. In some embodiments, the sample is blood. In some embodiments, the sample is plasma. In some embodiments, the sample is blood, plasma, serum, or urine. In certain embodiments, the sample is a serum sample. In particular embodiments, the sample is a urine sample.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, sputum, synovial fluid, sweat, tears, urine, vaginal secretion, and vomit. Extracellular bodily fluids include intravascular fluid (blood plasma), interstitial fluids, lymphatic fluid and transcellular fluid. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device, a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMSIM) samples can be stored and mailed, and an assay can be performed remotely.

The term “reference” means a standard or control condition.

By “binding assay” is meant a biochemical assay wherein the Resistin biomarker is detected by binding to an agent, such as an antibody, through which the detection process is carried out. The detection process may involve fluorescent or radioactive labels, and the like. The assay may involve immobilization of the biomarker, or may take place in solution.

“Immunoassay” is an assay that uses an antibody to specifically bind an antigen (e.g., a marker such as Resistin). The immunoassay is characterized by the use of specific binding properties of a particular antibody to isolate, target, and/or quantify the antigen. Non-limiting examples of immunoassays include ELISA (enzyme-linked immunosorbent assay), immunoprecipitation, SISCAPA (stable isotope standards and capture by anti-peptide antibodies), Western blot, etc.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers, detecting peptides, detecting proteins, or of detecting a condition, detecting a disease or a disorder (e.g., when positive assay results are obtained). In the latter context, “detecting” and “diagnosing” are considered synonymous when mere detection indicates the diagnosis.

The terms “marker” or “biomarker” are used interchangeably herein, and in the context of the present invention refer to a protein or peptide (for example, protein or peptide associated with an NLRP3 inflammasome mediated disease as described herein) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by, for example, antibodies or mass spectroscopy. In some applications, for example, a mass spectroscopy or other profile of multiple antibodies may be used to determine multiple biomarkers, and differences between individual biomarkers and/or the partial or complete profile may be used for diagnosis. In some embodiments, the biomarkers may be detected by antibodies, mass spectrometry, or combinations thereof.

The term “differentially present” or “change in level” refers to differences in the quantity and/or the frequency of a marker present in a sample taken from patients having a specific disease or disorder as compared to a control subject. For example, a marker can be present at an elevated level or at a decreased level in samples of patients with the disease or disorder compared to a control value (e.g., determined from samples of control subjects). Alternatively, a marker can be detected at a higher frequency or at a lower frequency in samples of patients compared to samples of control subjects. In particular embodiments, a marker can be differentially present in patients having an NLRP3 inflammasome mediated disease as compared to a control subject including patients having, for example, no disease.

A marker, compound, composition or substance is differentially present in a sample if the amount of the marker, compound, composition or substance in the sample (a patient having an NLRP3 inflammasome mediated disease) is statistically significantly different from the amount of the marker, compound, composition or substance in another sample (a patient having no NLRP3 inflammasome mediated disease or a less severe or early form thereof), or from a control value (e.g., an index or value representative of non-severe NLRP3 inflammasome mediated disease or no NLRP3 inflammasome mediated disease). For example, a compound is differentially present if it is present at least about 50%, at least about 60%, at least about 70%, at least about 80%, at least about 90%, at least about 100%, at least about 110%, at least about 120%, at least about 130%, at least about 150%, at least about 180%, at least about 200%, at least about 300%, at least about 500%, at least about 700%, at least about 900%, or at least about 1000% greater or less than it is present in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease or disorder, is statistically significantly higher or lower than in the control samples or control values obtained from controls such as a subject having non-severe disease and the like, or otherwise healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples than the other set of samples. These exemplary values notwithstanding, it is expected that a skilled practitioner can determine cut-off points, etc., that represent a statistically significant difference to determine whether the marker is differentially present.

The term “one or more of” refers to combinations of various biomarkers. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . . N, where “N” is the total number of biomarker proteins in the particular embodiment. The term also encompasses, and is interchangeably used with, at least 1, at least 2, at least 3, at least 4, at least 5, at least 6, at least 7, at least 8, at least 9, at least 10, at least 11, at least 12, at least 13, at least 15, 16,17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . . N. It is understood that the recitation of biomarkers herein includes the phrase “one or more of” the biomarkers and, in particular, includes the “at least 1, at least 2, at least 3” and so forth language in each recited embodiment of a biomarker panel.

“Detectable moiety” or a “label” refers to a composition detectable by spectroscopic, photochemical, biochemical, immunochemical, or chemical means. For example, useful labels include 32P, 35S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, or direct analysis by mass spectrometry of intact protein or peptides. In some embodiments, the detectable moiety is a stable isotope. In some embodiments, the stable isotope is selected from the group consisting of 15N, 13C, 180 and 2H.

### B. Measurement/Detection of Resistin by Immunoassays

In specific embodiments, Resistin can be detected and/or measured by immunoassay. Immunoassay requires biospecific capture reagents/binding agent, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. The biospecific capture reagents can, in some embodiments, bind all forms of the biomarker, e.g., PSA and its post-translationally modified forms (e.g., glycosylated form). In other embodiments, the biospecific capture reagents bind the specific biomarker and not similar forms thereof. In particular embodiments, an anti-Resistin antibody or antigen-binding fragement thereof described here is used to detect/measure Resistin.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker (e.g., Resistin) is attached to the surface of an MS probe, such as a pre-activated protein chip array. The biomarker is then specifically captured on the biochip through this reagent, and the captured biomarker is detected by mass spectrometry.

In certain embodiments, the expression levels of the protein biomarkers employed herein (e.g., Resistin) are quantified by immunoassay, such as enzyme-linked immunoassay (ELISA) technology. In specific embodiments, the levels of expression of Resistin is determined by contacting the biological sample with an antibody, or antigen binding fragment thereof, that selectively bind to the Resistin biomarker; and detecting binding of the antibody, or antigen binding fragment thereof, to the Resistin biomarker. In certain embodiments, the binding agents employed in the disclosed methods and compositions are labeled with a detectable moiety. In other embodiments, a binding agent and a detection agent are used, in which the detection agent is labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

For example, the level of a Resistin biomarker in a sample can be assayed by contacting the biological sample with an antibody, or antigen binding fragment thereof, that selectively binds to the target protein (referred to as a capture molecule or antibody or a binding agent), and detecting the binding of the antibody, or antigen-binding fragment thereof, to the protein. The detection can be performed using a second antibody to bind to the capture antibody complexed with its target biomarker. A target biomarker can be an entire protein, or a variant or modified form thereof. Kits for the detection of proteins as described herein can include pre-coated strip/plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods for detecting proteins (including, e.g., Resistin) in a sample obtained from a subject, wherein the levels of expression of the proteins in a biological sample are determined simultaneously. For example, in one embodiment, methods are provided that comprise: (a) contacting a biological sample obtained from the subject with a plurality of binding agents that each selectively bind to one or more biomarker proteins for a period of time sufficient to form binding agent-biomarker complexes; and (b) detecting binding of the binding agents to the one or more biomarker proteins. In further embodiments, detection thereby determines the levels of expression of the biomarkers in the biological sample; and the method can further comprise (c) comparing the levels of expression of the one or more biomarker proteins in the biological sample with predetermined threshold values, wherein levels of expression of at least one of the biomarker proteins above or below the predetermined threshold values indicates, for example, the subject has an NLRP3 inflammasome mediated disease, the severity thereof, and/or is/will be responsive to therapy. Examples of binding agents that can be effectively employed in such methods include, but are not limited to, antibodies or antigen-binding fragments thereof, aptamers, lectins and the like.

Although antibodies are useful because of their extensive characterization, any other suitable agent (e.g., a peptide, an aptamer, or a small organic molecule) that specifically binds a biomarker of the present invention is optionally used in place of the antibody in the above described immunoassays. For example, an aptamer that specifically binds a biomarker and/or one or more of its breakdown products might be used. Aptamers are nucleic acid-based molecules that bind specific ligands. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

In specific embodiments, the assay performed on the biological sample can comprise contacting the biological sample with one or more capture agents (e.g., antibodies, lectins, peptides, aptamer, etc., combinations thereof) to form a biomarker: capture agent complex. The complexes can then be detected and/or quantified. A subject can then be identified as having an NLRP3 inflammasome mediated disease based on a comparison of the detected/quantified/measured levels of biomarkers to one or more reference controls as described herein. The biomarker levels can also be utilized with other biomarker measurements.

In one method, a first, or capture, binding agent, such as an antibody that specifically binds the Resistin protein biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker (or to the bound capture antibody) is then used to detect binding of the polypeptide biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In another embodiment, the assay is a competitive binding assay, wherein labeled protein biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different protein biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, can be used. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis.

In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers including Resistin at the corresponding spots on the plate.

In several embodiments, the Resistin biomarker of the present invention may be detected by means of an electrochemicaluminescent assay developed by Meso Scale Discovery (Gaithersburg, MD). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ~620 nm, eliminating problems with color quenching. See U.S. Pat. No. 7,497,997; 7,491,540; 7,288,410; 7,036,946; 7,052,861; 6,977,722; 6,919,173; 6,673,533; 6,413,783; 6,362,011; 6,319,670; 6,207,369; 6,140,045; 6,090,545; and 5,866,434. See also U.S. Patent Applications Publication No. 2009/0170121; 2009/006339; No. 2009/0065357; 2006/0172340; 2006/0019319; 2005/0142033; 2005/0052646; No. 2004/0022677; 2003/0124572; 2003/0113713; 2003/0003460; No. 2002/0137234; 2002/0086335; and No. 2001/0021534.

### C. Measurement/Detection By Other Detection Methods

In other embodiments, Resistin can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In particular embodiments, Resistin can be captured and concentrated using nano particles. In a specific embodiment, the proteins can be captured and concentrated using Nanotrap® technology (Ceres Nanosciences, Inc. (Manassas, VA)). Briefly, the Nanotrap platform reduces pre-analytical variability by enabling biomarker enrichment, removal of high-abundance analytes, and by preventing degradation to highly labile analytes in an innovative, one-step collection workflow. Multiple analytes sequestered from a single sample can be concentrated and eluted into small volumes to effectively amplify, up to 100-fold or greater depending on the starting sample volume (Shafagati, 2014; Shafagati, 2013; Longo, et al., 2009), resulting in substantial improvements to downstream analytical sensitivity.

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, CA.), Invitrogen Corp. (Carlsbad, CA), Affymetrix, Inc. (Fremong, CA), Zyomyx (Hayward, CA), R&D Systems, Inc. (Minneapolis, MN), Biacore (Uppsala, Sweden) and Procognia (Berkshire, UK). Examples of such protein biochips are described in the following patents or published patent applications: U.S. Pat. Nos. 6,537,749; 6,329,209; 6,225,047; 5,242,828; PCT International Publication No. WO 00/56934; and PCT International Publication No. WO 03/048768.

In a particular embodiment, the present invention comprises a microarray chip. More specifically, the chip comprises a small wafer that carries a collection of binding agents bound to its surface in an orderly pattern, each binding agent occupying a specific position on the chip. The set of binding agents specifically bind to Resistin and one or more other biomarkers. In particular embodiments, a few micro-liters of blood, serum or plasma are dropped on the chip array. Protein biomarkers present in the tested specimen bind to the binding agents specifically recognized by them. Subtype and amount of bound mark is detected and quantified using, for example, a fluorescently-labeled secondary, subtype-specific antibody. In particular embodiments, an optical reader is used for bound biomarker detection and quantification. Thus, a system can comprise a chip array and an optical reader. In other embodiments, a chip is provided.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

In the following Examples, all data included in the preliminary findings and the proposed results will be reported as mean±SEM. Differences between multiple groups will be compared by analysis of variance (ANOVA) following by Bonferroni's multiple comparison test. Two group analysis will be performed by Student's t-test. A value of p<0.05 will be considered significant. Sample size calculation was based on pilot studies and on previous studies in rodents using similar hypoxia settings and confirmed by power analysis^{6,53,54} To achieve statistical significance, no less than six mice will be used in each group. Detailed animal welfare, number justification and power analysis are described in the Vertebrate Animals section. Sex as a biological variable: In the following rodent models, whether sex differences have an impact on PH pathogenesis remains unclear.55 The present inventors will include both sexes with equal numbers in the vertebrate animal studies for the present proposal. For human studies, PH is a female predominant disease which is also reflected in the present inventors' clinical cohort.⁵⁶ Similar to what the present inventors have reported previously,⁵⁶ in addition to the analysis performed on the entire dataset, the present inventors will also conduct the analysis in subgroups, with consideration of race and gender, PH subtype, proximity of blood draw to most recent catheterization, and to demographics.

### Example 1: Resistin is a Critical Regulator of Nlrp3 Inflammasome Activation

Human resistin (Hresistin) and its rodent homolog resistin-like molecule (RELM)-a are cysteine rich secretory proteins, with a proinflammatory cytokine effect, that activates immune cells and promotes the secretion of other proinflammatory cytokines.¹ Elevated levels of Hresistin have been linked to multiple diseases including insulin resistance, atherosclerosis, diabetes, cardiovascular diseases (CVD), pulmonary hypertension (PH), autoimmune diseases, asthma and others.² Nucleotide-binding domain (NOD)-like receptor protein 3 (NLRP3) is a protein complex that mediates inflammation in both infectious and sterile conditions and contributes to the progression of multiple diseases including diabetes, obesity, and cardiovascular diseases, cancers and etc.5-7 Multiple signals such as DAMPs, PAMPs, K+efflux, Ca2+influx and ROS/mitochondrial dysfunction are already known regulators of NLRP3 inflammasome.⁶⁻⁹ The NLRP3 inflammasome is composed of three components: the sensor protein NLRP3, the adaptor protein ASC, and the effector protein caspase-1. Activation of NLRP3 inflammasome is tightly regulated at two stages known as the priming stage and activation stage.⁷ The priming stage is accomplished by the activation of the NF-κB pathway, which increases the transcription of NLRP3 protein, pro interleukin-1β (pro-IL-1β), pro-interleukin-18 (pro-IL-18) and pro-Caspase-1. The activation stage involves the assembly of the NLRP3 inflammasome complex and the subsequent cleavage of caspase-1, cleavage and secretion of pro-inflammatory cytokines, such as interleukin-1β (IL-1β) and interleukin-18 (IL-18).

The present inventor reported that, Hresistin/RELMα initiates the inflammatory response, through activating damage-associated molecular pattern (DAMP) pathway high-mobility group box-1 (HMGB1).¹⁰⁻¹² HMGB-1 is released from cells in response to injury or inflammation and acts as a danger signal to activate immune cells and promote an inflammatory response. It is still unclear to date, how Hresistin/RELMα's diverse inflammatory effects are integrated or how they amplify and sustain inflammation during different disease progression. Hresistin/RELMα activates Th2 (T-helper type 2) differentiation of macrophages by activating HMGB-1.¹⁰ Th2 stimuli also can induce RELMα expression through IL-4, IL-13, and STAT-6 pathways.¹³⁻¹⁵ It suggests that Hresistin/RELMα signaling may act as a crucial hub of a positive feedback loop to trigger, amplify, and sustain inflammation through its immunoregulatory activities in Hresistin linked disease progression. The present inventor also reported Bruton's Tyrosine Kinase (BTK) as a binding partner of RELMα.¹⁶ BTK is a tec kinase that plays a crucial role in B cell development and activation.¹⁷ BTK deficient mice showed to have diminished macrophage activation in response to hypoxia,^{18,19} The present inventor's recent studies further revealed that BTK mediates the chemokine activity of Hresistin/RELMα in macrophages in mouse PH lung in vivo and in human cells in vitro.¹⁰ RELMα has been found to stimulate the autophosphorylation of BTK.^{16,20} RELMα-induced macrophage secretion of Th2 cytokines in the lung has been shown to be BTK dependent.²² Recent research has shown the link between BTK and NLRP3 signaling, in which BTK can activate the NLRP3 inflammasome by directly phosphorylating it, leading to the production of IL-1β and IL-18.^{8,23} In addition, inhibition of BTK has been shown to reduce NLRP3 inflammasome activation and the production of cytokines in response to various stimuli.²⁴ Collective evidence suggests that the link between BTK and Hresistin/RELMα may play a role in the pathogenesis of multiple diseases.²¹

Macrophages are the main cellular source of Hresistin.^{3,10} Activation and accumulation of macrophages at early time points have been observed to play an important role in multiple diseases. 9,25 Collective evidence on previous literature, considered the NLRP3 inflammasome activation in macrophages as the key mechanism of inflammation driven macrophage-induced inflammatory diseases.^{27,28} Increasing evidence suggested the pro-inflammatory role of Hresistin in regulating the NLRP3 inflammasome activation.³⁰⁻³² As a major regulator and effector of macrophages and a critical driver of its pro-inflammatory phenotype, Hresistin/RELMα signaling activates the damage-associated molecular pattern (DAMP) and Bruton's tyrosine kinase (BTK) signaling to drive the inflammation. Additionally, both HMGB-1 and BTK have been identified as regulators of the NLRP3 inflammasome activation through priming and activation stages, respectively.^{8,34} However, it is still unclear whether and how Hresistin-HMGB1-BTK signaling regulates the NLRP3 inflammasome. Moreover, to understand the immunoregulatory pathogenesis of Hresistin linked inflammatory diseases the present inventor selected PH as a disease model PH. This paper describes the present inventor's novel work proving that human resistin is critical to the priming and the activation of the NLRP3 inflammasome in the macrophage and other immune and structural cells in disease states where the NLRP3 inflammasome is involved and demonstrates that Hresistin is a highly effective and focused target to reduce NLRP3 action at the site of excessive inflammation using the present inventor's highly focused human monoclonal antibody against human resistin.

### Results

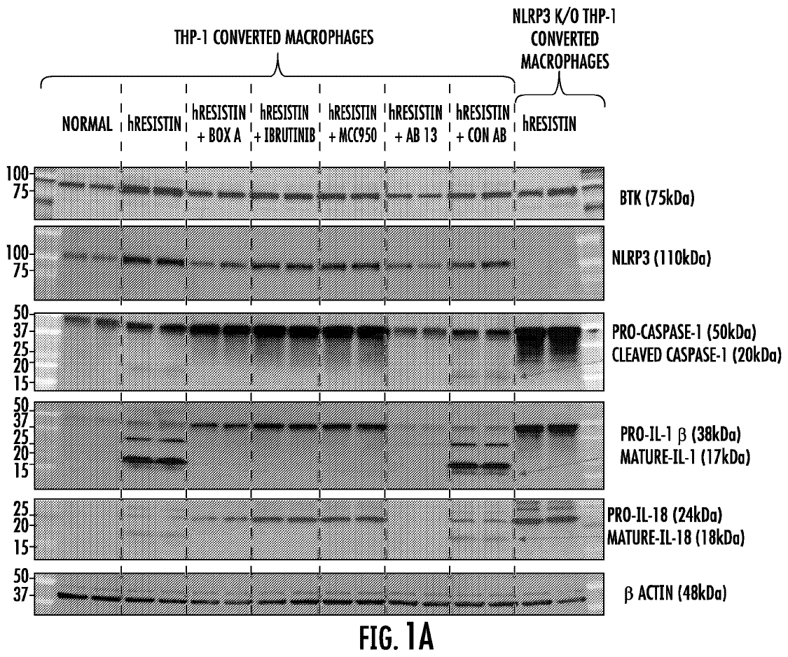

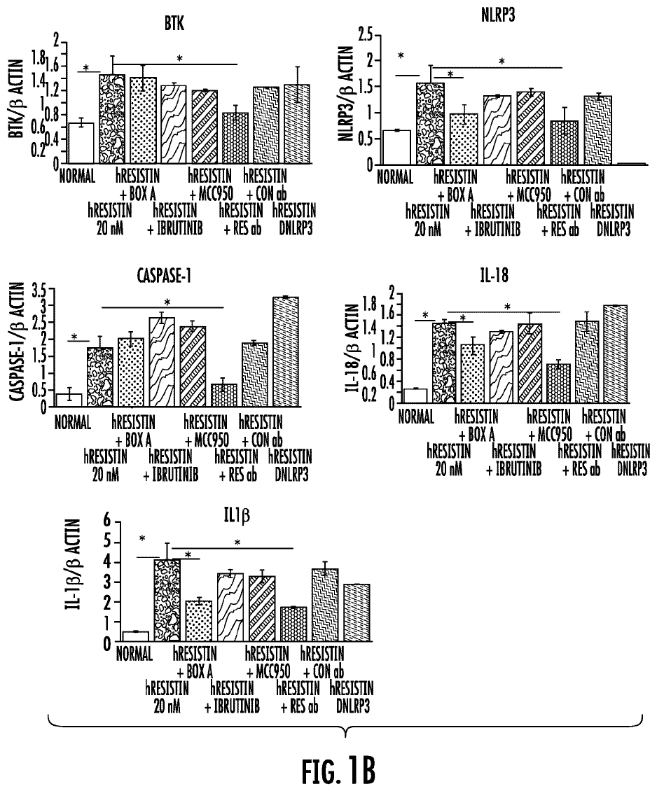

Hresistin-HMGB1 signaling induces priming stage of NLRP3 inflammasome in human macrophages. In order to understand the detailed mechanism of Hresistin on NLRP3 inflammasome activation, the present inventor separated the study into two parts. Since, the NLRP3 inflammasome activation is regulated at two steps, at first, the present inventor checked the involvement of Hresistin on the priming stage. Given the fact that Hresistin stimulates the expression and secretion of HMGB-1 which is critical to the priming pathway, the present inventor hypothesized Hresistin regulates the priming stage through HMGB-1. THP-1 converted macrophages were treated with Hresistin. To understand the involvement of HMGB-1 on priming stage, the present inventor blocked HMGB-1 with box A inhibitor in these Hresistin treated samples. Similarly, the present inventor used Hresistin blocking antibody to bind and block Hresistin and control antibody. The present inventor used control ab in this experiment as an isotype of Hresistin antibody, which doesn't have any functional properties of Hresistin antibody. After 24 hours incubation western blot analysis was done to both whole cell pellet and the supernatant to check the proteins involved in suggested pathway. Hresistin was found to stimulate the expression level of NLRP3, pro forms of Caspase-1, IL-1β, and IL-18 (FIG. 1). Interestingly, this activity was blocked in both HMGB-1 Box A inhibitor and Hresistin antibody treated samples, indicate both HMGB-1 and Hresistin are linked regulators of the priming stage. Blocking Hresistin reduced the expression level of HMGB-1 in the macrophages, thus reducing the downstream expression and signaling of priming NLRP3. To check the involvement of BTK and NLRP3 in priming stage, the present inventor used Ibrutinib and MCC950 to block BTK and NLRP3 respectively. However, compared to Hresistin treated samples, there were no significant difference in the samples treated with Ibrutinib or MCC950 treated samples, show blocking NLRP3 activation does not have any impact on priming stage. Here the present inventor have proved Hresistin prime NLRP3 inflammasome through HMGB-1.

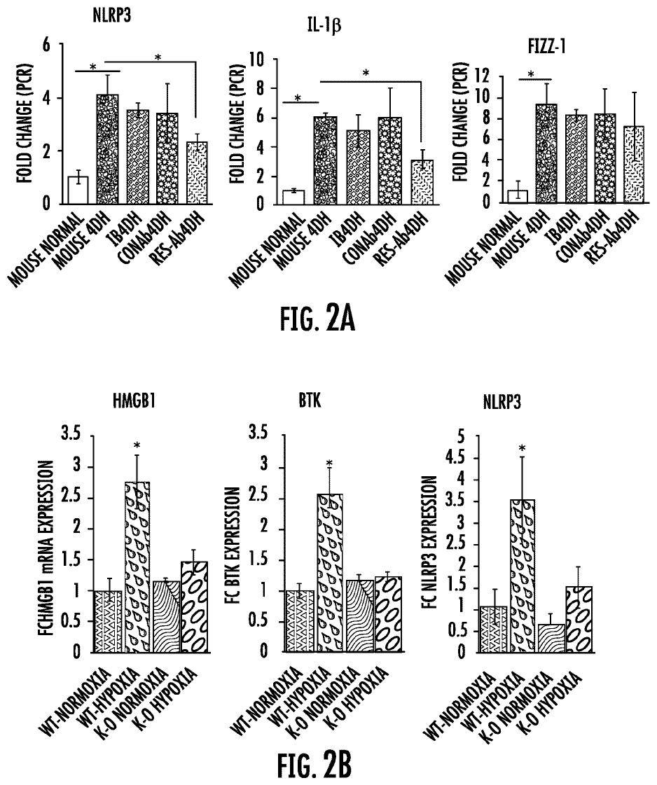

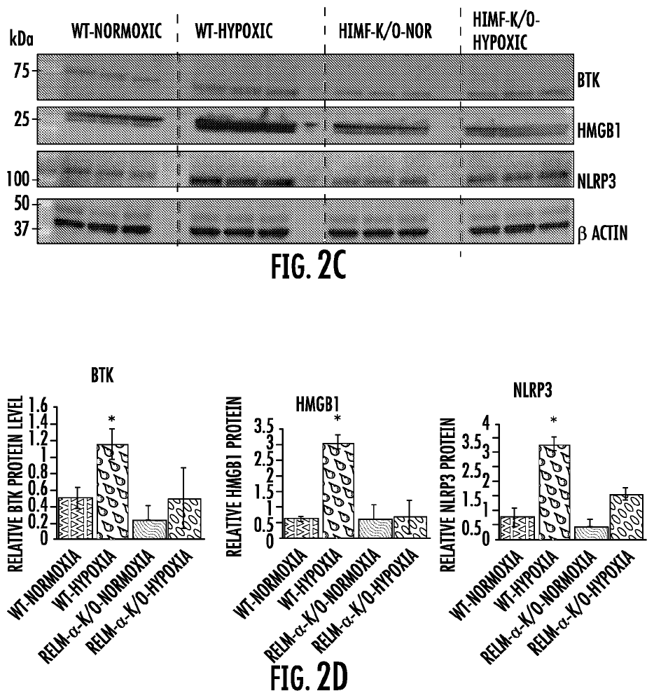

RELMα upregulates pro-inflammatory phenotypes in hypoxic mice lungs. Next, the present inventor checked this hypothesis in an in-vivo system where NLPR3 is active. For this the present inventor used C57/BL6 mice, both wild type (WT) and RELMα knock outs. The present inventor have previously shown and published, 4 days in hypoxic condition increase lung RELMα in mice and initiate the process of vascular remodeling. So here the present inventor kept both WT and RELMα K/O mice in 4 days of hypoxia and isolated their lungs to perform immunoblot analysis for BTK, HMGB-1 and NLRP3. Similar to in-vitro experiment, the present inventor saw a significant increased levels of BTK, HMGB-1 and NLRP3 in WT mice that were kept in hypoxia, compared to WT normoxic mice (FIG. 2). Interestingly, RELMα K/O hypoxic mice didn't show any significant difference compared to normoxic mice, indicating the importance of RELMα in the inflammasome.

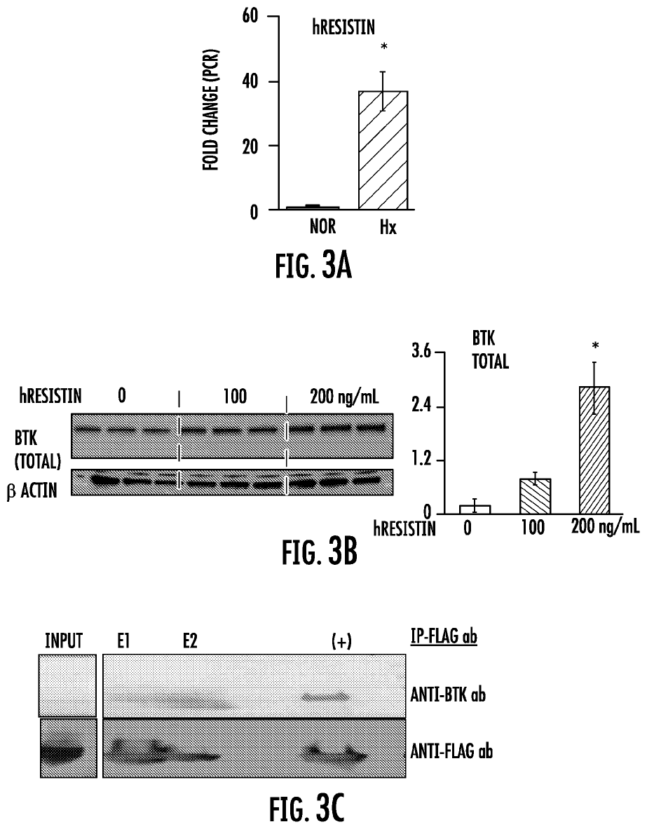

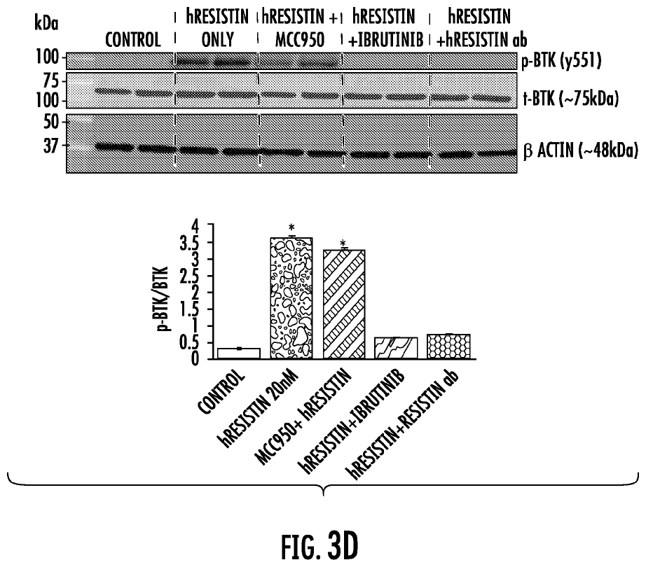

Hresistin binds to BTK in human macrophages and initiates the autophosphorylation of BTK. As described before, the present inventor hypothesized Hresistin regulates NLRP3 activation via BTK. Therefore, first the present inventor checked the effect of Hresistin on BTK. The present inventor have previously reported mouse RELMα as a binding partner of mouse BTK. Since RELMα is a mouse homolog of human resistin, the present inventor checked the binding ability of human BTK with Hresistin. In order to explore this pulldown assays were performed as previously described [REF}. The present inventor used human THP-1 converted macrophages kept in hypoxic condition and used the cell lysate to pull down BTK along with Hresistin. Hypoxic condition caused nearly 40-fold increase in the expression level of Hresistin compared to non-hypoxic condition (FIG. 3A). The present inventor saw the increased level of BTK expression in human B cells when the present inventor add Hresistin in dose dependent manner (FIG. 3B). Moreover, the present inventor saw BTK pulled down with Hresistin (FIG. 3C). Next, the present inventor studied how this binding affected the autophosphorylation of BTK. For this the present inventor stimulated the THP-1 converted macrophages with 20 nM Hresistin for 15 minutes and checked the BTK phosphorylation using immunoblots. Interestingly, the present inventor observed phospho-BTK in Hresistin stimulated samples and that activity was blocked by Hresistin blocking antibody, indicating Hresistin involvement in BTK autophosphorylation (FIG. 3D).

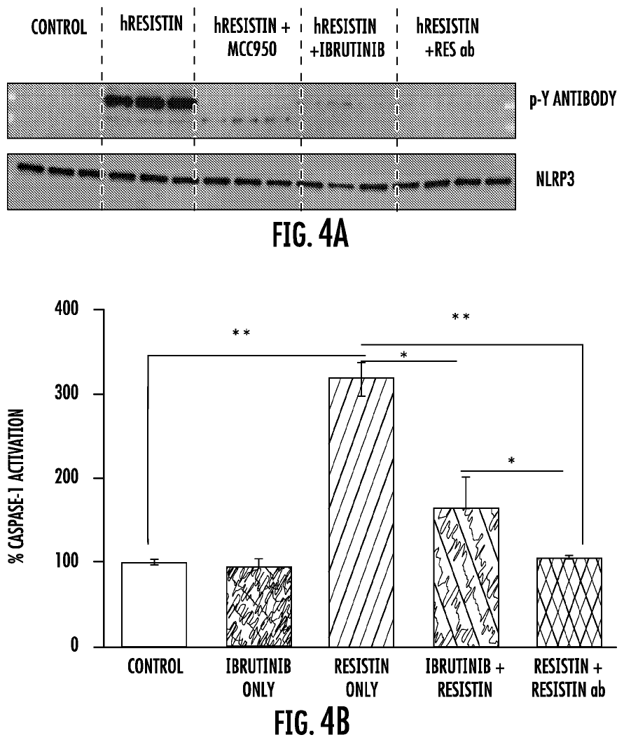

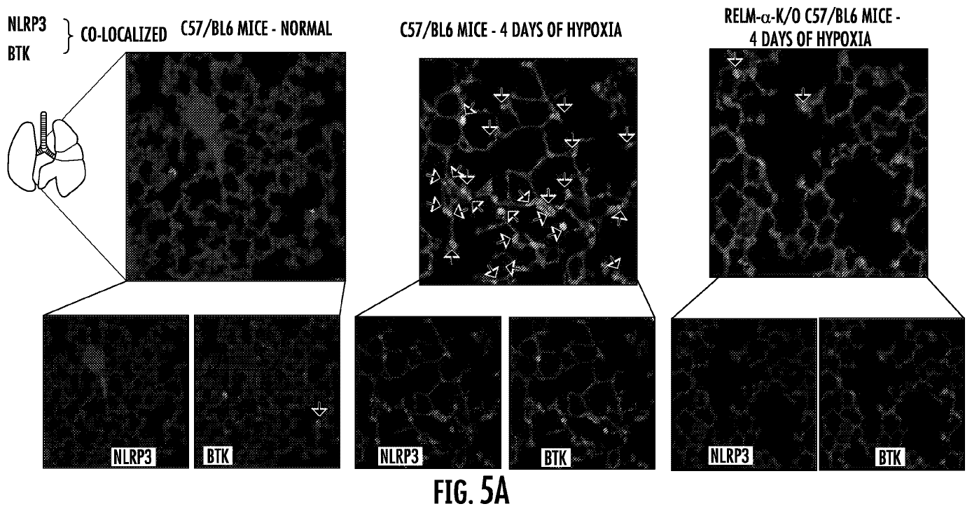

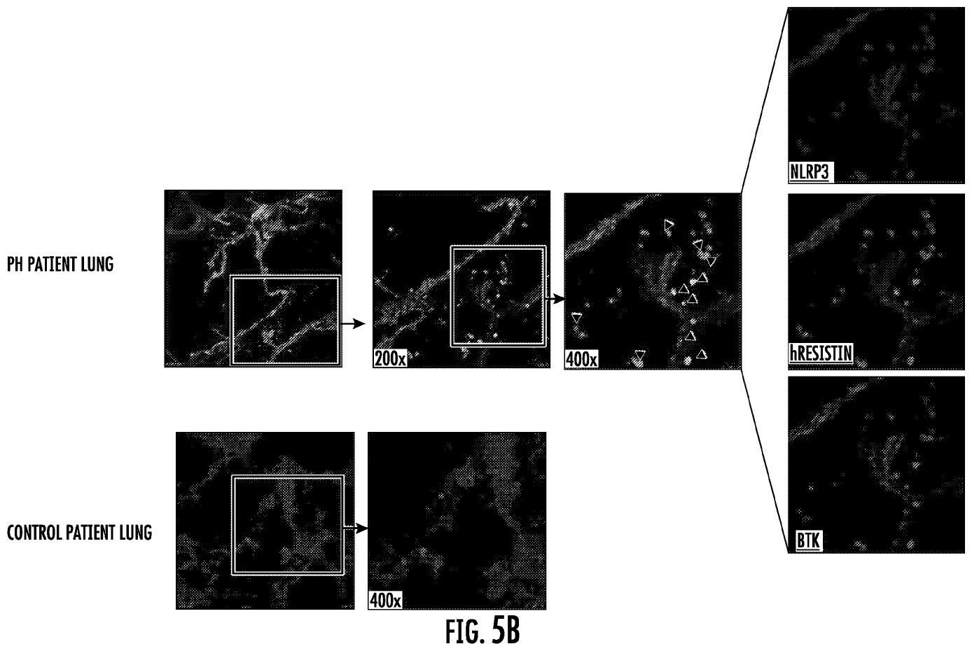

Hresistin stimulates NLRP3 phosphorylation, which was blocked by Ibrutinib, MCC950 and Hresistin ab. After the production of primed inflammasome components in the priming step, the next activation step causes the assembly of active NLRP3, cleavage of pro-capsase-1 to active capsase-1 which in turn cleaves the pro-IL-1β and proIL 18 to their active forms, and facilitates the secretion of IL-1β and IL-18.7 NLRP3 phosphorylation of four tyrosines by BTK is a key step in NLRP3 activation.8,23 As Hresistin was shown to be an upstream activator of BTK, the present inventor hypothesized Hresistin activated BTK was critical to NLRP3 phosphorylation and activation. The present inventor used THP-1 converted macrophages treated with Hresistin for 24 hrs with and without antibody to Hresistin, Ibrutinib (a BTK inhibitor), as well as MCC950 a NLRP3 inhibitor. Since there are no NLRP3 specific phosphor antibodies the present inventor used phosphor tyrosine antibodies used by other to assess changes in phosphorylation status of NLRP3.8 Hresistin treated samples showed two phosphor bands corresponding to the molecular weight of NLRP3 and BTK (FIG. 4). The treatment with Ibrutinib completely blocked both bands confirming the upstream role of BTK on NLRP3 phosphorylation. Hresistin antibody treated samples did not show phosphor BTK or phosphor NLRP3 bands in the immunoblot, confirming the proactive role of Hresistin on NLRP3 activation. The present inventor further investigated this using caspase-1 activation assay and as expected blocking by both Ibrutinib and Hresistin ab reduce the Caspase-1 activation significantly. To further confirm the role of mouse RELM-α on inflammasome activation in an in-vivo system, the present inventor used lung sections from WT and RELM-α K/O mice kept in 4 days of hypoxic condition. Lung tissues were collected for immunohistology to check NLRP3 and BTK colocalization. Similar to previous findings, 4 days of hypoxic mice had significantly high levels of NLRP3 and BTK co-localization, compared to normoxic mice (FIG. 5). However, even in the hypoxic condition the present inventor didn't see significantly increased levels of BTK and NLRP3 co-localization in RELM-α k/O mice, further proving the present inventor's hypothesis that Hresistin/Relm-α is critical to the activation of NRP3 through BTK phosphorylation. Similarly, high levels of BTK, NLRP3 and Hresistin levels were observed in PH patients' lungs compared to healthy controls (FIG. 5B).

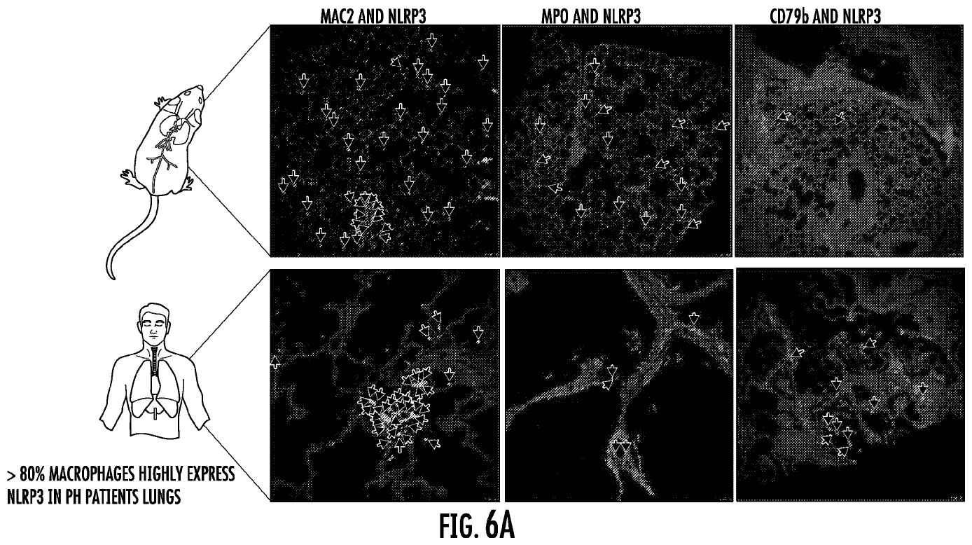

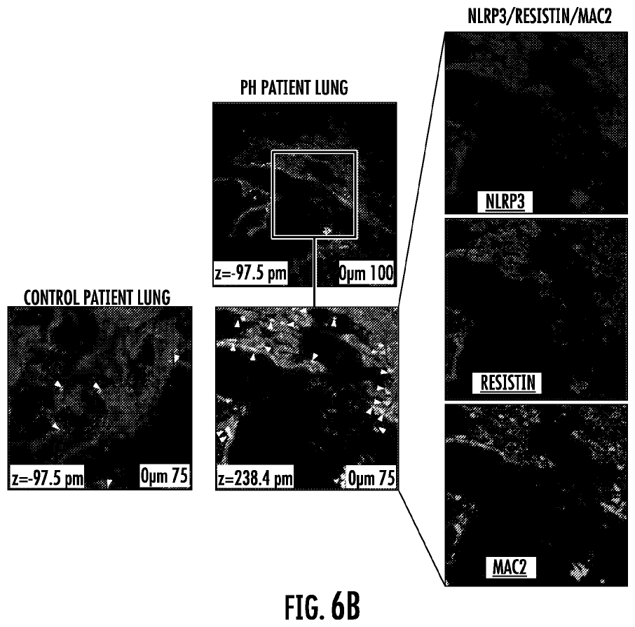

Macrophages are the main source of Hresistin and NLRP3 in mice hypoxic lungs and PH patients' lungs Al-Qazazi and the team recently showed macrophage-NLRP3 activation critical for right heart failure in PAH condition.27 To check which type of immune cells, express NLRP3 the most, the present inventor used lung sections from 4 days hypoxic mice and PH patients for immunohistology staining with NLRP3 and or MAC2, MPO and CD79b markers. According to previous literature macrophages found to be the main source of Hresistin in humans. Interestingly, all most all macrophages express NLRP3 in mice lungs and nearly 80% NLPR3 expressing cells are macrophages in human lungs (FIG. 6). This confirms macrophages as the main source of Hresistin and NLRP3 in mice hypoxic lungs and PH patients' lungs.

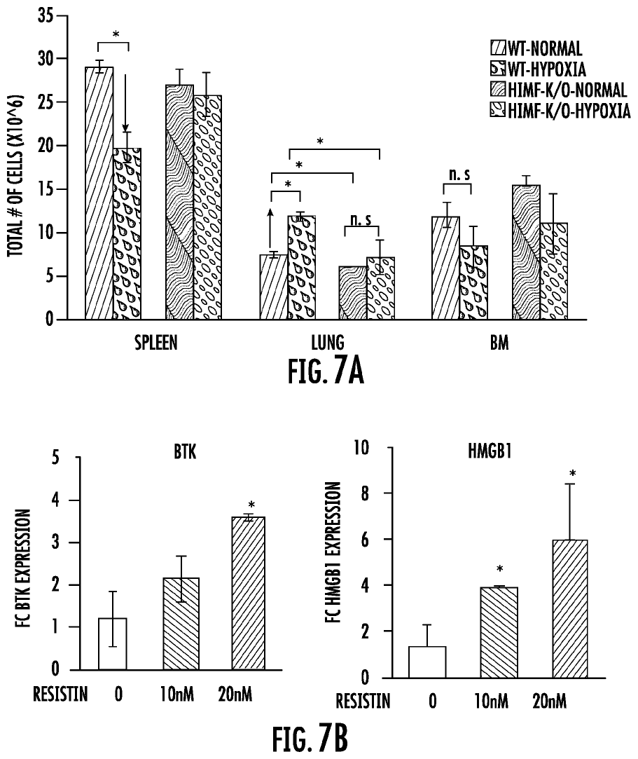

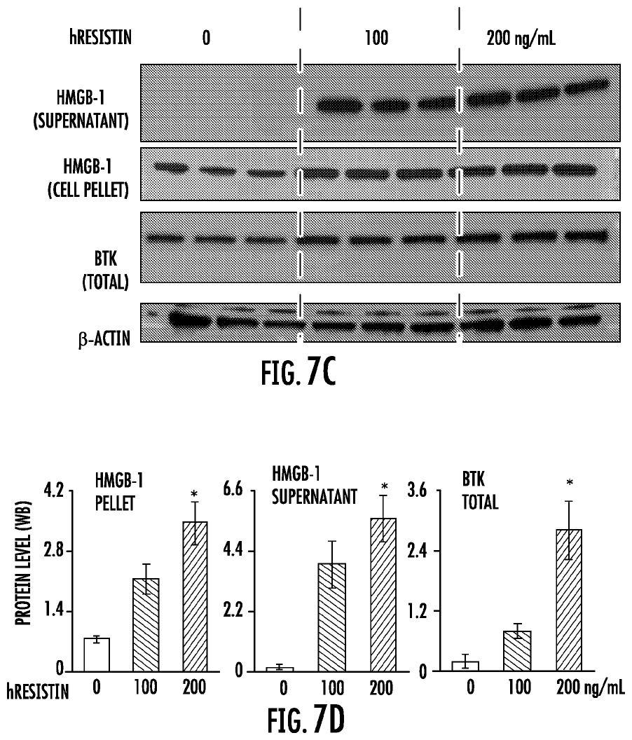

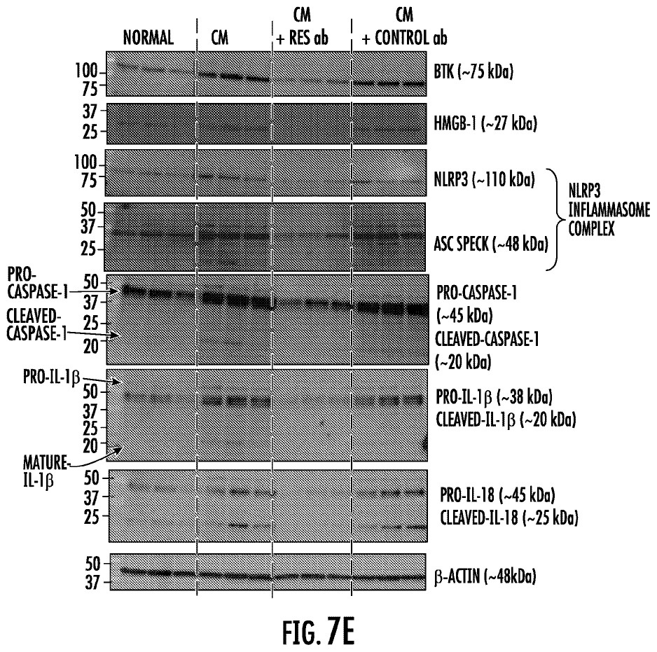

Macrophage-derived Hresistin activates NLRP3 signaling in B cells. For inflammation to play a significant role in chronic diseases, a transition from innate to adaptive immunity would likely be required. Macrophages are considered as early responders and are a major source of NLRP3 and are present throughout chronic disease such as pulmonary hypertension. The present inventor considered, however a role for adaptive immunity through activated macrophage cross talk with other immune cells. Since BTK is an essential mediator of B-cell-receptor signaling in the functioning of adaptive immunity and the present inventor found Hresistin as a binding partner of BTK, the present inventor selected B cells for this study. The present inventor did both in vitro and in-vivo experiments with B cells. In hypoxic mice lungs the present inventor observed RELMα activation induced the recruitment of B cells to the lungs from their spleens (FIG. 7A).

Similar to section 3.4, in human B cells the present inventor saw Hresistin increase the expression as well as the secretion of HMGB1 and phosphorylation of BTK (FIG. 7B-7D). However, the present inventor did not find Hresistin in human B cells, leading to us to hypothesize B cells required macrophages to produce Hresistin. The present inventor observed the macrophage-derived Hresistin activates NLRP3 inflammasome in B cells (FIG. 7E). This suggests that through regulating B cells, Hresistin drives a transition to a long-term adaptive response to sustain the vascular inflammation in PH.

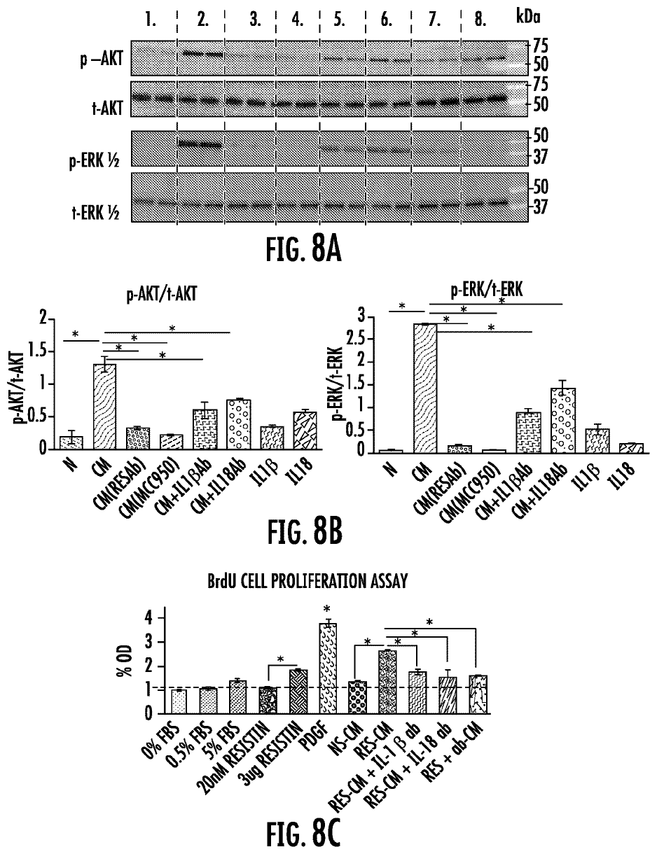

Hresistin promotes HPVSMC proliferation through macrophage-derived mature IL-1β and IL-18. So far, it is clear how Hresistin initiates early immune responses through the activation of NLRP3 inflammasome. However, there is a huge knowledge gap on understanding how this early activation lead the pathogenesis of PH. Hence, next the present inventor explored how the expression of components of Hresistin-BTK-NLRP3 signaling, lead the development of pulmonary vascular smooth muscle cell (PVSMC) proliferation. The present inventor hypothesized Hresistin stimulates AKT and ERK1/2 phosphorylation in Human SMCs through mature IL-1β and IL-18. Therefore, the present inventor used Hresistin treated macrophage conditioned media to treat PVSMC and checked phosphor AKT and ERK1/2 levels using immunoblots. The present inventor saw phosphorylated AKT and ERK1/2 in Hresistin treated macrophage conditioned media. This activity was blocked in the Hresistin ab or Ibrutinib or MCC950 treated conditioned media treated PVSMCs. Further, when the present inventor add either IL-1β blocking antibody or IL-18 blocking antibody the phosphorylation of AKT as well as ERK ½ significantly downregulated. To conform, whether IL-1β or IL-18 directly have any effect on phosphorylation, the present inventor used IL-1β and IL-18 cleaved proteins. As expected, the present inventor saw both active IL-1β and IL-18 protein upregulate the phosphorylation of AKT and ERK ½ (FIG. 8A).

Since the present inventor confirmed that Hresistin promotes phosphorylation of AKT and ERK ½ through macrophage-derived mature IL-1β and IL-18, next the present inventor checked how it affects HPVSMC proliferation. So, the present inventor also performed BrdU cell proliferation assay to further confirm this concept (FIG. 8C). The present inventor have previously found high concentration of Hresistin can stimulate HPVSMC proliferation. Therefore, the present inventor used Hresistin in high and low concentrations. The same amount of Hresistin that the present inventor used to stimulate macrophages was used as low concentration to opt out any effects from that. The present inventor used PDGF as a positive control for the BrdU assay. Since the conditioned media contains 5% FBS, the present inventor used 5% FBS to check activity of it on cell proliferation. Similar to previous results, the present inventor saw Hresistin treated conditioned media significantly increase the smooth muscle cell proliferation. Cell proliferation was significantly downregulated in the Hresistin ab, IL-1β blocking ab and IL-18 blocking ab treated samples, further confirming Hresistin regulated macrophage-derived mature IL-1β and IL-18 to mediate the post-injury proliferative responses in the lungs. To further confirm the effect of Hresistin on SMC proliferation levels of matrix metalloproteinases (MMP-1) were also analyzed in western blot. MMP-1 was induced by Hresistin treated conditioned media and were significantly reduced by blocking IL-1β or IL-18 (FIG. 8D).

### Discussion

Inflammasomes are key components of macrophage-mediated immunity.^{36,37} It first undergoes the priming step for the production of pro-caspase-1, pro-IL-1β, pro-IL-18 and NLRP3, followed by the activation step for NLRP3 assembly and cleavage of these pro forms.³⁸ The present inventor have found Hresistin/RELMα activates HMGB1 that activates NF-κB in macrophages leading to the priming of NLRP3 and its associated proteins. The present inventor also found Hresistin/RELMα binds and activates BTK, allowing it to activate the NLRP3 inflammasome through phosphorylation of four critical tyrosines on NLRP3. This study, represents an entirely novel understanding of the regulation of the NLRP3 inflammasome in the macrophage and in inflammatory diseases.

Following the stimulation of Hresistin at 200 ng/ml for 24 hours, the present inventor saw increased levels NLRP3, pro-forms of Caspase-1, IL-1b and IL-18 in both mRNA and protein levels. Although previous literature suggests the role of HMGB1 in the priming step, it hasn't been clearly demonstrated in detail. Since, Hresistin is an upstream regulator of HMGB-1, the present inventor proved Hresistin activates NF-κB through HMGB-1. Inhibiting HMGB-1 with Box A inhibitor, significantly downregulate the expression levels, proved the present inventor's concept of HMGB-1 being intermediate mediator after Hresistin/RELM-α in the priming pathway. Acetylated HMGB-1 secretes out of the cell and activates NF-κB through Toll-like receptor 4 (TLR4) or RAGE receptors (ref). When the present inventor did loss-of-function study, with all these macrophages were pre-treated with the specific HMGB1 antagonist recombinant Box-A protein followed by Hresistin/RELMα treatment, the present inventor saw significantly downregulated protein levels of NLRP3 inflammasome components, including NLRP3, caspase-1, IL-1β and IL-18. Few studies have suggested that Hresistin/RELM-α may act by activating the NF-κB pathway, which is necessary for the priming of the NLRP3 protein.³³ In addition, Hresistin has been shown to induce the expression of pro-inflammatory cytokines, such as IL-1β, which may contribute to the activation of the NLRP3 inflammasome.³⁰ In this study, the present inventor proved Hresistin-HMGB1 signaling as the mechanism of NLRP3 inflammasome priming in macrophages, for the first time.

After the production of inflammasome components in the priming step, the next activation step is to assemble NLRP3 and facilitate the secretion of IL-1β and IL-18.⁷ BTK was recently identified as the multifunctional direct regulator of NLRP3 inflammasome.³⁸⁻⁴⁰ Phosphorylated BTK induces the phosphorylation of four tyrosines of NLRP3 which facilitates the subsequent subcellular re-localization, oligomerization, ASC polymerization, and full NLRP3 assembly, leading to the cleavage and secretion of IL-1β and IL-18.³⁸⁻⁴⁰ BTK phosphorylation is an indicator of its NLRP3-regulating activity.^{38,41} The present inventor have identified Hresistin/RELMα as the binding partner and activator of BTK.¹⁶ RELMα induces BTK phosphorylation to promote the migration of myeloid cells.¹⁶ The present inventor proved that binding of Hresistin to BTK causes confirmational changes of BTK which facilitate it's phosphorylation and present this phopspho-BTK to NLRP3. The present inventor's data showed that Hresistin activates BTK to regulate caspase-1 activity, suggests the role of Hresistin/BTK signaling in full NLRP3 inflammasome activation. Treatment of human macrophages with 60 μM BTK kinase inhibitor ibrutinib, confirmed BTK activation is required for the downstream NLRP3 inflammasome complex assembly and for the cleavage and secretion of IL-1β and IL-18. Using MCC950 as a NLRP3 inhibitor further confirmed the caspase-1 activation is NLRP3 dependent. Similar to the priming pathway, for the first time, the present inventor proved activating BTK and Hresistin/RELMα-BTK signaling exhibits regulatory activities to induce the NLRP3 tyrosine phosphorylation, leading to the assembly and full activation of NLRP3 inflammasome in macrophages and the IL-1β/IL-18 secretion from these cells.

In this study the present inventor proved how macrophages were activated by Hresistin/RELM-α to secrete active IL-1β/IL-18 to initiate early immune responses. The present inventor propose that this priming and activation of the inflammasome is the mechanism of how elevated levels of Hresistin link to diseases including insulin resistance, atherosclerosis, diabetes, cardiovascular diseases (CVD), pulmonary hypertension (PH), autoimmune diseases, asthma and etc. The present inventor have found high levels of Hresistin in the lungs of PH patients,^{10,42} and Hresistin expression levels correlate with the severity of PH in humans and predicts mortality.⁴³ Therefore, to understand the how these early responses affect disease progression the present inventor selected PH as a disease model. In sterile inflammation like hypoxia, the endogenous DAMPs have been found to engage their receptors such as Toll-like receptors (TLRs), which leads to NF-κB activation and gene transcription of the NLRP3 inflammasome components. The present inventor now proved that Hresistin both primes and activates the NLRP3 inflammasome through its activation of HMGB1 and BTK, respectively, to initiate the inflammation that stimulates and maintains vascular remodeling in PH. Hresistin induced phosphorylation of AKT and ERK½, which are common proliferation markers. Blocking Hresistin with Hresistin antibody significantly reduced this phosphorylation, indicates the direct involvement of Hresistin. This was further confirmed with BrdU cell proliferation assay. The present inventor also examined the expression of components of Hresistin-BTK-NLRP3 signaling in clinical PH patients, and their possible correlation with hemodynamic and diagnostic markers of PH. The present inventor examined the NLRP3 activation pathway in human B cells, since BTK is the key component of B-cell-receptor signaling.⁴⁰ The present inventor found Hresistin was not expressed by human B cells. However, macrophage derived Hresistin/RELMα induced B cell chemotaxis by activating BTK in hypoxia, which recruits more B cells to lungs from spleen during the hypoxic condition. Consistent with previous findings, the present inventor saw macrophage derived Hresistin induced the activation of NLRP3 inflammasome in B cells through HMGB1 and BTK pathways. These findings led us to believe that Hresistin-HMGB1-BTK-NLRP3 signaling axis activates macrophages and subsequently induces the pro-PH phenotypes of B cells, synergistically contributing to pulmonary vascular remodeling and the development of PH. This may reflect the Hresistin-driven pathways for transition from innate to adaptive inflammatory responses to injury in the lung.

This paper proved for the first time how Hresistin regulates the priming stage through HMGB-1 and activation stage through BTK in immune cells to drive inflammatory responses where the Hresistin and NLRP3 inflammasome are involved. Hence, this study would address the critical gaps in understanding the primary mechanism by which NLRP3 inflammasome is primmed and activated in the macrophage/immune cells. The present inventor's work demonstrates that Hresistin is a highly effective and focused target to reduce NLRP3 action at the site of excessive inflammation using the present inventor's highly focused human monoclonal antibody against human resistin.

### CONCLUSION

This paper indicates for the first time the novel concept of the essential regulation of priming and activation stages of NLRP3 inflammasome through Hresistin/RELMα. Hresistin activates HMGB1-NFκB pathway to trigger the priming of NLRP3 inflammasome in macrophages. Hresistin activates BTK to induce subsequent assembly and activation of NLRP3 and the secretion of IL-1β and IL-18 in macrophages. The macrophage-derived Hresistin also bind to BTK and induce NLRP3 inflammasome activation in B cells. The present inventor also proved Hresistin/RELMα-producing macrophages induce migration and proliferation of PV-SMCs through secreting the inflammasome cytokines IL-1β and IL-18 to induce vascular remodeling over time for PH development. This study sheds light on the Hresistin driven immune responses and develop a novel immunotherapeutic approach for a variety of auto inflammatory disorders, including PH.

### REFERENCES

1 Daniel Walcher et al. Resistin: a newly identified chemokine for human CD4-positive lymphocytes. Cardiovasc. Res. 85 (2010).
2 Li, Y. et al. Resistin, a Novel Host Defense Peptide of Innate Immunity. 12 Front. Immunol. 699807 (2021).
3 Lin, Q. & Johns, R. A. Resistin family proteins in pulmonary diseases. Am. J. Physiol. Lung Cell Mol. Physiol. 319 (2020).
4 Chiara Pellicano et al. Serum resistin is predictive marker of development of new digital ulcers in systemic sclerosis. Clin. Exp. Med. 22 (2022).
5 Cong Lin, Zhixing Jiang, Ling Cao, Hejian Zou & Zhu., X. Role of NLRP3 inflammasome in systemic sclerosis. Arthritis Res. Ther. (2022).
6 Pellegrini, C. et al. NLRP3 at the crossroads between immune/inflammatory responses and enteric neuroplastic remodelling in a mouse model of diet-induced obesity. Br. J. Pharmacol. 178 (2021).
Swanson, K. V., Deng, M. & Ting, J. P. The NLRP3 inflammasome: molecular activation and regulation to therapeutics. Nat. Rev.
   19, 477-489 (2019).
8 Bittner, Z. A. et al. BTK operates a phospho-tyrosine switch to regulate NLRP3 inflammasome activity. J. Exp. Med. 218 (2021).
9 Rabinovitch, M., Guignabert, C., Humbert, M. & Nicolls, M. R. Inflammation and immunity in the pathogenesis of pulmonary arterial hypertension. Circ. Res. 115, 165-175 (2014).
10 Lin, Q. et al. RELMalpha Licenses Macrophages for Damage-Associated Molecular Pattern Activation to Instigate Pulmonary Vascular Remodeling. J. Immunol. 203, 2862-2871 (2019).
11 Lin, Q. et al. HIMF (Hypoxia-Induced Mitogenic Factor) Signaling Mediates the HMGB1 (High Mobility Group Box 1)-Dependent Endothelial and Smooth Muscle Cell Crosstalk in Pulmonary Hypertension. Arterioscler. Thromb. Vasc. Biol. 39, 2505-2519 (2019).
12 Fan, C. et al. S100A11 mediates hypoxia-induced mitogenic factor (HIMF)-induced smooth muscle cell migration, vesicular exocytosis, and nuclear activation. Mol. Cell. Proteomics 10, M110 000901 (2011).
13 Nair, M. G., Cochrane, D. W. & Allen, J. E. Macrophages in chronic type 2 inflammation have a novel phenotype characterized by the abundant expression of Ym1 and Fizzl that can be partly replicated in vitro. Immunol Lett 85, 173-180 (2003).
14 Stutz, A. M. et al. The Th2 cell cytokines IL-4 and IL-13 regulate found in inflammatory zone 1/resistin-like molecule alpha gene expression by a STAT6 and CCAAT/enhancer-binding protein-dependent mechanism. J Immunol 170, 1789-1796 (2003).
15 Pesce, J. T. et al. Retnla (relmalpha/fizz1) suppresses helminth-induced Th2-type immunity. PLOS Pathog 5, e1000393 (2009).
16 Su, Q., Zhou, Y. & Johns, R. A. Bruton's tyrosine kinase (BTK) is a binding partner for hypoxia induced mitogenic factor (HIMF/FIZZ1) and mediates myeloid cell chemotaxis. FASEB J. 21, 1376-1382 (2007).
17 Smith, C. I. E. et al. X-linked agammaglobulinemia:
   lack of mature B lineage cells
   caused by mutations in the Btk kinase. Springer semin in immunopathology 19 (1998).
18 Mukhopadhyay, S., George, A., Bal, V., Ravindran, B. & Rath, S. Bruton's tyrosine kinase deficiency in macrophages inhibits nitric oxide generation leading to enhancement of IL-12 induction. J Immunol 163, 1786-1792 (1999).
19 Mukhopadhyay, S. et al. Macrophage effector functions controlled by Bruton's tyrosine kinase are more crucial than the cytokine balance of T cell responses for microfilarial clearance. J Immunol 168, 2914-2921 (2002).
20 Nair, M. G. et al. Alternatively activated macrophage-derived RELM-alpha is a negative regulator of type 2 inflammation in the lung. J Exp Med 206, 937-952 (2009).
21 Li, Z. et al. Resistin promotes CCL4 expression through toll-like receptor-4 and activation of the p38-MAPK and NF-kappaB signaling pathways: implications for intervertebral disc degeneration. Osteoarthritis Cartilage 25, 341-350 (2017).
22 Nair, M. G. et al. Alternatively activated macrophage-derived RELM-{alpha} is a negative regulator of type 2 inflammation in the lung. J Exp Med 206, 937-952 (2009).
23 Ito, M. et al. Bruton's tyrosine kinase is essential for NLRP3 inflammasome activation and contributes to ischaemic brain injury. Nature communications 6 (2015).
24 Purvis, G. S. D. et al. Inhibition of Bruton's TK regulates macrophage NF-kappaB and NLRP3 inflammasome activation in metabolic inflammation. Br J Pharmacol 177, 4416-4432 (2020).
25 Pugliese, S. C. et al. The role of inflammation in hypoxic pulmonary hypertension: from cellular mechanisms to clinical phenotypes. Am. J. Physiol. Lung Cell Mol. Physiol. 308, L229-252 (2015).
26 Lin, Q. et al. Human Resistin Induces Cardiac Dysfunction in Pulmonary Hypertension. J Am Heart Assoc 12, e027621 (2023)
27 Al-Qazazi, R. et al. Macrophage-NLRP3 Activation Promotes Right Ventricle Failure in Pulmonary Arterial Hypertension. Am. J. Respir. Crit. Care Med. 206, 608-624 (2022).
28 Parpaleix, A. et al. Role of interleukin-1 receptor 1/MyD88 signalling in the development and progression of pulmonary hypertension. Eur. Respir. J. 48, 470-483 (2016).
29 Anna Foley, Benjamin E. Steinberg & Goldenberg, N. M. Inflammasome Activation in Pulmonary Arterial Hypertension. Frontires in Medicine 8 (2022).
30 Chen, W.-C., Tsai, C.-H., Lu, Y.-C., Liu, S.-C. & Tang, C.-H. Resistin enhances IL-1β and TNF-α expression in human osteoarthritis synovial fibroblasts by inhibiting miR-149 expression via the MEK and ERK pathways. The Federation of American Societies for Experimental Biology journal 34 (2020).
31 Park, H. K. & Ahima, R. S. Resistin in Rodents and Humans. Diabetes Metab. 37 (2013).
32 Shin, J. H., Park, S., Cho, H., Kim, J. H. & Cho, H. Adipokine human Resistin promotes obesity-associated inflammatory intervertebral disc degeneration via pro-inflammatory cytokine cascade activation. Nature Scientific reports (2022).
33 Silswal, N. et al. Human resistin stimulates the pro-inflammatory cytokines TNF-alpha and IL-12 in macrophages by NF-kappaB-dependent pathway. Biochem Biophys Res Commun 334, 1092-1101 (2005).
34 Kim, E. J. et al. HMGB1 Increases IL-1beta Production in Vascular Smooth Muscle Cells via NLRP3 Inflammasome. Front. Physiol. 9, 313 (2018).
35 Giambelluca, S., Ochs, M. & Lopez-Rodriguez, E. Resting time after phorbol 12-myristate 13-acetate in THP-1 derived macrophages provides a non-biased model for the study of NLRP3 inflammasome. Front. Immunol. 13, 958098 (2022).
36 Martinon, F., Burns, K. & Tschopp, J. The inflammasome: a molecular platform triggering activation of inflammatory caspases and processing of proIL-beta. Mol Cell 10, 417-426 (2002).
37 Scott, T. E., Kemp-Harper, B. K. & Hobbs, A. J. Inflammasomes: a novel therapeutic target in pulmonary hypertension? Br J Pharmacol 176, 1880-1896 (2019).
38 Bittner, Z. A. et al. BTK operates a phospho-tyrosine switch to regulate NLRP3 inflammasome activity. J Exp Med 218 (2021).
39 Ito, M. et al. Bruton's tyrosine kinase is essential for NLRP3 inflammasome activation and contributes to ischaemic brain injury. Nat Commun 6, 7360 (2015).
40 Weber, A. N. R. et al. Bruton's Tyrosine Kinase: An Emerging Key Player in Innate Immunity. Front Immunol 8, 1454 (2017).
41 Duarte, D. P. et al. Btk SH2-kinase interface is critical for allosteric kinase activation and its targeting inhibits B-cell neoplasms. Nat Commun 11, 2319 (2020).
42 Angelini, D. J. et al. Resistin-like molecule-b in scleroderma-associated pulmonary hypertension. Am J Respir Cell Mol Biol 41, 553-561 (2009).
43 Lin, Q. & Johns, R. A. Resistin family proteins in pulmonary diseases. Am J Physiol Lung Cell Mol Physiol 319, L422-L434 (2020).

### Example 2: Development Anti-Human Resistin Monoclonal Antibodies

Proteins in the resistin-like molecule (RELM) family are critically involved in the pathogenesis of a variety of inflammation-related pathologies. The present inventor's previous rodent work strongly suggested that human resistin (hResistin) is mechanistically important to the etiology of human vascular inflammatory diseases and constitutes a therapeutic target. Therefore, the present inventor endeavored to develop human antibodies against hResistin. The anti-hResistin monoclonal antibodies were generated through phage screening of a human library, validated for their in vitro anti-proliferative function against hResistin in primary human pulmonary smooth muscle cells (SMCs), and further screened for immunogenicity, manufacturability, stability, and toxic effects. Their purity, antigen binding affinities and structural modeling were also validated. These human antibodies inhibited hResistin-stimulated proliferation of human primary SMCs and exhibited a cross-reactive ability to block rodent RELM actions in SMC assays. Thus, the present inventor successfully produced the monoclonal anti-hResistin therapeutic antibodies which exhibits anti-proliferative and immuno-modulatory activities. These neutralizing antibodies hold great promise as a novel therapy for PH and other hResistin/RELM-related inflammatory diseases.

### INTRODUCTION

Resistin-like molecule (RELM) signaling is an important component of the type II inflammatory response to tissue injury in the lung and other organs,^{1,2} and may be critically involved in inflammasome signaling and its downstream responses. Using gene array technology, the present inventor's laboratory discovered a molecule in the lungs of mice exposed to a chronic hypoxia-induced PH model and named it hypoxia-induced mitogenic factor [HIMF, which is also known as resistin-like molecule (RELM) a or FIZZ1) because it was upregulated in the hypoxic lung and showed potent mitogenic effects.³ Later studies from the present inventor's lab and others revealed that the RELM family of proteins comprises pleiotropic cytokines critically involved in the vascular remodeling and cardiac dysfunction and remodeling seen in animal and human pulmonary arterial hypertension (PH),^{1, 2, 4-7} strongly suggesting a causal role of resistin family proteins in human PH. RELMα also has been shown to play a critical role in the development of Th2 inflammatory models induced by ovalbumin sensitization, schistosomiasis, and HIV-related stimuli.^{1, 3, 4, 6-9} Human resistin (hResistin) is expressed by myeloid cells, especially macrophages, and its expression pattern shows a greater similarity to that of murine HIMF/RELMα than to that of murine resistin.¹⁰ The present inventor's mechanistic study of gene-modified mouse lines recently revealed that hResistin induces pulmonary vascular remodeling and PH development by mediating the endothelial and smooth muscle cell crosstalk and macrophage activation dependent on activation of damage-associated molecular pattern (DAMP) signaling._{11, 12} Moreover, the present inventor have found that the elevation of resistin in peripheral blood of patients with idiopathic PH and or scleroderma-associated PH correlates with severity of PH hemodynamic changes (unpublished observations). Additionally, research into the immuno-regulatory properties of RELMα and hResistin has expanded to other related pulmonary pathologies including fibrosis and cancer in lung.² The cardiac-specific effects of hResistin on modulating inflammation in heart also have been revealed in the present inventor's recent study.^{13, 14} Moreover, the hResistin-induced vascular lesions and inflammation might also lead to atherosclerosis, thrombosis, diabetes, pathological angiogenesis, cancers, and other vascular inflammatory diseases.² All these data strongly suggest that hResistin might contribute to the etiology of the related human vascular inflammatory diseases and that it might serve as a biomarker and therapeutic target for these diseases.

Thus, the present inventor began preclinical testing to identify specific agents that could inhibit the onset or progression of PH in humans by impeding the activity of hResistin. The present inventor previously observed that trans-tracheal administration of short hairpin (sh) RNA against rodent HIMF/RELMα prevented much of the vascular remodeling and hemodynamic changes that occur during development of hypoxia-induced PH.⁴ Similarly, recombinant elimination of rodent HIMF/RELMα had anti-PH effects.¹¹ As the role of hResistin in a wide variety of human diseases, especially the cardiothoracic and vascular pathologies, expands rapidly,² the potential application of a therapeutic antibody against hResistin widens and becomes increasingly important. Thus, the present inventor hypothesize that suppressing the actions of hResistin with a blocking antibody will delay, prevent, or reverse progress of PH. Because hResistin is significantly expressed only under pathologic conditions,¹⁵ successful targeting of this protein to treat PH is likely to prove highly specific actions and to have few side effects. After development and screening of the therapeutic antibodies against hResistin, the present inventor evaluated their effectiveness and their feasibility for use in humans with preclinical in vitro and vivo assays and validated their cross-reactivity for blocking rodent RELMα. The present inventor's results indicate that monoclonal anti-hResistin therapeutic antibodies have potential efficacy against PH and other related inflammatory pathologies in lung and other organs in rodents and humans.

### Materials and Methods

Anti-hResistin Antibody Development. Antibodies were developed in cooperation with the present inventor's commercial partners Creative Biolabs (Shirley, NY). Lonza (London, England) and Wuxi AppTec (Cambridge, MA, USA and Shanghai, China) provided scale-up production. Lonza, Wuxi, Antibody Solutions (Mountainview, CA), Charles River Laboratories (Frederick, MD) and the Johns Hopkins Department of Veterinary Medicine (Baltimore, MD) were contracted to provide validation studies of the antibodies in support of the validation work done in the present inventor's own laboratory. First, based on the FLAG-tag recombinant protein made and purified by the present inventor's lab,¹⁵ the present inventor identified the antibodies using a phage display approach with several human antibody libraries with Creative Biolabs. The sequences the present inventor selected then were used to generate antibody-producing hybridoma cells. After screening these cells through soluble ELISA, Lonza used their proprietary GS Xceed® TM/XS System in CHO cells to generate high-yield clones (>200 mg/L) of each of five lead antibody candidates, expanding them for use in the following in vivo animal experiments.

Development of In Vitro Human Smooth Muscle Cell (SMC) Proliferation Bioassays for Antibody Testing. Primary human bronchial or pulmonary vascular artery SMCs [from Lonza (Morristown, NJ), 2,000 cells/well in 96-well plates] were stimulated with lab-made recombinant rat RELMα (0.01-5 μg/mL) or hResistin (0.1 to 10 μg/mL) for 48 hours. Then 5-bromo-2′-deoxyuridine (BrdU) was added for 24 hours to label proliferating cells. Lastly, BrdU ELISA was performed to measure and quantify cell proliferation. For the blocking study, recombinant proteins of 3 μg/mL hResistin or 0.3 μg/mL rat RELMα were incubated with antibodies for 20 minutes before they were applied to human bronchial SMCs. The present inventor conducted BrdU ELISA (11647229001, Roche) to assess proliferation and to develop a human analytic bioassay for future use.

Statistical Analysis. Continuous variables are presented as the mean±SEM. Dichotomous variables are presented as number and percentage values. Preliminary data sets for all analyses presented were used for a power analysis to determine sample sizes necessary for adequate statistical power. Data were analyzed with Student's t test for comparisons between two groups and with one-way ANOVA followed by the Newman-Keuls post-hoc test for multiple comparisons. All analyses were performed with Prism 7.0e (GraphPad Software, La Jolla, CA). A p<0.05 was accepted as statistically significant.

Extended Materials and Methods for recombinant RELM protein production, phage display approach, immunoprecipitation (IP), ELISA evaluation, in silico screening, size exclusion HPLC (SE-HPLC), BIACORE plasmon resonance assay, endotoxin measurement, structural modeling, stability study, and toxicological assessment, are provided in the Supplemental Data. All animals, antibodies and cell lines used in this study are detailed in the Supplemental Data.

### Results

Selection and Initial Screening of Generated Antibodies. Of the initial 80 clones chosen from screening, the present inventor selected 17 hResistin scFv clones based on the capability of binding to their targeted recombinant hResistin proteins, as determined by antigen vs antibody dose response in soluble ELISAs. Each antibody was also evaluated by dose response of its association and dissociation binding kinetics assessed by plasmon resonance. The present inventor performed an additional dose-related ELISA to determine and validate the potency and specificity of binding of these prepared full human IgG1 antibodies to hResistin. In silico analysis of T-cell recognition were predicted for DRB 1 alleles and ranked against a test set of antibodies with a known immunogenicity response in a clinical setting. Results indicated that all 17 clones had a lower risk than therapeutic antibodies currently in clinical use. The present inventor thereby narrowed the present inventor's list of candidates to 10 antibodies to be further tested. All sequences of these short-listed antibodies were then subjected to immuno-profiling for the presence of Th-epitopes, by Lonza Inc. (Slough, England) through the Epibase platform's “HLA class II-Global v3.0” setting. Based on the critical epitope counts, the affected HLA allotypes and DRB1 risk score, the present inventor were able to initially rank the ten antibodies by increased immunogenic risk (Table I). Predictive computational tools were also employed to analyze theoretical molecular data (Table II), and identify possible post-translational modifications (PTMs) of these antibodies (Table III). The amino-acid sequences of the ten tested candidate antibodies were screened for the sequence motifs and features of a number of potential developability issues (Table IV) and for aggregation risk (Table V). Two antibodies (#6 and #10) were recommended for further development with no detected issues, and six antibodies (#1, 2, 4, 5, 7, and 8) can be considered for further development with a few potential issues. For antibodies #3 and #9, additional in vitro risk assessment and potentially protein engineering were recommended prior to further development. Next, these 10 antibodies were further tested in the in vitro functional assays.

In Vitro Efficacy Studies. The present inventor developed an effective cell-based assay of hResistin response that is robust, highly consistent, and relevant to lung biology as a model to test the blocking effects of the present inventor's antibodies, which was recommended by FDA and EMA. The present inventor's lab has published the proliferative and chemotactic actions of RELMα and hResistin on human pulmonary SMCs, bone marrow stem cells, leukocytes, and human mesenchymal stem cells¹⁶⁻¹⁸ and hResistin-induced activation of human lung ECs and SMCs.^{19, 20} These studies showed proof of principle for using cell proliferation and migration assays to assess antibody efficacy in isolated cell studies.

The present inventor have tried a variety of relevant human cell lines (PA-SMCs, B-SMCs, PMV-ECs, human lung fibroblasts, and the human THP-1 cell lines) to test the effect of antibody blockade of hResistin on a variety of parameters including expression of specific proteins/genes (endothelin, IL-6, IL-8, MCP-1, TIMP-1 urokinase plasminogen activator [uPA], collagen 1A1, collagen 3A, NF-κB, CAP1 adenylyl cyclase activation) and physiologic responses such as cell migration and cell proliferation. Several difficulties came out including: the fragility of cultured ECs, less highly consistent in response from passage to passage and batch to batch, and the ability of IgG to non-specifically induce migration and proliferation in immune cells (data not shown). By solving the above-listed issues, the bioassay based on hResistin-stimulated proliferation of human primary bronchial SMCs and pulmonary artery SMCs, as assessed by BrdU incorporation into cellular DNA during cell proliferation, was chosen as the key bioassay tool for assessing the present inventor's drugs. As the pulmonary bronchial and vascular SMC proliferation is the hub mechanism of the resistin/RELMα-induced lung Th2 inflammation and PH, this potency assay thus also represented the biological effect and therapeutic activity of the present inventor's generated antibodies.

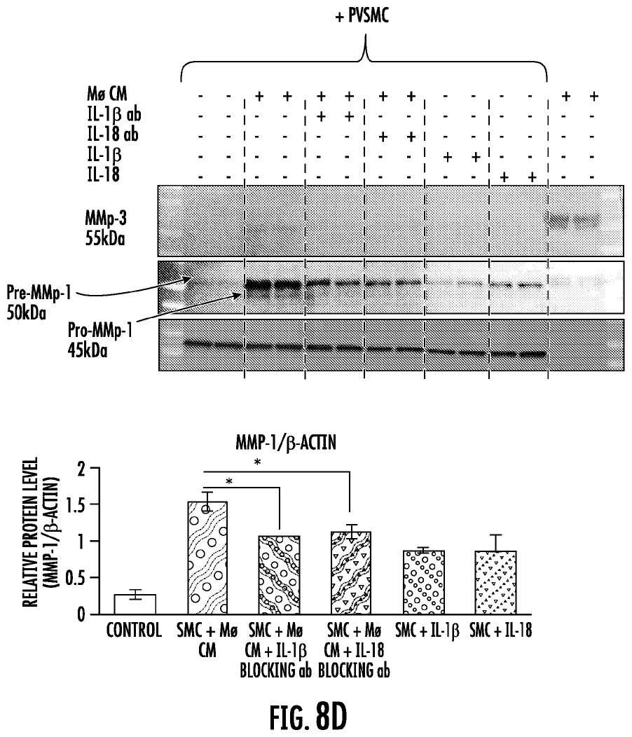

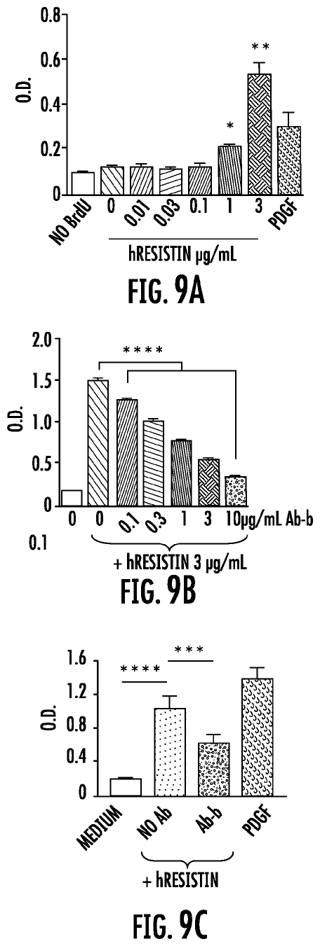

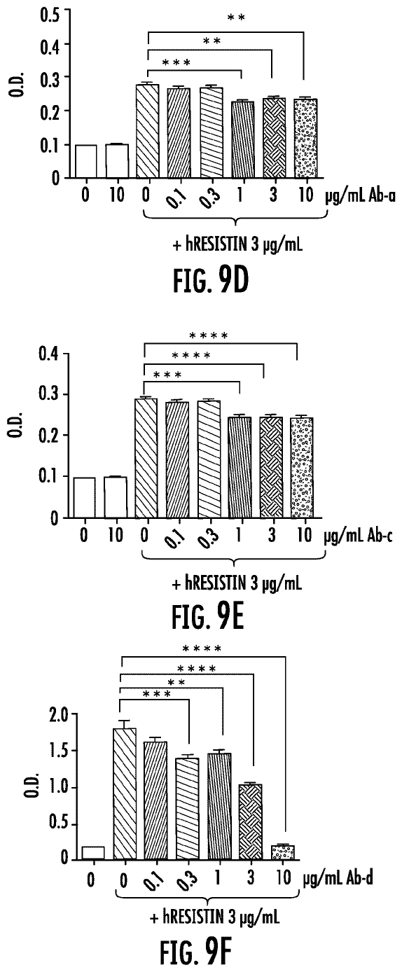

This cell assay was performed following the Guidance for Industry: Potency for cellular and gene therapy products issued by FDA in 2011 (the Part IV. Potency Assay Design and Validation). In this study, all the reagents, materials, standards, controls and calibrated equipment, as well as the training of operators, were adequately qualified. Sample randomization and quantitative analysis of the BrdU read-out with appropriate statistical methods, as well as sufficient number of replicates, demonstrated the accuracy, precision, specificity and consistency of this bioassay. Because preliminary experiments showed that the 3 μg/mL dose of hResistin was the most potent for activating proliferation of bronchial and pulmonary vascular SMCs, the present inventor chose this dose to evaluate the antibody capabilities (FIG. 9A-1C). PDGF at 20 ng/mL served as a positive control. With this assay, the present inventor further ranked the 17 antibodies (selected from the initial 80 clones as mentioned in the above section of Selection and Initial Screening) by potency and specificity in blocking function. The present inventor then chose four as lead antibody candidates based on their blocking potentials. The present inventor further labelled them as Ab-a, Ab-b, Ab-c, and Ab-d. Of these, Ab-b was the most potent (FIGS. 9B and 9D-9F). It exhibited marked blocking efficacy in human primary SMC bioassays as shown in FIG. 9B (bronchial) and 1C (pulmonary vascular). The human cell-based bioassay thus allowed us to rank, choose, and validate the continued efficacy of lead antibodies against hResistin, and formed the basis the following testing in PH animal models.

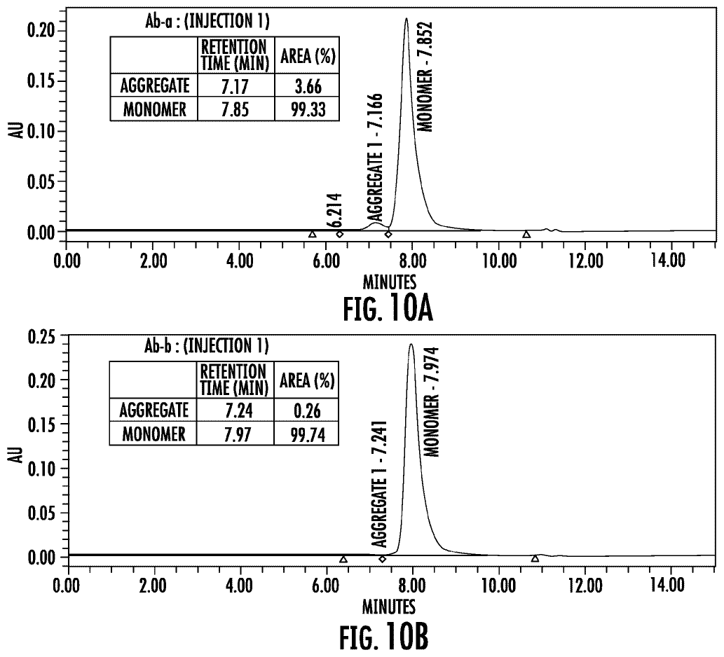

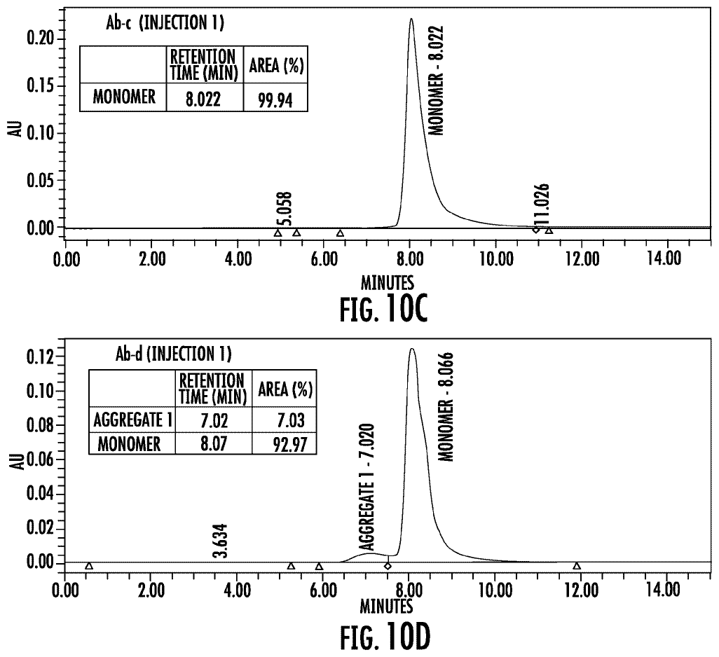

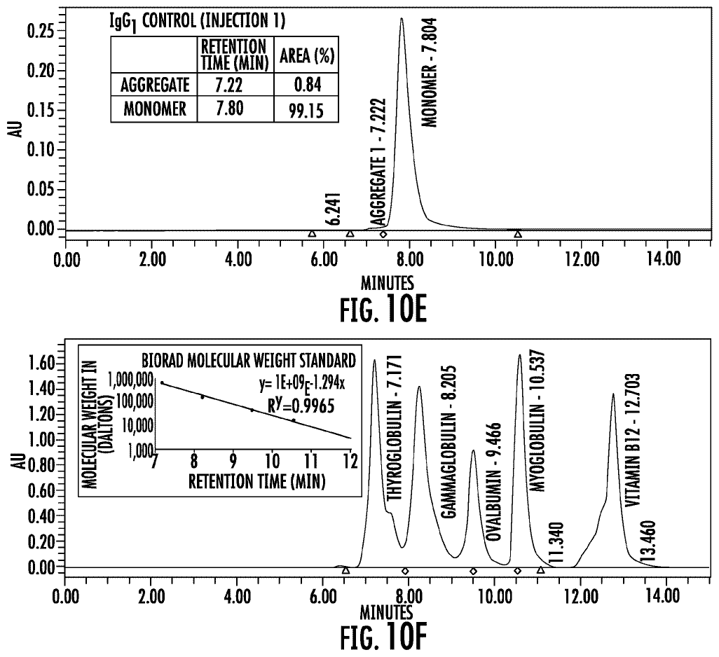

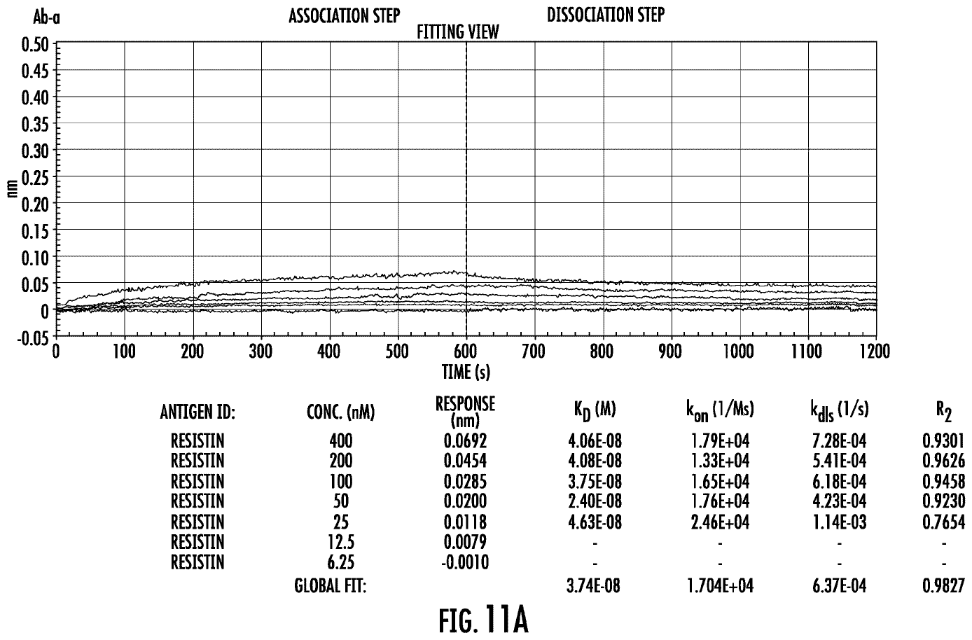

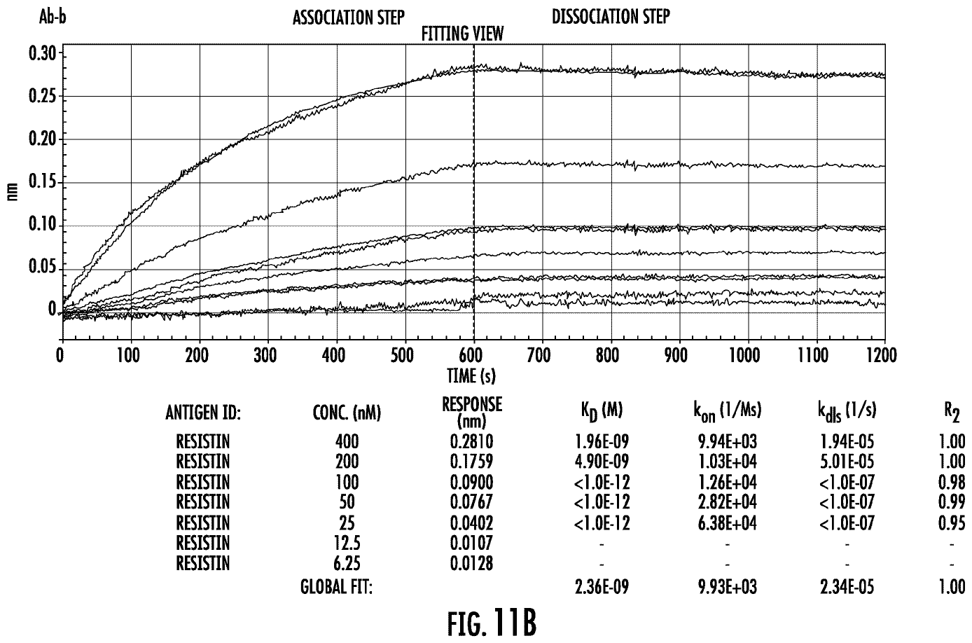

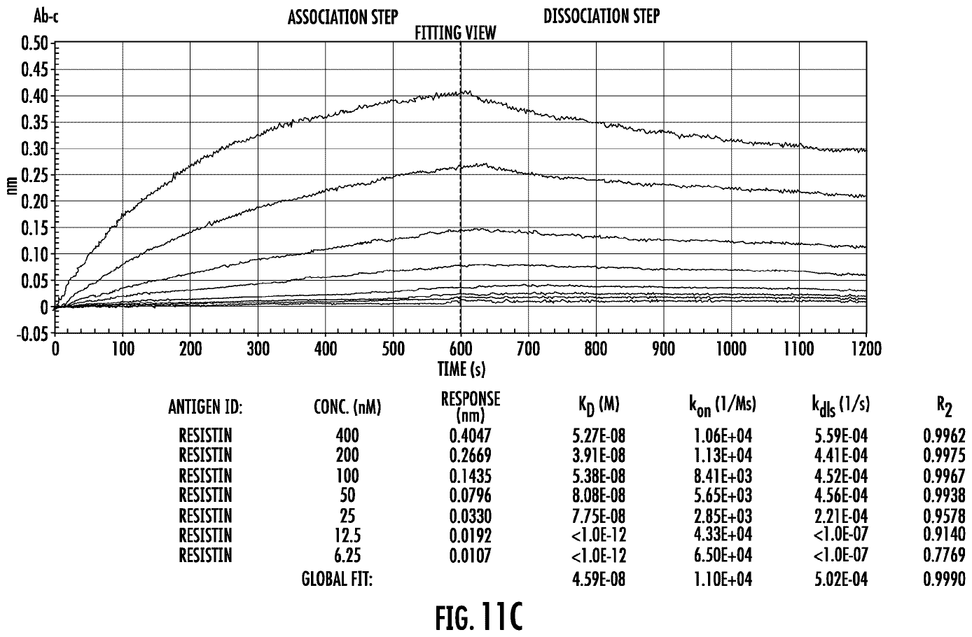

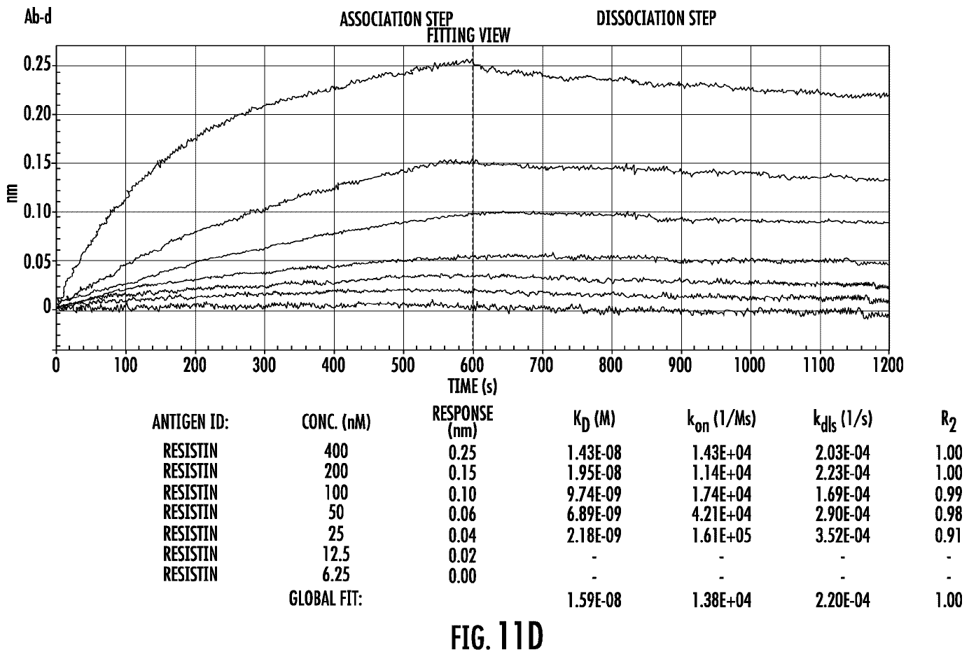

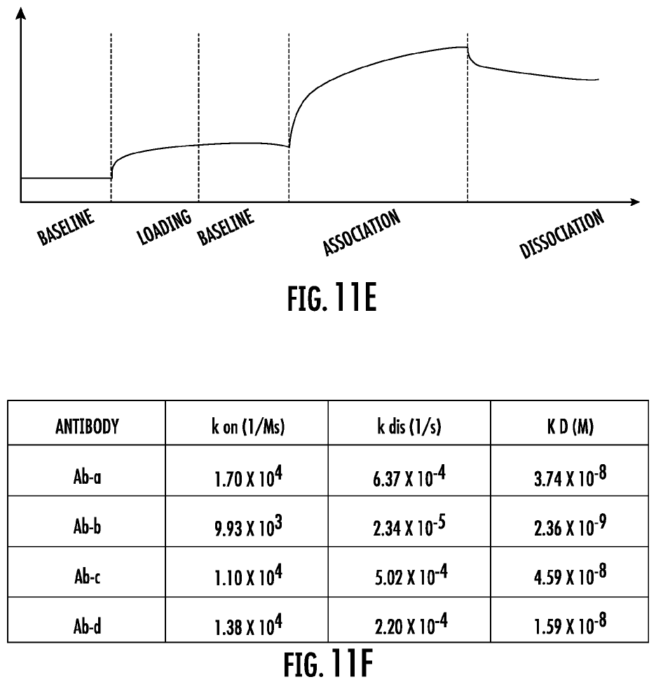

Binding Characteristics of the Lead Antibodies. These four above-selected lead antibodies were further synthesized by Lonza in small (1.2 L culture) and large (6 L culture) batches in CHO cells for use in in vivo assays. Single and double gene GS vectors (DGVs) using Lonza's GS Xceed™ Gene Expression System were constructed and transfected into CHOK1SV GS-KO cells to express those antibodies. Stable recombinant cells were propagated for the fed-batch overgrowth cultures. Protein titre in supernatant samples was determined by Octet. Purification of antibodies in medium was carried out by Protein A affinity chromatography, to yield between 11.5 and 187 mg of purified material for the small scale evaluation cultures (Table VI), and between 350 mg to 2,028 mg for the large scale material supply (Table VII). Comparably, Ab-b expressed from fast stable pool at 7L scale was evaluated bioreactor production on CHO cell culture process platform by Wuxi AppTec, another one of the present inventor's CRO's. The yield of Ab-b was 1.17 g/L final titers with a purity of 96.07% after purification and less than 1EU/mg for endotoxin, demonstrating the promise of successful manufacturability. The product quality was further validated by SE-HPLC analysis, and the generated antibodies exhibited high purity and low aggregate levels (FIG. 10, and Table VIII). Endotoxin levels in the large-scale (1-2 grams) purified antibodies were below 0.53 EU/mg. These lead antibodies exhibited potency and selectivity of binding to hResistin protein as assessed by the BIACORE plasmon resonance assay (FIG. 11). The C-terminal FLAG-tagged hResistin was utilized as the immobilized protein and each individual antibody was added to the mobile phase. The present inventor examined a dose range from 6.25 nM to 400 nM for antibodies that show a strong initial response, and the Kd for binding was calculated to assess the kinetics of the BIACORE curve, particularly seeking a slow descent during washout, suggestive of a high affinity and long duration of action, with characteristics that can be improved with affinity modifications. The data showed rapid high affinity binding and a slow dissociation (FIG. 11), optimal responses for a therapeutic antibody. Especially the antibody Ab-b exhibited high affinity for binding of the targeted antigen, with a K_{D} of 2.36×10⁻⁹ M (FIGS. 11B and 11F), which was consistent with its potent anti-proliferative activities (FIG. 9).

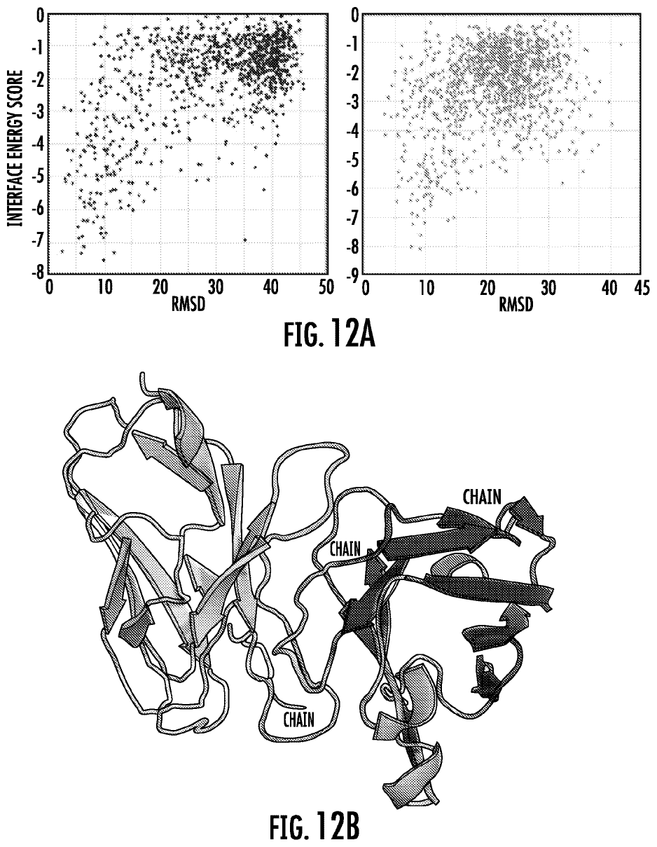

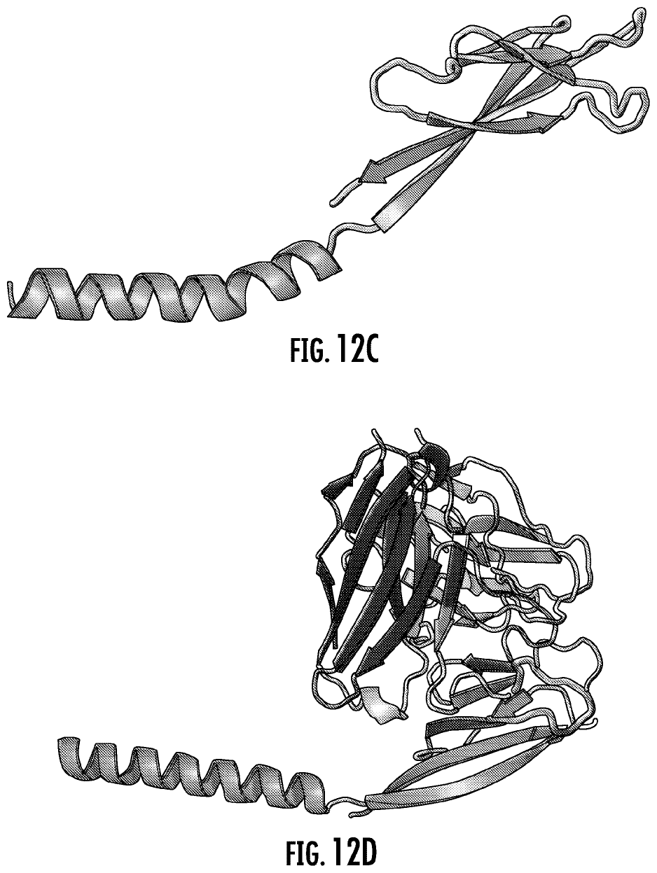

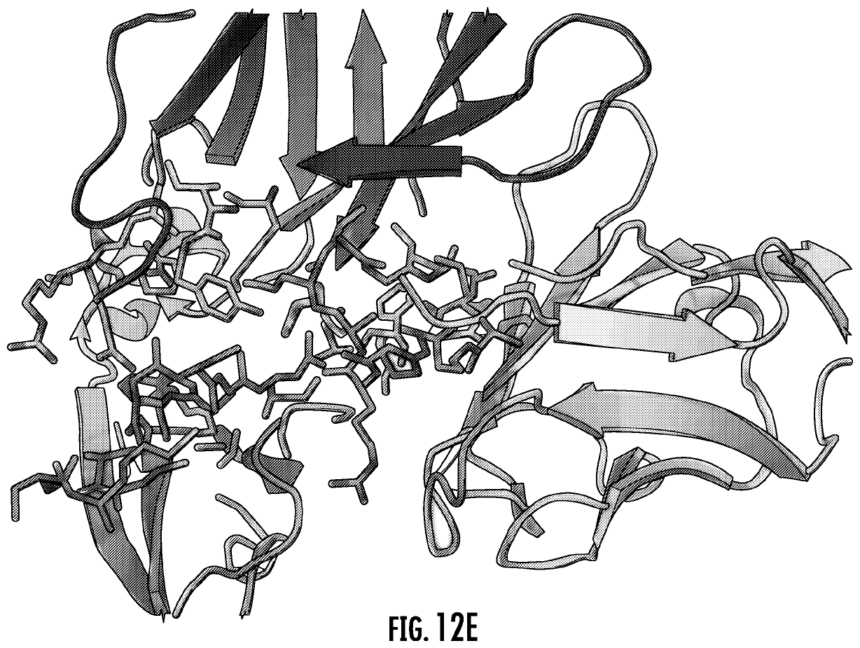

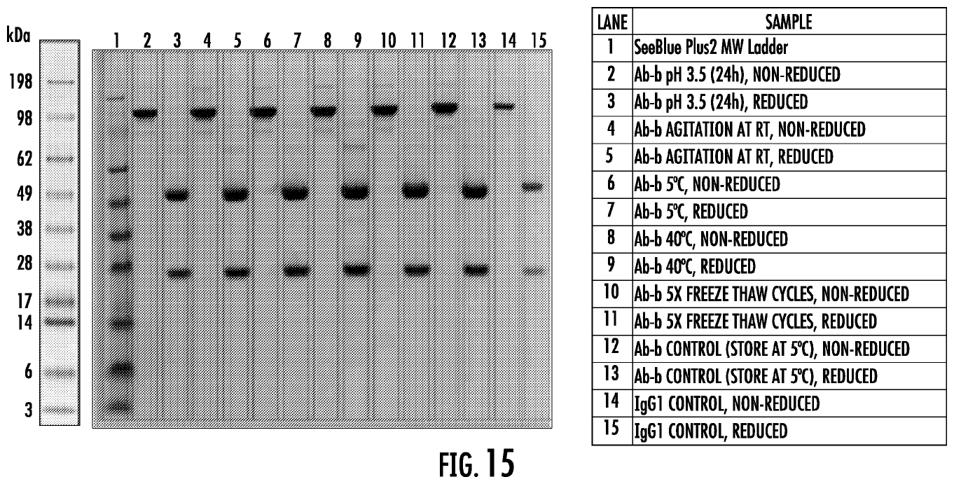

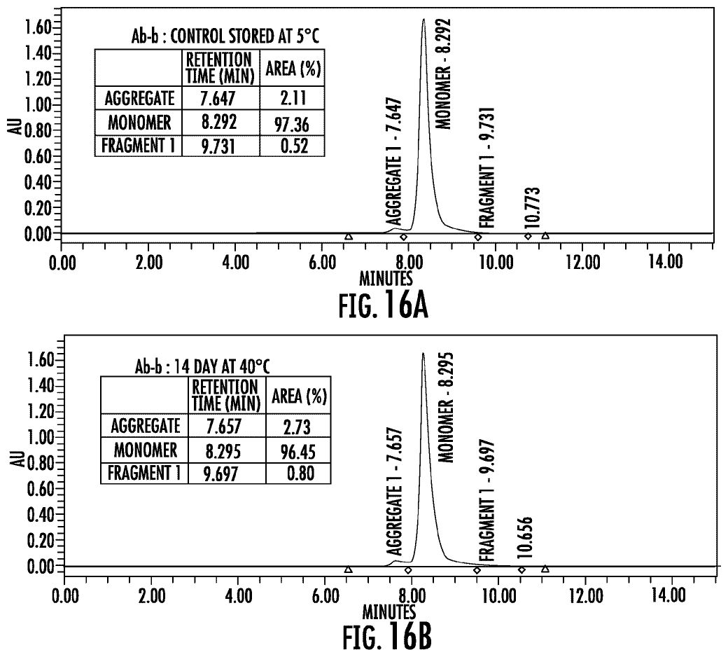

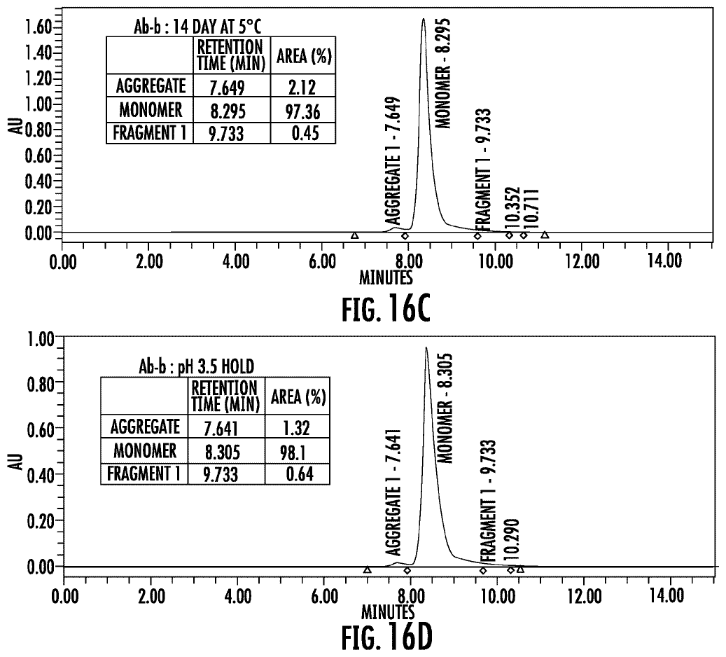

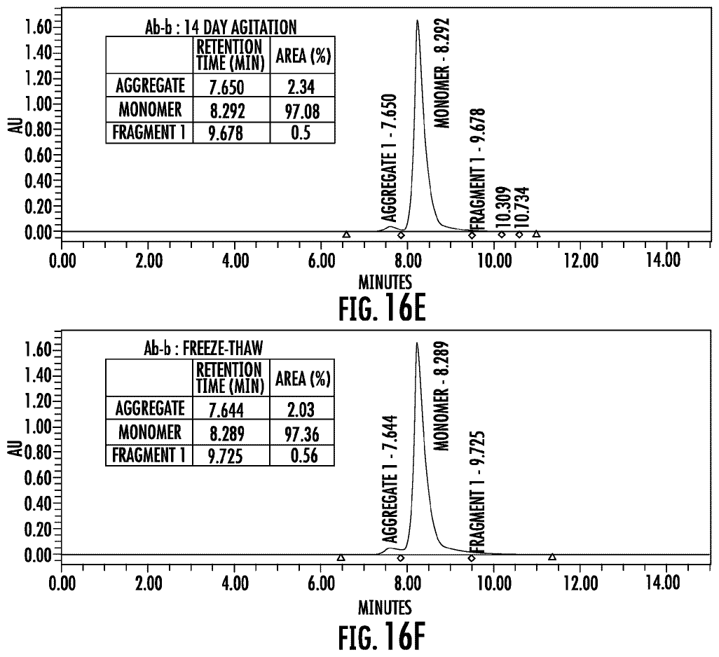

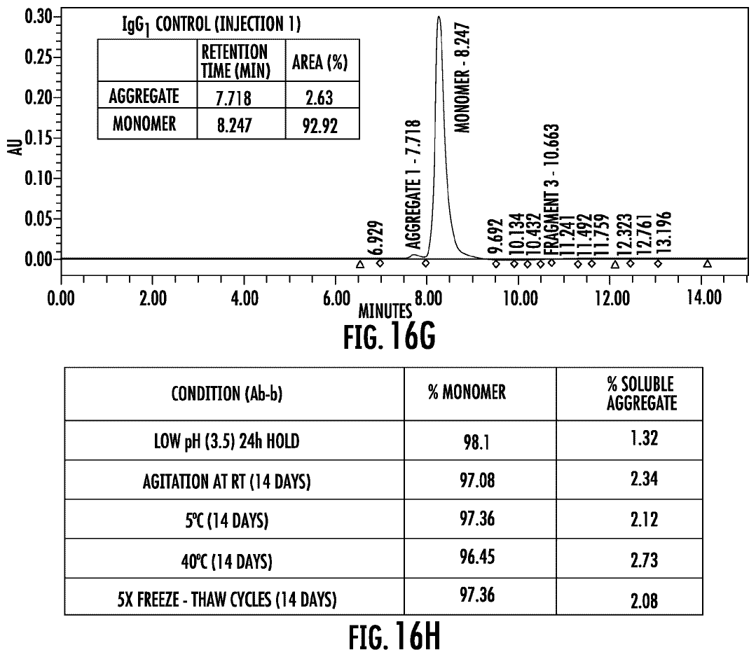

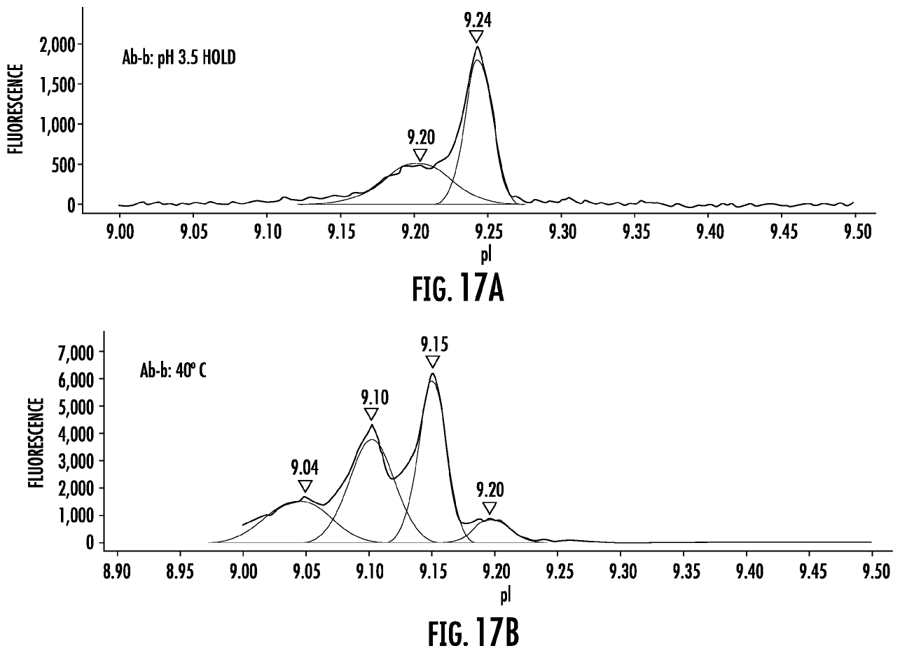

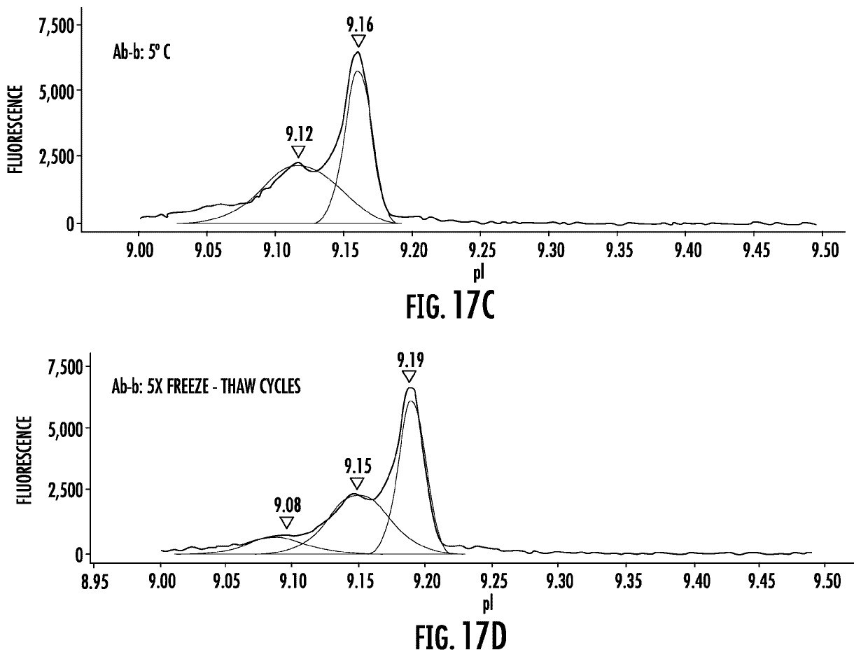

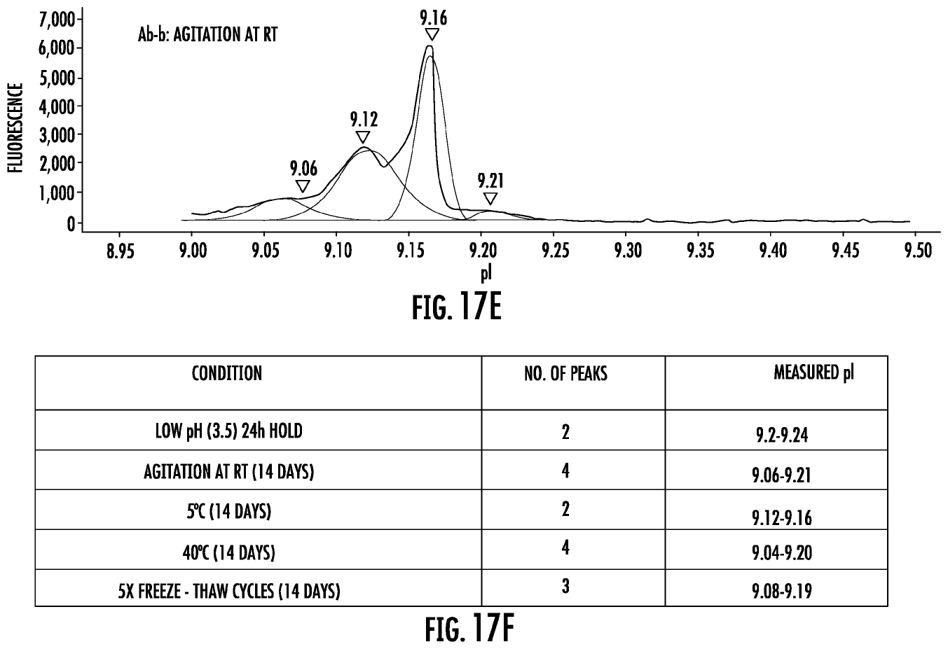

The present inventor thus employed structural modeling with RosettaAntibody and SnugDock²¹ to further analyze the binding sites on hResistin interacting with Ab-b. On the hResistin protein, as reported previously,²² two active site epitope regions were predicted: residue 50-65 and 78-93. The present inventor thus focused the present inventor's docking on these two regions. Using a local docking run, the present inventor separately docked both the epitope regions of hResistin with the antibody Ab-b (FIG. 12A). Docking indicated that the putative epitope regions are binding sites for the hResistin protein when present in monomer state (FIG. 12A) which is the most functional form of hResistin exhibiting pro-proliferative capabilities (data not shown). Structural modeling further suggested that the lead antibody Ab-b interacts with the active binding epitopes in the globular head region of hResistin (FIG. 12B-12E)²³, thereby blocking the pro-mitogenic effects of hResistin. Stability of the generated Ab-b exposed to different conditions (Table IX) was also assessed via SDS-PAGE (FIG. 15), SH-HPLC (FIG. 16), and cIEF (FIG. 17). No major degradation and aggregation of the Ab-B was tested under conditions at low PH, high temperature, agitation or freeze-thaw (FIG. 16). cIEF analysis showed no significant PI isoform changes under tested conditions (FIG. 17). Data indicated that Ab-b remained stable over different conditions of stresses. Collectively, along with the above human cell-based bioassay, these data from plasmon resonance, aggregation, structural, and epitope assessment demonstrated the efficacy and quality of the present inventor's antibody, which is essential for testing their therapeutic effects in the in vivo PH animal models, and later human.

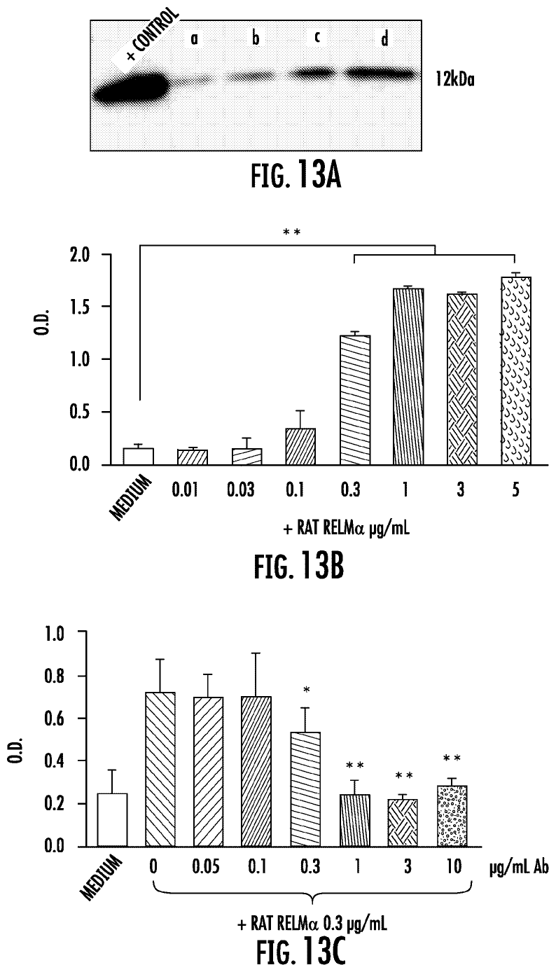

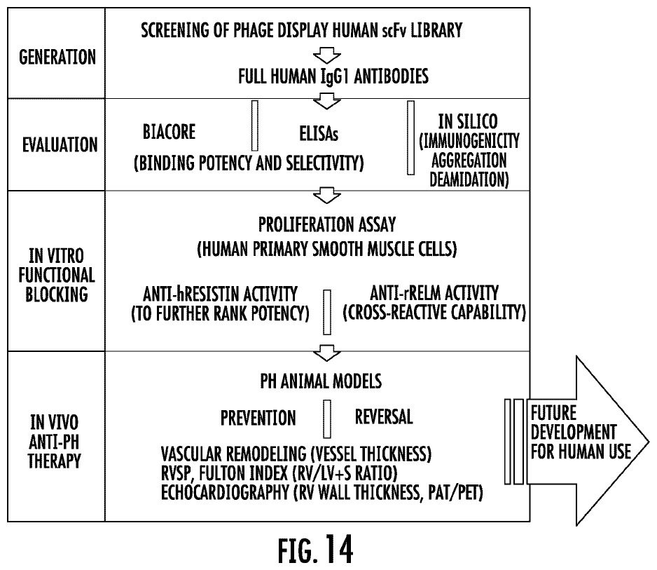

Determination of the Cross-reactive Efficiency of Anti-hResistin Antibodies for Blocking Rodent RELMα-induced Cell Proliferation. Next, the present inventor used an IP assay to confirm that the present inventor's human lead antibodies were capable of binding to rat and mouse RELMα (FIG. 13A). Rodent RELMα dose-dependently stimulated the proliferation of human bronchial SMCs at a minimum dose of 0.3 μg/mL (FIG. 13B). Intriguingly the present inventor's anti-hResistin human antibodies blocked the SMC proliferation induced by rodent RELMα (FIG. 13C), indicating that rat RELMα can activate human RELM responses. Based on these results, the present inventor went on to examine whether antibody application could be therapeutically efficacious in the rodent PH models (FIG. 14), a critical step in moving these antibodies toward human use.

Assessment of systematic and organ-specific toxicity of anti-hResistin antibody. To further exclude the immunotoxicity and potential systemic side effects of the antibody treatment, standard toxicity study to access leukocyte counts, gross changes in lymphoid tissue and histologic changes in critical organs, etc. was conducted. The present inventor first analyzed hematological parameters of CBC (Table X), and tested serum chemistry (Table XI) reflecting the function of liver (ALAT and ASAT), kidney (creatinine) and muscle (creatine kinase), as well as lipid metabolism, electrolyte status, iron concentration, albumin, bilirubin etc. High potassium and uric acid, and low platelets, were observed in two of the control vehicle-treated rats (Table X-XI), which could be attributed to post mortem blood collection in these animals. All other serum test values were within the standard range in both control and Ab-b-injected groups (Table X-XI). These data excluded the functional organ/system impairment in the antibody (Ab-b)-treated animals. Gross examination, body weight and subjective body condition score (BCS) were also recorded and measured (Table XII-XIII). In one of the Ab-b-treated rat, slightly and unremarkably lower body weight was observed and the cause was not identified. Fat in all these experimental rats was adequate/ample on gross and histology examination.

To further investigate possible organ-specific toxicity, the present inventor assessed various organs. Macroscopically, after two-week continuous treatment with the antibody Ab-b, heart, kidney, liver and spleen exhibit no sign of edema, hemorrhage, or other irregular morphology. The weights of these organs after perfusion were also recorded and adjusted by their body weight, and the present inventor failed to find significant differences in weight of these organs between the treatment groups (Table XIII). Microscopically, histological analyses of the above-mentioned organs as well as lung, mesentery/pancreas, stomach/cecum and intestine were also performed (Table XIV-XV). Minimal inflammation was observed in the heart of a vehicle-treated rat and in the liver of one antibody-treated rat, which was identified as an unsurprising background finding in rats. Hydronephrotic changes were also very mild in both groups and both right and left kidneys. Some infiltration of macrophages were found in the mesenteric lymph nodes, which was expected in animals receiving multiple IP injections. Collectively, the differences of pathology findings in the two tested groups are generally unremarkable (Table XII-XIII), indicating that the present inventor's generated anti-hResistin antibody is nontoxic and can moved forward for clinical development.

### DISCUSSION

In this study, the present inventor generated therapeutic monoclonal antibodies that inhibit the actions of hResistin protein. The present inventor's lead antibodies had anti-proliferative properties in human cell assays and were able to block the activities of rodent RELMα which also was able to induce the proliferative phenotype in human SMCs. These results indicates that antibody in vivo application could be therapeutically efficacious in the future rodent PH models, a critical step in moving these antibodies toward human use (FIG. 14). Because hResistin and rodent RELM have incomplete homology and the lead antibody was at most 50% as effective against the rodent isoform in vitro as it was against hResistin, the present inventor expect that the antibody will be even more potent in humans than in rats. This likelihood is supported by the present inventor's finding that the minimal functional blocking dose of the antibody to hResistin-stimulated SMCs was 3-fold lower than that for the RELMα-treated SMCs (0.1 vs. 0.3 μg/mL, FIGS. 1 and 5). Thus, developing these neutralizing anti-hResistin antibodies could initiate a novel clinical therapy for PH in humans.

Growing evidence indicates that inflammation plays a key role in triggering and maintaining pulmonary vascular remodeling.²⁴ Thus, therapies targeting immunity have recently become a promising anti-PH approach.²⁵ Previously in rodents, the present inventor found that RELMs cause PH by initiating lung vascular inflammation.^{19, 20} It thus suggested the immunomodulatory properties of these selected antibodies which would be the main mechanism of their possible anti-PH beneficial actions in vivo. In clinical investigation, patients with PH have been found to have a dysfunctional immune response during disease development.^{25, 26} Several immunomodulatory therapies are currently being assessed in clinical trials,²⁵ including an inhibitor of the inflammatory mediator leukotriene B427 and a monoclonal antibody to the IL-6 receptor.²⁸ Other treatment strategies in development²⁵ include therapies targeting mitochondrial dysfunction,^{29, 30} BMPR2 defects,^{31, 32} iron deficiency,³³ the neurohormonal axis,^{34, 35} epigenetic abnormalities, tyrosine kinase inhibitors (REFS), microRNA modulation,³⁶ and stem cell therapy.³⁷ In line with these various therapeutic strategies,²⁵ the present inventor's studies have revealed that RELMs activate damage-associated-molecular-pattern (DAMP) molecules, including HMGB1^{11, 12} and S100A11,¹⁷ and are responsible for impairments in mitochondrial function¹³ and BMPR2 signaling¹¹. RELMs may also alter miRNAs in hypoxic lung tissue.³⁸ Furthermore, RELM signaling is a regulator for stem cell proliferation, differentiation, mobilization, and recruitment related to PH.^{16, 18} Thus, the anti-RELM antibodies that the present inventor generated may have the potential to integrate these immune-, gene- and cell-based treatment strategies for a comprehensive anti-PH therapy.

Because PH in humans is a complex and multifactorial disease that is often identified at a late stage, combination therapy would allow distinct pathogenic pathways to be targeted simultaneously, leading to additive or synergistic beneficial effects.³⁹⁻⁴¹ In the future animal in vivo studies, the present inventor may combine the present inventor's antibodies with other current investigational approaches such as treatments that target tyrosine kinase,⁴² G-protein-coupled chemokine receptor,⁴² microRNAs,³⁶ endothelin signaling,⁴² IL-6,²⁵ or DAMPs.^{43, 44} All of these pathways are involved in the inflammation-mediating properties of RELMs and thus would reinforce the functions of anti-RELM antibodies.^{17, 19, 24, 45, 46} These projects will propel the therapeutic antibody studies forward for human development and use in clinical phase trials. Currently the present inventor have completed the tissue cross-reactivity testing with normal human tissues, and results indicated that the present inventor's monoclonal antibody has no toxicologic significance in that model,⁴⁷. This provides initial human support for the safety seen in the present inventor's rodent micro- and macro-pathology studies and in vivo utilization studies. The present inventor are also performing the bioanalytic assay and pharmacokinetics studies. Development of such novel therapeutics may generate a more comprehensive understanding of the PH disease mechanisms and open the door for precision medicine. The mechanistic insights might also be useful for improving outcomes of a variety of other vasculature-related disorders, especially cancers. Based on striking pathogenic analogies between cancer and PH, the recent cancer theory of PH suggests that anti-neoplastic drugs have therapeutic potential in PH⁴⁸ and vice versa. Therefore, the present inventor's antibodies may have additional benefits for patients with some cancers. Moreover, given the roles of hResistin in a variety of pulmonary, cardiac, and other related inflammatory pathologies including atherosclerosis, thrombosis, diabetes, pathological angiogenesis, cancers, etc., as mentioned above,² the present inventor's developed antibody also can be a novel potential therapeutic for these diseases.

### REFERENCES

1. Johns R A. Th2 inflammation, hypoxia-induced mitogenic factor/fizz1, and pulmonary hypertension and vascular remodeling in schistosomiasis. Am J Respir Crit Care Med. 2010; 181:203-205.
2. Lin Q, Johns R A. Resistin family proteins in pulmonary diseases. Am J Physiol Lung Cell Mol Physiol. 2020; 319: L422-L434.
3. Teng X, Li D, Champion H C, Johns R A. Fizzl/relma, a novel hypoxia-induced mitogenic factor in lung with vasoconstrictive and angiogenic properties. Circ Res. 2003; 92:1065-1067.
4. Angelini D J, Su Q, Yamaji-Kegan K, Fan C, Skinner J T, Champion H C, Crow M T, Johns R A. Hypoxia-induced mitogenic factor (himf/fizz1/relma) induces the vascular and hemodynamic changes of pulmonary hypertension. Am J Physiol Lung Cell Mol Physiol. 2009; 296: L582-593.
5. Angelini D J, Hyun S W, Grigoryev D N, Garg P, Gong P, Singh I S, Passaniti A, Hasday J D, Goldblum S E. Tnf-alpha increases tyrosine phosphorylation of vascular endothelial cadherin and opens the paracellular pathway through fyn activation in human lung endothelia. Am J Physiol Lung Cell Mol Physiol. 2006; 291: L1232-1245.
6. Daley E, Emson C, Guignabert C, de Waal Malefyt R, Louten J, Kurup V P, Hogaboam C, Taraseviciene-Stewart L, Voelkel N F, Rabinovitch M, Grunig E, Grunig G. Pulmonary arterial remodeling induced by a th2 immune response. J Exp Med. 2008; 205:361-372
7. Swain S D, Han S, Harmsen A, Shampeny K, Harmsen A G. Pulmonary hypertension can be a sequela of prior pneumocystis pneumonia. Am J Pathol. 2007; 171:790-799
8. Angelini D J, Su Q, Yamaji-Kegan K, Fan C, Teng X, Hassoun P M, Yang S C, Champion H C, Tuder R M, Johns R A. Resistin-like molecule-b in scleroderma-associated pulmonary hypertension. Am J Respir Cell Mol Biol. 2009; 41:553-561.
9. Fan C, Meuchel L W, Su Q, Angelini D J, Zhang A, Cheadle C, Kolosova I, Makarevich O D, Yamaji-Kegan K, Rothenberg M E, Johns R A. Resistin-like molecule alpha in allergen-induced pulmonary vascular remodeling. Am J Respir Cell Mol Biol. 2015; 53:303-313.
10. Nair M G, Guild K J, Artis D. Novel effector molecules in type 2 inflammation: Lessons drawn from helminth infection and allergy. J Immunol. 2006; 177:1393-1399.
11. Lin Q, Fan C, Gomez-Arroyo J, Van Raemdonck K, Meuchel L W, Skinner J T, Everett A D, Fang X, Macdonald A A, Yamaji-Kegan K, Johns R A. Himf (hypoxia-induced mitogenic factor) signaling mediates the hmgb 1 (high mobility group box 1)-dependent endothelial and smooth muscle cell crosstalk in pulmonary hypertension. Arterioscler Thromb Vasc Biol. 2019; 39:2505-2519.
12. Lin Q, Fan C, Skinner J T, Hunter E N, Macdonald A A, Illei P B, Yamaji-Kegan K, Johns R A. Relmalpha licenses macrophages for damage-associated molecular pattern activation to instigate pulmonary vascular remodeling. J Immunol. 2019; 203:2862-2871.
13. Tao B, Kumar S, Gomez-Arroyo J, Fan C, Zhang A, Skinner J, Hunter E, Yamaji-Kegan K, Samad I, Hillel A T, Lin Q, Zhai W, Gao W D, Johns R A. Resistin-like molecule alpha dysregulates cardiac bioenergetics in neonatal rat cardiomyocytes. Front Cardiovasc Med. 2021; 8:574708.
14. Lin Q, Skinner J T, Yang W, Yang X, Gao W D, Johns R A. Human resistin signaling induces cardiac dysfunction in pulmonary hypertension. Am J Respir Crit Care Med. (The American Thoracic Society International Conference 2020) 2020; 201: A6077.
15. Fan C, Johns B A, Su Q, Kolosova I A, Johns R A. Choosing the right antibody for resistin-like molecule (relm/fizz) family members. Histochem Cell Biol. 2013; 139:605-613.
16. Angelini D J, Su Q, Kolosova I A, Fan C, Skinner J T, Yamaji-Kegan K, Collector M, Sharkis S J, Johns R A. Hypoxia-induced mitogenic factor (himf/fizz1/relma) recruits bone marrow-derived cells to the murine pulmonary vasculature. PLOS One. 2010; 5: e11251.
17. Fan C, Fu Z, Su Q, Angelini D J, Van Eyk J, Johns R A. S100all mediates hypoxia-induced mitogenic factor (himf)-induced smooth muscle cell migration, vesicular exocytosis, and nuclear activation. Mol Cell Proteomics. 2011; 10: M110 000901.
18. Kolosova I A, Angelini D, Fan C, Skinner J, Cheadle C, Johns R A. Resistin-like molecule a stimulates proliferation of mesenchymal stem cells while maintaining their multipotency. Stem Cells Dev. 2013; 22:239-247.
19. Yamaji-Kegan K, Takimoto E, Zhang A, Weiner N C, Meuchel L W, Berger A E, Cheadle C, Johns R A. Hypoxia-induced mitogenic factor (fizzl/relma) induces endothelial cell apoptosis and subsequent interleukin-4-dependent pulmonary hypertension. Am J Physiol Lung Cell Mol Physiol. 2014; 306: L1090-1103.
20. Yamaji-Kegan K, Su Q, Angelini D J, Myers A C, Cheadle C, Johns R A. Hypoxia-induced mitogenic factor (himf/fizz1/relma) increases lung inflammation and activates pulmonary microvascular endothelial cells via an il-4-dependent mechanism. J Immunol. 2010; 185:5539-5548.
21. Weitzner B D, Jeliazkov J R, Lyskov S, Marze N, Kuroda D, Frick R, Adolf-Bryfogle J, Biswas N, Dunbrack R L, Jr., Gray J J. Modeling and docking of antibody structures with rosetta. Nat Protoc. 2017; 12:401-416.
22. Blake S, Carton J, Lee J, Ma K, Marsters P, Picha K, Song Xiao-Yu R, Farrell F, Murray L, Teplyakov A, Ort T. Resistin antagonists and their use. 2008.
23. Patel S D, Rajala M W, Rossetti L, Scherer P E, Shapiro L. Disulfide-dependent multimeric assembly of resistin family hormones. Science. 2004; 304:1154-1158.
24. Johns R A, Takimoto E, Meuchel L W, Elsaigh E, Zhang A, Heller N M, Semenza G L, Yamaji-Kegan K. Hypoxia-inducible factor 1alpha is a critical downstream mediator for hypoxia-induced mitogenic factor (fizz1/relmalpha)-induced pulmonary hypertension. Arterioscler Thromb Vasc Biol. 2016; 36:134-144.
25. Simonneau G, Hoeper M M, Mclaughlin V, Rubin L, Galie N. Future perspectives in pulmonary arterial hypertension. Eur Respir Rev. 2016; 25:381-389.
26. Humbert M, Lau E M, Montani D, Jais X, Sitbon O, Simonneau G. Advances in therapeutic interventions for patients with pulmonary arterial hypertension. Circulation. 2014; 130:2189-2208.
27. Tian W, Jiang X, Sung Y K, Qian J, Yuan K, Nicolls M R. Leukotrienes in pulmonary arterial hypertension. Immunol Res. 2014; 58:387-393.
28. Hernandez-Sanchez J, Harlow L, Church C, Gaine S, Knightbridge E, Bunclark K, Gor D, Bedding A, Morrell N, Corris P, Toshner M. Clinical trial protocol for transform-uk: A therapeutic open-label study of tocilizumab in the treatment of pulmonary arterial hypertension. Pulm Circ. 2018; 8:2045893217735820.
29. Wilkins M R. Pulmonary hypertension: The science behind the disease spectrum. Eur Respir Rev. 2012; 21:19-26.
30. Yu Q, Chan S Y. Mitochondrial and metabolic drivers of pulmonary vascular endothelial dysfunction in pulmonary hypertension. Adv Exp Med Biol. 2017; 967:373-383.
31. Spiekerkoetter E, Tian X, Cai J, Hopper R K, Sudheendra D, Li C G, El-Bizri N, Sawada H, Haghighat R, Chan R, Haghighat L, de Jesus Perez V, Wang L, Reddy S, Zhao M, Bernstein D, Solow-Cordero D E, Beachy P A, Wandless T J, Ten Dijke P, Rabinovitch M. Fk506 activates bmpr2, rescues endothelial dysfunction, and reverses pulmonary hypertension. J Clin Invest. 2013; 123:3600-3613.
32. Spiekerkoetter E, Sung Y K, Sudheendra D, Scott V, Del Rosario P, Bill M, Haddad F, Long-Boyle J, Hedlin H, Zamanian R T. Randomised placebo-controlled safety and tolerability trial of fk506 (tacrolimus) for pulmonary arterial hypertension. Eur Respir J. 2017; 50.
33. van Empel V P, Lee J, Williams T J, Kaye D M. Iron deficiency in patients with idiopathic pulmonary arterial hypertension. Heart Lung Circ. 2014; 23:287-292.
34. Rothman A M, Arnold N D, Chang W, Watson O, Swift A J, Condliffe R, Elliot C A, Kiely D G, Suvarna S K, Gunn J, Lawrie A. Pulmonary artery denervation reduces pulmonary artery pressure and induces histological changes in an acute porcine model of pulmonary hypertension. Circ Cardiovasc Interv. 2015; 8: e002569.
35. Chen S L, Zhang H, Xie D J, Zhang J, Zhou L, Rothman A M, Stone G W. Hemodynamic, functional, and clinical responses to pulmonary artery denervation in patients with pulmonary arterial hypertension of different causes: Phase ii results from the pulmonary artery denervation-1 study. Circ Cardiovasc Interv. 2015; 8: e002837.
36. Thompson A A R, Lawrie A. Targeting vascular remodeling to treat pulmonary arterial hypertension. Trends Mol Med. 2017; 23:31-45.
37. Zhu Z, Fang Z, Hu X, Zhou S. Micrornas and mesenchymal stem cells: Hope for pulmonary hypertension. Rev Bras Cir Cardiovasc. 2015; 30:380-385.
38. Liu S X, Zheng F, Xie K L, Xie M R, Jiang L J, Cai Y. Exercise reduces insulin resistance in type 2 diabetes mellitus via mediating the Incrna malat1/microrna-382-3p/resistin axis. Mol Ther Nucleic Acids. 2019; 18:34-44.
39. Griffin M, Trow T K. The evolving landscape of combination therapy for pulmonary arterial hypertension. Ther Adv Respir Dis. 2017; 11:91-95.
40. Burks M, Stickel S, Galie N. Pulmonary arterial hypertension: Combination therapy in practice. Am J Cardiovasc Drugs. 2018.
41. Sitbon O, Gaine S. Beyond a single pathway: Combination therapy in pulmonary arterial hypertension. Eur Respir Rev. 2016; 25:408-417.
42. Vaidya B, Gupta V. Novel therapeutic approaches for pulmonary arterial hypertension: Unique molecular targets to site-specific drug delivery. J Control Release. 2015; 211:118-133.
43. Sadamura-Takenaka Y, Ito T, Noma S, Oyama Y, Yamada S, Kawahara K, Inoue H, Maruyama I. Hmgb 1 promotes the development of pulmonary arterial hypertension in rats. PLOS One. 2014; 9: e102482.
44. Goldenberg N M, Hu Y, Hu X, Volchuk A, Zhao Y D, Kucherenko M M, Knosalla C, de Perrot M, Tracey K J, Al-Abed Y, Steinberg B E, Kuebler W M. Therapeutic targeting of high mobility group box-1 in pulmonary arterial hypertension. Am J Respir Crit Care Med. 2019.
45. Fan C, Su Q, Li Y, Liang L, Angelini D J, Guggino W B, Johns R A. Hypoxia-induced mitogenic factor/fizz1 induces intracellular calcium release through the plc-ip (3) pathway. Am J Physiol Lung Cell Mol Physiol. 2009; 297: L263-270.
46. Su Q, Zhou Y, Johns R A. Bruton's tyrosine kinase (btk) is a binding partner for hypoxia induced mitogenic factor (himf/fizz1) and mediates myeloid cell chemotaxis. FASEB J. 2007; 21:1376-1382.
47. Lin Q, Price S A, Skinner J T, Hu B, Fan C, Yamaji-Kegan K, Johns R A. Systemic evaluation and localization of resistin expression in normal human tissues by a newly developed monoclonal antibody. PLOS One. 2020; 15: e0235546.
48. Boucherat O, Vitry G, Trinh I, Paulin R, Provencher S, Bonnet S. The cancer theory of pulmonary arterial hypertension. Pulm Circ. 2017; 7:285-299.
49. Jamaluddin M S, Weakley S M, Yao Q, Chen C. Resistin: Functional roles and therapeutic considerations for cardiovascular disease. Br J Pharmacol. 2012; 165:622-632.

### Supplemental Materials and Methods

Production of recombinant hResistin/RELMα proteins in cell lines. The present inventor's laboratory produces all four mouse RELM isoforms and the two human isoforms in eukaryotic cell lines (T-REX™ or CHO).1 For hResistin, briefly, the pcDNA5/FRT/TOPO TA vector containing C-terminal FLAG-tagged hResistin cDNA was integrated into the genome of the Flp-In™ T-REX™ 293 cell line in a Flp recombinase-dependent manner (Invitrogen, Carlsbad, CA). Production of recombinant hResistin in T-REx 293 cells was induced by tetracycline (1 μg/mL) in DMEM supplemented with 5% fetal bovine serum, 100 μg/mL hygromycin B, and 7.5 μg/mL blasticidin. hResistin then was purified from the cell culture medium with anti-FLAG M2 antibody agarose (Sigma, St. Louis, MO) column chromatography.

Phage display approach to initial screening of the generated antibodies. The present inventor initiated an antibody search using hResistin to select scFv binders from a phage display human scFv library. An initial 80 clones were chosen from the screen. Soluble ELISA was used to select hResistin scFv clones that bound to their target positively. These selected scFv fragments were subsequently made into full human IgG1 antibodies for use in initial lead selection studies.

ELISA evaluation for potency and selectivity. A sandwich-type ELISA approach was created to evaluate the binding efficacy of each anti-hResistin antibody. The antigen concentration and the antibody concentration were varied to assist in selecting the initial antibodies for further screening.

In silico screening. In silico evaluations included deamidation risk, oxidation risk, isoelectric point, and immunogenicity. To compare the epitope content of the tested candidate antibodies, all sequences were analyzed for the presence of putative HLA class II restricted epitopes, also known as Th-epitopes. An approximate score expressing a worst-case immunogenic risk can be calculated as: DRB1 score=> (epitope count×allotype frequency). In short, the number of critical epitopes affecting a particular HLA allotype is multiplied by the allele frequency of the affected allotype. For a given sequence, the products were summed for all DRB1 allotypes used in the study. Global DRB1 risk scores and epitope counts for the tested antibodies, and a selection of antibody variable domains and full-length antibodies for marketed therapeutic antibodies, were analyzed.

For manufacturability assessment, predictive computational tools were used to identify structural or sequence elements that have the potential to result in aggregation and post-translational modifications (PTMs) such as glycosylation, deamidation, oxidation and variation of the N- and C-termini. The antibody aggregation platform was developed using machine learning algorithms based on sequence and structural features of antibodies as described.2 Asparagine deamidation, aspartate isomerisation and fragmentation, and C-terminal lysine processing were predicted by detecting the targeted and succeeding residues. Analyses of possible influences from structural features were also performed. The isoelectric point was calculated based on the number of charged residues in the primary amino-acid sequence using EMBOSS pKa values. For N-glycosylation the motif N—X-S/T where X is any residue except Proline generally served to detect sites. A boosting decision tree ensemble algorithm was trained on experimentally determined glycosylation sites in order to predict O-glycosylation. N-terminal Glycine-Proline motifs were detected to predict cyclization. Methionine and Tryptophan residues were identified for the prediction of oxidation. And N-terminal Glutamine or Glutamate residues were detected for pyroglutamate formation analysis.

Stable pooled production, purification and product analysis of antibodies. For single and double gene vector construction, heavy and light chain genes were synthesized by Life Technologies and sub-cloned into Lonza Biologics GS Xceed™ gene expression system vectors. Stable pooled transfections of CHOK1SV GS-KO cells with the established double gene vector DNA plasmids were carried out via electroporation using the Gene Pulse XCell. For the expansion of stable pools, recombinant cells were cultured in CD-CHO media supplemented with 50 μM MSX and SP4, and propagated for the fed-batch overgrowth cultures. Protein A purification of the clarified supernatant was performed using HiTrap MabSelect SuRE columns in tandem (GE Healthcare) on an AKTA purifier. Eluted fractions were immediately pH adjusted by neutralizing with 2×PBS buffer, pH 7.4 and titrated to approximately pH 7.2-7.6 by the addition of dilute sodium hydroxide solution. Antibody protein yield was determined by 280 nm absorbance on a Nanodrop instrument. Samples of clarified cell culture supernatant were also analyzed on an Octet QKe using Protein A Biosensors (ForteBio, 18-5010). 200 μL aliquots of supernatant samples were loaded into a 96-well plate and quantified against an eight-point standard curve.

Fast Stable Pool production. Ab-b was generated from fast stable pool cultured in 7L bioreactors based on WuXi Biologics Chinese Hamster Ovary (CHO) cell culture process. Cells in pools showed acceptable cell growth rates in exponential phase and reached equivalent peak viable cell densities, ranged in 20-25×10⁶ cells/mL. Whereas, the monitored metabolites, glucose and lactate, were observed to follow the normal trends. Bioreactor run was cultivated for 14 days using four-bonus feed-batch process, and the obtained final titers were analyzed by HPLC as the results of productivity.

Size exclusion high-performance liquid chromatography (SE-HPLC). Duplicate samples were analyzed by SE-HPLC on an Agilent 1200 series HPLC system with a Zorbax GF-250 9.4 mm ID×25 cm column (Agilent). Eighty-microliter aliquots of 1 mg/mL samples were injected and run in 50 nM sodium phosphate, 150 mM sodium chloride, 500 mM arginine (pH 6.0) at 1 mL/min for 15 minutes. Soluble aggregate levels were analyzed by Chemstation software. Signals arising from buffer constituents were analyzed by blank buffer injection and are omitted in the data analyzed.

Plasmon resonance assay. The present inventor developed a plasmon resonance (BIACORE) assay using a BIACORE 1000 (GE Healthcare) for in vitro assessment of antibody potency, selectivity, and kinetics of binding. Anti-FLAG antibody was immobilized on a CM5 chip by primary amine coupling. After overnight washing, this surface was then used to capture FLAG-tagged hResistin. hResistin antibodies were then passed over the captured antigen at varying concentrations. Chip regeneration was carried out with carbonate buffer (pH 11.55, 50 mM) or CAPS buffer (pH 11.4, 0.1 M). Binding of antigen (hResistin) to the antibodies was monitored in real time. From the observed apparent on-rates (ka) and off-rates (kd), the equilibrium affinity KD (kd/ka) was determined. The binding curves were referenced with negative (human IgG) and positive (R&D goat anti-hResistin polyclonal Ab) controls, and the data were fitted to the Biacore 1000 analysis software.

Endotoxin measurement. Endotoxin levels of the purified proteins at 1 mg/ml concentration were measured with the Endosafe-PTS instrument, a cartridge-based method based on the Limulus Amebocyte Lysate (LAL) assay (Charles River, Frederick, MD).

Structural modeling for the assessment of antibody-protein binding. The ClusPro online server was used to check whether the present inventor's predictive epitope regions (residues 50-65 and 78-93)³ can be identified.^{4,5} Using the known crystal structure for resistin in mice as a basis (PDB ID 1RFX, 58% sequence identity),^{6, 7} the present inventor modeled the three-dimensional structure of a monomeric hResistin. antibody three-dimensional structure was predicted from sequence by using RosettaAntibody.^{8, 9} Using SnugDock,′ the antibody was then docked with hResistin to generate candidate antibody-antigen complex structures. The present inventor separately locally docked the antibody to each of the two suspected epitope regions of the hResistin protein. SnugDock generated 1,000 decoys, and the lowest-scoring (low-energy/most stable) docked structure was chosen as the final model depicted in FIG. 13. The score was calculated as the difference in the Rosetta energy of the antibody-hResistin complex structure and the sum of the energies of the separated component structures (i.e., the interface score).

Stability study. Antibody (Ab-b) was concentrated to 5 mg/mL using Amicon centrifugal filters with a molecular weight cut off of 30 kDa (UFC903024, Merck). 500 μl of the concentrated product was aliquoted per test condition (Table IX) into glass vials for each condition to be tested. After a 14 day incubation period the stability of Ab-b in each condition was assessed via SDS-PAGE, SE-HPLC and cIEF. SE-HPLC analysis was performed as mentioned above. Capillary IEF analysis was performed using the Maurice Capillary Electrophoresis (iCE) system (Protein Simple). Briefly, samples were diluted in sample buffer to a final concentration of 1 mg/mL (and a final salt concentration <50 mM). The sample buffer was prepared by mixing deionised water with 1% methyl cellulose, Pharmalyte 3-10, 500 mM arginine and pI markers, to give a final volume of 200 μl. Running conditions were as follows: 1 min at 1500V followed by 4.5 min at 3000V for each sample. Data analysis was performed using Compass for iCE (Protein Simple). As to the SDS-PAGE analysis, reduced samples were prepared for analysis by mixing with NuPage 4×LDS sample buffer

(Life Technologies, NP0007) and NuPage 10× sample reducing agent (Life Technologies, NP0009), and incubated at 70° C., 10 min. For non-reduced samples, the reducing agent and heat incubation were omitted. Samples were electrophoresed on 1.5 mm NuPage 4-12% Bis-Tris Novex pre-cast gels (Life Technologies, NP0315/6) with NuPage MES SDS running buffer under denaturing conditions. 10 μL aliquot of SeeBlue Plus 2 pre-stained molecular weight standard (Life Technologies, LC5925) and of a control antibody at 5 mg/mL were included on the gel. 5 μg of each sample was loaded onto the gel. Once electrophoresed, gels were stained with InstantBlue (TripleRed, ISB01L) for 30 min at room temperature. Images of the stained gels were analyzed on a CHemiDoc XRS Gel Imager (BioRad).

ELISA detection of human IgG level in rat serum The present inventor measured human IgG levels in the serum of anti-hResistin antibody-injected rats with a commercial ELISA kit (E88-104, Bethyl Laboratories, Montgomery, TX) according to the manufacturer's instructions.

Immunoprecipitation (IP). The present inventor used IP to confirm that the generated human antibodies bind to rodent RELMα. Two micrograms of generated anti-hResistin antibodies were incubated with 100 ng of lab-made, recombinant, FLAG-tagged rat RELMα and mixed with 10 μL of Dynabeads® Protein A (10001D, Thermo Fisher, Waltham, MA). The binding of RELMα to hResistin antibodies was detected by western blotting with anti-FLAG® M2 antibody (F1804, Sigma).

Toxicological assessment. Clinical chemistry and hematology, and the necropsy and histopathology, were performed by Johns Hopkins Phenotyping (Pathology for Phenotyping & Preclinical Research) Core. Adult male SD rats received I.P. injection of anti-hResistin antibody Ab-b 2 times a week for 2 weeks at the concentration of 4 mg/kg in sterile saline. Ab-b diluted to concentration in sterile 0.9% saline. The vehicle saline-treated group was served as control. For sample harvest, the experimental rats were euthanized by I.P. injection of overdose Ketamine/Xylazine. Before sample collection, body weight and subjective body condition score (BCS) for each animal were recorded. Through cardiocentesis 3.0 ml blood was collected for each rat, and it was made sure that no hemolysis occurred in serum. Rat sera (n=5 per treatment group) were obtained from the harvested blood, and chemistry including AST, ALT, BUN, ALP, Ca, Glu, LDH, GGT, Tprot, Alb, TBil, Creat, CK, Phos, MG, CHOL, TRIG, AMYL, UA, HDL, DBILI, Na, K and CL; and CBC including RBC, Hb, HCT, MCV, MCH, RET, RET, PLT, WBC, NE, LY, MO and EO, were analyzed with the IDEXX ProCyte Dx® Hematology Analyzer (IDEXX Laboratories Inc; Westbook, ME). As to the histopathological analyses, anesthetized animals were perfused via left ventricle with heparin saline followed by 10% NBF. The weight of the liver heart kidneys and spleen after perfusion were measured and adjusted by corresponding body weight. After fixation, paraffin embedding, slicing and Hematoxylin & Eosin (H&E) staining, organ morphology was assessed for anatomic diagnosis and necropsy record.

Animals. Male wild-type rat (Sprague-Dawley) (Charles River Laboratories).

Antibodies. hResistin/RELM (Creative Biolabs, customized): In vitro blockade: 0.1-10 ug/mL. hResistin/RELM (Lonza, customized): In vitro blockade: 0.1-10 μg/mL. FLAG M2 (Sigma, F1804): WB: 1 mg/mL. BrdU (kit) (Roche, 11647229001): ELISA: 0.075 U/mL. WB, western blot; ELISA, enzyme-linked immunosorbent assay.

Cultured Cells. Human bronchial SMCs (Lonza, CC-2576): sex unknown. Human pulmonary artery (vascular) SMCs (Lonza, CC_2581): sex unknown. SMCs, smooth muscle cells.

### SUPPLEMENTAL REFERENCES

1. Fan C, Johns B A, Su Q, Kolosova I A, Johns R A. Choosing the right antibody for resistin-like molecule (relm/fizz) family members. Histochem Cell Biol. 2013; 139:605-613.
2. Obrezanova O, Arnell A, de la Cuesta R G, Berthelot M E, Gallagher T R, Zurdo J, Stallwood Y. Aggregation risk prediction for antibodies and its application to biotherapeutic development. MAbs. 2015; 7:352-363.
3. Blake S, Carton J, Lee J, Ma K, Marsters P, Picha K, Song Xiao-Yu R, Farrell F, Murray L, Teplyakov A, Ort T. Resistin antagonists and their use. 2008.
4. Kozakov D, Hall D R, Xia B, Porter K A, Padhorny D, Yueh C, Beglov D, Vajda S. The cluspro web server for protein-protein docking. Nat Protoc. 2017; 12:255-278.
5. Brenke R, Hall D R, Chuang G Y, Comeau S R, Bohnuud T, Beglov D, Schueler-Furman O, Vajda S, Kozakov D. Application of asymmetric statistical potentials to antibody-protein docking. Bioinformatics. 2012; 28:2608-2614.
6. Jamaluddin M S, Weakley S M, Yao Q, Chen C. Resistin: Functional roles and therapeutic considerations for cardiovascular disease. Br J Pharmacol. 2012; 165:622-632.
7. Patel S D, Rajala M W, Rossetti L, Scherer P E, Shapiro L. Disulfide-dependent multimeric assembly of resistin family hormones. Science. 2004; 304:1154-1158.
8 Long X, Jeliazkov J R, Gray J J. Non-h3 cdr template selection in antibody modeling through machine learning. PeerJ. 2019; 7: e6179.
9. Weitzner B D, Jeliazkov J R, Lyskov S, Marze N, Kuroda D, Frick R, Adolf-Bryfogle J, Biswas N, Dunbrack R L, Jr., Gray J J. Modeling and docking of antibody structures with rosetta. Nat Protoc. 2017; 12:401-416.
10. Girgis R E, Mozammel S, Champion H C, Li D, Peng X, Shimoda L, Tuder R M, Johns R A, Hassoun P M. Regression of chronic hypoxic pulmonary hypertension by simvastatin. Am J Physiol Lung Cell Mol Physiol. 2007; 292: L1105-1110.
11. Fan C, Meuchel L W, Su Q, Angelini D J, Zhang A, Cheadle C, Kolosova I, Makarevich O D, Yamaji-Kegan K, Rothenberg M E, Johns R A. Resistin-like molecule alpha in allergen-induced pulmonary vascular remodeling. Am J Respir Cell Mol Biol. 2015; 53:303-313.
12. Satwiko M G, Ikeda K, Nakayama K, Yagi K, Hocher B, Hirata K, Emoto N. Targeted activation of endothelin-1 exacerbates hypoxia-induced pulmonary hypertension. Biochem Biophys Res Commun. 2015; 465:356-362.

TABLE I
Global DRB1 risk scores and epitope counts for the tested antibodies

| | | DRB1 | DRB1 | | DRB3/4/5. | | DQ | | DP | |
|---|---|---|---|---|---|---|---|---|---|---|
| Type | Chain | risk score | S | M | S | M | S | M | S | M |
| Ab #1 | HC | 197.6 | 2 (33) | 4 (78) | 0 (9) | 5 (54) | 2 (5) | 5 (65) | 0 (6) | 2 (43) |
| | LC | 89.9 | 2 (12) | 3 (42) | 1 (1) | 3 (26) | 0 (2) | 1 (39) | 0 (3) | 0 (19) |
| | Full Ab | 287.5 | 4 (45) | 7 (120) | 1 (10) | 8 (80) | 2 (7) | 6 (104) | 0 (9) | 2 (62) |
| Ab #2 | HC | 131.6 | 1 (33) | 6 (78) | 2 (9) | 3 (54) | 0 (5) | 5 (64) | 0 (6) | 6 (43) |
| | LC | 377.8 | 6 (8) | 4 (41) | 2 (1) | 6 (21) | 0 (2) | 5 (34) | 0 (3) | 3 (17) |
| | Full Ab | 509.4 | 7 (41) | 10 (119) | 4 (10) | 9 (75) | 0 (7) | 10 (98) | 0 (9) | 9 (60) |
| Ab #3 | HC | 299.7 | 3 (33) | 6 (78) | 0 (9) | 7 (54) | 0 (5) | 11 (64) | 0 (6) | 2 (43) |
| | LC | 438.9 | 6 (8) | 5 (41) | 1 (1) | 4 (21) | 0 (2) | 3 (33) | 0 (3) | 6 (16) |
| | Full Ab | 738.6 | 9 (41) | 11 (119) | 1 (10) | 11 (75) | 0 (7) | 14 (97) | 0 (9) | 8 (59) |
| Ab #4 | HC | 190.0 | 2 (33) | 9 (78) | 0 (9) | 3 (54) | 0 (5) | 4 (65) | 0 (6) | 4 (44) |
| (Ab-a) | LC | 312.6 | 3 (8) | 7 (40) | 2 (1) | 3 (21) | 0 (2) | 1 (33) | 0 (3) | 5 (16) |
| | Full Ab | 502.6 | 5 (41) | 16 (118) | 2 (10) | 6 (75) | 0 (7) | 5 (98) | 0 (9) | 9 (60) |
| Ab #5 | HC | 314.3 | 4 (33) | 7 (78) | 2 (9) | 4 (54) | 0 (5) | 7 (64) | 0 (6) | 6 (43) |
| | LC | 56.4 | 0 (12) | 3 (42) | 0 (1) | 1 (26) | 2 (2) | 3 (40) | 0 (3) | 0 (19) |
| | Full Ab | 370.7 | 4 (45) | 10 (120) | 2 (10) | 5 (80) | 2 (7) | 10 (104) | 0 (9) | 6 (62) |
| Ab #6 | HC | 331.8 | 6 (33) | 7 (79) | 2 (9) | 5 (55) | 1 (5) | 5 (64) | 0 (6) | 6 (43) |
| (Ab-b) | LC | 402.6 | 6 (8) | 5 (40) | 1 (1) | 4 (21) | 0 (2) | 3 (33) | 0 (3) | 2 (16) |
| | Full Ab | 734.4 | 12 (41) | 12 (119) | 3 (10) | 9 (76) | 1 (7) | 8 (97) | 0 (9) | 8 (59) |
| Ab #7 | HC | 322.7 | 3 (33) | 7 (78) | 0 (9) | 5 (54) | 1 (5) | 9 (64) | 0 (6) | 5 (43) |
| | LC | 597.1 | 5 (8) | 11 (38) | 2 (1) | 7 (20) | 0 (2) | 8 (32) | 0 (3) | 6 (16) |
| | Full Ab | 919.8 | 8 (41) | 18 (116) | 2 (10) | 12 (74) | 1 (7) | 17 (96) | 0 (9) | 11 (59) |
| Ab #8 | HC | 687.6 | 5 (31) | 18 (75) | 1 (9) | 7 (51) | 1 (3) | 15 (60) | 3 (5) | 14 (40) |
| (Ab-c) | LC | 245.8 | 2 (12) | 7 (32) | 0 (2) | 2 (20) | 1 (6) | 6 (36) | 2 (1) | 3 (12) |
| | Full Ab | 933.4 | 7 (43) | 25 (107) | 1 (11) | 9 (71) | 2 (9) | 21 (96) | 5 (6) | 17 (52) |
| Ab #9 | HC | 314.9 | 5 (31) | 6 (83) | 0 (11) | 4 (50) | 0 (4) | 3 (61) | 1 (6) | 5 (43) |
| | LC | 532.8 | 8 (10) | 11 (32) | 3 (2) | 10 (16) | 1 (8) | 17 (29) | 2 (1) | 9 (11) |
| | Full Ab | 847.7 | 13 (41) | 17 (115) | 3 (13) | 14 (66) | 1 (12) | 20 (90) | 3 (7) | 14 (54) |
| Ab #10 | HC | 209.0 | 3 (33) | 4 (78) | 2 (9) | 2 (54) | 0 (5) | 4 (64) | 0 (6) | 4 (43) |
| (Ab-d) | LC | 394.9 | 5 (8) | 5 (41) | 1 (1) | 4 (21) | 0 (2) | 2 (34) | 0 (3) | 4 (16) |
| | Full Ab | 603.9 | 8 (41) | 9 (119) | 3 (10) | 6 (75) | 0 (7) | 6 (98) | 0 (9) | 8 (59) |
| Human | VH | 639.5 | 6 (12) | 9 (12) | 0 (2) | 10 (15) | 3 (1) | 10 (19) | 0 (0) | 7 (8) |
| | VL | 245.0 | 2 (6) | 8 (17) | 0 (0) | 2 (14) | 1 (0) | 5 (15) | 1 (1) | 3 (6) |
| | VH + VL | 884.5 | 8 (18) | 17 (29) | 0 (2) | 12 (29) | 4 (1) | 15 (34) | 1 (1) | 10 (14) |
| Humanized | VH | 588.2 | 8 (5) | 13 (9) | 2 (0) | 12 (8) | 2 (0) | 20 (6) | 2 (0) | 9 (6) |
| A | VL | 469.0 | 3 (1) | 18 (8) | 0 (0) | 10 (5) | 0 (1) | 14 (10) | 0 (1) | 10 (2) |
| | VH + VL | 1057.2 | 11 (6) | 31 (17) | 2 (0) | 22 (13) | 2 (1) | 34 (16) | 2 (1) | 19 (8) |
| Humanized | VH | 436.1 | 6 (8) | 12 (6) | 2 (1) | 7 (7) | 1 (1) | 14 (9) | 1 (0) | 10 (5) |
| B | VL | 694.3 | 7 (1) | 15 (8) | 1 (0) | 10 (6) | 2 (1) | 11 (8) | 0 (1) | 10 (2) |
| | VH + VL | 1130.4 | 13 (9) | 27 (14) | 3 (1) | 17 (13) | 3 (2) | 25 (17) | 1 (1) | 20 (7) |
| Humanized | VH | 897.8 | 12 (2) | 27 (2) | 1 (0) | 21 (3) | 0 (0) | 20 (5) | 1 (0) | 11 (0) |
| C | VL | 567.0 | 9 (1) | 7 (11) | 1 (0) | 9 (6) | 0 (0) | 3 (9) | 1 (1) | 8 (2) |
| | VH + VL | 1464.8 | 21 (3) | 34 (13) | 2 (0) | 30 (9) | 0 (0) | 23 (14) | 2 (1) | 19 (2) |
| Chimeric | VH | 1023.3 | 15 (1) | 23 (1) | 1 (1) | 21 (0) | 2 (0) | 19 (2) | 1 (0) | 13 (0) |
| A | VL | 972.8 | 11 (0) | 25 (1) | 3 (0) | 12 (0) | 1 (0) | 21 (1) | 0 (0) | 12 (0) |
| | VH + VL | 1996.1 | 26 (1) | 48 (2) | 4 (1) | 33 (0) | 3 (0) | 40 (3) | 1 (0) | 25 (0) |
| Chimeric | HC | 1107.9 | 11 (23) | 26 (62) | 3 (8) | 17 (36) | 6 (2) | 18 (52) | 2 (5) | 12 (32) |
| B | LC | 795.1 | 10 (5) | 18 (23) | 4 (1) | 9 (12) | 3 (1) | 20 (20) | 1 (1) | 5 (9) |
| | Full Ab | 1903.0 | 21 (28) | 44 (85) | 7 (9) | 26 (48) | 9 (3) | 38 (72) | 3 (6) | 17 (41) |

Table I showed global DRB1 risk scores and epitope counts for the tested ten antibodies and a selection of antibody variable domains and full-length antibodies for marketed therapeutic antibodies. Values between brackets refer to self-epitopes. Based on the critical epitope counts, the affected HLA allotypes and DRB 1 risk score, the ten antibodies can beranked by increased immunogenic risk as follows: Ab #1< #5< [#2, #4]< #10< [#3, #6]< [#7, #8, #9].

TABLE II
Theoretical molecular data of the ten tested candidate antibodies

| | Iso- | Molecular | Extinction | Extinction |
|---|---|---|---|---|
| | electric | weight | coefficient | Coefficient E^{1%} |
| Antibodies | Point | (Da) | (M⁻¹ cm⁻¹) | (M⁻¹ cm⁻¹) |
| Ab #1 | 8.0 | 146191 | 186750 | 1.3 |
| Ab #2 | 8.1 | 146620 | 186750 | 1.3 |
| Ab #3 | 8.1 | 146469 | 197750 | 1.4 |
| Ab #4 (Ab-a) | 8.2 | 146539 | 189730 | 1.3 |
| Ab #5 | 8.0 | 145861 | 186750 | 1.3 |
| Ab #6 (Ab-b) | 8.1 | 146461 | 192710 | 1.3 |
| Ab #7 | 8.1 | 146367 | 186500 | 1.3 |
| Ab #8 (Ab-c) | 7.8 | 147642 | 237380 | 1.6 |
| Ab #9 | 7.8 | 148470 | 243590 | 1.6 |
| Ab #10 (Ab-d) | 8.3 | 146341 | 186750 | 1.3 |

Determined computationally based on the primary sequence information only. Data does not take into account any potential post-translational modifications, which would be expected in this production system.

TABLE III
All potential PTMs counted in the tested candidate antibodies

| | | | | Free | | | | | |
|---|---|---|---|---|---|---|---|---|---|
| | | | C-Terminal | Cysteine | | | | | |
| | | Isomerisation/ | Lysine | Thiol | N- | N-Terminal | | O- | Pyroglutamate |
| Antibodies | Deamidation | Fragmentation | Clipping | Groups | Glycosylation | Cyclisation | Oxidation | Glycosyltion | Formation |
| Ab #1 | 14 | 3 | 1 | 4 | 1 | 0 | 17 | 27 | 1 |
| Ab #2 | 14 | 4 | 1 | 4 | 1 | 0 | 17 | 26 | 1 |
| Ab #3 | 15 | 3 | 1 | 4 | 1 | 0 | 19 | 25 | 1 |
| Ab #4 (Ab-a) | 14 | 3 | 1 | 4 | 1 | 0 | 17 | 25 | 1 |
| Ab #5 | 14 | 3 | 1 | 4 | 1 | 0 | 18 | 28 | 1 |
| Ab #6 (Ab-b) | 14 | 3 | 1 | 4 | 1 | 0 | 17 | 26 | 1 |
| Ab #7 | 14 | 3 | 1 | 4 | 1 | 0 | 17 | 27 | 1 |
| Ab #8 (Ab-c) | 13 | 3 | 1 | 4 | 1 | 0 | 19 | 32 | 2 |
| Ab #9 | 11 | 3 | 1 | 6 | 1 | 0 | 21 | 30 | 2 |
| Ab #10 (Ab-d) | 14 | 3 | 1 | 4 | 1 | 0 | 17 | 25 | 1 |

TABLE IV
In silico analysis of potential developability issues of the tested candidate antibodies

| | | | Isomerisation/ | C-Terminal | Free Cysteine | | Pyroglutamate |
|---|---|---|---|---|---|---|---|
| Antibodies | Aggregation | Deamidation | Fragmentation | Lysine Clipping | Thiol Groups | Oxidation | Formation |
| Ab #4 (Ab-a) | High | 0 (0) | 0 (0) | 0 (1) | 0 (4) | 0 (1) | 0 (1) |
| Ab #1 | High | 0 (0) | 0 (0) | 0 (1) | 0 (4) | 0 (1) | 0 (1) |
| Ab #2 | High | 0 (0) | 1 (0) | 0 (1) | 0 (4) | 0 (1) | 0 (1) |
| Ab #3 | High | 1 (0) | 0 (0) | 0 (1) | 0 (4) | 1 (2) | 0 (1) |
| Ab #4 (Ab-a) | High | 0 (0) | 0 (0) | 0 (1) | 0 (4) | 0 (1) | 0 (1) |
| Ab #5 | Low | 0 (0) | 0 (0) | 0 (1) | 0 (4) | 1 (1) | 0 (1) |
| Ab #6 (Ab-b) | Low | 0 (0) | 0 (0) | 0 (1) | 0 (4) | 0 (1) | 0 (1) |
| Ab #7 | High | 0 (0) | 0 (0) | 0 (1) | 0 (4) | 0 (1) | 0 (1) |
| Ab #8 (Ab-c) | High | 0 (1) | 0 (0) | 0 (1) | 0 (4) | 0 (3) | 0 (2) |
| Ab #9 | Low | 1 (1) | 0 (0) | 0 (1) | 2 (4) | 0 (3) | 0 (2) |
| Ab#10 (Ab-d) | Low | 0 (0) | 0 (0) | 0 (1) | 0 (4) | 0 (1) | 0 (1) |

The Table listed the overview of the number of highlighted Developability Issues and in parenthesis the number of highlighted Lesser Developability Issues. IgG2 disulfide scrambling was added to the Lesser Developability count.

TABLE V
Manufacturability Assessment of the ten tested antibodies

| Antibodies | Protein Engineering | Process Control and Monitoring only |
|---|---|---|
| Ab #1 | Not recommended. | Increased risk of aggregation. Potential issues likely |
| | | manageable. |
| Ab #2 | Mitigation by removing CDR H2 Aspartic | Increased risk of aggregation and Aspartic acid |
| | acid isomerisation site is possible by | isomerisation in CDR. Potential issues likely |
| | evaluating up to 3 single residue variants. | manageable |
| Ab #3 | Up to 3 single residue variants for each of | Risk of increased cost and longer development timeline. |
| | the two higher risk PTMs and a | CDR Asparagine deamidation and Methionine |
| | combination of the most successful. | oxidation will likely occur. Purification of single |
| | Alternatively, up to 16 variants to | monomeric species may be more complex. Formulation |
| | evaluate all single and double residue | design may be more complex; shelf-life may be shorter. |
| | variants in one round of expression. | Regulators may ask for in vitro and/or in vivo evidence |
| | | that PTMs do not affect affinity or potency. |
| Ab #4 (Ab-a) | Not recommended. | Increased risk of aggregation. Potential issues likely |
| | | manageable. |
| Ab #5 | Mitigation by removing CDR H2 | Increased risk of Methionine oxidation in CDR. |
| | Methionine oxidation site is possible by | Potential issues likely manageable. |
| | evaluating up to 3 single residue variants. | |
| Ab #6 (Ab-b) | Not needed. | Standard. |
| Ab #7 | Not recommended. | Increased risk of aggregation. Potential issues likely |
| | | manageable. |
| Ab #8 (Ab-c) | Not recommended. | Increased risk of aggregation. Potential issues likely |
| | | manageable. |
| Ab #9 | Up to 3 single residue variants for the | High aggregation risk. CDR Asparagine deamidation |
| | deamidation site. A single double | risk. Purification of single monomeric species may be |
| | Cysteine substitution for the free Cysteine | more complex. Formulation design may be more |
| | thiols. Combination of successful | complex; shelf-life may be shorter. Regulators may ask |
| | variants. Alternatively, up to 6 variants | for in vitro and/or in vivo evidence that PTMs do not |
| | tackling both issues. Substitute IgG2 | affect affinity or potency. |
| | isoform for e.g. IgG4 isoform with | |
| | Proline stabilised hinge. | |
| Ab #10 (Ab-d) | Not needed. | Standard. |

Lonza's in silico manufacturability assessment tools were used to identify structural or sequence elements which have the potential to result in aggregation and post-translational modifications (PTMs) such as glycosylation, deamidation, oxidation and variation of the N- and C-termini. The Protein Engineering column outlines the approximate scope of a protein engineering project to mitigate the identified risks. The scope is set to explore the most likely successful substitutions that remove the PTM whilst retaining binding affinity in a small number of variants.

TABLE VI
Yields and titres of 1.2 L fed batch cultures
for the anti-hResistin antibodies

| | Concen- | | | | | |
|---|---|---|---|---|---|---|
| | tration | Volume | Yield | Titre | Monomer | Endotoxin |
| Antibodies | (mg/mL) | (ml) | (mg) | (mg/L) | (%) | (EU/mg) |
| Ab-a | 1.39 | 100 | 139 | 115.8 | 96.31 | <0.055 |
| Ab-b | 1.35 | 70 | 94.5 | 78.75 | 98.95 | ~1.5 |
| Ab-c | 0.23 | 50 | 11.5 | 9.5 | 99.94 | ~0.89 |
| Ab-d | 0.77 | 150 | 115.5 | 96.25 | 100 | ~0.055 |

TABLE VII
Yields and titres of 6 L fed batch cultures
for the anti-hResistin antibodies

| | Concen- | | | | | |
|---|---|---|---|---|---|---|
| | tration | Volume | Yield | Titre | Monomer | Endotoxin |
| Antibodies | (mg/mL) | (ml) | (mg) | (mg/L) | (%) | (EU/mg) |
| Ab-a | 5.07 | 400 | 2028 | 338 | 96.84 | <0.050 |
| Ab-b | 3.53 | 400 | 1412 | 235.3 | 98.93 | <0.053 |
| Ab-c | 1.4 | 250 | 350 | 31 | 99.75 | <0.050 |
| (10 L) | | | | | | |
| Ab-d | 0.9 | 1300 | 1178 | 196.3 | 99.99 | <0.050 |

TABLE VIII
HPLC summary component (for % area)
in the generated antibodies

| | | Total | Total | Total |
|---|---|---|---|---|
| Antibodies | Injection | Fragments | Aggregates | Monomer |
| Ab-a | 1 | — | 3.66 | 96.33 |
| Ab-a | 2 | — | 3.62 | 96.37 |
| Ab-b | 1 | — | 0.26 | 99.74 |
| Ab-b | 2 | — | 0.25 | 99.75 |
| Ab-c | 1 | — | — | 99.94 |
| Ab-c | 2 | — | 0.15 | 99.75 |
| Ab-d | 1 | — | 7.03 | 92.97 |
| Ab-d | 2 | — | 7.12 | 92.88 |
| IgG1 Control | 1 | — | 0.84 | 99.15 |
| IgG1 Control | 2 | — | 0.84 | 99.14 |

TABLE IX
Stability Study Conditions

| Condition | Temperature | Duration |
|---|---|---|
| Agitation | Room Temperature | 14 Days |
| Stationary | 5° C. | 14 Days |
| Stationary | 40° C. | 14 Days |
| 5x sequential Freeze Thaw | −20° C. followed by thaw at RT | 1 Day |
| Cycles | | |
| Low pH hold (pH 3.5) | Room Temperature | 1 Day |
| followed by neutralization | | |
| in PBS pH 7.2 | | |

TABLE X
Serum CBC parameters of the antibody-treated rats

| | CBC | | | | | | |
|---|---|---|---|---|---|---|---|
| | Vehicle (Con)-injected | | | Ab-b-injected | | | Con vs. Ab |
| Item | Mean | SEM | Range | Mean | SEM | Range | p value |
| RBC | 8.132 | 0.22 | 7.43-8.6 | 8.186 | 0.16 | 7.81-8.75 | 0.84 |
| (M/uL) | | | | | | | |
| Hb (g/dL) | 15.68 | 0.30 | 14.9-16.6 | 15.44 | 0.32 | 14.8-16.6 | 0.60 |
| HCT (%) | 46.26 | 1.26 | 43.1-49.3 | 45.26 | 1.22 | 41.9-49.4 | 0.58 |
| MCV (fL) | 56.94 | 1.25 | 54-61.1 | 55.26 | 0.59 | 53.6-56.5 | 0.26 |
| MCH (pg) | 19.34 | 0.48 | 18.8-20.6 | 18.88 | 0.17 | 18.4-19.4 | 0.39 |
| RET | 312.06 | 20.80 | 272.6-367.2 | 320.84 | 34.59 | 216.5-431.2 | 0.83 |
| (K/uL) | | | | | | | |
| RET (%) | 3.854 | 0.29 | 3.17-4.55 | 3.728 | 0.52 | 2.61-5.37 | 0.84 |
| PLT | 801.2 | 169.83 | 211-1193 | 1015.6 | 32.55 | 933-1097 | 0.25 |
| (K/uL) | Comment: Two rats are relatively low in the Con group, which could be | | | | | | |
| | attributed to post mortem blood collection. | | | | | | |
| WBC | 12.684 | 1.70 | 17.85-8.03 | 11.428 | 2.06 | 4.4-15.93 | 0.65 |
| (K/uL) | Comment: Two rats are relatively high in the Con group, which could be | | | | | | |
| | attributed to post mortem blood collection. | | | | | | |
| NE (K/uL) | 1.304 | 0.21 | 0.83-2.07 | 1.558 | 0.38 | 0.39-2.7 | 0.57 |
| LY (K/uL) | 9.978 | 1.56 | 6.96-15.72 | 8.812 | 1.79 | 2.51-12.05 | 0.64 |
| MO | 0.59 | 0.14 | 0.21-0.91 | 0.678 | 0.17 | 0.11-1.09 | 0.69 |
| (K/uL) | | | | | | | |
| EO (K/uL) | 0.794 | 0.43 | 0.01-2.06 | 0.356 | 0.26 | 0.01-1.37 | 0.41 |

TABLE XI
Serum chemistry parameters of the antibody-treated rats

| | Chemistry | | | | | | |
|---|---|---|---|---|---|---|---|
| | Vehicle (Con)-injected | | | Ab-b-injected | | | Con vs. Ab |
| Item | Mean | SEM | Range | Mean | SEM | Range | p value |
| AST (U/L) | 127.6 | 13.76 | 83-166 | 122.4 | 10.62 | 97-144 | 0.77 |
| ALT (U/L) | 51 | 6.07 | 37-66 | 40.4 | 1.63 | 36-44 | 0.13 |
| BUN | 14.8 | 0.73 | 13-16 | 16.2 | 1.02 | 13-19 | 0.30 |
| (mg/dL) | | | | | | | |
| ALP (U/L) | 270.4 | 25.54 | 188-325 | 266.8 | 19.74 | 221-331 | 0.91 |
| Ca (mg/dL) | 12.28 | 0.69 | 10.7-14 | 10.7 | 0.40 | 9.9-12.2 | 0.08 |
| Glu (mg/dL) | 457 | 98.26 | 284-702 | 315.2 | 75.75 | 130-566 | 0.29 |
| LDH (U/L) | 665 | 128.07 | 429-1161 | 1074.4 | 169.54 | 710-1692 | 0.09 |
| GGT (U/L) | 3.2 | 0.66 | 1.0-5.0 | 5.2 | 0.97 | 43564 | 0.13 |
| Tprot (g/dL) | 6.9 | 0.11 | 6.6-7.2 | 6.4 | 0.10 | 6.2-6.7 | 0.01 |
| Alb (g/dL) | 3.64 | 0.04 | 3.5-3.7 | 3.36 | 0.02 | 3.3-3.4 | 0.00 |
| TBil (mg/dL) | 0.3 | 0.00 | 0.3-0.3 | 0.28 | 0.02 | 0.2-0.3 | 0.35 |
| Creat | 0.58 | 0.07 | 0.4-0.8 | 0.58 | 0.02 | 0.5-0.6 | 1.00 |
| (mg/dL) | | | | | | | |
| CK | 540 | 58.96 | 395-699 | 1407.8 | 396.79 | 457-2517 | 0.06 |
| (U/L) | Comment: two rats are relatively high in the Ab group, which is non- | | | | | | |
| | specific tissue damage - blood collection. | | | | | | |
| Phos (mg/dL) | 10.02 | 1.48 | 7.2-13.9 | 8.38 | 0.82 | 6.9-11.6 | 0.36 |
| MG (mg/dL) | 3.42 | 0.63 | 2.0-5.0 | 2.86 | 0.40 | 2.2-4.3 | 0.48 |
| CHOL | 109.8 | 8.48 | 82-133 | 94.8 | 8.68 | 69-117 | 0.25 |
| (mg/dL) | | | | | | | |
| TRIG | 168.8 | 9.87 | 148-204 | 147.4 | 23.39 | 81-211 | 0.42 |
| (mg/dL) | | | | | | | |
| AMYL (U/L) | 1022.4 | 45.42 | 879-1129 | 947.4 | 45.81 | 818-1046 | 0.28 |
| UA | 5.36 | 1.52 | 1.7-9.2 | 3 | 0.48 | 2.1-4.6 | 0.18 |
| (mg/dL) | Comment: Two rats are relatively high in the Con group, which could be | | | | | | |
| | attributed to post mortem blood collection. | | | | | | |
| HDL | 35 | 2.21 | 27-40 | 31 | 2.35 | 24-37 | 0.25 |
| (mg/dL) | | | | | | | |
| DBILI | 0.34 | 0.02 | 0.3-0.4 | 0.3 | 0.03 | 0.2-0.4 | 0.35 |
| (mg/dL) | | | | | | | |
| Na (mmol/L) | 145 | 1.48 | 142-150 | 144 | 2.59 | 136-152 | 0.75 |
| K | 9.22 | 2.27 | 5-14.9 | 6.46 | 0.50 | 5.6-8.4 | 0.27 |
| (mmol/L) | Comment: Two rats are relatively high in the Con group, which could be | | | | | | |
| | attributed to post mortem blood collection. | | | | | | |
| CL (mmol/L) | 101.6 | 0.87 | 99-104 | 102.8 | 1.80 | 98-109 | 0.57 |

TABLE XII
Gross evaluation of the antibody-treated rats

| Item | Vehicle (Con)-injected | Ab-b-injected |
|---|---|---|
| Gross | BCS 4/5 (5 rats; 3 unremarkable) | BCS 4/5 (4 rats; one unremarkable) |
| (BCS) | | BCS 3/5 (1 rat; unremarkable) |
| Right | WNL (4 rats) | WNL (2 rats) |
| Kidney | Hydronephrosis 1-3 mm (1 rat) | Hydronephrosis 1-3 mm (1 rat) |
| | | Hydronephrosis 1-4 mm (1 rat) |
| | | Hydronephrosis 3-6 mm (1 rat) |
| Left | WNL (3 rats) | WNL (4 rats) |
| Kidney | Hydro-nephrosis <2 mm (2 rats) | Hydro-nephrosis <2 mm (1 rat) |
| Comment | Hydronephrotic changes are very mild in both groups and both kidneys. | |
| Other | WNL (4 rats) | WNL (4 rats) |
| | Hemorrhage retroperitoneal <5 mm (1 rat) | Hemorrhage R medial thigh 4-8 mm (1 rat) |

| | |
|---|---|
| Comment | Mild hemorrhage at these sites may be related to IP injections. |

TABLE XIII
Organ Weights of the antibody-treated rats

| | Organ Weights (g) perfused | | | | | | |
|---|---|---|---|---|---|---|---|
| | Vehicle (Con)-injected | | | Ab-b-injected | | | Con vs. Ab |
| Item | Mean | SEM | Range | Mean | SEM | Range | p value |
| Liver | 20.12 | 0.57 | 18.00-21.16 | 17.09 | 0.63 | 14.85-18.52 | 0.01 |
| LIVER: % of | 4.42 | 0.05 | 4.32-4.59 | 4.09 | 0.10 | 3.85-4.45 | 0.02 |
| body weight | | | | | | | |
| Spleen | 0.84 | 0.04 | 0.78-0.94 | 0.89 | 0.10 | 0.56-1.149 | 0.60 |
| SPLEEN: % | 0.18 | 0.01 | 0.16-0.21 | 0.21 | 0.02 | 0.16-0.25 | 0.20 |
| of body | | | | | | | |
| weight | | | | | | | |
| Heart | 1.54 | 0.04 | 1.41-1.63 | 1.52 | 0.13 | 1.272-2.027 | 0.91 |
| HEART: % of | 0.34 | 0.00 | 0.33-0.35 | 0.36 | 0.02 | 0.33-0.45 | 0.33 |
| body weight | | | | | | | |
| Right Kidney | 1.86 | 0.05 | 1.74-2.00 | 1.75 | 0.07 | 1.544-1.958 | 0.23 |
| Left Kidney | 1.92 | 0.05 | 1.78-2.08 | 1.67 | 0.09 | 1.378-1.893 | 0.04 |
| KIDNEYS: % | 0.83 | 0.02 | 0.78-0.86 | 0.82 | 0.04 | 0.7-0.95 | 0.82 |
| of body | | | | | | | |
| weight | | | | | | | |
| Body | 455.35 | 12.38 | 410.86-481.85 | 418.81 | 16.28 | 357.53-451.2 | 0.11 |

| | |
|---|---|
| weight | Comment: A cause for lower body weight in one of the Ab-injected rat |
| | (357.53 g) is not identified. There is ample fat. |

TABLE XIV
Histological analysis of tissues from the antibody-treated rats

| Item | Vehicle (Con)-injected | Ab-b-injected |
|---|---|---|
| Heart | WNL cw perfusion (4 rats) | WNL cw perfusion (5 rats) |
| | MDF focal mild <1 mm (1 rat) | |
| Comment | MDF = Myocardial Degeneration loss fibrosis; inflam minimal <1; Unsurprising | |
| | background finding. | |
| Lung (left) | WNL (2 rats) | WNL (3 rats) |
| | inflam <1 min subpleural (1 rat) | inflam <1 min interstitial (1 rat) |
| | inflam <1 min interstitial (1 rat) | Inflam ++ 1 Interstit alveolar, rare |
| | Inflam ++ 1 Interstit alveolar, rare | pmn; scant pigment (1 rat) |
| | pmn; scant pigment (1 rat) | |
| BALT | MF <<1 mm <1 (3 rats) | MF <1 mm + 1 (5 rats) |
| | MF <1 mm + 1 (2 rats) | |
| Hemorrhage Cw | Mild MF (5 rats) | Mild MF (4 rats); Mild focal (1 rat) |
| cardiocentesis | | |
| Histiocytosis | <<1 (4 rats); Mild + 1 MF (1 rat) | <<1 (3 rats); <1 mild focal (1 rat); |
| Aleolar | | Mild + 1 MF (1 rat) |
| Comment | Mild non-specific inflammatory changes suggest pneumocystosis. | |
| Kidney RX | WNL (4 rats) | WNL (2 rats) |
| | Hydronephrosis 1-3 mm (1 rat) | Hydronephrosis 1-3 mm (1 rat) |
| | | Hydronephrosis 1-4 mm (1 rat) |
| | | Hydronephrosis 3-6 mm (1 rat) |
| Tubules | WNL (3 rats) | WNL (5 rats) |
| | Degen/Regeneration mild + 1 | |
| | (2 rats) | |
| Glomeruli | WNL (3 rats) | WNL (5 rats) |
| | QNL (2 rats) | |
| Inflammation | 0/<<1 (3 rats); <1 | 0/<<1 (5 rats) |
| | min Multifocal (2 rats) | |
| Comment | Mild degenerative & regenerative tubule changes and minimal mild inflammatory | |
| | changes are unsurprising background findings. | |
| Liver | WNL (5 rats) | WNL (4 rats) |
| Median Lobe | | Loss/fibrosis focal scar (1 rat) |
| Hepatocytes | WNL (5 rats) | WNL (5 rats) |
| Inflammation | <1 Minimal MF portal etc (4 rats) | <1 Minimal MF portal etc (1 rat) |
| | <1 min focal <<1 mm (1 rat); | <1 min focal <<1 mm w necrosis loss (1 rat) + |
| | Adhesion capsule <1 mm Focal | 1 mild focal <<1 mm w necrosis loss (1 rat) |
| | fibrous (1 rat); | Mild + 1 MF portal etc; <1 min focal <<1 mm |
| | Hemorrhage focal mild cw | (2 rats) |
| | perimortem (1 rat) | |

| | |
|---|---|
| Comment | Scar cw IP injection; Mild inflammatory changes are unsurprising background |
| | findings; Mild adhesions attributed to IP injections. |

TABLE XIV
Histological analysis of tissues from the antibody-treated rats (continued)

| Item | Vehicle (Con)-injected | Ab-b-injected |
|---|---|---|
| Spleen | WNL (5 rats) | WNL (5 rats) |
| Red/White | ~1 WNL (5 rats) | ~1 WNL (5 rats, one rat prom MZ) |
| Mesentery/Pancreas | | |
| Pancreas Exocrine | WNL (5 rats) | WNL (5 rats) |
| Pancreas Endocrine | WNL (5 rats) | WNL (5 rats) |
| Mesentery | WNL inflam <1 perivasc (5 rats) | WNL inflam <1 perivasc (5 rats) |
| L nodes | WNL <3 mm + GC (3 rats); <<3 | WNL <3 mm + GC (5 rats) |
| | mm + GC (2 rats) | |
| | Siderophages + foamy | Siderophages ++ foamy mac/sinuses (1 |
| | mac/sinuses + erythrophag | rat); +erythrophag (4 rats) |
| | (4 rats); +RBC (1 rat) | |
| Comment | cw IP injections? | |
| Stomach/Cecum | | |
| Forestomach | WNL (5 rats) | WNL (5 rats) |
| Stomach | WNL (5 rats) | WNL (5 rats) |
| Cecum | WNL (5 rats) | WNL (5 rats) |
| GALT | NT (2 rats) | NT (1 rat) |
| | 1-3 mm (WNL, 1 rat) | 1-2 mm (WNL + GC, 1 rat) |
| | 1-4 mm (WNL, 1 rat) | 1-4 mm (WNL + GC, 1 rat) |
| | 1-6 mm (WNL + GC, 1 rat) | 1-6 mm (WNL + GC, 1 rat) |
| | | 2-3 mm (WNL + GC, 1 rat) |
| Intestine | | |
| Small Intestine | WNL (5 rats) | WNL (5 rats) |
| Colon | WNL (5 rats) | WNL (5 rats) |
| | MALT + GC WNL (2 rats) | |
| | GALT + Lnode <@mm WNL | |
| | (1 rat) | |

Abbreviations: cw, consistent/compatible with; GC, Germinal center; Inflam, inflammation (mononuclear unless otherwise qualified); LN, Lymph node; MF, Multifocal; NSF, No significant findings (unremarkable); NT/NP, No tissue/Not present; QNS, Quantity not sufficient; WNL, Within normal limits/No significant findings (unremarkable).

TABLE XV
Criterion of scoring for the histological (toxicological) analysis
Scoring: 0-3

| | |
|---|---|
| 0 | Unremarkable WNL NSF |
| 1 | Minimal mild |
| 2 | Moderate |
| 3 | Marked -Severe |

### Example 3: Resistin Predicts Disease Severity and Survival in Patients with Pulmonary Arterial Hypertension

The present inventor's previous work suggested that resistin, a pleiotropic cytokine involved in inflammatory diseases, plays a critical role in the pathogenesis of human pulmonary arterial hypertension (PAH). In this study, the present inventor evaluated the potential of resistin as a genetic and biological marker for disease severity and survival in a large cohort of patients with PAH.

The present inventor obtained biospecimens, as well as clinical and genetic data, from 1121 adults with PAH. Of those, 808 had idiopathic PAH (IPAH) and 313 scleroderma-associated PAH (SSc-PAH). Serum resistin levels were measured by ELISA, and associations between resistin levels, clinical variables, and single nucleotide polymorphism genotypes were examined with multivariable regression models. The present inventor applied machine-learning (ML) algorithms to develop and compare risk models for mortality prediction in PAH.

Circulating resistin levels were significantly higher in samples from all PAH and subtypes (IPAH and SSc-PAH) than in controls (P<0.0001) and had superior discriminative abilities (AUCs of 0.84, 0.82 and 0.91, respectively. P<0.001). PAH patients exhibiting high resistin levels (above 4.54 ng/ml) were associated with older age (P=0.001), shorter 6-minute walk distance (P=0.001) and reduced cardiac performance (i.e., cardiac index, P=0.016). Interestingly, mutant carriers of either rs3219175 or rs3745367 had higher resistin levels (adjusted P=0.0002 and 0.0003, respectively). PAH patients with higher resistin levels were also associated with increased risk of death (hazard ratio: 3.14; 95% CI: 1.52-6.49; P<0.002). Comparisons of ML-derived survival models confirmed satisfactory prognostic value of the random forest model (AUC=0.70, 95% CI: 0.62-0.79).

Serum resistin could serve as a reliable, noninvasive biomarker for diagnosis and prognostication of PAH. ML-derived survival models highlighted the importance of including biomarkers such as resistin level to improve model performance. Future studies are needed to develop multi-marker assays that improve noninvasive risk stratification in patients with PAH.

### INTRODUCTION

Pulmonary arterial hypertension (PAH) is a multifactorial and life-threatening condition characterized by abnormal remodeling of distal pulmonary arteries. This remodeling leads to a progressive increase in pulmonary vascular resistance and subsequent right ventricular hypertrophy and failure. 1 Current therapies fail to substantially reduce PAH progression and mortality. Mechanistic biomarkers, by serving as reliable predictors of PAH severity and survival, could be crucial for the development of treatment strategies.

One potential biomarker candidate is resistin, a member of the resistin-like molecule (RELM) family of pleiotropic cytokines.2 Resistin, which was first identified as an adipokine in mice with insulin resistance properties,3 is predictive of poor clinical outcomes in patients with cardiovascular disease and heart failure.4-7 The present inventor have shown that mRELMα, the mouse homolog of resistin, is dramatically upregulated in hypoxic lungs and produces potent mitogenic effects.8 In rodent models, transtracheal delivery of mRELMα gene by adeno-associated virus causes vascular remodeling and hemodynamic changes like those of PAH.9 Conversely, in vivo knockdown of mRELMα markedly reduces PAH development caused by chronic hypoxia or Th2 inflammatory stimuli,9-11 indicating an etiologic role for mRELMα in PAH. However, the utility of using resistin to assess PAH disease severity and predict survival has not been studied in humans.

The present inventor sought to assess the relationship of serum resistin levels with PAH disease severity and survival in a large cohort of PAH patients mainly comprised of two subtypes: IPAH and SSc-PAH. Because right ventricular hypertrophy and failure is the major cause of mortality in patients with PAH, the present inventor hypothesized that resistin levels would be associated with PAH severity (i.e., hemodynamic measures) and mortality. Models combining resistin levels with clinical indicators have enhanced the ability to predict mortality compared with models that use clinical indicators alone. In this study, the present inventor show that resistin is a biochemical and genetic marker for PAH. Based on the present inventor's findings, future studies to develop resistin-targeted therapy are warranted.

### Materials and Methods

Study Subjects. The National Biological Sample and Data Repository for PAH (also known as the PAH Biobank) is a National Institutes of Health-funded repository of biologic samples and clinical data collected from 36 enrolling PAH centers across North America. Biorepository data collection was approved by the institutional review board at each participating center, and all patients gave informed consent at the time of enrollment. Inclusion and exclusion criteria have been described elsewhere, 12, 13 and details are in the online supplement. The present inventor received clinical data and biologic samples, including serum, from all patients who had idiopathic PAH (IPAH) or scleroderma-associated PAH (SSc-PAH) and were 18 years of age or older (n=1121). Samples from 50 healthy control subjects were obtained from Innovative Research to use as controls. This study was approved by the Johns Hopkins University Institutional Review Board.

Serum resistin levels in PAH patients. ELISAs for resistin levels were successfully performed on serum from all patients. Briefly, serum resistin was analyzed in duplicate using the mesoscale discovery plate assay (see Supplementary Methods). To compare values between and among groups, the present inventor used the Mann-Whitney U-test and Kruskal-Wallis test, where appropriate.

Genotyping. DNA was extracted according to standard protocols. Genotyping for single nucleotide polymorphisms (SNPs) was carried out by using a genome-wide genotyping array (Illumina HumanOmni5, Illumina Inc., San Diego, CA, USA), with an average completion rate of 98%, 9 Three S NPs within the gene encodes resistin (RETN) and ~2 kb upstream (rs7408174 [T>C, upstream of RETN], rs3219175 [G>A,-2 kb variant], and rs3745367 [G>A, intron variant]) were covered by the array and analyzed for association with serum resistin level (Table 3).

Statistical Analyses. Baseline characteristics are presented as median and interquartile range (IQR), number and percentage, or median and range, where appropriate. Resistin level was not normally distributed; therefore, a nonparametric test was performed with log-transformed data. The chi-square test, Mann-Whitney U test, or Kruskal-Wallis test was used for comparisons between groups. Correlation analyses were carried out utilizing linear regression with adjustment for age, sex and body mass index (BMI). To evaluate the performance of resistin level as a discriminator of PAH presence, the present inventor calculated the area under the curve (AUC) of the receiver operating characteristic (ROC) curve. Survival curves were computed with Kaplan-Meier estimates to determine time-to-death, and log-rank tests were performed to compare survival distributions. The association between resistin level and survival was also tested with multivariable Cox regression models. The present inventor used logistic regression models to test for genetic marker association with resistin level, adjusting for age, sex, ethnicity, and BMI. A P value <0.05 was considered statistically significant. Statistical analysis was carried out in MedCalc Statistical Software version 18.11.3 (MedCalc Software, Ostend, Belgium).

Mortality model construction and assessment. For variable selection, initially the present inventor selected 21 variables (see Supplement). These included demographics (age, sex, BMI, and body surface area (BSA)), clinical classification of PAH; and hemodynamic measurements (n=10). Additionally, the present inventor included REVEAL 2.0 risk score, serum resistin level and the genotypes of three RETN SNPs (rs7408174, rs3219175, rs3745367). Then, the present inventor applied Lasso regression to the 13 quantitative variables. At last, 15 variables including the 6 quantitative variables selected by Lasso, 4 additional hemodynamic variables and 5 categorical variables were utilized for the full model. For quality control process of the data, 902 PAH patients (IPAH-654) were enrolled after removing subjects with missing values for quantitative variables. For ML analysis, first, the present inventor randomly selected 70% (n=631, IPAH=455) of the patients as the training set for model construction. Next, the present inventor balanced the dataset using SMOTE-NC.14 Then, 5 commonly adopted predictive model types were established to predict PAH mortality: random forest (RF), XGBoost, support vector machine (SVM), multilayer perceptron (MLP), and a stacking classifier model. To get optimal prediction performance, K-folder cross validation (k=5) was used to train, construct, and compare the 5 predictive models. The confusion matrix, area under the ROC curve (AU-ROC), sensitivity, positive predictive value (PPV), and F1 score (which is the harmonic mean of the recall and precision) were used to evaluate and compare the comprehensive performance of model types. Lastly, 30% of the entire cohort were included in the test set (n=271, IPAH=199) to validate the training set.

### Results

Patient Characteristics. Demographics and clinical characteristics of PAH patients in this study are presented in Table 1. The cohort was composed mainly of patients with IPAH (808); the second largest disease subtype was SSc-PAH (n=313). As in previous studies, 13 most patients were white women in the sixth decade of life, with New York Heart Association functional class (NYHA FC) III/IV symptoms. Subjects had moderate to severe disease, with mean pulmonary artery pressure (mPAP) of 49 mm Hg (IQR: 19), pulmonary vascular resistance (PVR) of 8.95 Wood units (IQR: 7.03), and cardiac index (CI) of 2.54 L/min/m2 (IQR: 1.16). At enrollment, most patients were being treated with a phosphodiesterase-5 (PDE5) inhibitor or endothelin receptor antagonist (ERA) therapy. The control cohort was 50% male and had a median age of 38 years (range: 18-57).

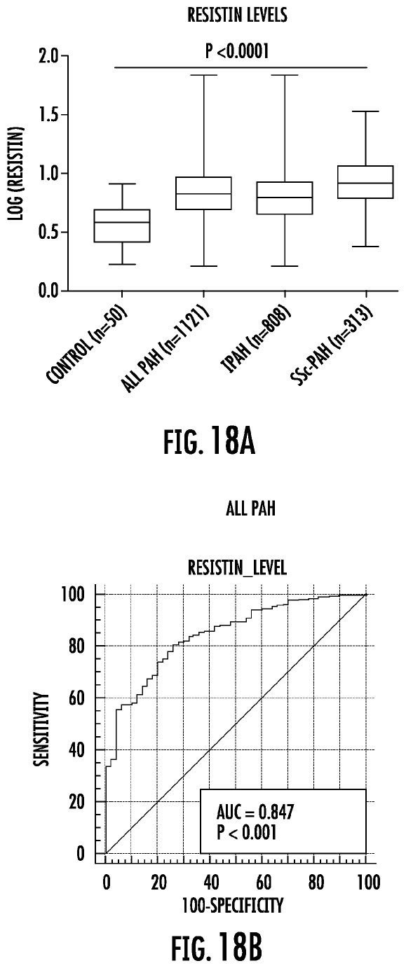

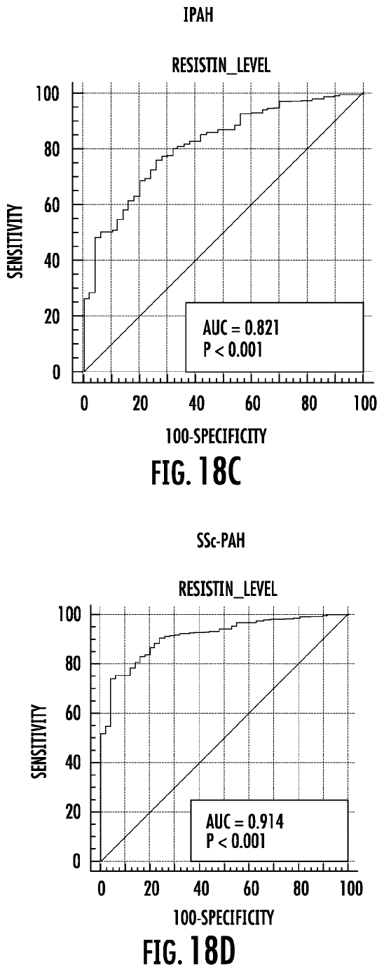

Serum resistin levels were significantly elevated in patients with PAH and had potential for predicting PAH. Serum samples from 808 IPAH patients, 313 SSc-PAH patients, and 50 healthy control subjects were available for analysis. When compared to circulating resistin levels in healthy control subjects (median [IQR]=3.84 ng/ml [2.14]), levels were significantly higher in samples of the overall PAH cohort (n=1122; 6.63 ng/mL [4.34]), in IPAH patients (6.2 ng/mL [3.67]), and in SSc-PAH patients (8.28 ng/ml [5.59]), all Ps<0.0001 (FIG. 18A).

To evaluate the specificity and sensitivity of serum resistin as a predictor for PAH diagnosis, the present inventor used resistin levels from PAH patients and controls to generate an ROC curve. Serum resistin had the ability to discriminate all PAH, IPAH or SSc-PAH from control subjects with AUCs of 0.85, 0.82 and 0.91 (FIG. 18B-18D), respectively (P<0.001). Based on this ROC curve analysis, the present inventor established a serum resistin threshold value of 4.54 ng/ml (defined by the Youden index) to distinguish healthy individuals from those with PAH or IPAH, and 6.30 ng/ml for SSc-PAH.

Serum resistin levels were associated with metrics of PAH disease severity. Utilizing multiple linear regressions adjusting for age, sex and BMI, the present inventor evaluated the relationship between serum resistin levels (log transformed) and continues clinical variables including invasive resting hemodynamics and exercise tolerance assessed by the 6-minute walk distance (6MWD). In PAH patients, serum resistin was significantly associated with right atrial pressure (RAP, P<0.026) and inversely associated with CI (P<0.014, Table 2). Additionally, the present inventor observed a significant correlation between resistin level and PAH severity measured by REVEAL Registry PAH risk score 2.0 (P<0.001), each log-unit higher resistin was associated with a 0.1-point higher risk score. The present inventor observed similar trends for RAP (P<0.032) and CI (P<0.01) for the IPAH subtype, but not in the SSc-PAH patients.

The present inventor further dichotomized PAH patients into resistin-level low and resistin-level high subgroups based on whether their serum resistin levels were below or above the identified threshold defined by the Youden index (4.54 ng/mL). As shown in Table 3, patients within the resistin-level high group were older (median [IQR]: 59 vs. 53 [23.75]y, P=0.001) and had shorter 6MWD (median [IQR]: 340 vs. 378.5 meters, P=0.008), consistent with poor exertional tolerance and symptoms. Additionally, resistin-level high patients had worse invasive hemodynamic parameters, mainly CI (median [IQR]: 2.51 [1.13] vs. 2.69 [1.13] L/min/m2, P-0.016), and fewer received ERA (54.16% vs. 66.98%, P=0.001). Thus, PAH patients with higher resistin levels had diminished functional capacity that may contribute to the high mortality rate (19.81% vs. 11.82%, P=0.008). Similar trend was observed for IPAH: patients within the resistin-level high group were older (median=54 vs. 51.5 [23.25] y, P=0.021), had decreased CI (median [IQR]: 2 (1) vs. 3 (1) L/min/m2, P=0.004), but increased NTproBNP levels (501 vs. 472 μg/mL, P=0.03). In contrast, when the present inventor stratified SSc-PAH patients into resistin-level high and resistin-level low groups, demographics and clinical characteristics were comparable between the two groups (Supplementary Table 1).

Serum Resistin Levels were Associated with Outcomes in PAH Patients.

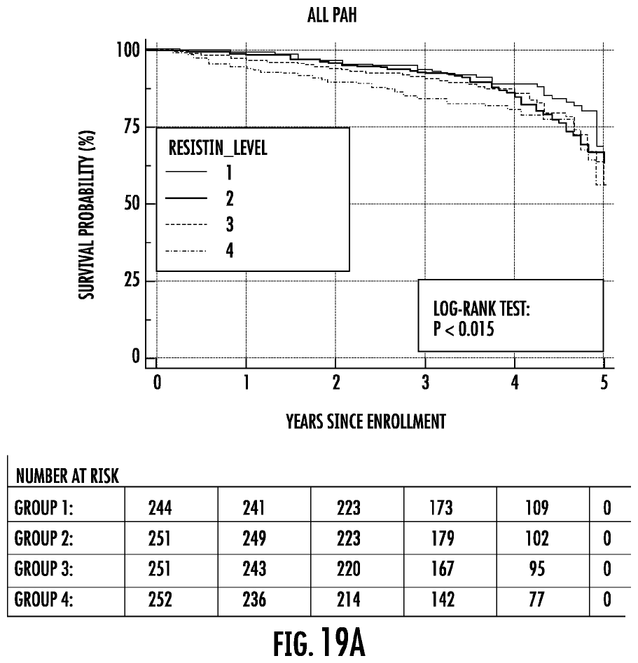

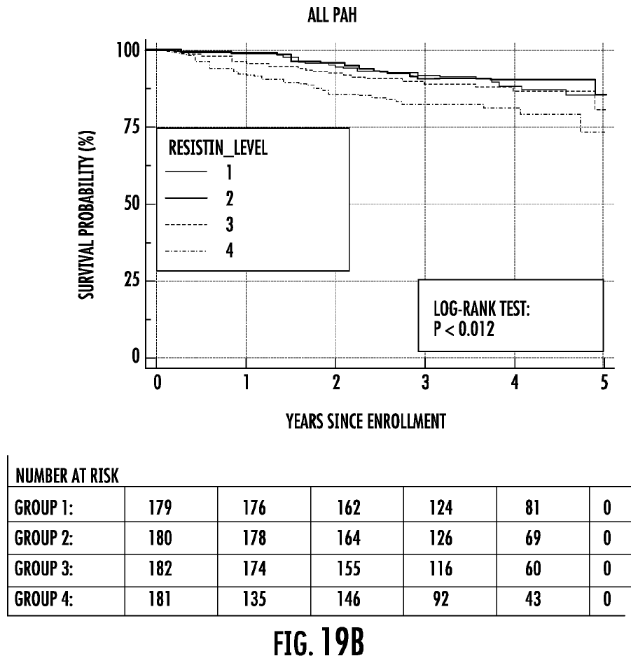

Kaplan-Meier curves. The present inventor generated Kaplan-Meier curves to assess the relationship between elevated resistin levels and mortality. The present inventor arranged resistin levels in PAH patients (n=1064) by quartiles: group 1 (<25th percentile, n=255; median log (resistin)=0.609); group 2 (25th to 50th percentile, n=233; median log (resistin)=0.761); group 3 (50th to 75th percentile, n=266; log (resistin)=0.885); and group 4 (>75th percentile, n=241; median log (resistin)=1.14). FIG. 19 demonstrated significantly shorter survival for subjects with higher resistin levels (chi-square=23.5; P<0.015 by log-rank test). Similar trend was observed for IPAH patients (chi-square=10.94; P<0.012 by log-rank test), but not in the SSc-PAH subgroup.

Univariable Cox proportional hazard modeling. Given the strong predictive value of serum resistin for PAH outcome, the present inventor further constructed Cox proportional hazard models to examine this relationship. Resistin levels were significantly associated with mortality in univariable Cox proportional hazard modeling. A high resistin level (log transformed) was a significant predictor of adverse outcomes, with an unadjusted hazard ratio (HR) of 6.04 (95% CI: 3.20-11.39; P<0.0001). Univariate analysis also showed associations between mortality and age, 6MWD, RAP, and mPAP, consistent with published data in other PAH cohorts. 15-17 When survival analyses were repeated in the two subgroups, the significance remained the same in IPAH (HR-8.41; 95% CI: 3.41-20.71; P<0.0001), but attenuated in SSc-PAH.

Multivariable Cox proportional hazards models. Multivariable models were built with adjustment for demographics (age, sex, and BMI), PAH-specific therapy, and individual hemodynamic variables (specifically mPAP, PVR, and CI) previously linked to adverse outcomes, 13, 15, 16 and variables associated with increased mortality in univariate analysis (RAP and 6MWD). In multivariable Cox proportional hazards models (Table 4 and Supplementary Table 2), the relationship between resistin level and outcome only persisted in IPAH patients (HR, 9.97; 95% CI: 1.29-76.96; P<0.027). When multivariable analyses were repeated, excluding 6MWD (which had a missing rate of 49%), the biomarker association with survival remained significant in the overall cohort (HR, 2.87; 95% CI: 1.25-6.58; P<0.013) and in IPAH (HR, 6.36; 95% CI: 2.08-19.46; P<0.001). When both 6MWD and NYHA FC (covariates with significant missingness) were excluded, the magnitude of biomarker associations with survival persisted in the overall cohort (HR, 3.14; 95% CI: 1.52-6.49; P<0.002) and IPAH (HR, 4.11; 95% CI: 1.53-11.07; P<0.005). Thus, the sensitivity analyses performed to account for missing covariate data have demonstrated 314% higher risk of mortality for each log-unit increase in resistin.

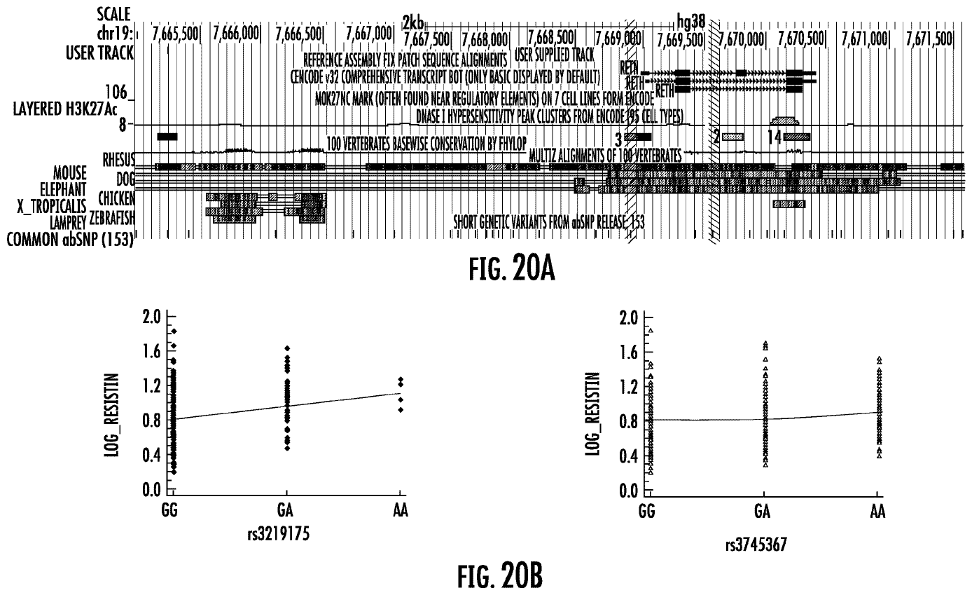

RETN genetic variants were associated with serum resistin level in PAH patients. The present inventor evaluated three RETN SNPs (rs7408174, rs3219175 and rs3745367) on the Omni5 GWAS panel (FIG. 20A) for association with serum resistin level and clinical metrics for PAH severity. In 776 IPAH patients, two SNPs located in the proximal upstream (rs3219175) and intronic region (rs3745367) of RETN were associated with resistin level. The coefficient r values were 0.218 (95% CI: 0.150-0.284; P=0.0001) for rs3219175 and 0.134 for rs3745367 (95% CI: 0.065-0.203; P=0.0002; FIG. 20B). The present inventor further adjusted the models with age, sex, ethnicity and BMI in logistic regression and observed significant adjusted P values of 0.0001 and 0.0003, respectively (Table 3). Tested under a dominant model, homozygous and heterozygous mutant carriers of rs3219175 (AA and GA genotypes) had higher resistin levels (n=41, 11.69±7.91 ng/mL) than did carriers of the wild-type GG genotype (n=735, 7.05±4.70 ng/ml). Similarly, the homozygous mutant carriers of rs3745367 (AA genotype, recessive model) had higher resistin levels (n=89, 9.19±5.73 ng/mL) than did the non-AA genotype carriers (n=688, 7.04±4.87 ng/mL).

In SSc-PAH patients, the present inventor observed significant adjusted P values of 0.0007 only for the promoter variant rs3219175. However, both variants were associated with resistin levels in the overall cohort of PAH patients with an adjusted P value of 0.0001 (Table 3).

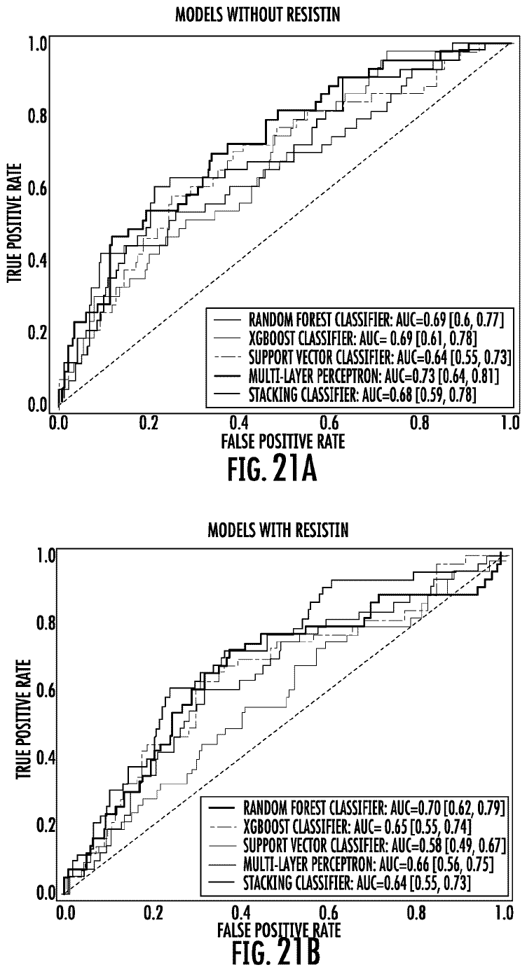

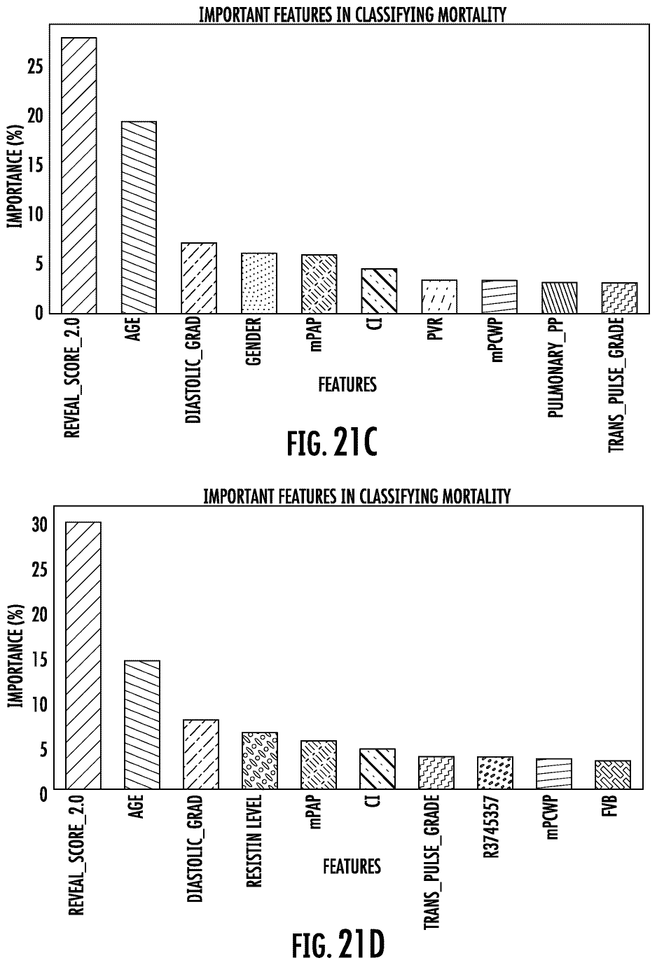

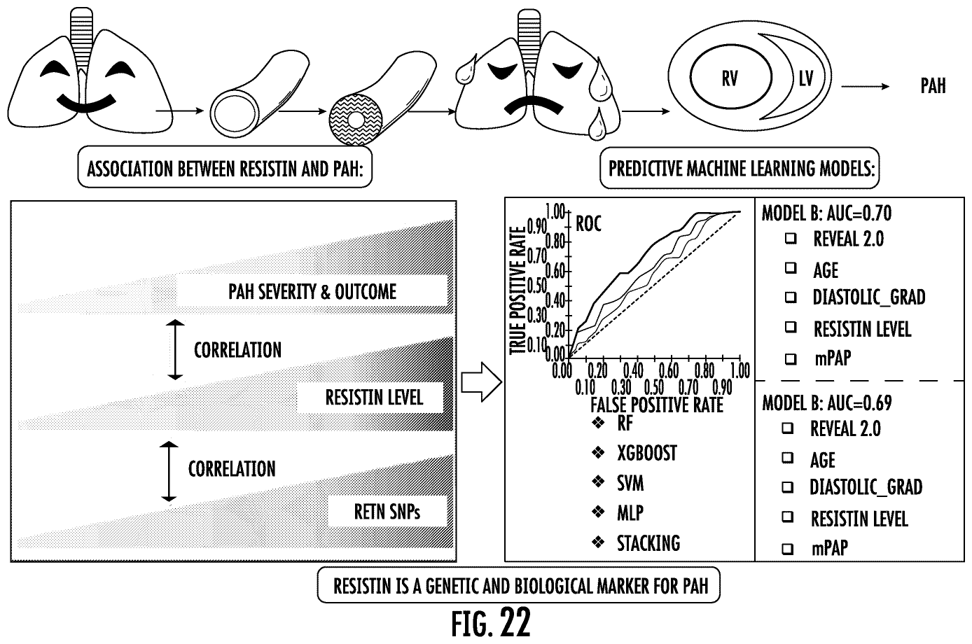

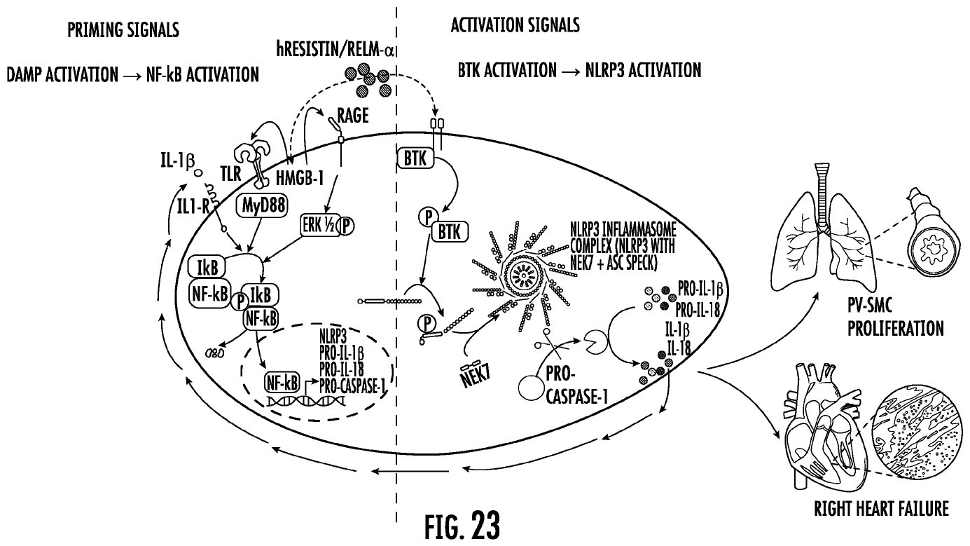

Comparison of five mortality prediction models in the test set. First, the present inventor constructed models utilizing REVEAL 2.0 risk score, demographics (age and sex), clinical classification of PAH and 7 hemodynamic measurements. Five classifiers were established and the average AU-ROC and 95% CI for each classifier are shown in FIG. 21A. All five classifiers had AUC values above 0.60 (the acceptable cutoff for accuracy) and the MLP classifier obtained the highest AUC value of 0.73 (95% CI: 0.64-0.81). As shown in Supplementary Table 3, the five classifying models demonstrated varying performances for classifying non-survivors. The RF classifier was also the best-performing in the test set (AUC=0.69; 95% CI: 0.60-0.77), with the highest sensitivity (0.58), precision (0.29), and F1 score (0.38). Second, the present inventor further constructed a full model to include resistin levels and SNPs, in addition to the REVEAL 2.0 risk score and clinical variables mentioned above. Indeed, this full model (highest AUC=0.70 from the RF classifier; 95% CI: 0.62-0.79) outperformed the model excluding resistin levels and SNPs demonstrating improved sensitivity (0.60), precision (0.29), and F1 score (0.39, Supplementary Table 3). Then, the present inventor used the RF model to analyze the importance of features in predicting mortality and FIG. 22 panels C & D show the 10 most important features. The present inventor's results suggested that REVEAL 2.0 risk score, age, diastolic pulmonary gradient [DPG], gender and mPAP were the top 5 features playing important roles in the model without resistin. In contrast, serum resistin level was among the top 5 important features in the full model. Of note, several other hemodynamic parameters (CI, PVR, mean pulmonary capillary wedge pressure [mPCWP], transpulmonary pressure gradient [TPG]) also showed varying importance in models with and without considering resistin. Carrier status of RETN SNP rs10402974 was also among the top features in the full model.

### DISCUSSION

Circulating resistin levels have an emerging role as biomarkers for a variety of diseases, including glucose metabolism and obesity, 18, 19 diabetes,20 cancer,21 inflammatory diseases such as inflammatory bowel disease,22 and cardiovascular diseases.4, 5 Because lung is the primary location of most RELM isoforms,2, 8, 23 research into the association between RELMs and the pathogenesis of cardiothoracic and respiratory diseases is now beginning to expand rapidly. In the present inventor's study, the present inventor discovered that resistin levels were significantly higher in PAH patients and subtypes compared to that in controls (P<0.0001). Furthermore, when the present inventor used the AUC values of the ROC curve as a criterion to evaluate the performance of resistin level to discern the presence of PAH, all three tests had excellent discriminative ability (AUCs were 0.84, 0.82, and 0.91 for all PAH, IPAH and SSc-PAH, respectively). More strikingly, when the present inventor further dichotomized IPAH patients into two subgroups using the identified threshold (serum resistin level of 4.54 ng/ml) derived from the ROC analysis, the present inventor found that higher resistin level was associated with older age, worsening NYHA FC, and reduced functional capacity. Additional evidence was found in survival analyses, supporting circulating resistin as a robust predictor of mortality in patients with PAH. The Kaplan-Meier curve analysis showed that elevated resistin level (above the highest quartile) is significantly associated with increased risk of death in the overall cohort (P<0.015 by log-rank test) and also in IPAH (P<0.012). Of note, this is a prevalent cohort, with the majority of patients receiving PAH-specific therapy at the time of biomarker assessment. To mitigate this limitation, the present inventor adjusted for the presence and class of PAH therapies along with other known risks, and the relationship between resistin level and outcomes persisted in multivariable Cox models (P<0.0003 in a reduced model excluding 6MWD and NYHA FC). Thus, the present inventor's study demonstrated that serum resistin can serve as a biomarker for PAH prognosis and survival in a large cohort composed solely of patients with IPAH and SSc-PAH.

Resistin expression appears to be controlled in part by genetic programming, as genotypes of RETN gene correlated with both level and disease state in some populations. Several SNPs have been shown to correlate with increased circulating resistin levels, and estimates suggest that approximately 70% of resistin expression can be attributed to genetic effects.24 Gene variants in the promoter region upstream of RETN (−420 C>G and −638 G>A) appear to have the strongest effect. The −420 C>G SNP (rs1862513) associated with increased circulating resistin levels has been associated with type 2 diabetes in several studies of Asian populations.25-27 Additionally, the −420 C>G polymorphism was significantly associated with hypertrophic cardiomyopathy in a Pakistani population.28 In the present inventor's study, subjects who carried the mutant allele of either the promoter variant rs3219175 or intronic variant rs3745367 had significantly higher resistin levels than did non-carriers; those with the promoter variant rs3219175 exhibited the strongest effects. Thus, the present inventor's genetic analysis provides insight into the variation and complexity of resistin's role in PAH.

The use of artificial intelligence in diagnosing respiratory diseases is rapidly evolving for prediction of sepsis, lung cancer prognosis, risk of hospital admission with chronic obstructive lung disease, and diagnosis of PAH.29-32 To interpret the complex data for risk stratification in patients with PAH, the present inventor adapted common machine language techniques by training the algorithm on a cohort of 631 PAH patients (training data) to accurately predict PAH mortality. The reproducibility of the predictive performance quality was further verified on the test data composed of 271 PAH patients. The results indicated that RF classifier generated the best-performing predictive classifying model (obtained the highest predictive performance as indicated by an AU-ROC value of 0.70) in the test set. Random forest is one of the ensemble models with advantages in handling mixed variable types, and it is robust to outlying observations. Based on the superior predictive performance in RF models, the present inventor determined the relative importance of each attribute. Intriguingly, serum resistin level ranked as the fourth most important feature after REVEAL 2.0 risk score, age and diastolic pulmonary gradient (DPG) for predicting mortality in PAH patients. The present inventor have utilized several hemodynamic parameters derived from the primary data including DPG (defined as: diastolic PAP-mPCWP [mm Hg]). DPG previously has been reported to be associated with survival in group 1 pulmonary hypertension patients and portends poor prognosis in heart failure.33 Another hemodynamic parameter mPAP also played important roles in the model and recent evidence suggests that even mildly elevated mPAP is associated with morbidity and mortality. Therefore, in 2018, the hemodynamic definition of pulmonary hypertension was revised by lowering the threshold from mPAP>25 mmHg to >20 mmHg.34 Thus, the present inventor's results clearly show that REVEAL 2.0 risk score is a robust predictor of mortality in PAH and that addition of resistin to survival models may improve model fit and predictive capacity.

The large sample size and complex clinical features within this cohort enabled important feature selection and extensive machine learning-based multivariable modeling and model comparisons. To the present inventor's knowledge, this study is among the very few to attempt ML-based risk stratification in patients with PAH.35 However, the study had several limitations. First, because this multicenter registry relies on separate reports from different centers for data collection, some covariates had missing data, notably 6MWD and NYHA FC. Indeed, when the present inventor repeated the multivariable survival analyses without these two covariates (Table 4), the predictive performance of resistin level for mortality was strengthened. Second, some of the parameters included in the REVEAL Registry scoring tool for PAH risk prediction were unavailable in this cohort;36, 37 but this is unlikely to have affected the present inventor's results, as the REVEAL Registry risk score retains its predictive ability if at least seven of the 12 available risk parameters are available and included in the calculations. Third, most patients were receiving PAH-specific therapy at the time of biomarker assessment, which may have affected circulating biomarker levels. However, the association between resistin and mortality remained significant after adjusting for the presence and class of PAH therapy in multivariable models. Finally, serum collection was not contemporaneous with assessments of other clinical variables such as RHC measurements. Thus performing the analyses in a subset of patients with biomarkers obtained within 6 months of other clinical measures of disease severity, may strengthen the significance of biomarker associations with survival.

In conclusion, the present inventor's study provides evidence to support the use of circulating biomarkers as objective and accessible tools for noninvasive PAH risk stratification. Additional clinical, genetic, and epidemiologic studies are warranted to strengthen the association between resistin and the prevalence, severity, and outcome of PAH.

### REFERENCES

1. Swift A J, Lu H, Uthoff J, et al. A machine learning cardiac magnetic resonance approach to extract disease features and automate pulmonary arterial hypertension diagnosis. European heart journal Cardiovascular Imaging 2021; 22 (2): 236-45.
2. Fan C, Johns B A, Su Q, Kolosova I A, Johns R A. Choosing the right antibody for resistin-like molecule (RELM/FIZZ) family members. Histochemistry and cell biology 2013; 139 (4): 605-13.
3 Steppan C M, Bailey S T, Bhat S, et al. The hormone resistin links obesity to diabetes. Nature 2001; 409 (6818): 307-12.
4. Schwartz D R, Lazar M A. Human resistin: found in translation from mouse to man. Trends in endocrinology and metabolism: TEM 2011; 22 (7): 259-65.
5 Ruscica M, Baragetti A, Catapano A L, Norata G D. Translating the biology of adipokines in atherosclerosis and cardiovascular diseases: Gaps and open questions. Nutrition, metabolism, and cardiovascular diseases: NMCD 2017; 27 (5): 379-95.
6. Cheng J M, Akkerhuis K M, Battes L C, et al. Biomarkers of heart failure with normal ejection fraction: a systematic review. European journal of heart failure 2013; 15 (12): 1350-62.
7 Brankovic M, Akkerhuis K M, Mouthaan H, et al. Cardiometabolic Biomarkers and Their Temporal Patterns Predict Poor Outcome in Chronic Heart Failure (Bio-SHIFT Study). The Journal of clinical endocrinology and metabolism 2018; 103 (11): 3954-64.
8. Teng X, Li D, Champion H C, Johns R A. FIZZ1/RELMalpha, a novel hypoxia-induced mitogenic factor in lung with vasoconstrictive and angiogenic properties. Circulation research 2003; 92 (10): 1065-7.
9. Angelini D J, Su Q, Yamaji-Kegan K, et al. Hypoxia-induced mitogenic factor (HIMF/FIZZ1/RELMalpha) induces the vascular and hemodynamic changes of pulmonary hypertension. American journal of physiology Lung cellular and molecular physiology 2009; 296 (4): L582-93.
10. Daley E, Emson C, Guignabert C, et al. Pulmonary arterial remodeling induced by a Th2 immune response. The Journal of experimental medicine 2008; 205 (2): 361-72.
11. Mishra A, Wang M, Schlotman J, et al. Resistin-like molecule-beta is an allergen-induced cytokine with inflammatory and remodeling activity in the murine lung. American journal of physiology Lung cellular and molecular physiology 2007; 293 (2): L305-13.
12. Zhu N, Pauciulo M W, Welch C L, et al. Novel risk genes and mechanisms implicated by exome sequencing of 2572 individuals with pulmonary arterial hypertension. Genome medicine 2019; 11 (1): 69.
13. Simpson C E, Damico R L, Hassoun P M, et al. Noninvasive Prognostic Biomarkers for Left-Sided Heart Failure as Predictors of Survival in Pulmonary Arterial Hypertension. Chest 2020; 157 (6): 1606-16.
14. Gok E C, Olgun M O. SMOTE-NC and gradient boosting imputation based random forest classifier for predicting severity level of covid-19 patients with blood samples. Neural computing & applications 2021:1-15.
15. Mathai S C, Bueso M, Hummers L K, et al. Disproportionate elevation of N-terminal pro-brain natriuretic peptide in scleroderma-related pulmonary hypertension. The European respiratory journal 2010; 35 (1): 95-104.
16. Benza R L, Miller D P, Gomberg-Maitland M, et al. Predicting survival in pulmonary arterial hypertension: insights from the Registry to Evaluate Early and Long-Term Pulmonary Arterial Hypertension Disease Management (REVEAL). Circulation 2010; 122 (2): 164-72.
17. Benza R L, Gomberg-Maitland M, Naeije R, Arneson C P, Lang I M. Prognostic factors associated with increased survival in patients with pulmonary arterial hypertension treated with subcutaneous treprostinil in randomized, placebo-controlled trials. The Journal of heart and lung transplantation: the official publication of the International Society for Heart Transplantation 2011; 30 (9): 982-9.
18. Fallah A A, Sarmast E, Jafari T. Effect of dietary anthocyanins on biomarkers of glycemic control and glucose metabolism: A systematic review and meta-analysis of randomized clinical trials. Food research international 2020; 137:109379.
19. Biscetti F, Nardella E, Cecchini A L, Flex A, Landolfi R. Biomarkers of vascular disease in diabetes: the adipose-immune system cross talk. Internal and emergency medicine 2020; 15 (3): 381-93.
20. Catalina M O, Redondo P C, Granados M P, et al. New Insights into Adipokines as Potential Biomarkers for Type-2 Diabetes Mellitus. Current medicinal chemistry 2019; 26 (22): 4119-44.
21. Sudan S K, Deshmukh S K, Poosarla T, et al. Resistin: An inflammatory cytokine with multi-faceted roles in cancer. Biochimica et biophysica acta Reviews on cancer 2020; 1874 (2): 188419.
22. Morshedzadeh N, Rahimlou M, Asadzadeh Aghdaei H, Shahrokh S, Reza Zali M, Mirmiran P. Association Between Adipokines Levels with Inflammatory Bowel Disease (IBD): Systematic Reviews. Digestive diseases and sciences 2017; 62 (12): 3280-6.
23. Gerstmayer B, Kusters D, Gebel S, et al. Identification of RELMgamma, a novel resistin-like molecule with a distinct expression pattern. Genomics 2003; 81 (6): 588-95.
24. Menzaghi C, Coco A, Salvemini L, et al. Heritability of serum resistin and its genetic correlation with insulin resistance-related features in nondiabetic Caucasians. The Journal of clinical endocrinology and metabolism 2006; 91 (7): 2792-5.
25. Cho Y M, Youn B S, Chung S S, et al. Common genetic polymorphisms in the promoter of resistin gene are major determinants of plasma resistin concentrations in humans. Diabetologia 2004; 47 (3): 559-65.
26. Osawa H, Onuma H, Ochi M, et al. Resistin SNP-420 determines its monocyte mRNA and serum levels inducing type 2 diabetes. Biochemical and biophysical research communications 2005; 335 (2): 596-602.
27. Osawa H, Tabara Y, Kawamoto R, et al. Plasma resistin, associated with single nucleotide polymorphism-420, is correlated with insulin resistance, lower HDL cholesterol, and high-sensitivity C-reactive protein in the Japanese general population. Diabetes care 2007; 30 (6): 1501-6.
28. Hussain S, Asghar M, Javed Q. Resistin gene promoter region polymorphism and the risk of hypertrophic cardiomyopathy in patients. Translational research: the journal of laboratory and clinical medicine 2010; 155 (3): 142-7.
29. Burki T K. The role of AI in diagnosing lung diseases. The Lancet Respiratory medicine 2019; 7 (12): 1015-6.
30. Angelini E, Dahan S, Shah A. Unravelling machine learning: insights in respiratory medicine. The European respiratory journal 2019; 54 (6).
31. Leha A, Hellenkamp K, Unsold B, et al. A machine learning approach for the prediction of pulmonary hypertension. PloS one 2019; 14 (10): e0224453.
32. Ong M S, Klann J G, Lin K J, et al. Claims-Based Algorithms for Identifying Patients With Pulmonary Hypertension: A Comparison of Decision Rules and Machine-Learning Approaches. Journal of the American Heart Association 2020; 9 (19): e016648.
33. Mazimba S, Mejia-Lopez E, Black G, et al. Diastolic pulmonary gradient predicts outcomes in group 1 pulmonary hypertension (analysis of the NIH primary pulmonary hypertension registry). Respiratory medicine 2016; 119:81-6.
34. Hoeper M M, Humbert M. The new haemodynamic definition of pulmonary hypertension: evidence prevails, finally! The European respiratory journal 2019; 53 (3).
35. Bauer Y, de Bernard S, Hickey P, et al. Identifying early pulmonary arterial hypertension biomarkers in systemic sclerosis: machine learning on proteomics from the DETECT cohort. The European respiratory journal 2021; 57 (6).
36. Benza R L, Farber H W, Frost A, et al. REVEAL risk score in patients with chronic thromboembolic pulmonary hypertension receiving riociguat. The Journal of heart and lung transplantation: the official publication of the International Society for Heart Transplantation 2018; 37 (7): 836-43.
37. Benza R L, Gomberg-Maitland M, Elliott C G, et al. Predicting Survival in Patients With Pulmonary Arterial Hypertension: The REVEAL Risk Score Calculator 2.0 and Comparison With ESC/ERS-Based Risk Assessment Strategies. Chest 2019; 156 (2): 323-37.

TABLE 1
Demographics and Clinical Characteristics of
Patients with Pulmonary Arterial Hypertension

| | Overall (n = 1,121) | | IPAH (n = 808) | | SSc-PAH (n = 313) | |
|---|---|---|---|---|---|---|
| Age, yr, median (IQR) | 58 | (22) | 53 | (23) | 65.5 | (14) |
| Female sex, n (%) | 919 | (81.91) | 645 | (79.83) | 274 | (87.54) |
| Race, n (%) | | | | | | |
| EA | 947 | (85.39) | 676 | (84.08) | 271 | (88.85) |
| AA | 111 | (10.00) | 83 | (10.32) | 28 | (9.18) |
| Other | 51 | (4.60) | 45 | (5.60) | 6 | (1.97) |
| Deaths during follow-up, n (%) | 191 | (18.26) | 96 | (12.66) | 95 | (32.99) |
| 6MWD, m, median (IQR) | 343 | (164.75) | 355 | (155.75) | 313.50 | (169.5) |
| NYHA FC, n (%) | | | | | | |
| I/II | 299 | (36.96) | 203 | (35.43) | 96 | (40.68) |
| III/IV | 510 | (63.04) | 370 | (64.57) | 140 | (59.32) |
| Hemodynamics, median (IQR) | | | | | | |
| RAP, mm Hg | 8 | (7) | 8 | (7) | 8 | (8) |
| mPAP, mm Hg | 49 | (19) | 52 | (19) | 43 | (17) |
| PAWP, mm Hg | 10 | (6) | 10 | (6) | 10 | (6) |
| PVR, WU | 8.95 | (7.03) | 10 | (7) | 7.31 | (5.62) |
| CO, L/min | 4 | (1) | 4 | (2) | 4 | (2) |
| Cardiac index, L/min/m² | 2.54 | (1.16) | 2.48 | (1.17) | 2.61 | (2.07) |
| REVEAL Registry 2.0 risk score, | 7 | (13) | 6 | (13) | 8 | (11) |
| median (range) | | | | | | |
| Biomarker values, median (IQR) | | | | | | |
| NTproBNP, pg/mL | 719 | (2091) | 490 | (1428) | 1796 | (4434) |
| Resistin, ng/mL | 6.63 | (4.34) | 6.2 | (3.67) | 8.28 | (5.59) |
| Therapies, n (%) | | | | | | |
| PDE5 inhibitor | 794 | (71.86) | 568 | (71.36) | 226 | (73.14) |
| ERA | 626 | (56.65) | 455 | (57.16) | 171 | (55.34) |
| IV/SC prostacyclin | 297 | (26.88) | 249 | (31.28) | 48 | (15.53) |
| CCB | 125 | (11.31) | 98 | (12.31) | 27 | (8.74) |

6MWD denotes 6-minute walk distance; AA, African American; CCB, calcium channel blocker; CO, cardiac output; EA, European American; ERA, endothelin receptor antagonist; IPAH, idiopathic pulmonary arterial hypertension; IQR, interquartile range; mPAP, mean pulmonary arterial pressure; NTproBNP, N-terminal pro-brain natriuretic peptide; NYHA FC, New York Heart Association functional class; PAWP, pulmonary artery wedge pressure; PDE5, phosphodiesterase-5; PVR, pulmonary vascular resistance; RAP, right atrial pressure; REVEAL Registry, Registry to Evaluate Early and Long-Term PAH Disease Management; SSc-PAH, scleroderma-associated pulmonary arterial hypertension; WU, Wood units.

TABLE 2
Correlation of log(resistin) levels with continues clinical variables adjusting for age, sex and BMI.

| Clinical variables | All PAH | IPAH | SSc-PAH |
|---|---|---|---|
| 6MWD, m | −.077 (−96.9 to 1.60; .058) | −.062 (−104.973 to 22.096; .201) | −.073 (−122.842 to 47.325; .382) |
| Hemodynamics | | | |
| Heart rate, beats/min | .065 (1.014 to 23.225; .032) | .024 (−8.971 to 18.424; .498) | .155 (.410 to 19.204; .041) |
| RAP, mm Hg | .067 (.219 to 3.345; .026) | .076 (.189 to 4.133; .032) | .062 (−1.342 to 4.366; .298) |
| mPAP mm Hg | −.005 (−4.135 to 3.419; .853) | .014 (−3.873 to 5.874; .687) | .067 (−2.491 to 9.205; .259) |
| PAWP, mm Hg | −.002 (−1.18 to 1.096; .943) | .028 (−.828 to 1.989; .419) | −.040 (−2.836 to 1.408; .508) |
| PVR, WU | −.006 (−1.853 to 1.497; .835) | −.006 (−2.361 to 2.006; .873) | .077 (−.854 to 4.096; .198) |
| CO, L/min | .015 (−.510 to .852; .622) | .019 (−.604 to 1.047; .598) | −.015 (−.928 to .708; .792) |
| Cardiac index, L/min/m² | −.075 (−.696 to −.079; .014) | −.091 (−.891 to −.119; .010) | −.065 (−.689 to .199; .278) |
| REVEAL Registry 2.0 risk score | .101 (.391 to 1.455; .001) | .031 (−.419 to 1.110; .375) | .105 (−.089 to 2.257; .070) |

All data are presented as follows: regression coefficient (95% CI; P value). See Table 1 legend for expansion of abbreviations.

TABLE 3
Demographics and Clinical Characteristics of PAH patients as a Function of Serum Resistin

| | All PAH | | | IPAH | | |
|---|---|---|---|---|---|---|
| | Resistin | Resistin | | Resistin | Resistin | |
| | level_{low}^{a} | level_{high}^{b} | P | level_{low} | level_{high}^{b} | P |
| Variables | (n = 219) | (n = 903) | Value^{c} | (n = 190) | (n = 618) | Value^{c} |
| Age, yr, median (IQR) | 53 (23.75) | 59 (22) | 0.001 | 51.5 (23.25) | 54 (24) | 0.021 |
| Female sex, n (%) | 172 (75.84) | 747 (82.72) | 0.149 | 146 (76.84) | 499 (80.74) | 0.241 |
| Race, n (%) | | | 0.008 | | | 0.059 |
| EA | 171 (80.28) | 776 (86.61) | | 152 (81.28) | 524 (84.93) | |
| AA | 24 (11.27) | 87 (9.71) | | 18 (9.63) | 65 (10.53) | |
| Other | 18 (8.45) | 33 (3.68) | | 17 (9.09) | 28 (4.54) | |
| Deaths during follow- | 24 (11.82) | 167 (19.81) | 0.008 | 18 (10.23) | 78 (13.43) | 0.264 |
| up, n (%) | | | | | | |
| 6MWD, m, median | 378.5 (156) | 340 (167) | 0.008 | 381.5 (149.5) | 34 (166.75) | 0.058 |
| (IQR) | | | | | | |
| NYHA FC, n (%) | | | 0.067 | | | 0.11 |
| I/II | 66 (43.42) | 233 (35.46) | | 54 (41.22) | 149 (33.71) | |
| III/IV | 86 (56.58) | 424 (64.54) | | 77 (58.78) | 293 (66.29) | |
| Hemodynamics, | | | | | | |
| median (IQR) | | | | | | |
| RAP, mm Hg | 8 (6) | 8 (7) | 0.225 | 8 (6) | 8 (7) | 0.142 |
| mPAP mm Hg | 49 (19) | 49 (19.75) | 0.925 | 50 (18.5) | 52 (19) | 0.112 |
| PAWP, mm Hg | 9 (5) | 10 (6) | 0.101 | 9 (5) | 10 (6) | 0.039 |
| PVR, WU | 9.46 (6.83) | 8.87 (7.2) | 0.405 | 10 (7) | 10 (7.75) | 0.764 |
| CO, L/min | 4 (2) | 4 (2) | 0.986 | 4 (2) | 4 (1) | 0.176 |
| Cardiac index, | 2.69 (1.13) | 2.51 (1.13) | 0.016 | 3 (1) | 2 (1) | 0.004 |
| L/min/m² | | | | | | |
| REVEAL Registry 2.0 | 6 (1-12) | 7 (1-12) | 0.089 | 6 (1-14) | 6 (1-13) | 0.92 |
| risk score, median | | | | | | |
| (range) | | | | | | |
| Biomarker value, | | | | | | |
| median (IQR) | | | | | | |
| NTproBNP, pg/mL | 590 (1546) | 745 (2243.75) | 0.155 | 472 (1396) | 501 (1434) | 0.03 |
| Therapies, n (%) | | | | | | |
| PDE5 inhibitor | 153 (71.16) | 641 (72.02) | 0.802 | 136 (73.12) | 432 (70.94) | 0.564 |
| ERA | 144 (66.98) | 482 (54.16) | 0.001 | 126 (67.74) | 328 (53.86) | 0.001 |
| IV/SC prostacyclin | 54 (25.12) | 243 (27.30) | 0.516 | 49 (26.34) | 199 (32.68) | 0.103 |
| CCB | 27 (12.56) | 98 (11.01) | 0.521 | 23 (12.37) | 75 (12.32) | 0.985 |
| Combination | | | 0.024 | | | 0.021 |
| therapy, n (%) | | | | | | |
| Monotherapy | 42 (19.53) | 255 (28.65) | | 33 (17.74) | 165 (27.09) | |
| 2 drugs | 89 (41.40) | 317 (35.62) | | 79 (42.47) | 208 (34.15) | |
| ≥3 drugs | 84 (39.07) | 318 (35.73) | | 74 (39.78) | 236 (38.75) | |

See Table 1 legend for expansion of abbreviations.
^{a}Resistin level_{low} = serum resistin level below 4.54 ng/mL cutoff
^{b}Resistin level_{high} = serum resistin level above 4.54 ng/mL cutoff
^{c}Chi-square test was used for categorical parameters, and non-parametric testing (Mann-Whitney) was performed for quantitative parameters. Bold font indicates statistical significance.

TABLE 4
Multivariable Cox Proportional Hazard Models for 60-Day Mortality

| | | | | | | | Reduced model (6MWD and | | |
|---|---|---|---|---|---|---|---|---|---|
| | Full model | | | Reduced model (6MWD excluded) | | | NYHA FC excluded) | | |
| Predictor: (log) | Hazard | | | Hazard | | | Hazard | | |
| Resistin | ratio | 95% CI | P value | ratio | 95% CI | P value | ratio | 95% CI | P value |
| All PAH (n = 1,121) | 2.33 | 0.62-8.68 | 0.21 | 2.87 | 1.25-6.58 | 0.013 | 3.14 | 1.52-6.49 | 0.002 |
| IPAH (n = 808) | 9.97 | 1.29-76.96 | 0.027 | 6.36 | 2.08-19.46 | 0.001 | 4.11 | 1.53-11.07 | 0.005 |
| SSc-PAH (n = 313) | 1.29 | 0.18-10.56 | 0.81 | 1.10 | 0.27-4.52 | 0.89 | 2.04 | 0.69-5.97 | 0.19 |

All data are presented as follows: hazard ratio, 95% CI; P value. See Table 1 legend for expansion of abbreviations.

Each model was adjusted for age, sex, BMI, PAH-specific therapy, NYHA FC, 6MWD, RAP, mPAP, cardiac index, and PVR.

TABLE 5
Association between serum resistin levels and genotypes of two RETN variants in PAH.

| | All PAH | | | IPAH | | | SSc-PAH | | |
|---|---|---|---|---|---|---|---|---|---|
| | | log | P_{adj} (coefficient, | | log | P_{adj} (coefficient, | | log | P_{adj} (coefficient, |
| | N | (resistin)^{a} | 95% CI)^{b} | N | (resistin)^{a} | 95% CI)^{b} | N | (resistin)^{a} | 95% CI)^{b} |
| rs3219175 (G/A) | 1122 | 0.84 ± 0.22 | 0.0001 (5.49, | 808 | 0.81 ± 0.21 | 0.0001 (5.66, | 313 | 0.92 ± 0.22 | 0.0007 (5.78, |
| GG | 1015 | 0.83 ± 0.21 | 55.53-1050.33) | 735 | 0.79 ± 0.20 | 52.55-1573.88) | 280 | 0.91 ± 0.22 | 11.40-9243.59) |
| GA + AA | 56 | 1.01 ± 0.25 | | 41 | 1.00 ± 0.24 | | 15 | 1.03 ± 0.29 | |
| rs3745367 (G/A) | 1122 | 0.84 ± 0.22 | 0.0001 (2.36, | 808 | 0.81 ± 0.21 | 0.0003 (2.48, | 313 | 0.92 ± 0.22 | n.s. |
| GG + GA | 944 | 0.83 ± 0.21 | 4.27-26.21) | 688 | 0.79 ± 0.20 | 4.15-34.46) | 256 | 0.92 ± 0.22 | |
| AA | 128 | 0.91 ± 0.24 | | 89 | 0.90 ± 0.24 | | 39 | 0.94 ± 0.24 | |

CI denotes confidence interval; PAH, pulmonary arterial hypertension; IPAH, idiopathic PAH; SSc-PAH, scleroderma-associated PAH.
^{a}log (resistin) values are shown as mean ± SD.
^{b}P values were adjusted for age, sex, ethnicity, and body mass index.
n.s., not significant

That which is claimed:

1. A method for treating an NLRP3 inflammasome mediated disease, disorder or condition in a patient comprises the step of administering to the patient an isolated, recombinant antibody or antigen-binding fragment thereof that binds human Resistin.

2. The method of claim 1, wherein the anti-Resistin antibody or antigen-binding fragment there of comprises a single chain variable fragment (scFv), a dimeric scFv, a Fab, a Fab′, a F(ab′) 2 fragment or a full length antibody.

3. The method of claim 1, wherein the anti-Resistin antibody or antigen-binding fragment thereof comprising a heavy chain variable region and a light chain variable region, wherein:
(a) the heavy chain variable region comprises SEQ ID NO:3 or a conservative substitution at up to 5 amino acid positions of SEQ ID NO:3, and the light chain variable region comprises SEQ ID NO:7 or a conservative substitution at up to 5 amino acid positions of SEQ ID NO:7;
(b) the heavy chain variable region comprises SEQ ID NO: 13 or a conservative substitution at up to 5 amino acid positions of SEQ ID NO:13, and the light chain variable region comprises SEQ ID NO:17 or a conservative substitution at up to 5 amino acid positions of SEQ ID NO: 17;
(c) the heavy chain variable region comprises SEQ ID NO:23 or a conservative substitution at up to 5 amino acid positions of SEQ ID NO:23, and the light chain variable region comprises SEQ ID NO:27 or a conservative substitution at up to 5 amino acid positions of SEQ ID NO:27;
(d) the heavy chain variable region comprises SEQ ID NO:33 or a conservative substitution at up to 5 amino acid positions of SEQ ID NO:33 and the light chain variable region comprises SEQ ID NO:37 or a conservative substitution at up to 5 amino acid positions of SEQ ID NO:37;
(e) the heavy chain variable region comprises SEQ ID NO:43 or a conservative substitution at up to 5 amino acid positions of SEQ ID NO:43, and the light chain variable region comprises SEQ ID NO:47 or a conservative substitution at up to 5 amino acid positions of SEQ ID NO:47;
(f) the heavy chain variable region comprises SEQ ID NO:53 or a conservative substitution at up to 5 amino acid positions of SEQ ID NO:53, and the light chain variable region comprises SEQ ID NO:57 or a conservative substitution at up to 5 amino acid positions of SEQ ID NO:57;
(g) the heavy chain variable region comprises SEQ ID NO:63 or a conservative substitution at up to 5 amino acid positions of SEQ ID NO:63 and the light chain variable region comprises SEQ ID NO:67 or a conservative substitution at up to 5 amino acid positions of SEQ ID NO:67;
(h) the heavy chain variable region comprises SEQ ID NO:73 or a conservative substitution at up to 5 amino acid positions of SEQ ID NO:73, and the light chain variable region comprises SEQ ID NO:77 or a conservative substitution at up to 5 amino acid positions of SEQ ID NO:77;
(i) the heavy chain variable region comprises SEQ ID NO:83 or a conservative substitution at up to 5 amino acid positions of SEQ ID NO:83, and the light chain variable region comprises SEQ ID NO:87 or a conservative substitution at up to 5 amino acid positions of SEQ ID NO:87;
(j) the heavy chain variable region comprises SEQ ID NO:93 or a conservative substitution at up to 5 amino acid positions of SEQ ID NO:93 and the light chain variable region comprises SEQ ID NO:97 or a conservative substitution at up to 5 amino acid positions of SEQ ID NO:97;
(k) the heavy chain variable region comprises SEQ ID NO: 103 or a conservative substitution at up to 5 amino acid positions of SEQ ID NO:103, and the light chain variable region comprises SEQ ID NO:107 or a conservative substitution at up to 5 amino acid positions of SEQ ID NO:107;
(1) the heavy chain variable region comprises SEQ ID NO:113 or a conservative substitution at up to 5 amino acid positions of SEQ ID NO:113 and the light chain variable region comprises SEQ ID NO:117 or a conservative substitution at up to 5 amino acid positions of SEQ ID NO:117;
(m) the heavy chain variable region comprises SEQ ID NO: 123 or a conservative substitution at up to 5 amino acid positions of SEQ ID NO:123, and the light chain variable region comprises SEQ ID NO:127 or a conservative substitution at up to 5 amino acid positions of SEQ ID NO: 127;
(n) the heavy chain variable region comprises SEQ ID NO: 133 or a conservative substitution at up to 5 amino acid positions of SEQ ID NO:133, and the light chain variable region comprises SEQ ID NO:137 or a conservative substitution at up to 5 amino acid positions of SEQ ID NO: 137;
(o) the heavy chain variable region comprises SEQ ID NO:143 or a conservative substitution at up to 5 amino acid positions of SEQ ID NO:143, and the light chain variable region comprises SEQ ID NO:147 or a conservative substitution at up to 5 amino acid positions of SEQ ID NO:147;
(p) the heavy chain variable region comprises SEQ ID NO:153 or a conservative substitution at up to 5 amino acid positions of SEQ ID NO:153, and the light chain variable region comprises SEQ ID NO:157 or a conservative substitution at up to 5 amino acid positions of SEQ ID NO: 157; or
(q) the heavy chain variable region comprises SEQ ID NO:163 or a conservative substitution at up to 5 amino acid positions of SEQ ID NO:163 and the light chain variable region comprises SEQ ID NO:167 or a conservative substitution at up to 5 amino acid positions of SEQ ID NO: 167.

4. The method of claim 3, wherein the anti-Resistin antibody or antigen-binding fragment thereof further comprises a heavy chain constant region comprising SEQ ID NO: 172 and a light chain constant region comprising SEQ ID NO: 174.

5. The method of claim 1, wherein the anti-Resistin antibody or antigen-binding fragment thereof comprises:
(a) a heavy chain variable region comprising complementarity determining regions (CDRs) 1, 2 and 3 comprising SEQ ID NOS: 4-6, respectively, or a conservative substitution at up to 2 amino acids of one or more of SEQ ID NOS: 4-6, and a light chain variable region comprising CDRs 1, 2, and 3 comprising SEQ ID NOS: 8-10, respectively, or a conservative substitution at up to 2 amino acids of one or more of SEQ ID NOS: 8-10;
(b) a heavy chain variable region comprising CDRs 1, 2 and 3 comprising SEQ ID NOS: 14-16, respectively, or a conservative substitution at up to 2 amino acids of one or more of SEQ ID NOS: 14-16, and a light chain variable region comprising CDRs 1, 2, and 3 comprising SEQ ID NOS: 18-20, respectively, or a conservative substitution at up to 2 amino acids of one or more of SEQ ID NOS: 18-20;
(c) a heavy chain variable region comprising CDRs 1, 2 and 3 comprising SEQ ID NOS: 24-26, respectively, or a conservative substitution at up to 2 amino acids of one or more of SEQ ID NOS: 24-26, and a light chain variable region comprising CDRs 1, 2, and 3 comprising SEQ ID NOS: 28-30, respectively, or a conservative substitution at up to 2 amino acids of one or more of SEQ ID NOS: 28-30;
(d) a heavy chain variable region comprising CDRs 1, 2 and 3 comprising SEQ ID NOS: 34-36, respectively, or a conservative substitution at up to 2 amino acids of one or more of SEQ ID NOS: 34-36, and a light chain variable region comprising CDRs 1, 2, and 3 comprising SEQ ID NOS: 38-40, respectively, or a conservative substitution at up to 2 amino acids of one or more of SEQ ID NOS: 38-40;
(e) a heavy chain variable region comprising CDRs 1, 2 and 3 comprising SEQ ID NOS: 44-46, respectively, or a conservative substitution at up to 2 amino acids of one or more of SEQ ID NOS: 44-46, and a light chain variable region comprising CDRs 1, 2, and 3 comprising SEQ ID NOS: 48-50, respectively, or a conservative substitution at up to 2 amino acids of one or more of SEQ ID NOS: 48-50;
(f) a heavy chain variable region comprising CDRs 1, 2 and 3 comprising SEQ ID NOS: 54-56, respectively, or a conservative substitution at up to 2 amino acids of one or more of SEQ ID NOS: 54-56, and a light chain variable region comprising CDRs 1, 2, and 3 comprising SEQ ID NOS: 58-60, respectively, or a conservative substitution at up to 2 amino acids of one or more of SEQ ID NOS: 58-60;
(g) a heavy chain variable region comprising CDRs 1, 2 and 3 comprising SEQ ID NOS: 64-66, respectively, or a conservative substitution at up to 2 amino acids of one or more of SEQ ID NOS: 64-66, and a light chain variable region comprising CDRs 1, 2, and 3 comprising SEQ ID NOS: 68-70, respectively, or a conservative substitution at up to 2 amino acids of one or more of SEQ ID NOS: 68-70;
(h) a heavy chain variable region comprising CDRs 1, 2 and 3 comprising SEQ ID NOS: 74-76, respectively, or a conservative substitution at up to 2 amino acids of one or more of SEQ ID NOS: 74-76, and a light chain variable region comprising CDRs 1, 2, and 3 comprising SEQ ID NOS: 78-80, respectively, or a conservative substitution at up to 2 amino acids of one or more of SEQ ID NOS: 78-80;
(i) a heavy chain variable region comprising CDRs 1, 2 and 3 comprising SEQ ID NOS: 84-86, respectively, or a conservative substitution at up to 2 amino acids of one or more of SEQ ID NOS: 84-86, and a light chain variable region comprising CDRs 1, 2, and 3 comprising SEQ ID NOS: 88-90, respectively, or a conservative substitution at up to 2 amino acids of one or more of SEQ ID NOS: 88-90;
(i) a heavy chain variable region comprising CDRs 1, 2 and 3 comprising SEQ ID NOS: 94-96, respectively, or a conservative substitution at up to 2 amino acids of one or more of SEQ ID NOS: 94-96, and a light chain variable region comprising CDRs 1, 2, and 3 comprising SEQ ID NOS: 98-100, respectively, or a conservative substitution at up to 2 amino acids of one or more of SEQ ID NOS: 98-100;
(k) a heavy chain variable region comprising CDRs 1, 2 and 3 comprising SEQ ID NOS: 104-106, respectively, or a conservative substitution at up to 2 amino acids of one or more of SEQ ID NOS: 104-106, and a light chain variable region comprising CDRs 1, 2, and 3 comprising SEQ ID NOS: 108-110, respectively, or a conservative substitution at up to 2 amino acids of one or more of SEQ ID NOS: 108-110;
(1) a heavy chain variable region comprising CDRs 1, 2 and 3 comprising SEQ ID NOS: 114-116, respectively, or a conservative substitution at up to 2 amino acids of one or more of SEQ ID NOS: 114-116, and a light chain variable region comprising CDRs 1, 2, and 3 comprising SEQ ID NOS: 118-120, respectively, or a conservative substitution at up to 2 amino acids of one or more of SEQ ID NOS: 118-120;
(m) a heavy chain variable region comprising CDRs 1, 2 and 3 comprising SEQ ID NOS: 124-126, respectively, or a conservative substitution at up to 2 amino acids of one or more of SEQ ID NOS: 124-126, and a light chain variable region comprising CDRs 1, 2, and 3 comprising SEQ ID NOS: 128-130, respectively, or a conservative substitution at up to 2 amino acids of one or more of SEQ ID NOS: 128-130;
(n) a heavy chain variable region comprising CDRs 1, 2 and 3 comprising SEQ ID NOS: 134-136, respectively, or a conservative substitution at up to 2 amino acids of one or more of SEQ ID NOS: 134-136, and a light chain variable region comprising CDRs 1, 2, and 3 comprising SEQ ID NOS: 138-140, respectively, or a conservative substitution at up to 2 amino acids of one or more of SEQ ID NOS: 138-140;
(o) a heavy chain variable region comprising CDRs 1, 2 and 3 comprising SEQ ID NOS: 144-146, respectively, or a conservative substitution at up to 2 amino acids of one or more of SEQ ID NOS: 144-146, and a light chain variable region comprising CDRs 1, 2, and 3 comprising SEQ ID NOS: 148-150, respectively, or a conservative substitution at up to 2 amino acids of one or more of SEQ ID NOS: 148-150;
(p) a heavy chain variable region comprising CDRs 1, 2 and 3 comprising SEQ ID NOS: 154-156, respectively, or a conservative substitution at up to 2 amino acids of one or more of SEQ ID NOS: 154-156, and a light chain variable region comprising CDRs 1, 2, and 3 comprising SEQ ID NOS: 158-160, respectively, or a conservative substitution at up to 2 amino acids of one or more of SEQ ID NOS: 158-160; or
(q) a heavy chain variable region comprising CDRs 1, 2 and 3 comprising SEQ ID NOS: 164-166, respectively, or a conservative substitution at up to 2 amino acids of one or more of SEQ ID NOS: 164-166, and a light chain variable region comprising CDRs 1, 2, and 3 comprising SEQ ID NOS: 168-170, or a conservative substitution at up to 2 amino acids of one or more of SEQ ID NOS: 168-170.

6. The method of claim 5, wherein the anti-Resistin antibody or antigen-binding fragment thereof further comprises a heavy chain constant region comprising SEQ ID NO: 172 and a light chain constant region comprising SEQ ID NO: 174.

7. The method of claim 1, wherein the anti-Resistin antibody or antigen-binding fragment thereof comprises an scfv or antigen-binding fragment thereof that binds Resistin, wherein the scFv comprises SEQ ID NO:2 or a conservative substitution at up to 5 amino acids of SEQ ID NO:2; SEQ ID NO: 12 or a conservative substitution at up to 5 amino acids of SEQ ID NO: 12; SEQ ID NO:22 or a conservative substitution at up to 5 amino acids of SEQ ID NO:22; SEQ ID NO:32 or a conservative substitution at up to 5 amino acids of SEQ ID NO: 32; SEQ ID NO:42 or a conservative substitution at up to 5 amino acids of SEQ ID NO: 42; SEQ ID NO:52 or a conservative substitution at up to 5 amino acids of SEQ ID NO: 52; SEQ ID NO:62 or a conservative substitution at up to 5 amino acids of SEQ ID NO: 62; SEQ ID NO:72 or a conservative substitution at up to 5 amino acids of SEQ ID NO: 72; SEQ ID NO:82 or a conservative substitution at up to 5 amino acids of SEQ ID NO: 82; SEQ ID NO:92 or a conservative substitution at up to 5 amino acids of SEQ ID NO: 92; SEQ ID NO:102 or a conservative substitution at up to 5 amino acids of SEQ ID NO: 102; SEQ ID NO:112 or a conservative substitution at up to 5 amino acids of SEQ ID NO: 112; SEQ ID NO:122 or a conservative substitution at up to 5 amino acids of SEQ ID NO: 122; SEQ ID NO:132 or a conservative substitution at up to 5 amino acids of SEQ ID NO: 132; SEQ ID NO:142 or a conservative substitution at up to 5 amino acids of SEQ ID NO: 142; SEQ ID NO:152 or a conservative substitution at up to 5 amino acids of SEQ ID NO: 152; or SEQ ID NO:162 or a conservative substitution at up to 5 amino acids of SEQ ID NO: 162.

8. The method of any of claims 1-7, wherein the NLRP3 inflammasome mediated disease, disorder or condition comprises autoimmune disease; age-related macular degeneration (AMD); autoinflammatory diseases; inflammatory responses; inflammatory skin diseases; sepsis; psoriasis; dermatitis; systemic scleroderma; sclerosis; inflammatory bowel disease; Crohn's disease; ulcerative colitis; respiratory distress syndrome; adult respiratory distress syndrome; meningitis; encephalitis; uveitis; colitis; glomerulonephritis; eczema; asthma; atherosclerosis; leukocyte adhesion deficiency; rheumatoid arthritis; systemic lupus erythematosus (SLE); lupus nephritis (LN); diabetes mellitus; multiple sclerosis; Reynaud's syndrome; autoimmune thyroiditis; allergic encephalomyelitis; Sjorgen's syndrome; juvenile onset diabetes; pernicious anemia; diseases involving leukocyte diapedesis; central nervous system (CNS) inflammatory disorder; multiple organ injury syndrome; hemolytic anemia; cryoglobinemia; Coombs positive anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenia purpura (ITP) or autoimmune thrombocytopenia; cryopyrin-associated periodic syndromes (CAPS); Alzheimer disease; myocardial infarction; gout; non-alcoholic fatty liver disease; nonalcoholic steatohepatitis; experimental autoimmune encephalitis; oxalate-induced nephropathy; hyperinflammation following influenza infection; stroke; silicosis; myelodysplastic syndrome; contact hypersensitivity; traumatic brain injury; familial cold autoinflammatory syndrome (FCAS); Muckle-Wells syndrome (MWS); neonatal onset multisystem inflammatory disorder (NOMID); crystalline arthropathies; silicosis; asbestosis; gout; pseudogout; diabetes mellitus; familial Mediterranean fever (FMF); pyogenic arthritis with pyodema gangrenosum and acne (PAPA) syndrome; hyperimmunoglobulinemia D with periodic fever syndrome (HIDS); Mevalonate kinase deficiency (MKD); Schnitzler's syndrome (SS); Aspergillus fumigatus keratitis; stargardt disease type 1; Alzheimer's disease; atherosclerosis; atrial fibrillation; osteoarthritis; cancer; pulmonary hypertension; right or left heart failure; and lung inflammation due to bacterial, viral or parasitic infections.

9. A ribonucleic acid (RNA) interfering (RNAi) composition comprising 18-25 nucleotide that is complementary to SEQ ID NO:244, wherein the RNAi composition is capable of inhibiting the expression of human Resistin.

10. The RNAi composition of claim 18, wherein he RNAi composition is a small interfering RNA (siRNA), a short hairpin RNA (shRNA), double stranded RNA (dsRNA), and RNA construct or an anti-sense oligonucleotide.

11. A shRNA for knocking down Resistin expression comprising SEQ ID NO:246 or SEQ ID NO:247.

12. A method for treating an NLRP3 inflammasome mediated disease, disorder or condition in a patient comprises the step of administering to the patient a pharmaceutical composition comprising the RNAi composition of claim 10 or the shRNA of claim 11.

13. A method for predicting disease severity in a patient having an NLRP3 inflammasome mediated disease, disorder or condition comprising the steps of:
(a) measuring the level of Resistin in a sample obtained from the patient;
(b) comparing the level measured in step (a) to a reference; and
(c) predicting disease severity in the patient.

14. A method for risk stratification of progressing to severe disease of a patient having an NLRP3 inflammasome mediated disease, disorder or condition comprising the steps of:
(a) measuring the level of Resistin in a sample obtained from the patient;
(b) comparing the level measured in step (a) to a reference; and
(c) stratifying the risk of the patient.

15. A method for monitoring disease progression in a patient having an NLRP3 inflammasome mediated disease, disorder or condition comprising the steps of:
(a) measuring the level of Resistin in a first sample obtained from the patient;
(b) measuring the level of Resistin in a second sample from the patient that has been obtained after the first sample;
(c) comparing the level measured in step (a) to the level measured in step (b); and
(d) monitoring disease progression in the patient based on the results of step (c).

16. The method of any one of claims 12-14, further comprising the step of screening DNA obtained from the patient for the presence of a Resistin polymorphism associated with the NLRP3 inflammasome mediated disease, disorder or condition.

17. The method of claim 15, wherein a Resistin polymorphism comprises rs10402265 (C>G).

18. The method of claim 15, wherein a Resistin polymorphism comprises rs12459044 (C>G).
